(19) United States
(12) Patent Application Publication
(10) Pub. No.: US 2024/0246956 A1
(43) Pub. Date: Jul. 25, 2024

(54) CLASS IIA HISTONE DEACETYLASE (HDAC) DEGRADER LIGANDS AND METHODS OF USE THEREOF

(71) Applicant: DANA-FARBER CANCER INSTITUTE, INC., Boston, MA (US)

(72) Inventors: Eric S. Fischer, Chestnut Hill, MA (US); Yuan Xiong, Waltham, MA (US); Katherine Donovan, Brookline, MA (US)

(73) Assignee: DANA-FARBER CANCER INSTITUTE, INC., Boston, MA (US)

(21) Appl. No.: 18/557,225

(22) PCT Filed: May 2, 2022
(86) PCT No.: PCT/US2022/027280

§ 371 Date: Oct. 25, 2023

Related U.S. Application Data
(60) Provisional application No. 63/316,167, filed on Mar. 3, 2022.
(60) Provisional application No. 63/183,358, filed on May 3, 2021.

(51) Int. Cl. C07D 417/14; A61K 31/427; A61K 31/454; A61K 31/4545; C07D 413/14

(52) U.S. Cl. CPC C07D 417/14; A61K 31/427; A61K 31/454; A61K 31/4545; C07D 413/14

(57) ABSTRACT

The present invention relates to bifunctional compounds, compositions, and methods for treat diseases or conditions mediated by aberrant activity of at least one class IIa histone deacetylase (HDAC4/5/7/9).

### RELATED APPLICATIONS

This application claims the benefit of priority under 35 U.S.C. § 119(e) to U.S. Provisional Application No. 63/183,358, filed on May 3, 2021 and to U.S. Provisional Application No. 63/316,167, filed on Mar. 3, 2022, each of which is incorporated herein by reference in its entirety.

### BACKGROUND

The modification of histones by acetylation/deacetylation plays a key role in the regulation of gene expression by changing the structure of chromatin and by modulating the accessibility of transcription factors to their target DNA sequences (Eckschlager, el al., Int. J. Mol. Sci. 18:1414 (2017)). The acetylation state of histones and other proteins is maintained by histone acetyltransferases (HAT) and histone deacetylases (HDAC). HATs add acetyl groups to lysine residues, while HDACs remove the acetyl groups. Generally, the acetylation of histone promotes a more relaxed chromatin structure which allows for transcriptional activation (Xu, et al., Oncogene 26:5541-5552 (2007)). In addition to regulating histone modification, HDACs also regulate the post-translational acetylation of many non-histone proteins, including transcription factors, chaperones, and signaling molecules, resulting in changes in protein stability, protein-protein interactions, and protein-DNA interactions (Glozak, et al., Gene 363:15-23 (2005)). The balance between histone acetylation and deacetylation is usually well regulated, but the balance is often upset in diseases such as cancer and neurodegenerative diseases.

HDACs, as chromatin modifying enzymes, are frequently recruited by co-repressors as a key component of large histone modifying complexes (Bantscheff, et al., Nat. Biotechnol. 29:255-265 (2011); Bradner et al., Nat. Chem. Biol. 6:238-243 (2010)). Some HDACs are also thought to exert non-enzymatic functions such as having a role in scaffolding these large complexes (Fischle, et al., Biol. Chem. 276:35826-35835 (2001); Fischle, et al., Mol. Cell 9(1):45-57 (2002); Magupalli, et al., Science 369(6510):eaas8995 (2020); McKinsey, el al., Proc. Natl. Acad. Sci. USA 97(26):14400-14405 (2000); Nguyen, et al., Nature 584:286-290 (2020); Verdin, et al., Trends Genet 19(5):286-93 (2003)).

The human HDAC family consists of 18 enzymes, 11 of which contain a divalent zinc cation in the catalytic site and 7 of which are Sirtuins with NAD+ dependent activity (Ruijter, el al., Biochem. J. 370:737-749 (2003); Seto and Yoshida. Cold Spring Harb. Perspect. Biol. 6(4):a018713. 2014). HDACs can be further classified into 5 classes: class I (HDAC1, 2, 3, and 8), class IIa (HDAC4, 5, 7, and 9), class lib (HDAC6 and 10), class III HDACs which consist of the Sirtuins, and class IV (HDAC 11).

Currently available inhibitors for the zinc dependent HDACs are used in the clinic to treat a variety of indications, including lymphoma. However, these drugs have limited selectivity, which has been suggested as a reason for off-target toxicities and adverse side effects (Suraweera, el al., Front. Oncol. 8:92 (2018)). Accordingly, there is a need for compounds that inhibit specific HDAC isoforms (e.g., class IIa HDACs) while minimizing off-target toxicity caused by binding to other unintended HDAC isoforms, for use in treating diseases such as cancer and neurodegenerative diseases.

### SUMMARY

A first aspect of the present disclosure is directed to a compound comprising a moiety that binds at least one class IIa histone deacetylase (HDAC) and a degron covalently attached to each other by a linker that comprises an alkylene chain or a polyethylene glycol (PEG) chain, wherein the compound has a structure represented by formula (I):

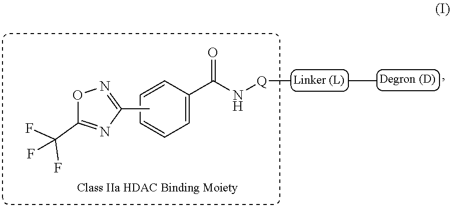

wherein:
Q represents

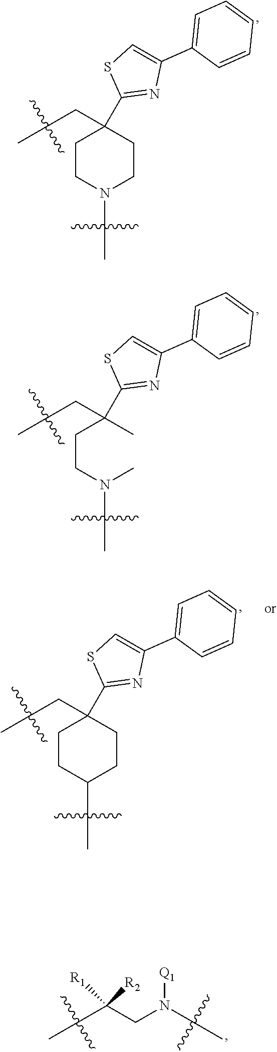

wherein R₁ and R₂ are independently H or C₁-C₄ alkyl and Q₁ is optionally C₁-C₄ alkyl;
   and the degron represents a ligand that binds cereblon (CRBN), von Hippel Landau tumor suppressor (VHL), or inhibitor of apoptosis protein (IAP), or a pharmaceutically acceptable salt or stereoisomer thereof.

Another aspect of the present disclosure is directed to a pharmaceutical composition containing a therapeutically effective amount of a compound of formula (I) or a pharmaceutically acceptable salt or stereoisomer thereof, and a pharmaceutically acceptable carrier

In another aspect of the present disclosure, methods of making the compounds are provided.

A further aspect of the present disclosure is directed to a method of treating a disease or disorder characterized or mediated by aberrant activity of at least one class IIa HDAC, that includes administering a therapeutically effective amount of a compound of formula (I) or a pharmaceutically acceptable salt or stereoisomer thereof, to a subject in need thereof.

As shown in working examples herein, the compounds of formula (I) (also referred to herein as degraders) cause degradation of at least one class IIa HDAC while substantially sparing other HDAC isoforms.

Accordingly, the compounds of the present disclosure may serve as a set of new chemical tools for class IIa HDACs knockdown, exemplify a broadly applicable approach to arrive at degraders that are selective relative to non-selective HDAC inhibitors, and may provide effective treatments for class IIa HDAC-mediated diseases and disorders such as neurodegenerative diseases (e.g., Parkinson's disease, Alzheimer's disease, and Huntington's disease), autoimmune diseases, alopecia, glucose homeostasis, muscular dystrophy and ischemic stroke.

### BRIEF DESCRIPTION OF THE DRAWINGS

FIG. 1 is a plot of cellular CRBN engagement assay for compounds 1 and 16.

FIG. 2A-2B are a set of plots of in vitro histone deacetylase (HDAC) enzymatic assays for compounds 1 (FIG. 2A) and 16 (FIG. 2B).

FIG. 3 is a heatmap showing expression downregulation of class IIa HDACs by indicated compounds by global quantitative proteomics.

FIG. 4A-FIG. 4C are scatterplots that show the change in relative protein abundance with treatment of Kelly cells with 3 (FIG. 4A), 16 (FIG. 4B), and 17 (FIG. 4C) compared to dimethyl sulfoxide (DMSO) control.

FIG. 5 is a scatterplot that shows the change in relative protein abundance with treatment of MM.1S cells with compound 17 compared to DMSO control.

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present disclosure, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ is group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, I-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl: bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —R-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R— heterocyclyl where R^{c} is an alkylene chain.

The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

To the extent not disclosed otherwise for any particular group(s), representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆. C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂. C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted proteins, which in this disclosure are class IIa histone deacetylases (i.e., HDAC4, 5, 7, and 9), typically refers to an inter-molecular interaction that is preferential (also referred to herein as “selective”) in that binding of the targeting ligand with other proteins present in the cell, including other HDAC isoforms, is substantially less and may be functionally insignificant. The terms “selective” and “selectivity” refer to the ability of the compound to discriminate between and among molecular targets. A selective class IIa histone deacetylase degrader described herein “substantially degrades at least one class IIa HDAC and “substantially spares other HDAC isoforms” in that it may have a DC₅₀ (half maximal degradation concentration) for at least one class IIa HDAC activity that is at least about 1, 2, 3, 4, 5, 6, 7, 8, 9, or 10-fold lower than the DC₅₀ for one or more of HDAC1, HDAC2, HDAC3, HDAC6, and/or HDAC10. Thus, even though various compounds of the present disclosure may exhibit non-negligible binding to other HDAC proteins, they cause selective degradation of at least one class IIa HDAC.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but is sufficient nonetheless to achieve recruitment of the ligase to the targeted proteins, which in this disclosure are class IIa HDACs, for selective degradation.

Broadly, the compounds comprise a moiety that binds at least one class IIa histone deacetylase (HDAC) and a degron covalently attached to each other by a linker that comprises an alkylene chain or a polyethylene glycol (PEG) chain, wherein the compound has a structure represented by formula (I):

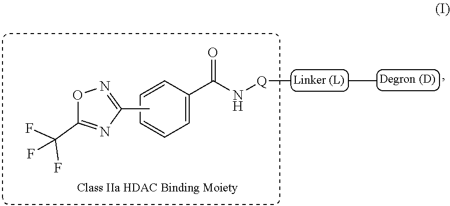

wherein:
Q represents

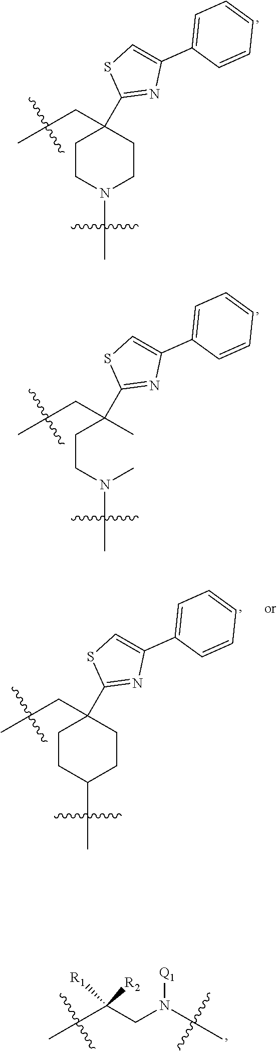

wherein R₁ and R₂ are independently H or C₁-C₄ alkyl and Q₁ is optionally C₁-C₄ alkyl; and the degron represents a ligand that binds cereblon (CRBN), von Hippel Landau tumor suppressor (VHL), or inhibitor of apoptosis protein (IAP), or a pharmaceutically acceptable salt or stereoisomer thereof.

In some embodiments, Q is

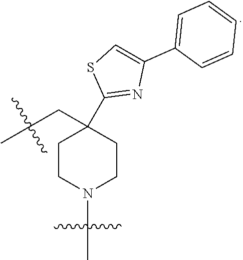 [SMILES: *CC1(c2nc(-c3ccccc3)cs2)CCN(*)CC1]

In some embodiments, Q is

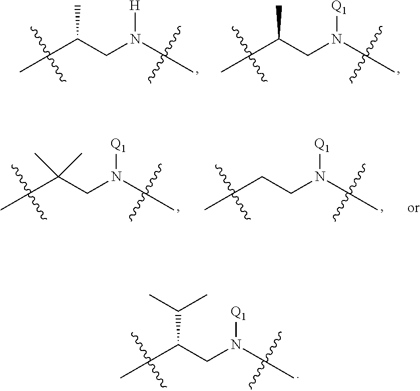

In some embodiments, Q is

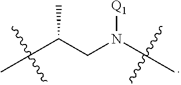

In some embodiments, Q₁ is ethyl or benzyl.

In some embodiments, compounds of the present disclosure may be represented by any one of structures (I-1) and (I-2):

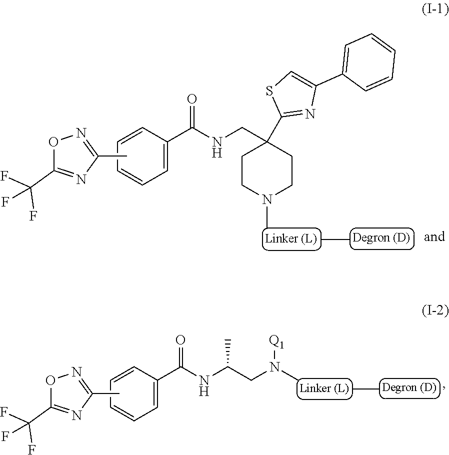

or a pharmaceutically acceptable salt or stereoisomer thereof.

### Linkers

The linker (“L”) provides a covalent attachment between the targeting ligand and the degron. The structure of linker may not be critical, provided it is substantially non-interfering with the activity of the class IIa HDAD targeting ligand or the degron. In some embodiments, the linker includes an alkylene chain (e.g., having 2-20 alkylene units). In other embodiments, the linker may include an alkylene chain or a bivalent alkylene chain, either of which may be interrupted by, and/or terminate (at either or both termini) at least one of —O—, —S—, —N(R′)—, —C≡C—. —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may include a C₁-C₁₂ alkylene chain terminating in NH-group wherein the nitrogen is also bound to the degron.

In some embodiments, the linker includes an alkylene chain having 1-10 alkylene units that is interrupted by and/or terminating in

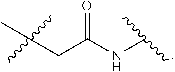 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present disclosure include the following:

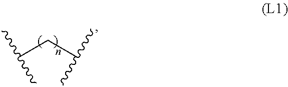

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

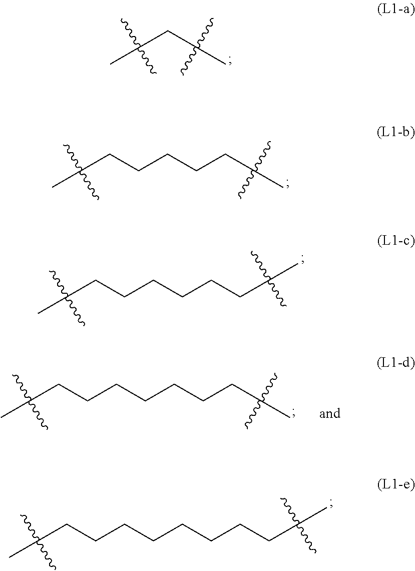

alkylene chains terminating in various functional groups (as described above), examples of which are as follows:

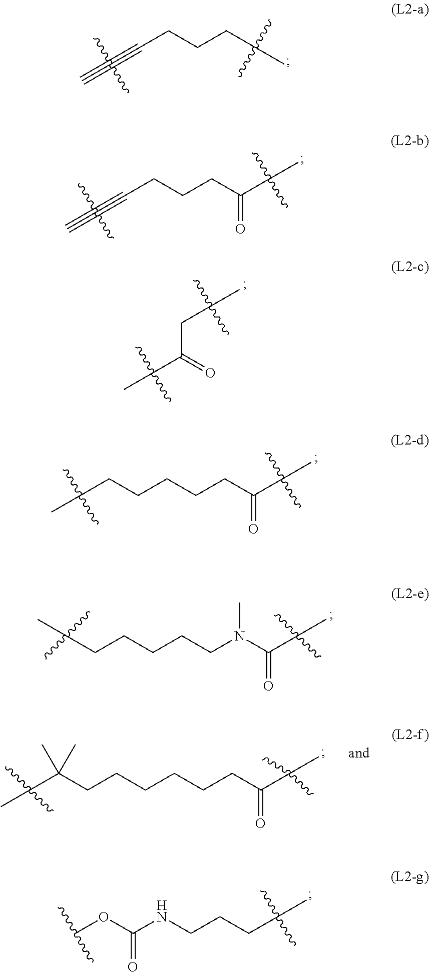

alkylene chains interrupted with various functional groups (as described above), examples of which are as follows:

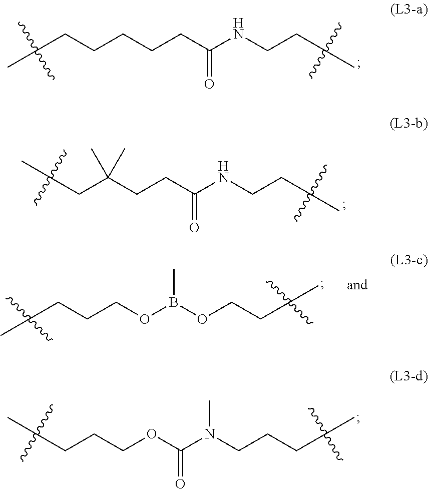

alkylene chains interrupted or terminating with a heterocyclene group, e.g.,

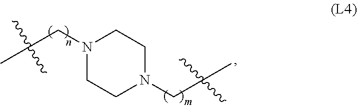

wherein m and n are independently integers of 0-10, examples of which include:

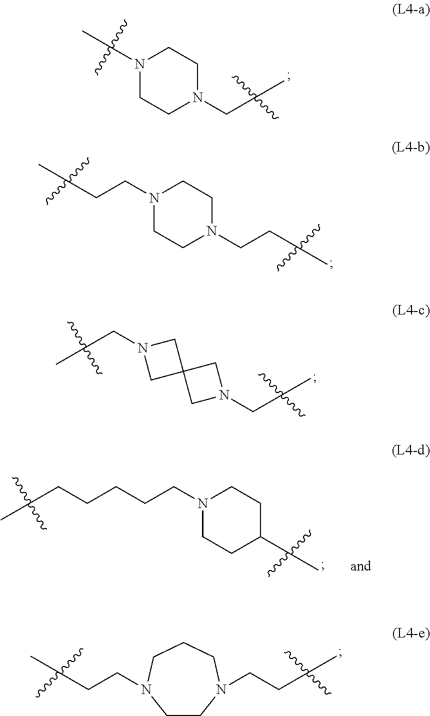 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by an amide, a heterocyclene and/or an aryl group, examples of which include:

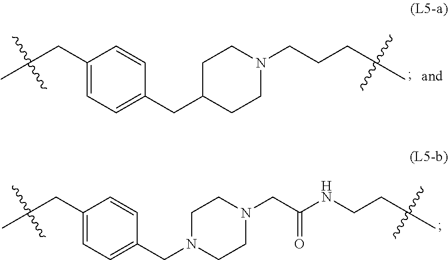 [SMILES: *CCCN1CCC(Cc2ccc(C*)cc2)CC1 ; *CCNC(=O)CN1CCN(Cc2ccc(C*)cc2)CC1]

alkylene chains interrupted by a heterocyclene, an aryl group, and a heteroatom, examples of which include:

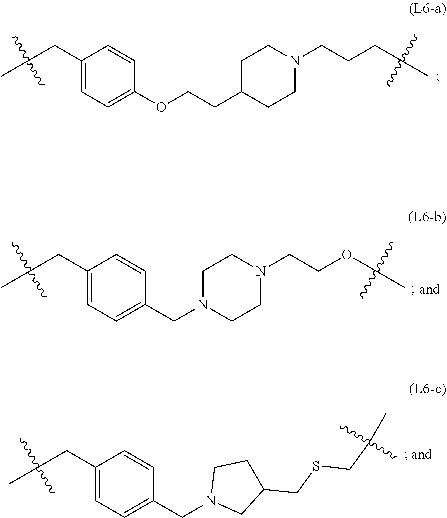 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

alkylene chains interrupted by a heteroatom such as N, O or B, e.g.,

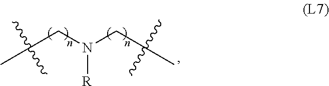

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

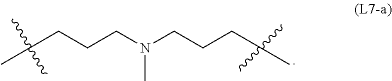 [SMILES: *CCCN(C)CCC*]

In some embodiments, the linker may include a polyethylene glycol chain that may terminate (at either or both termini) in at least one of —S—, —N(R′)—, —C≡C—, —C(O)—, —C(O)O—OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 2-8 PEG units and terminates at one or both termini in

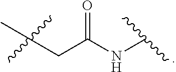 [SMILES: *CC(=O)N*]

Representative examples of linkers that include a polyethylene glycol chain include:

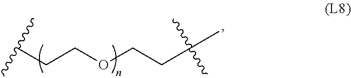

wherein n is an integer of 2-10, examples of which include:

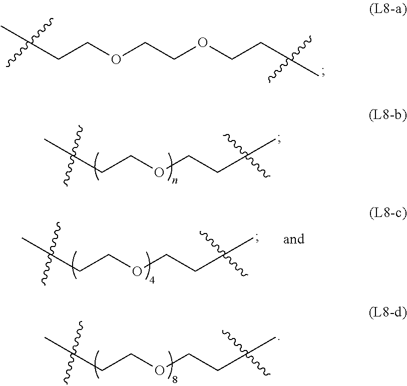

In some embodiments, the polyethylene glycol linker may terminate in a functional group, examples of which are as follows:

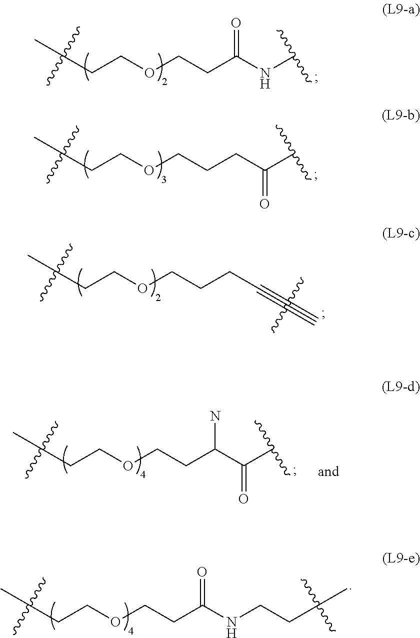

In some embodiments, the linker is represented by any one of structures:

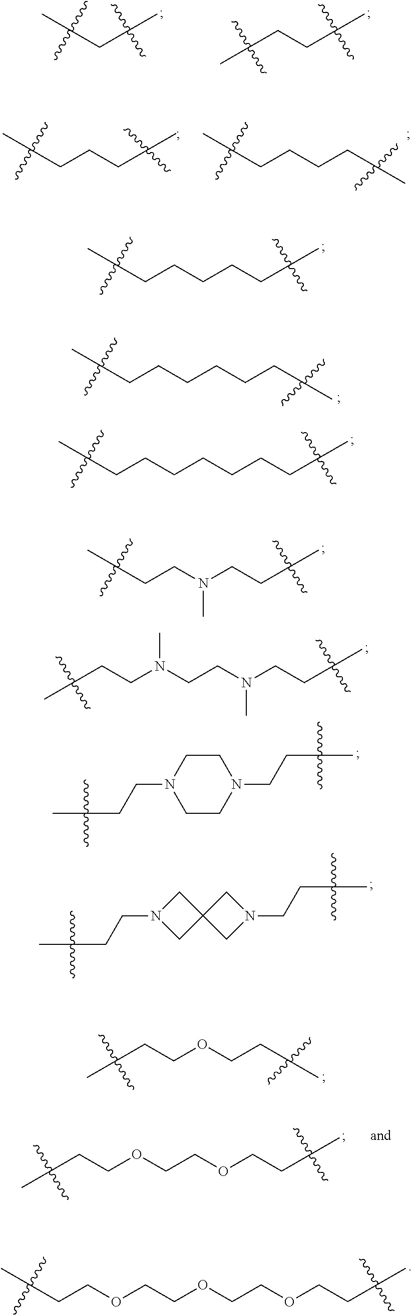

Therefore, in some embodiments, compounds of the present disclosure may be represented by any one of structures (I-3) to (I-12):

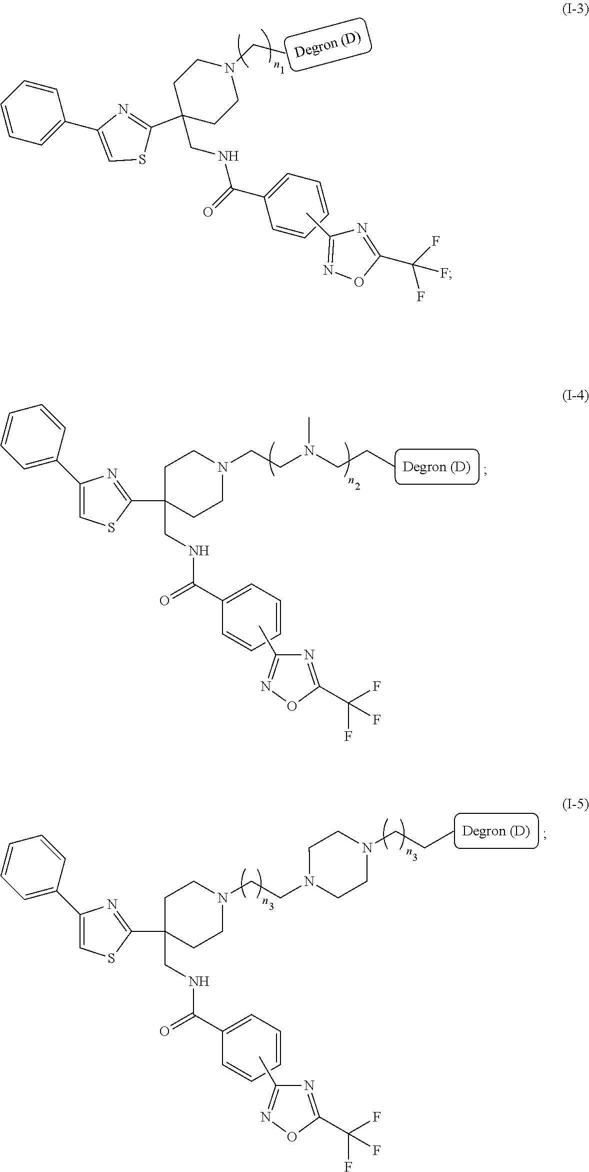

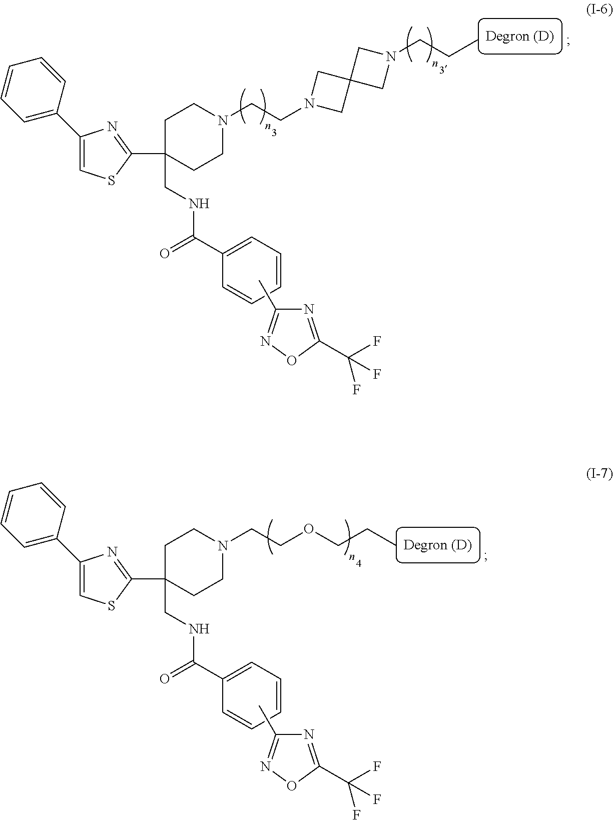

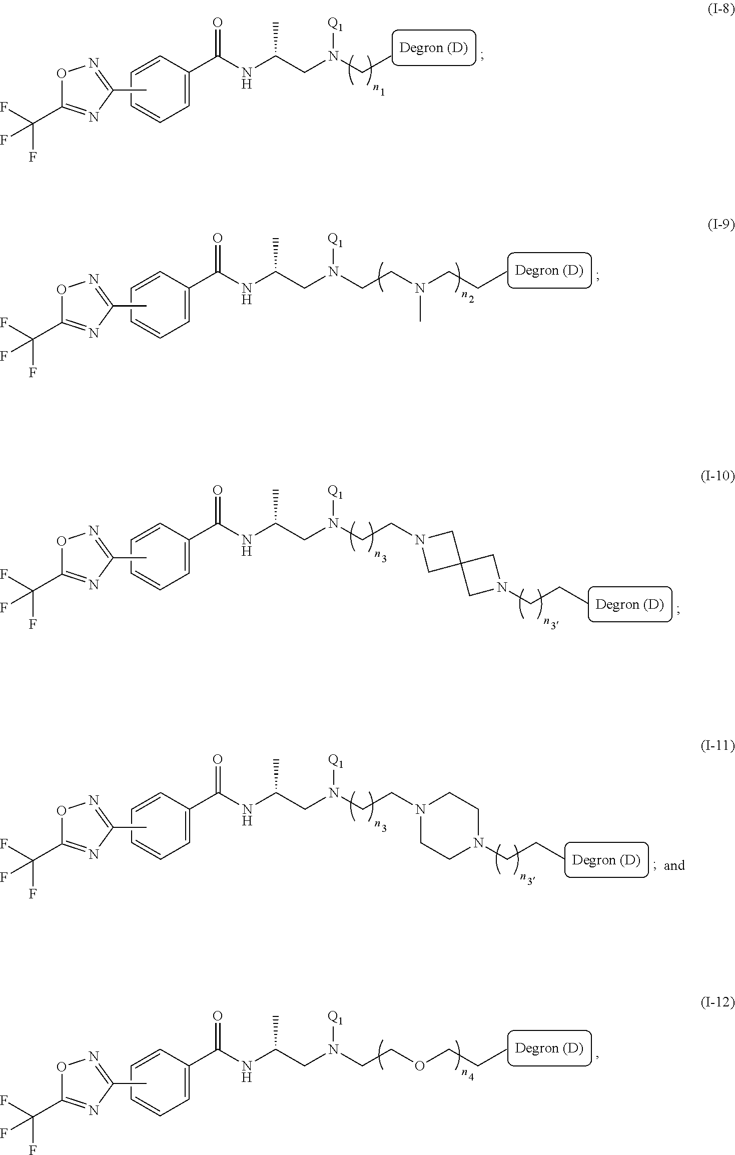

wherein n₁ is an integer from 0-12, n₂ is an integer from 1-2, n₃ and n_{3′} are independently an integer from 1-8, n₄ is an integer from 1-5, and Q₁ is optionally C₁-C₃ alkyl, or a pharmaceutically acceptable salt or stereoisomer thereof

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ligase which is cereblon. (CRBN).

Representative examples of such degrons are represented by any one of structures (D1a) to (D1d):

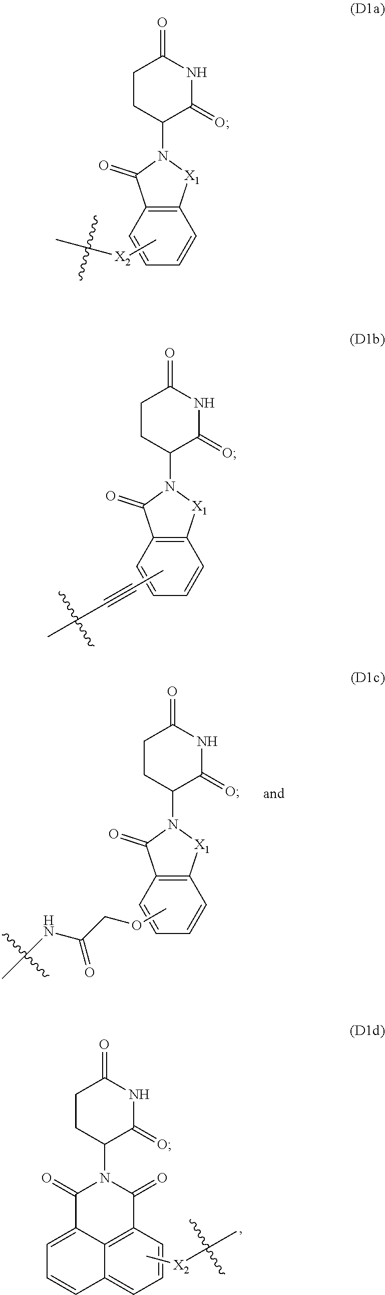

wherein X₁ is CH₂ or C(O) and X₂ is a bond, CH₂, NH, or O.

Yet other degrons that bind cereblon and which may be suitable for use in the present disclosure are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

Therefore, in some embodiments, the compounds of the present disclosure may be represented by any of structures (I-13) to (I-52):

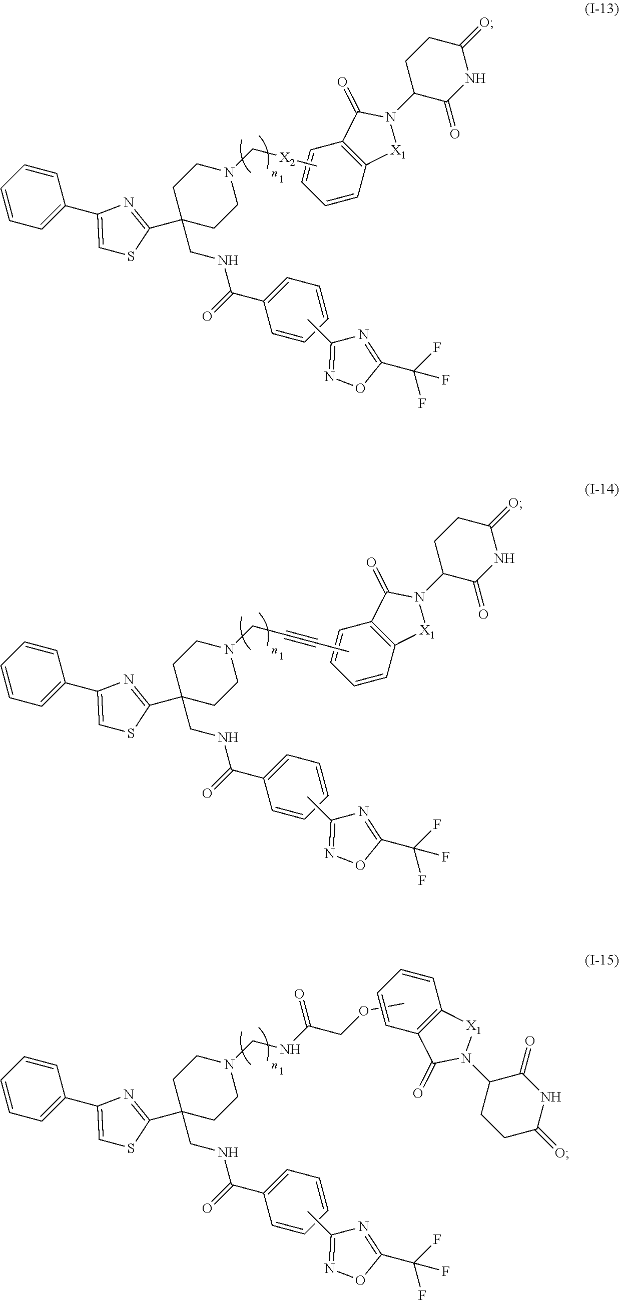

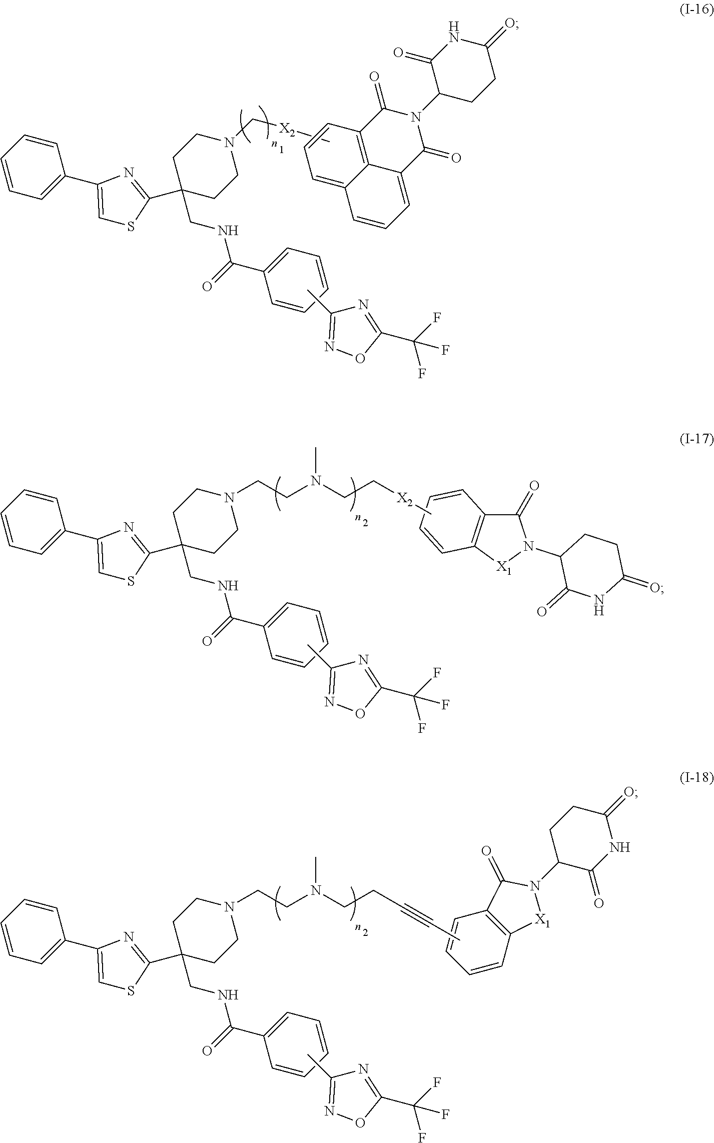

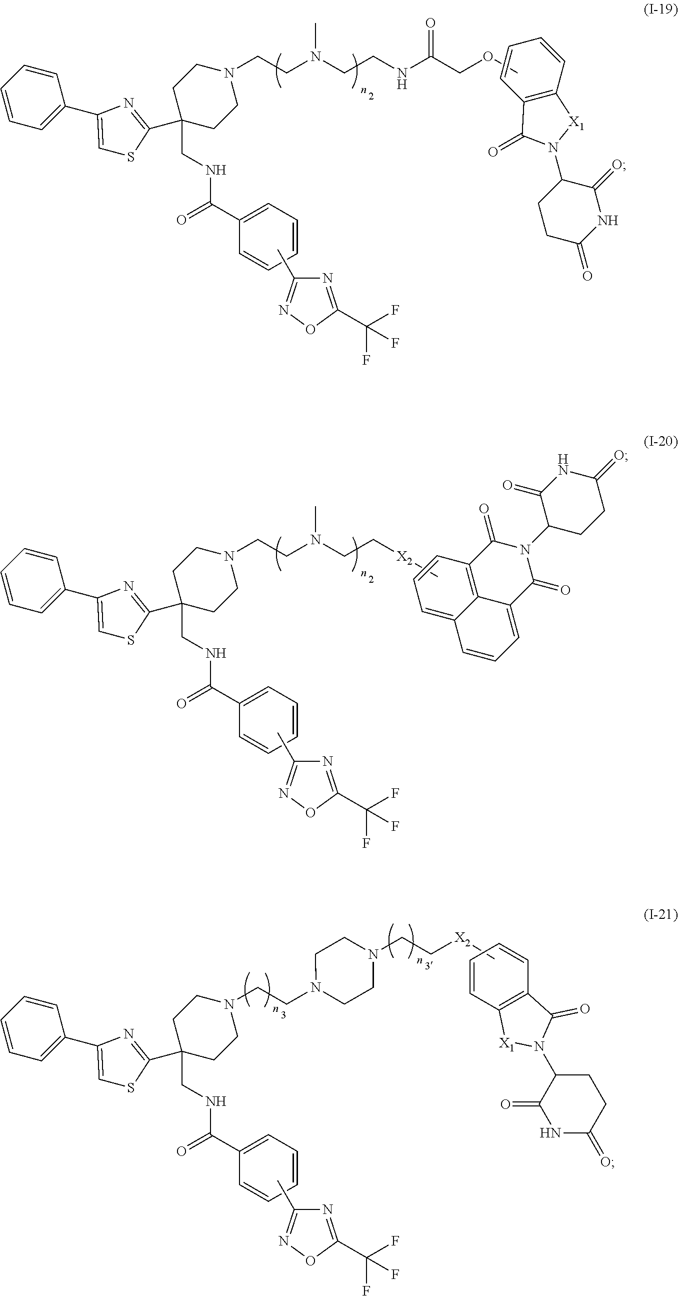

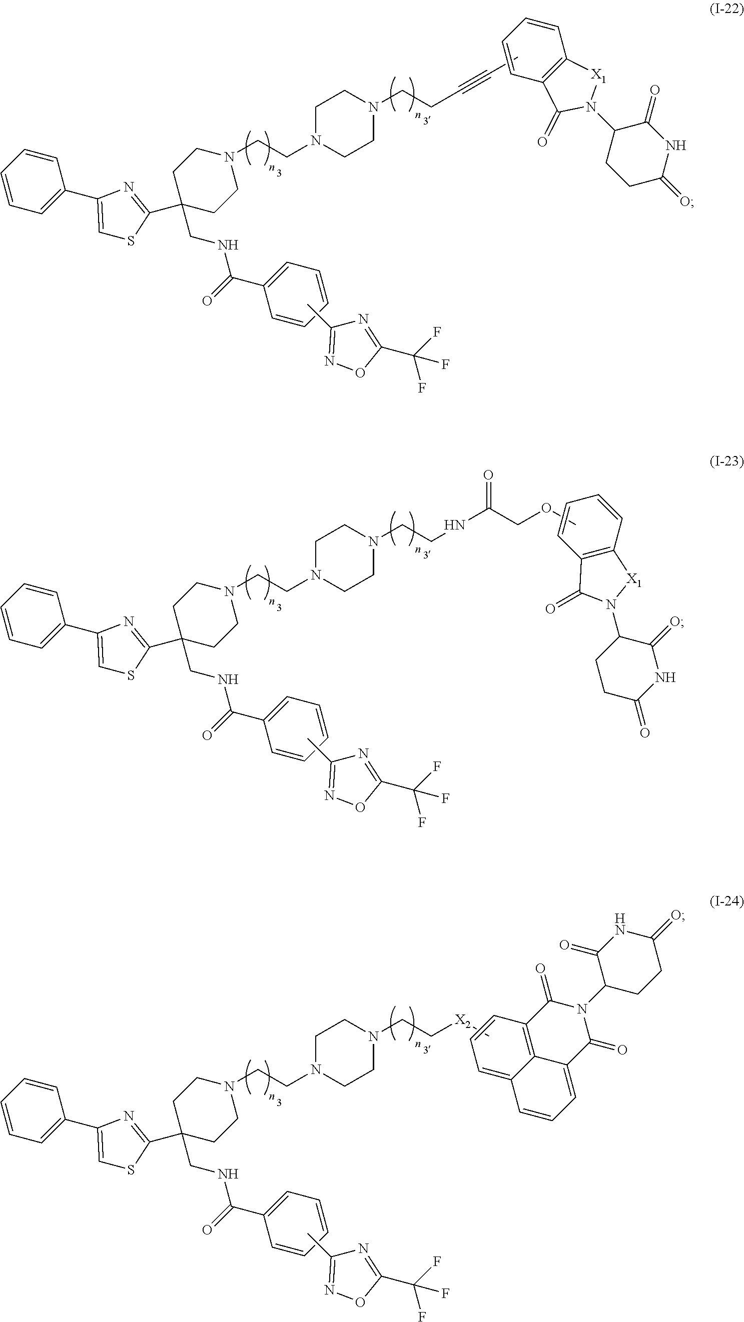

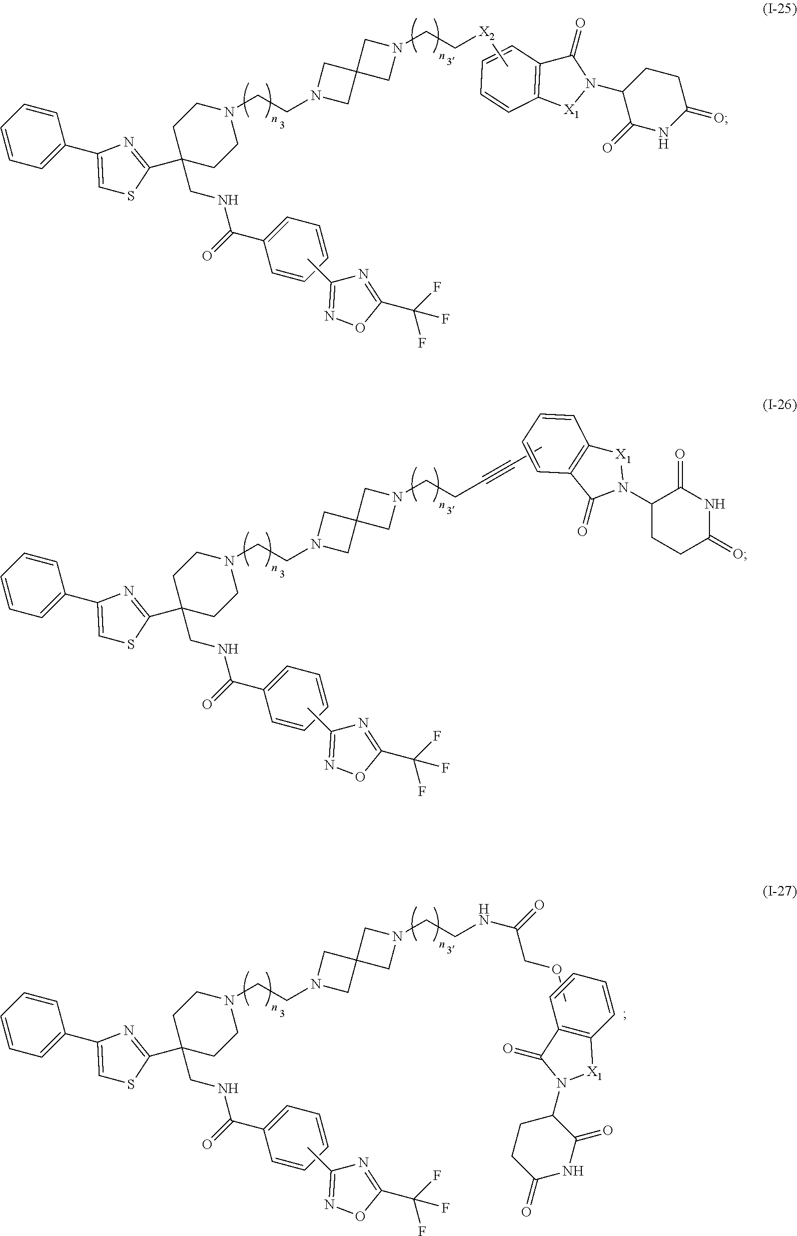

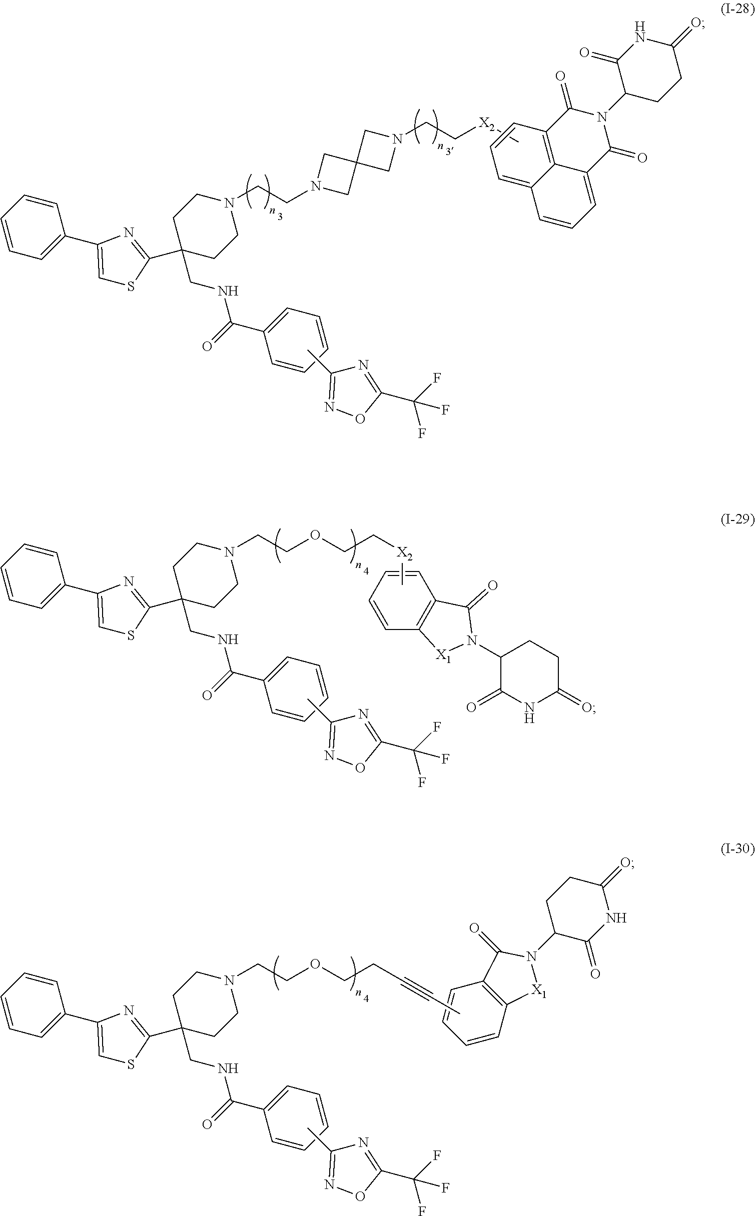

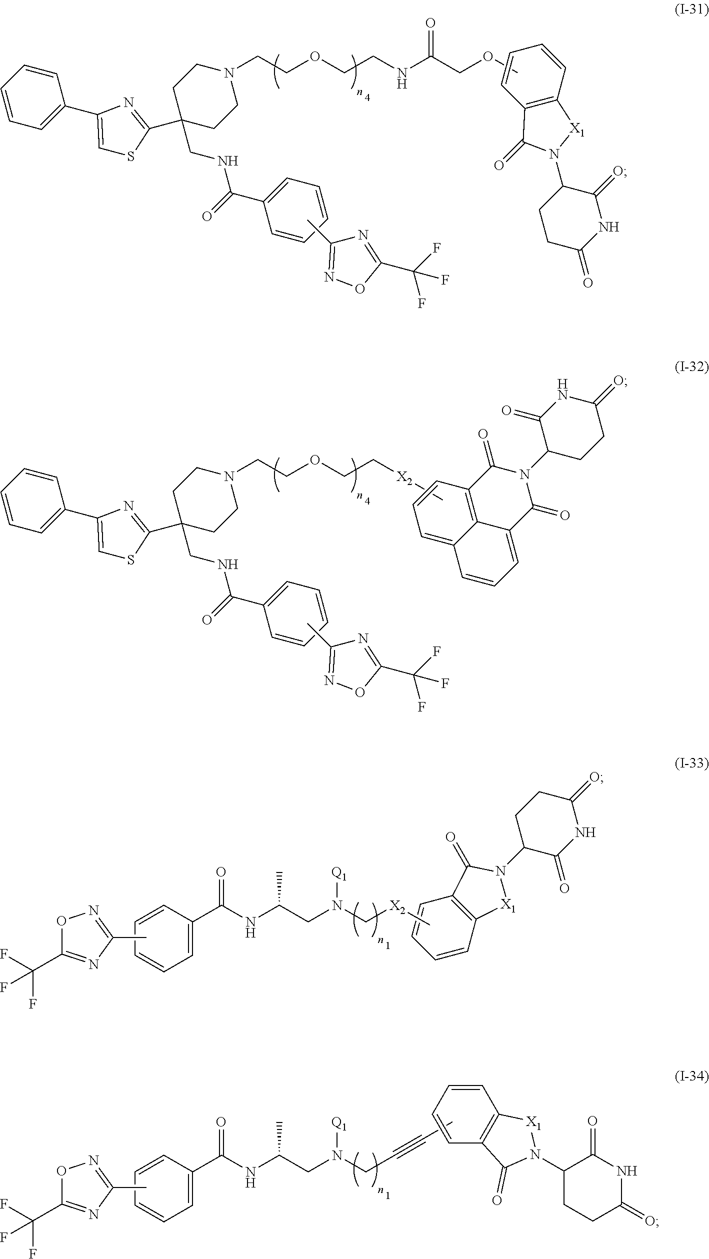

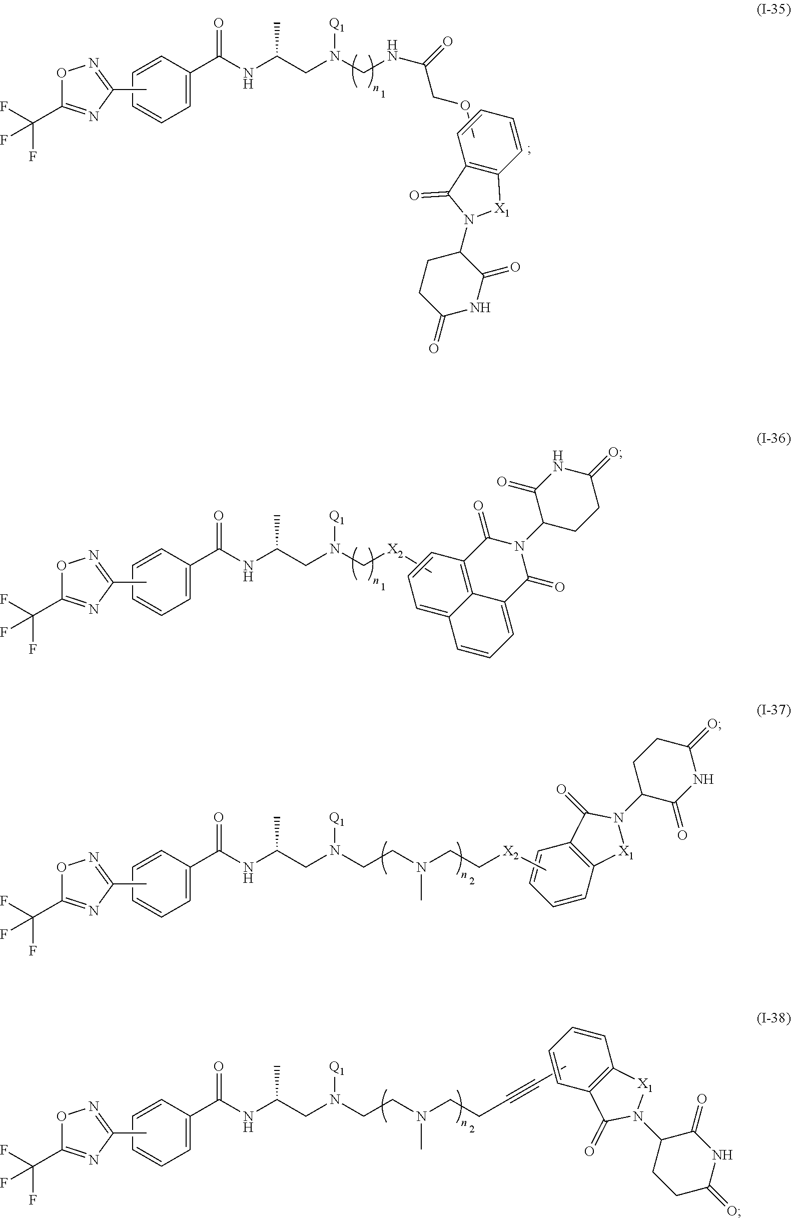

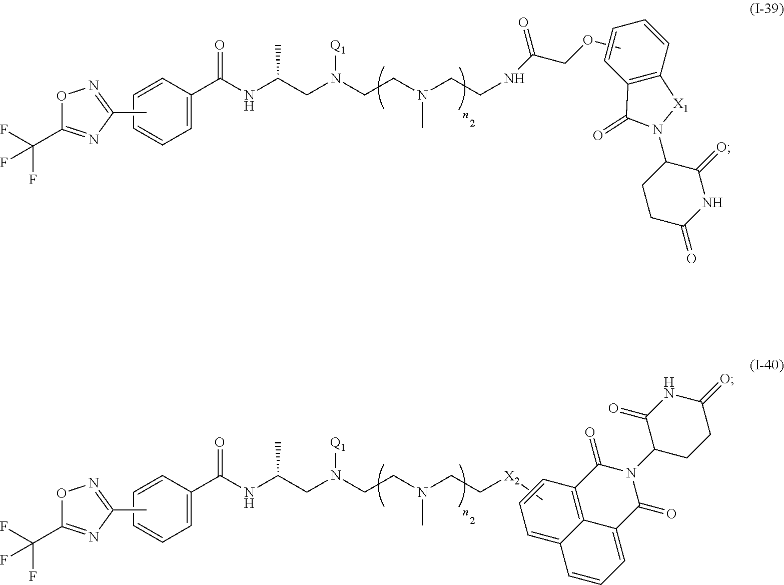

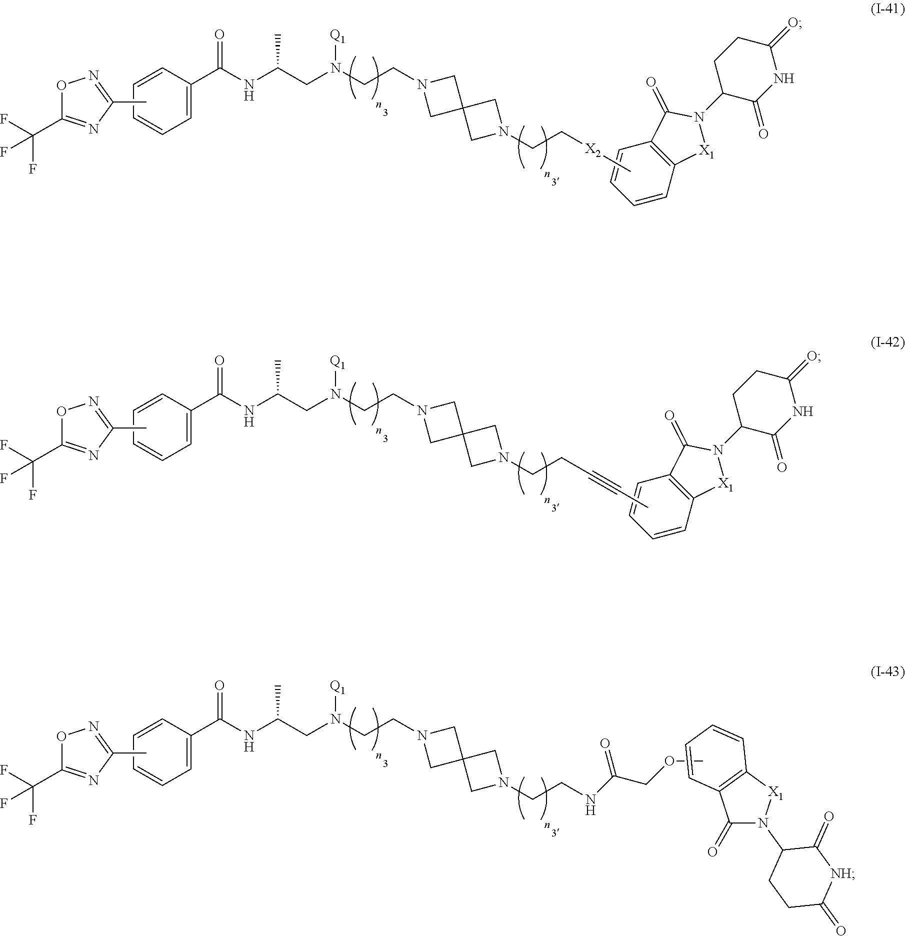

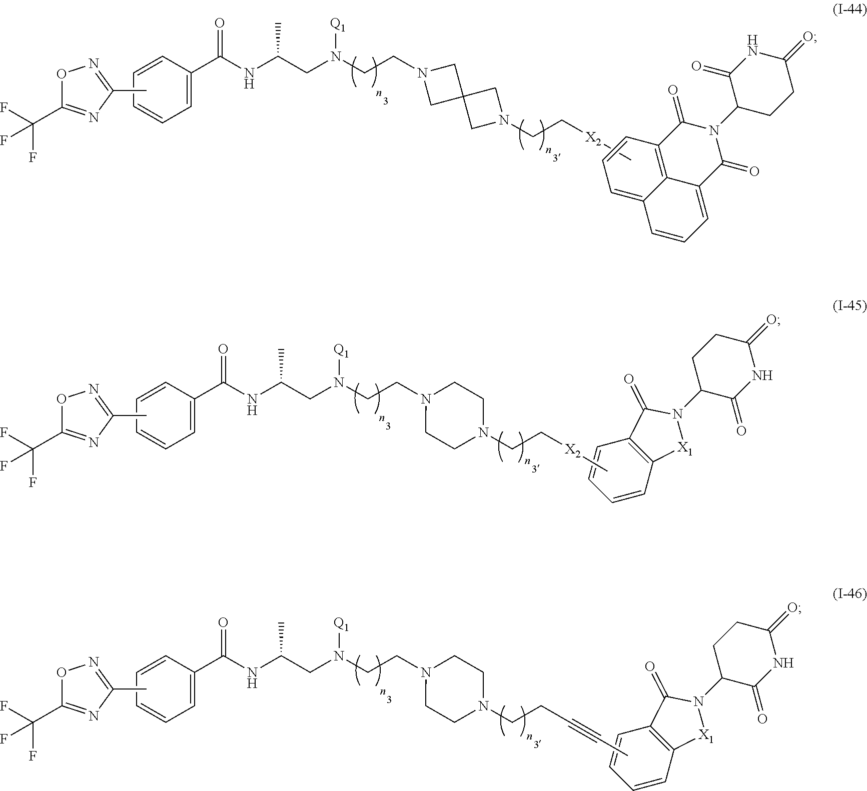

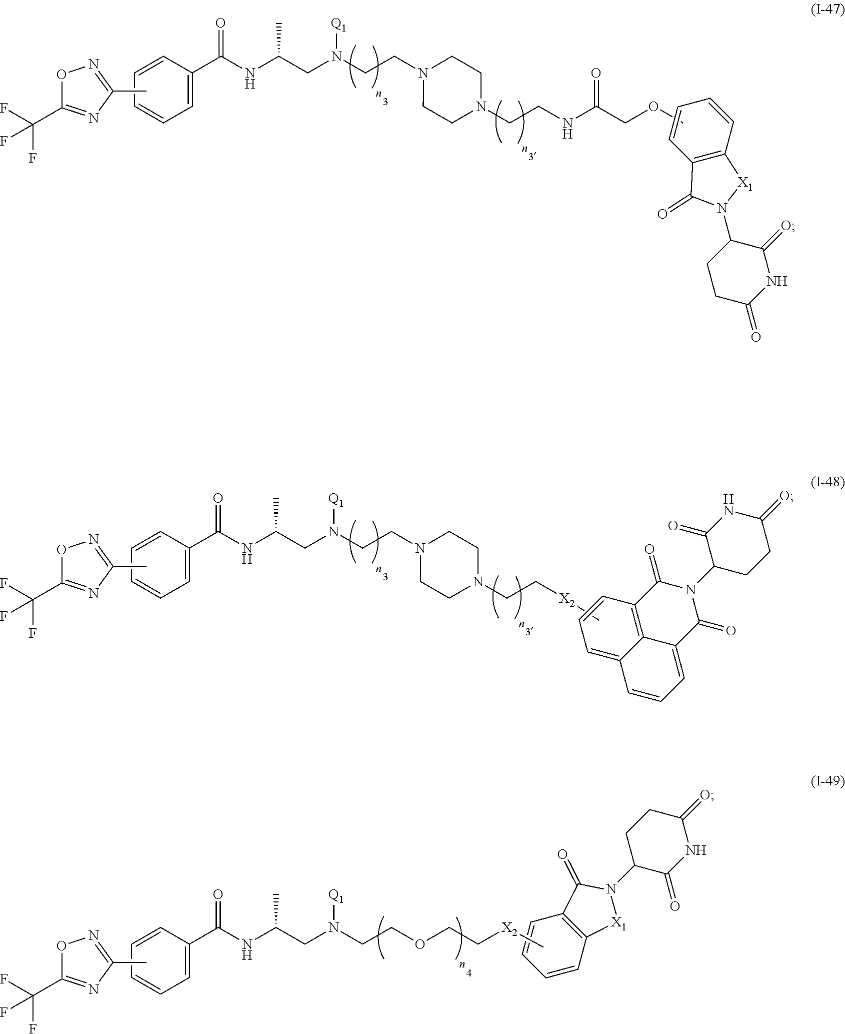

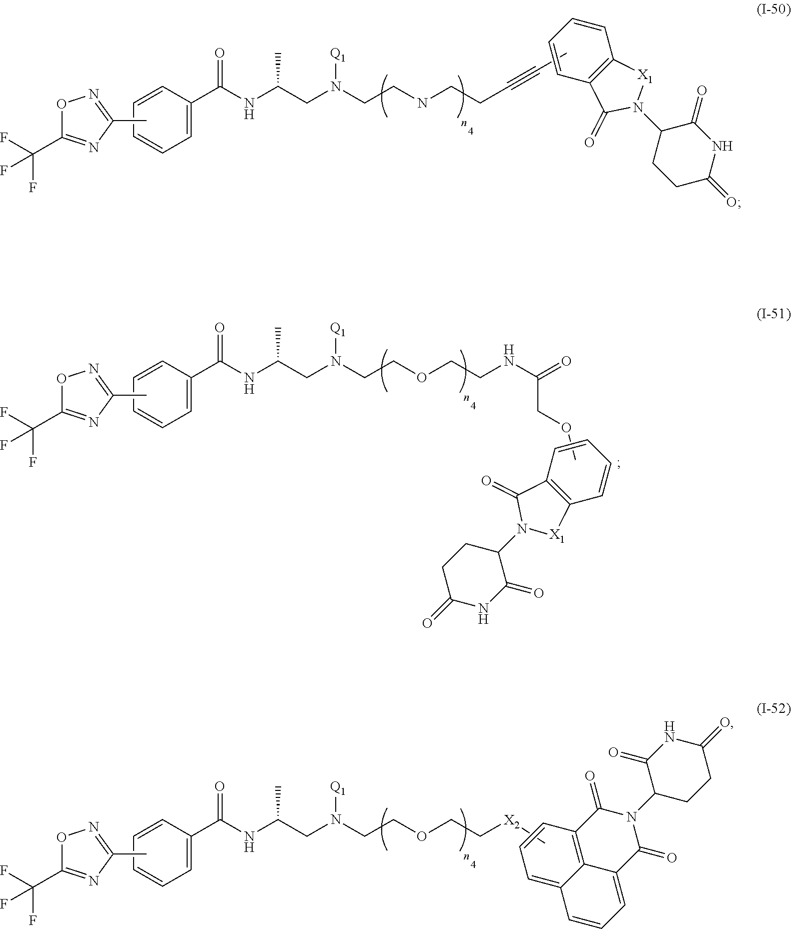

or a pharmaceutically acceptable salt or stereoisomer thereof.

In some embodiments, the degron binds the von Hippel-Lindau (VHL) E3 ubiquitin ligase.

Representative examples of such degrons are represented by any one of structures (D1-a) to (D1-f):

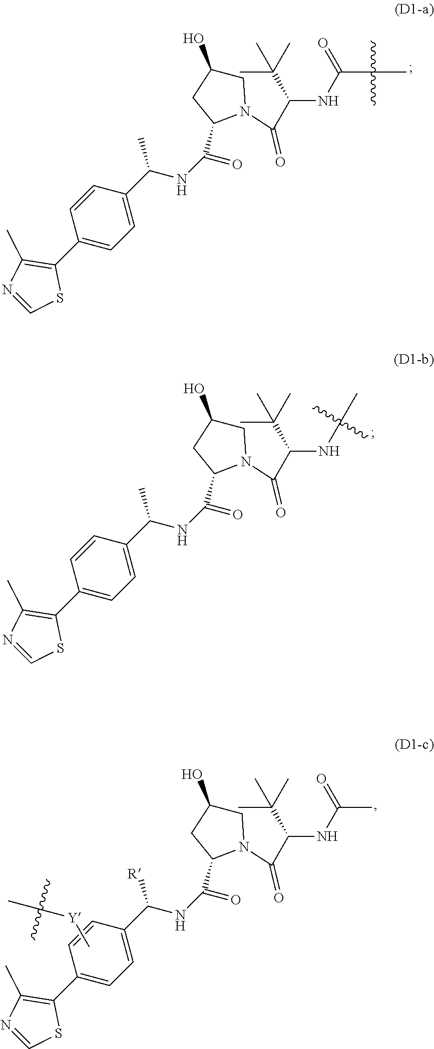

wherein Y′ is a bond, N, O or C and R′ is H or methyl;

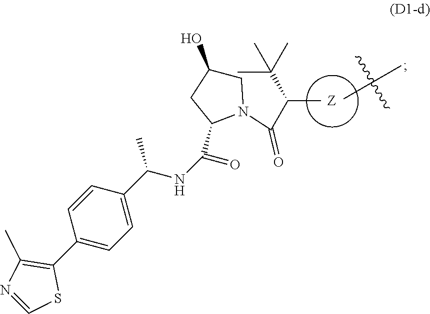

wherein Z is a C₅-C₆ carbocyclic or a C₅-C₆ heterocyclic group;

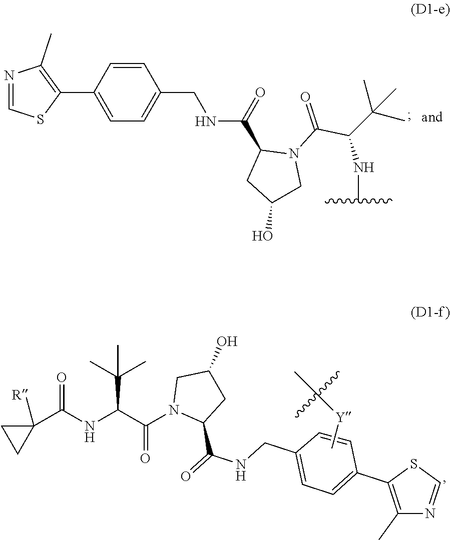

wherein Y″ is a bond, N, O or C and R″ is F or CN,
or a stereoisomer thereof.

In some embodiments, Z is

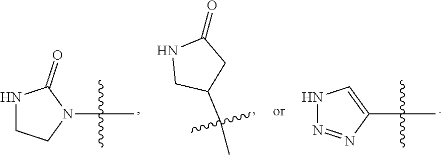 [SMILES: *N1CCNC1=O ; *C1CNC(=O)C1 ; *c1c[nH]nn1]

Yet other degrons that bind VHL and which may be suitable for use in the present disclosure are disclosed in U.S. Patent Application Publication 2017/0121321 A1.

Therefore, in some embodiments, the compounds of the present disclosure may be represented by any of structures (I-53) to (I-112):

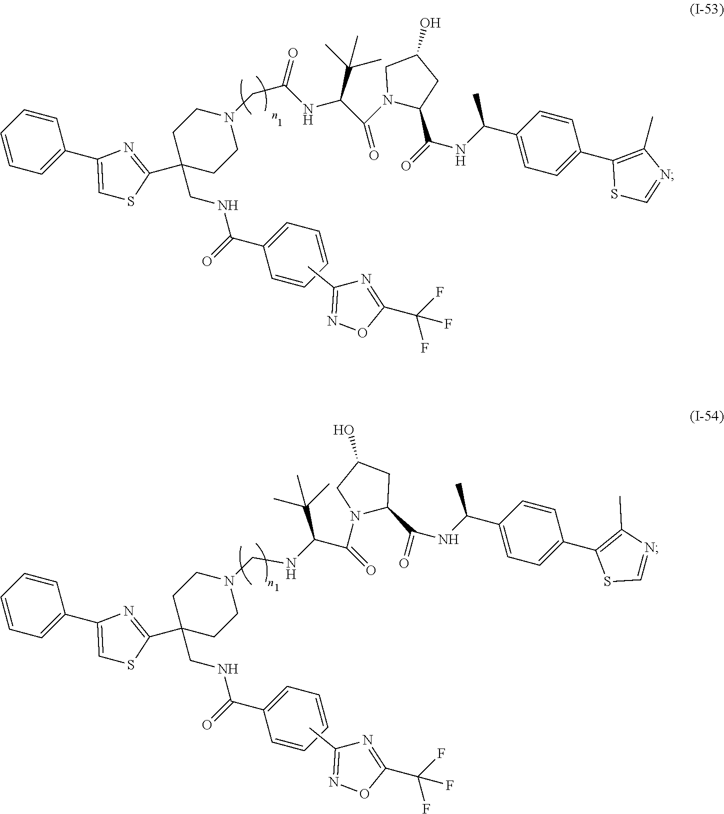

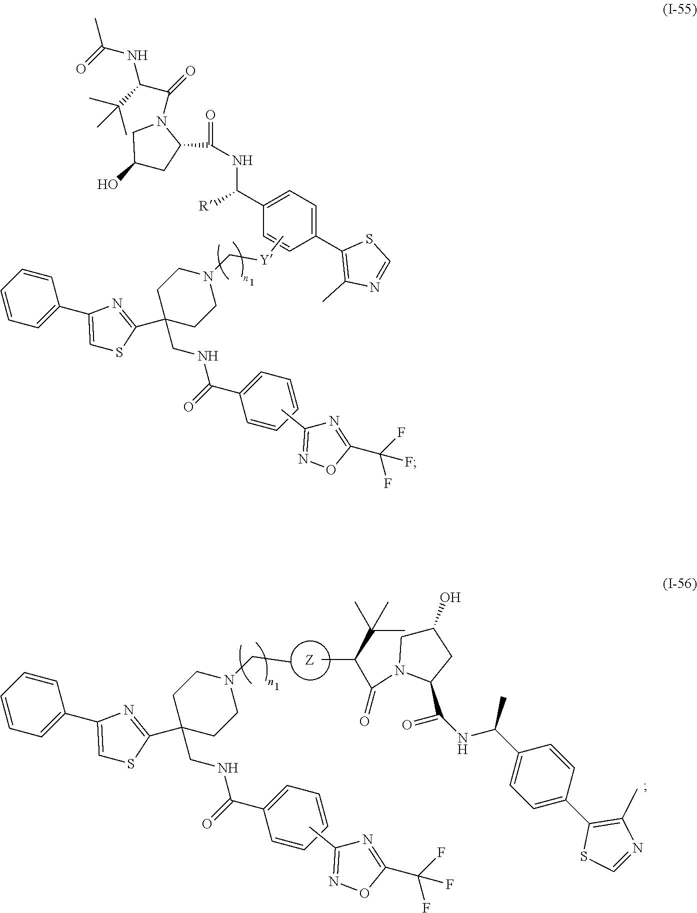

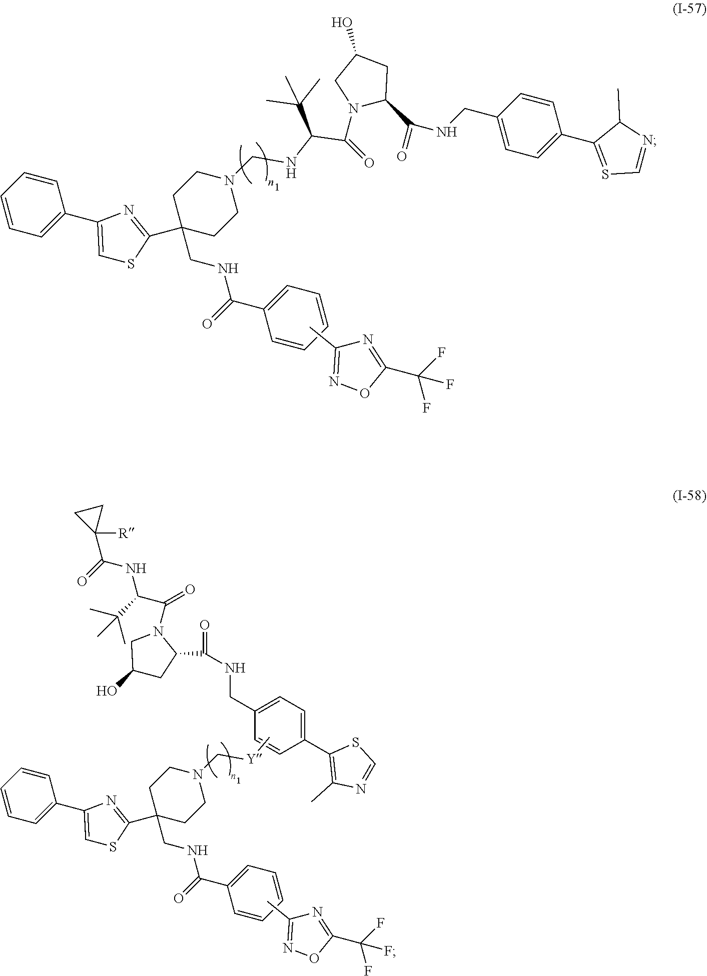

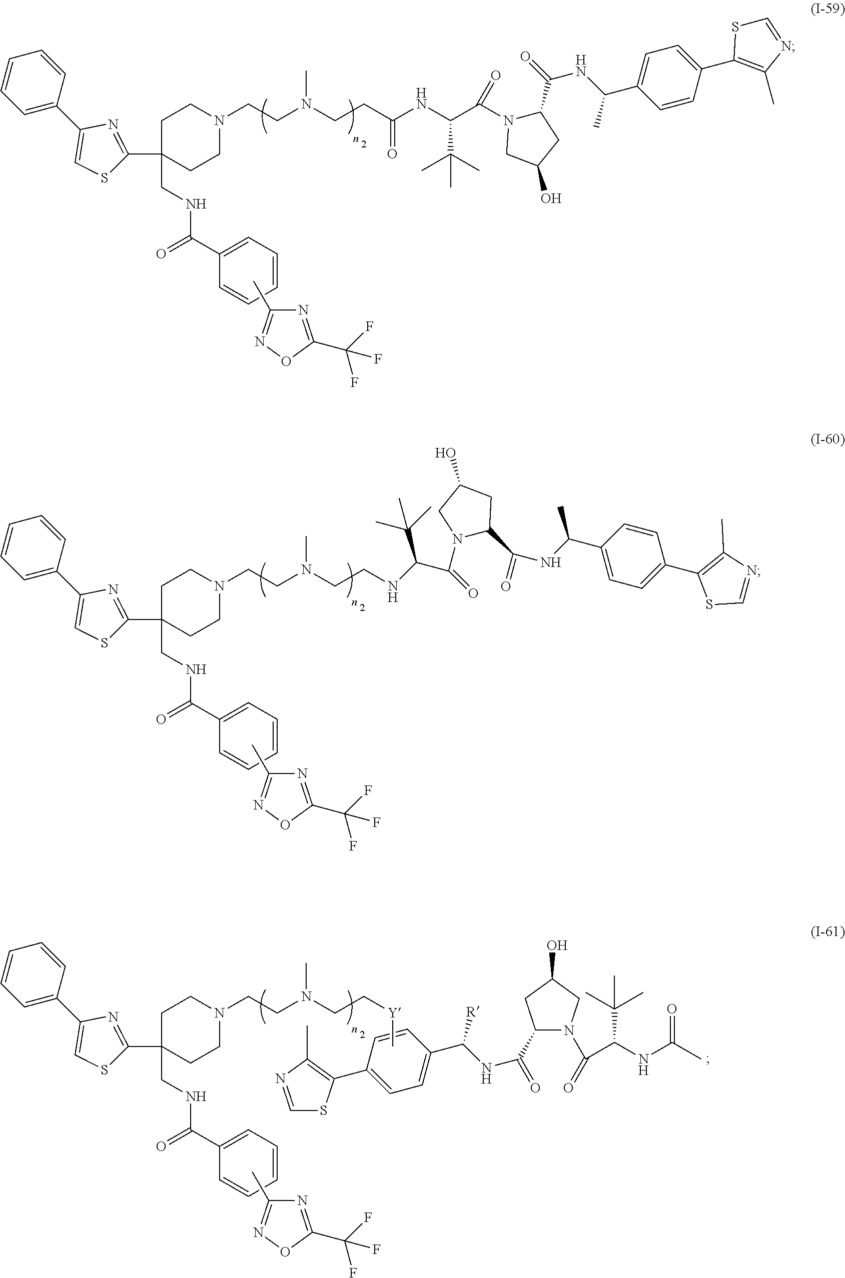

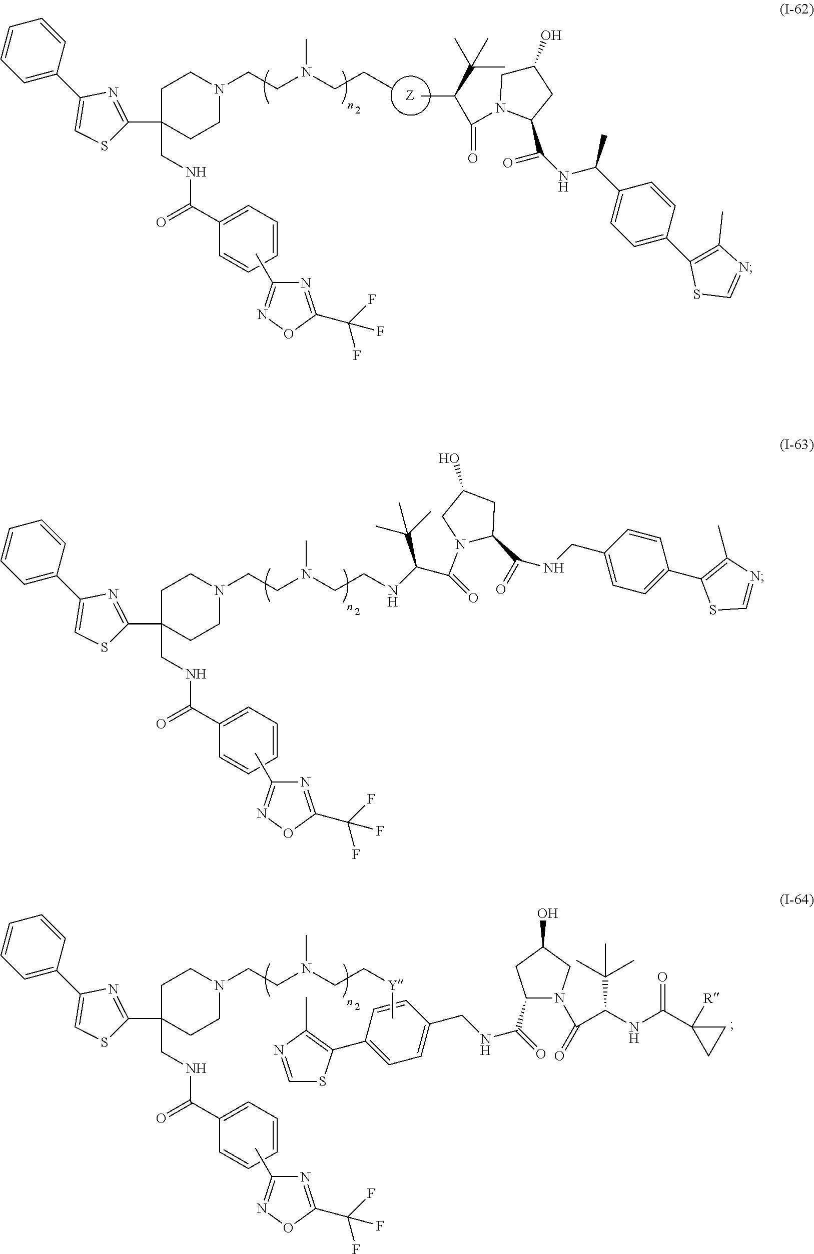

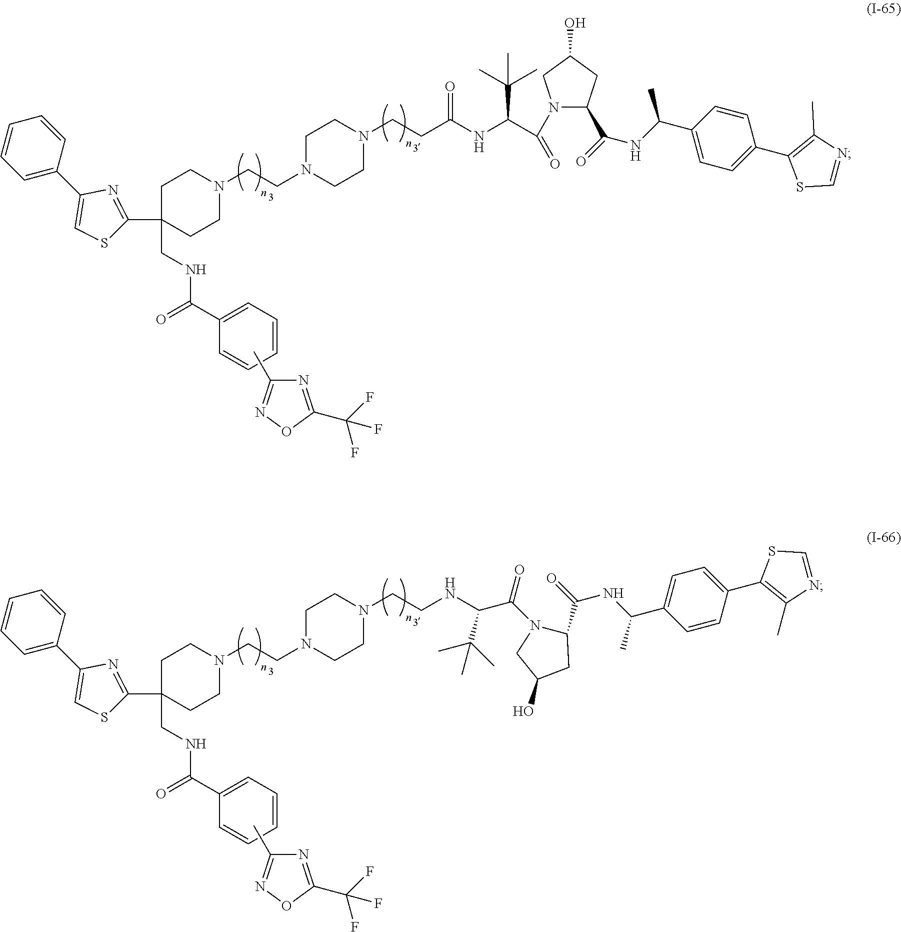

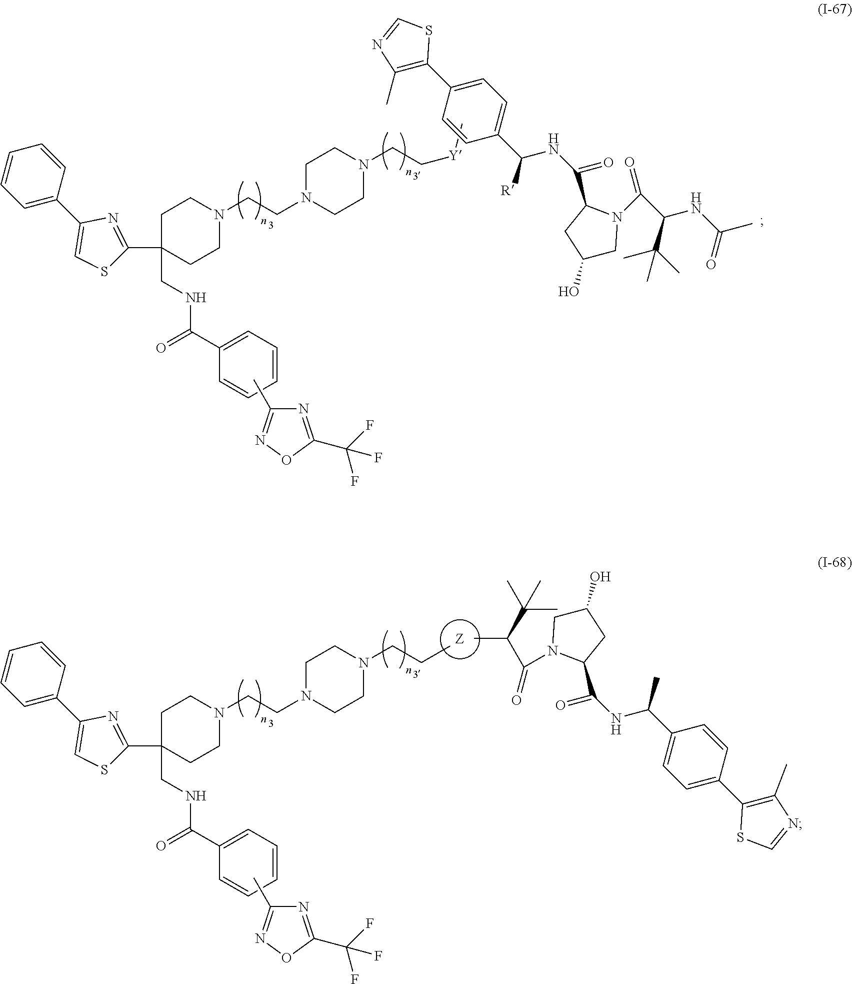

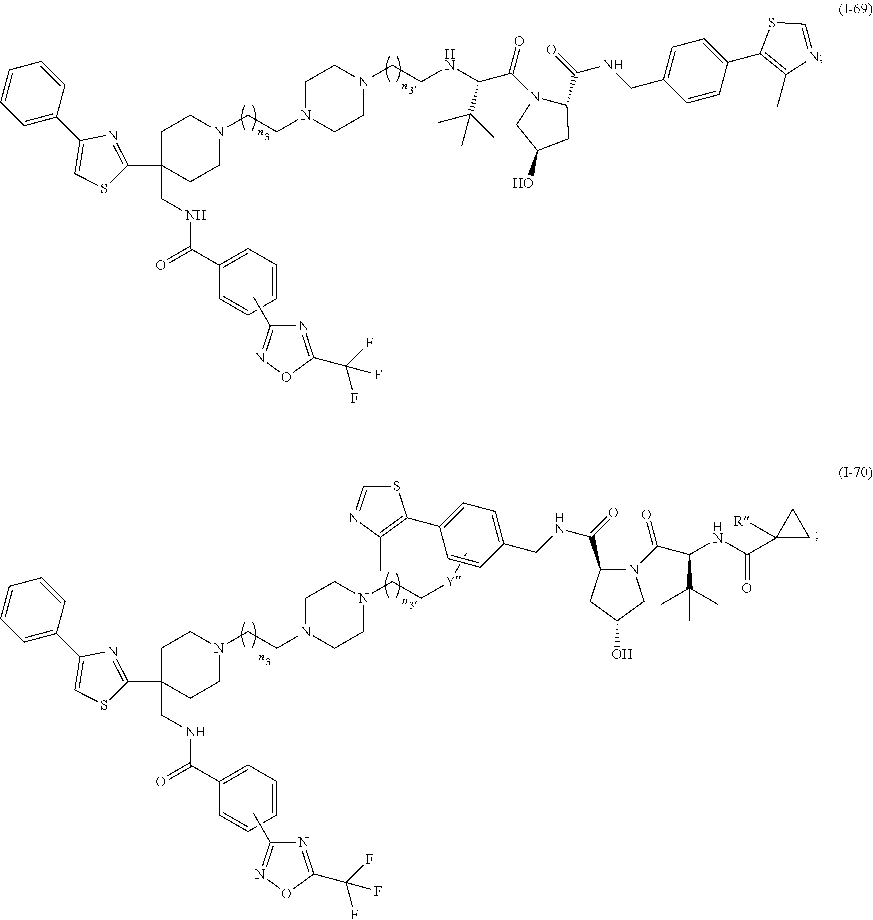

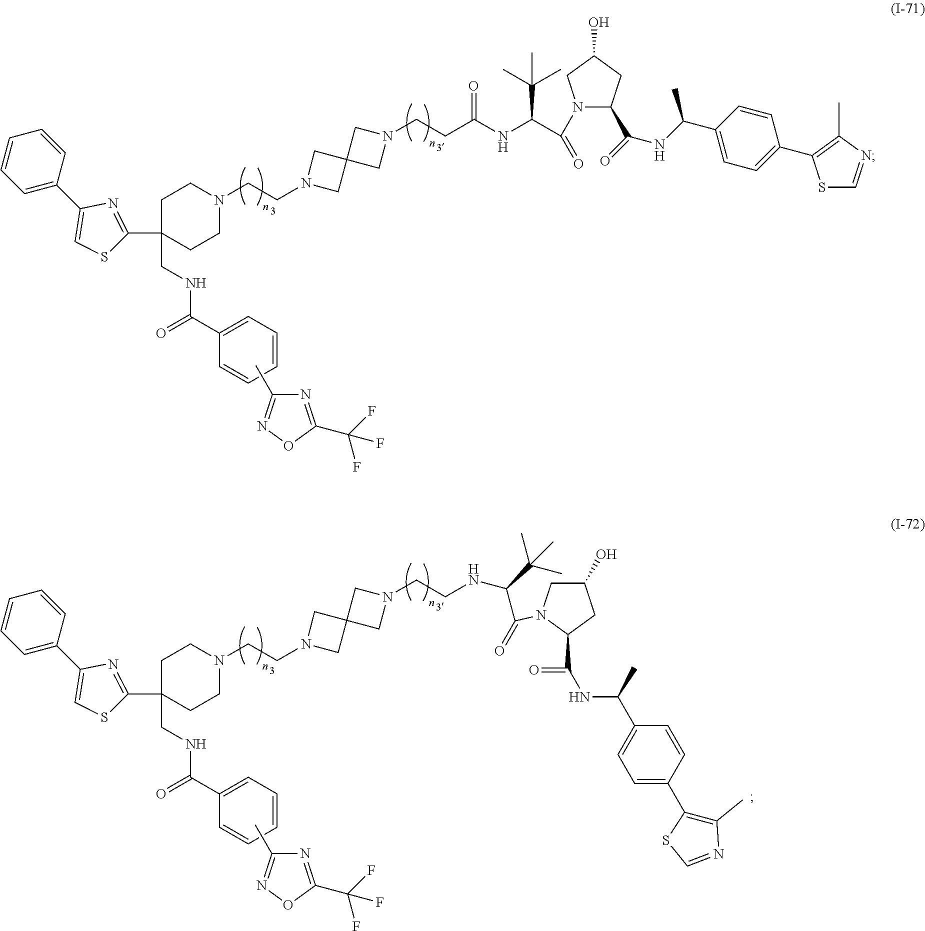

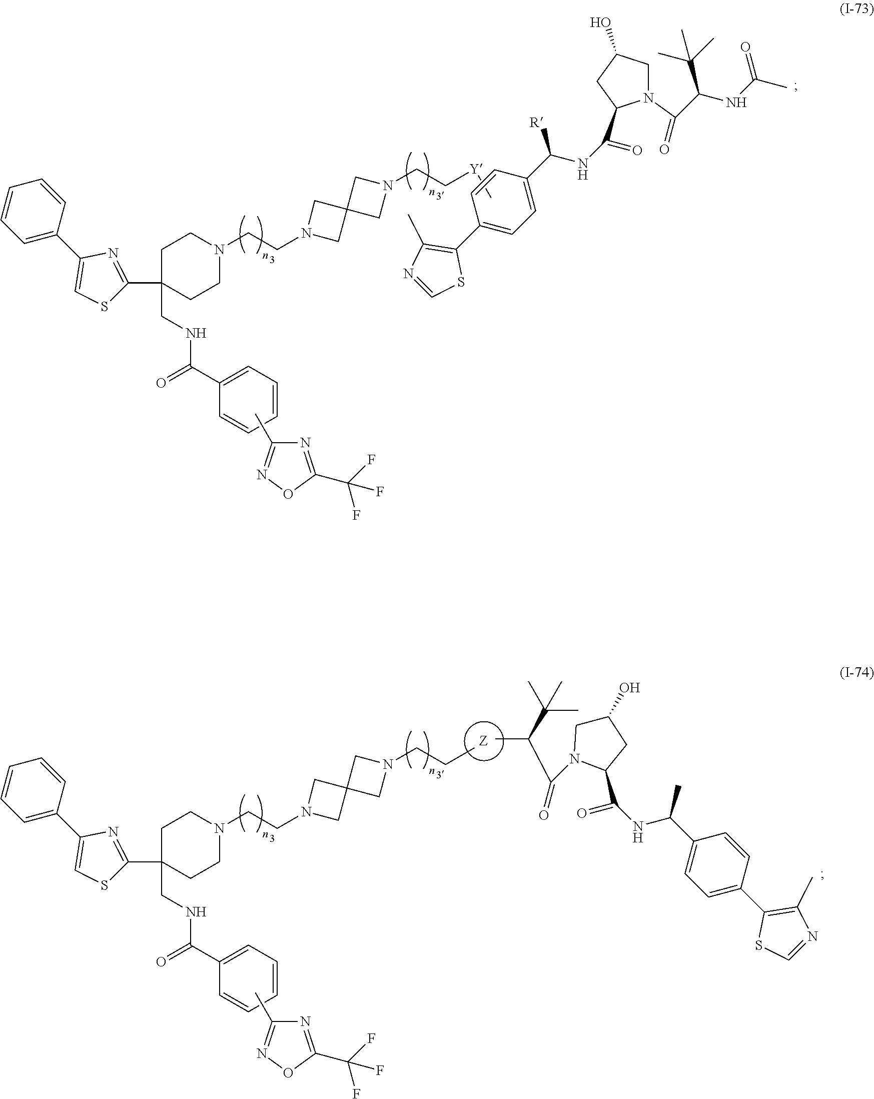

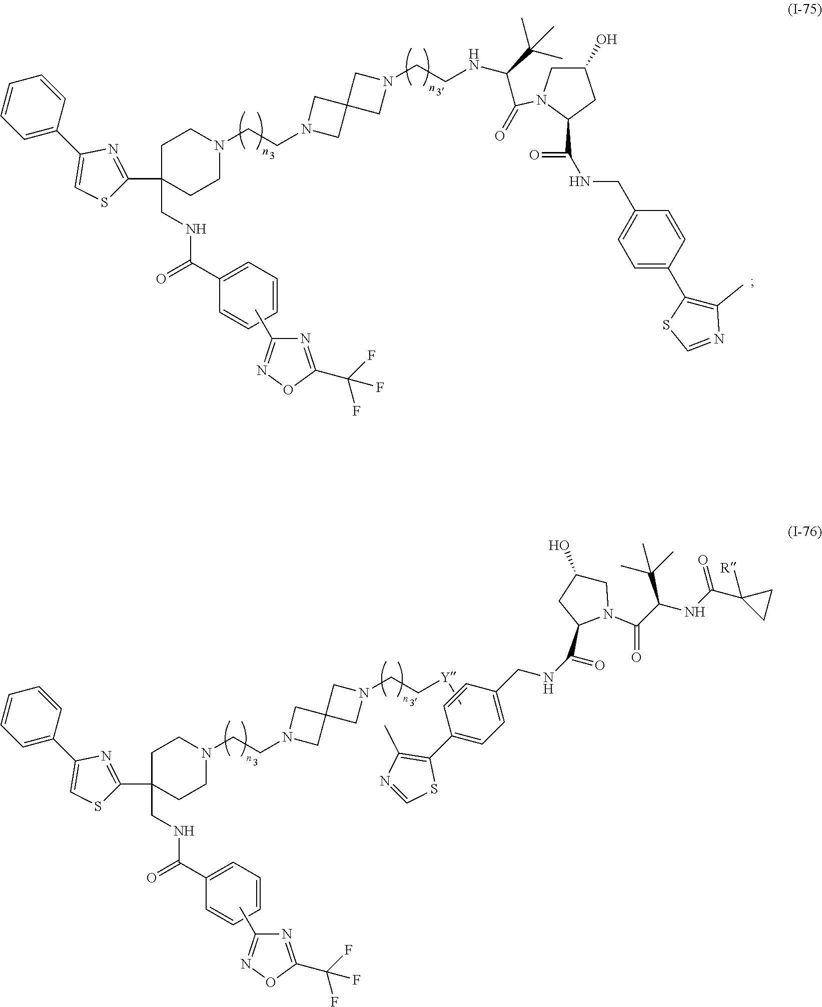

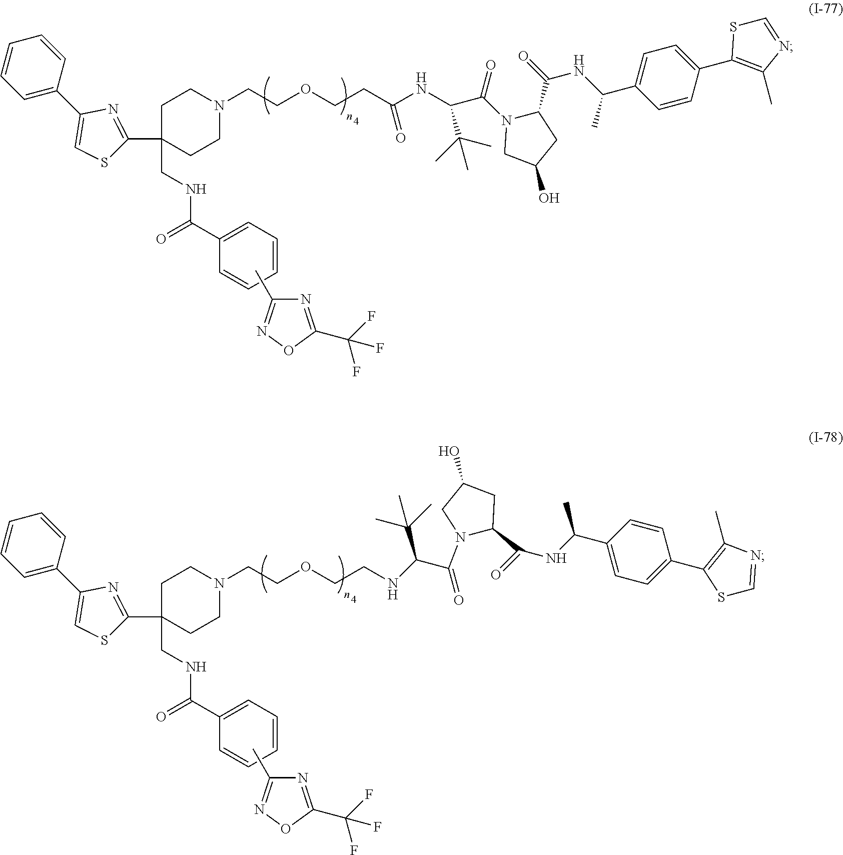

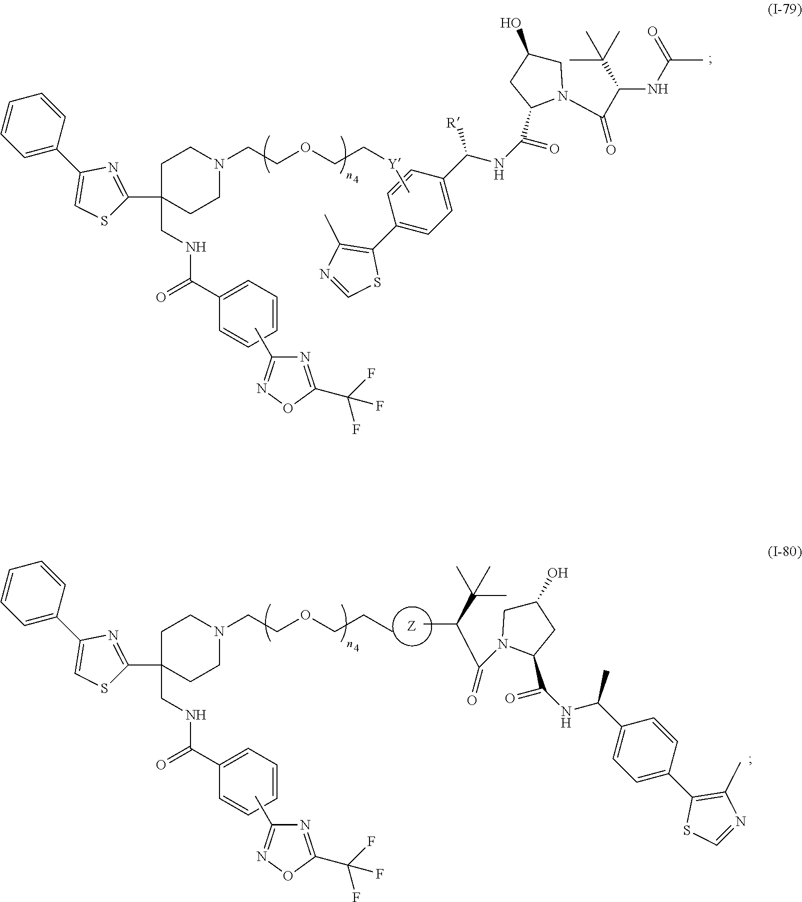

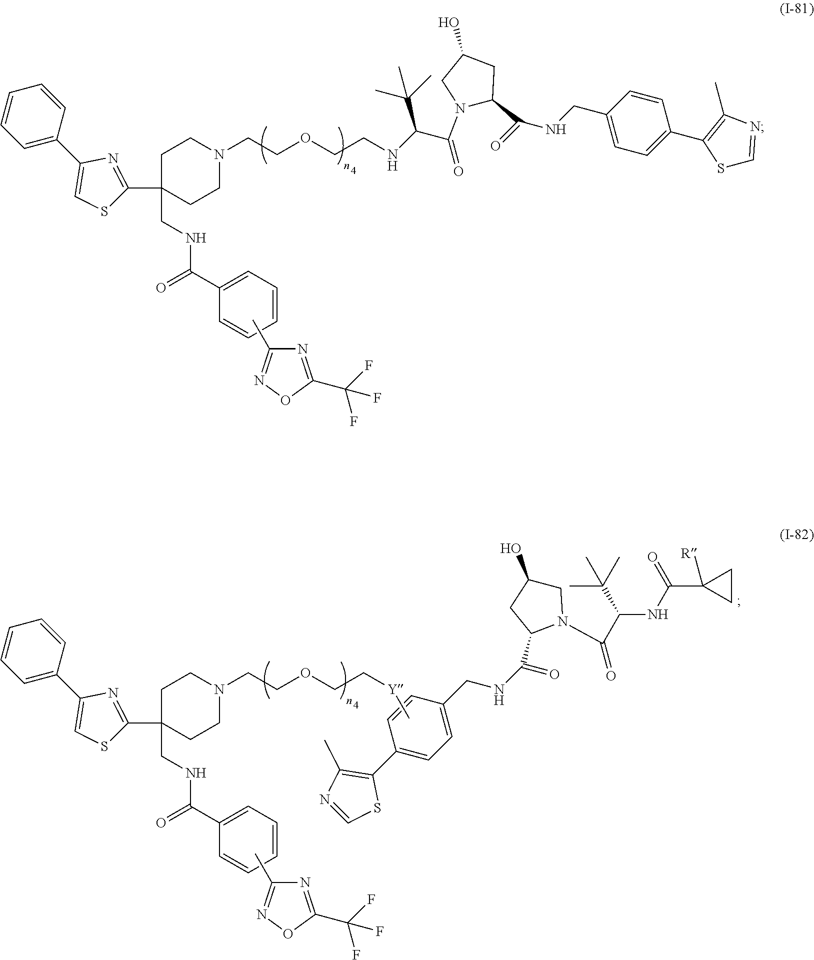

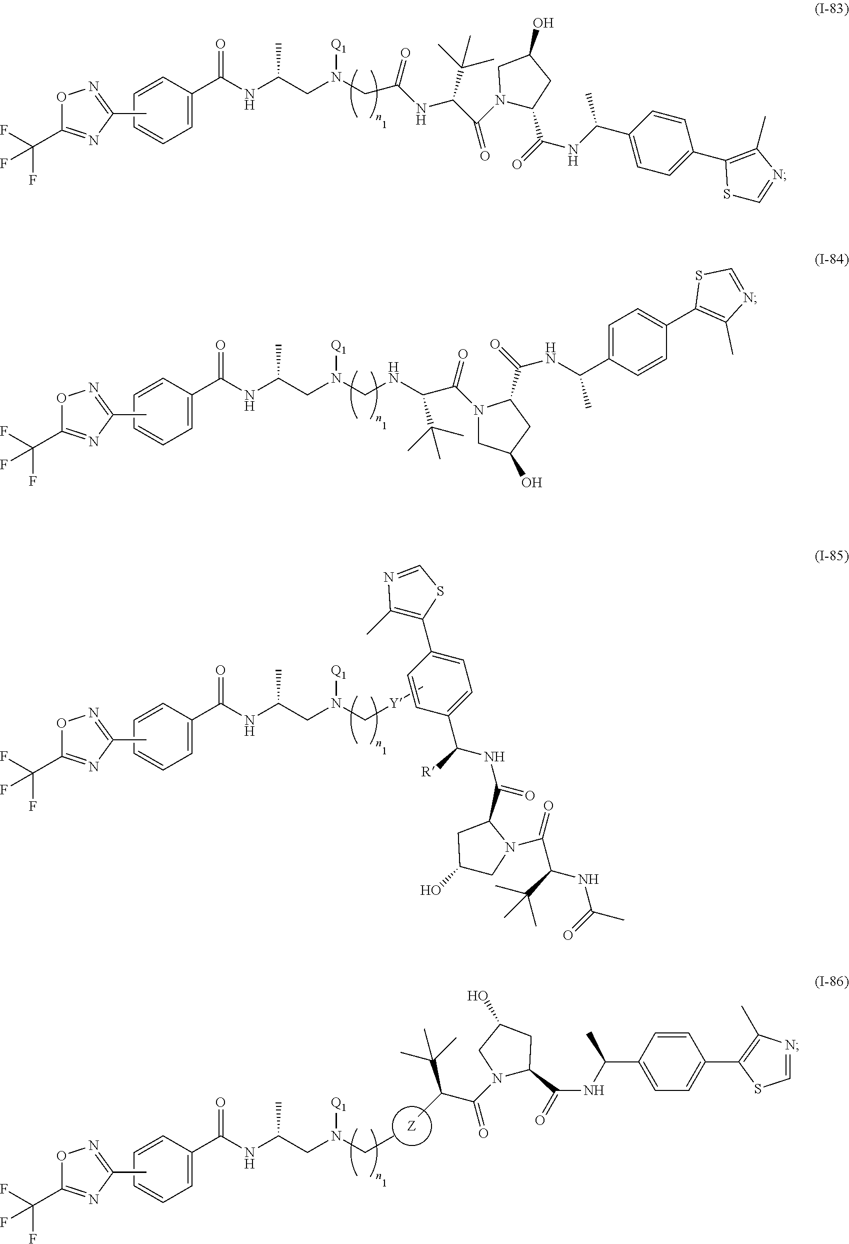

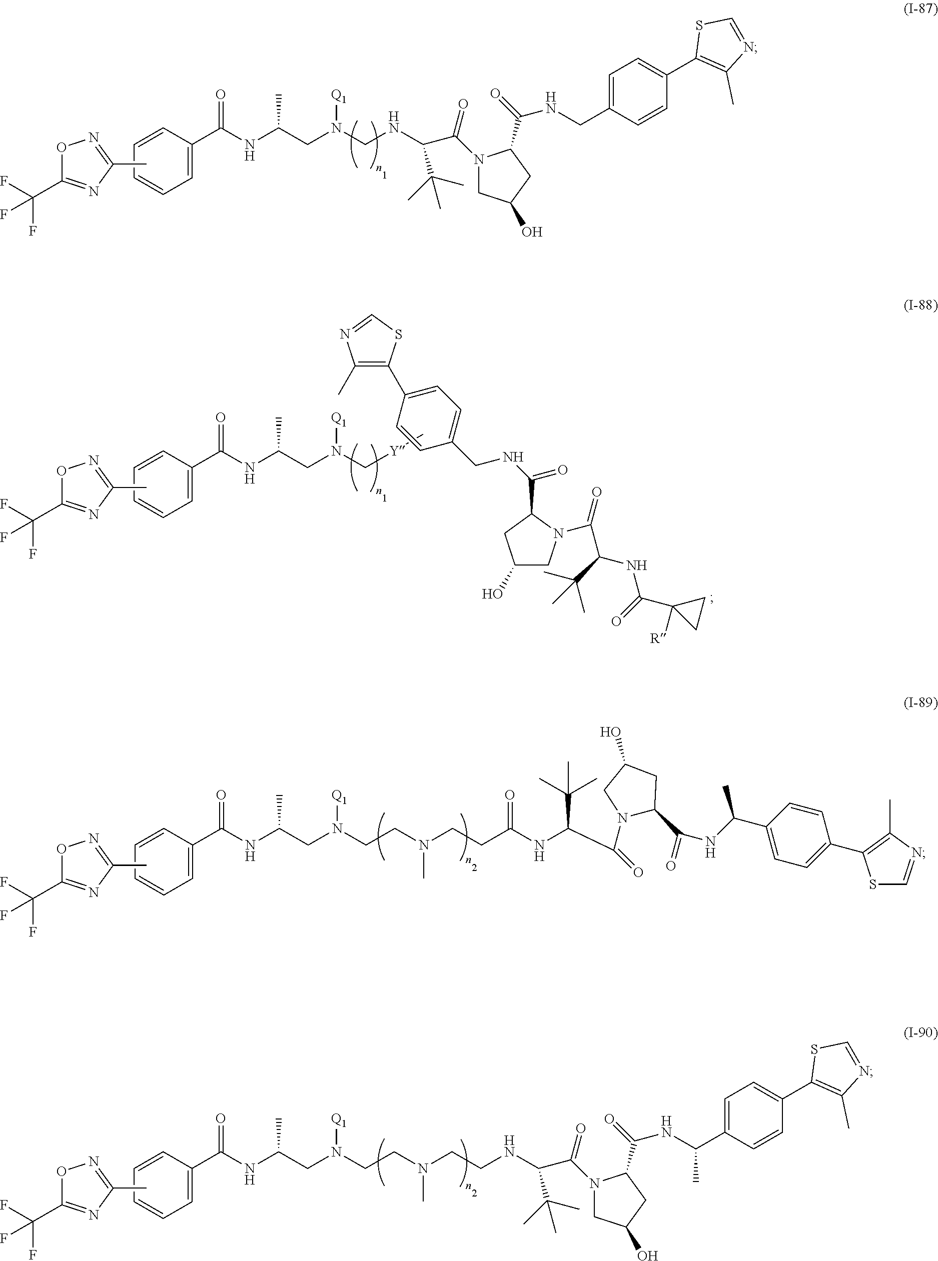

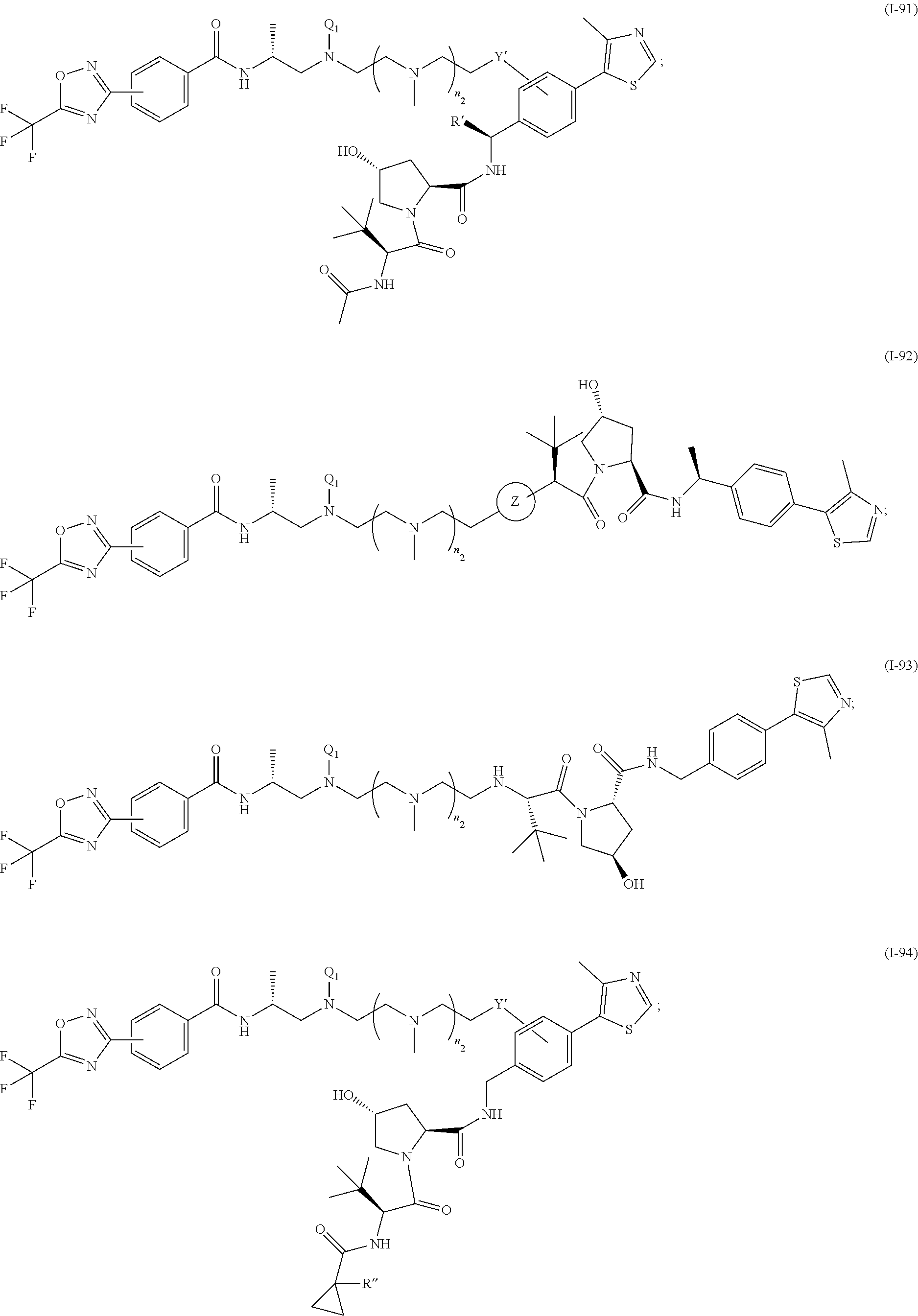

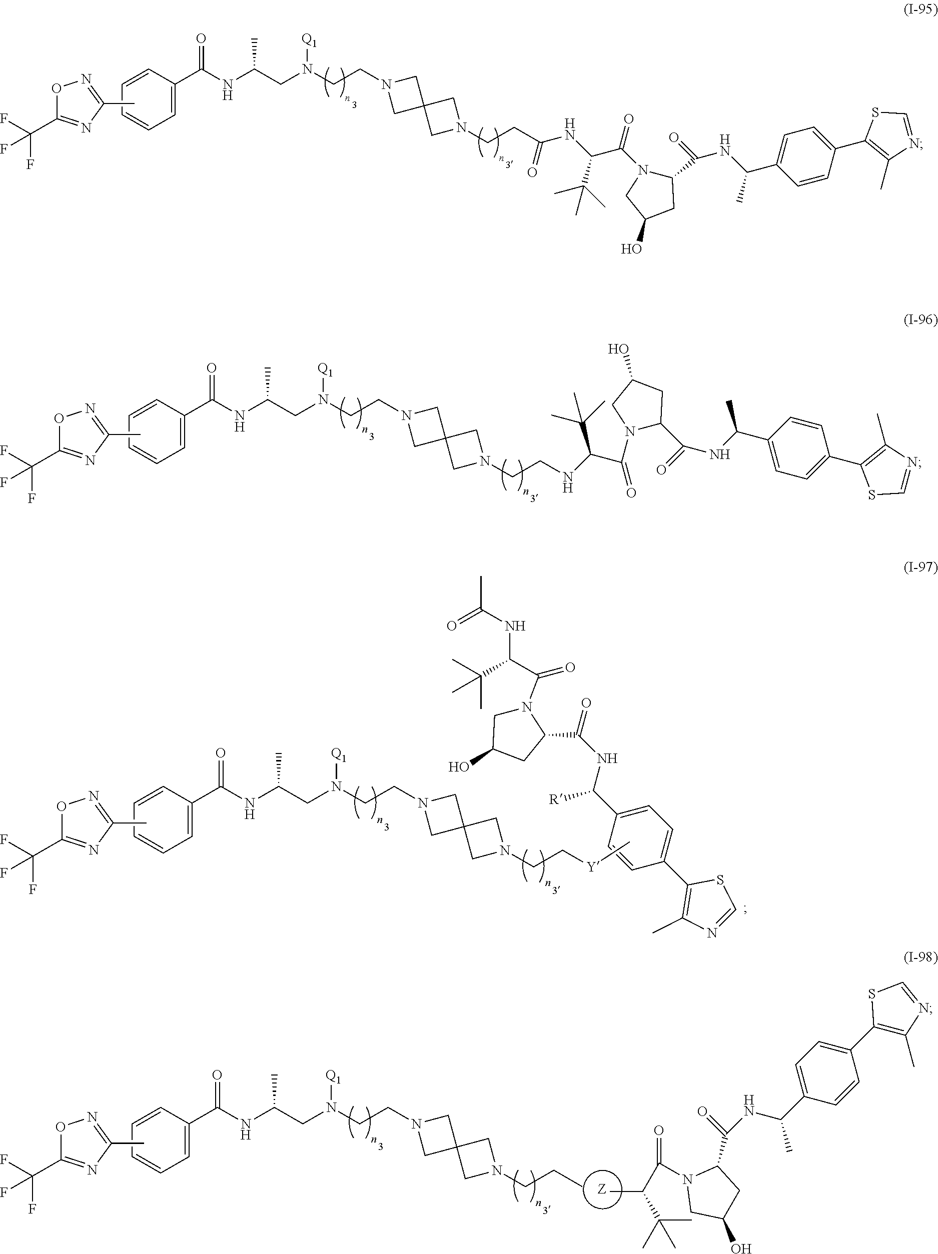

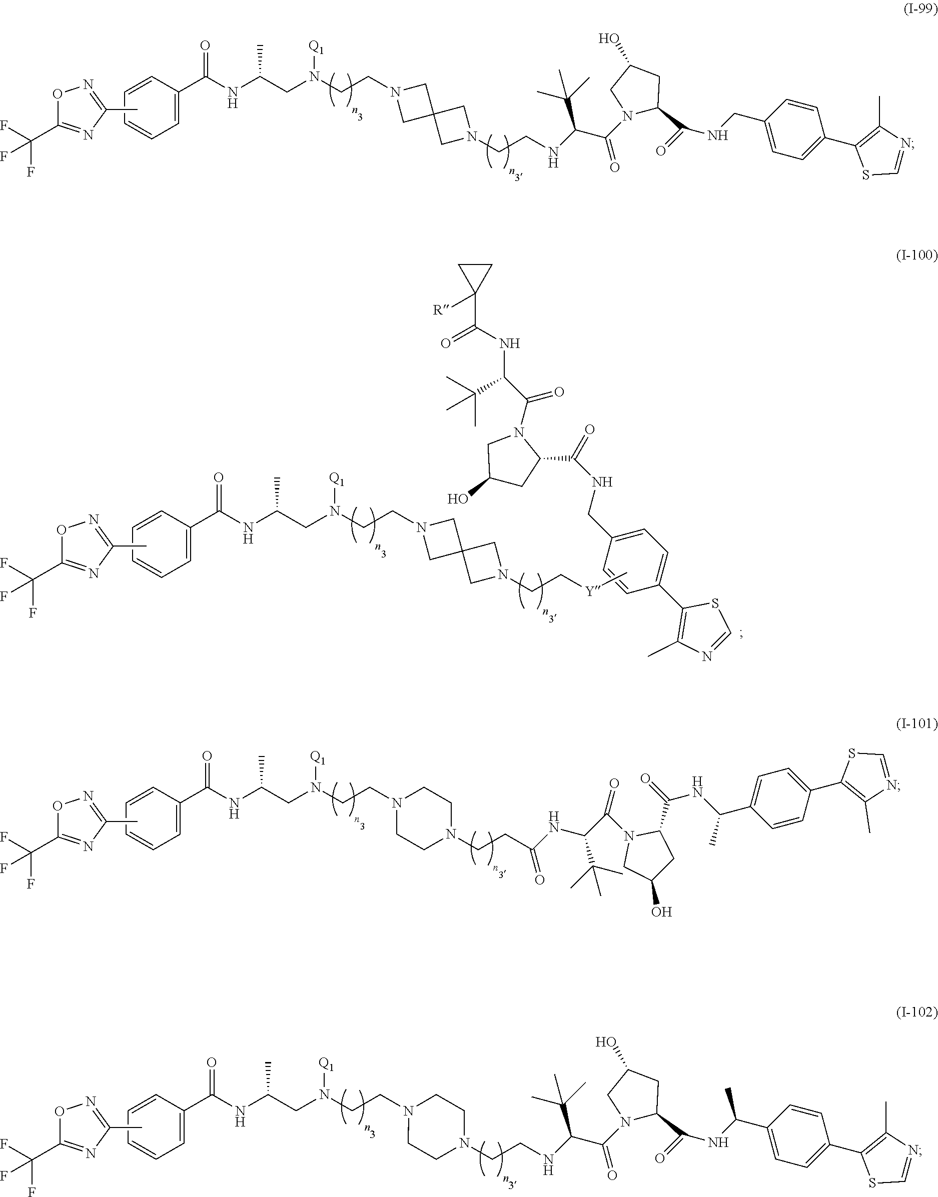

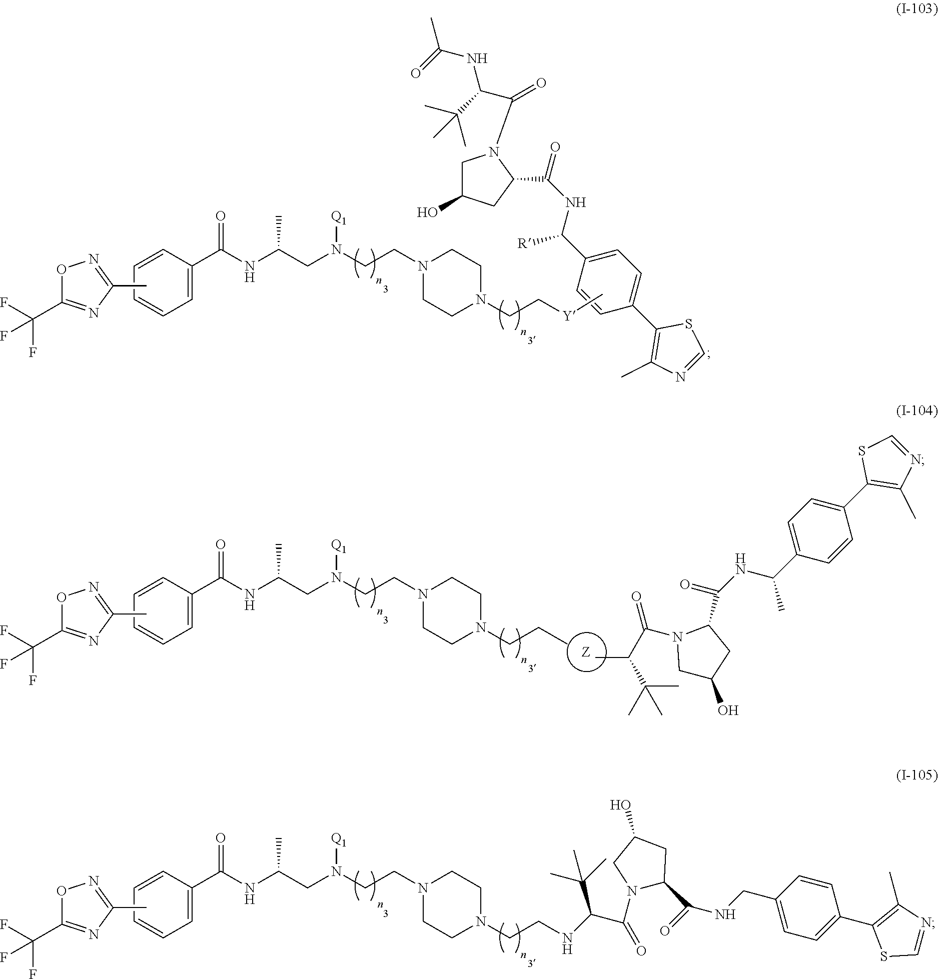

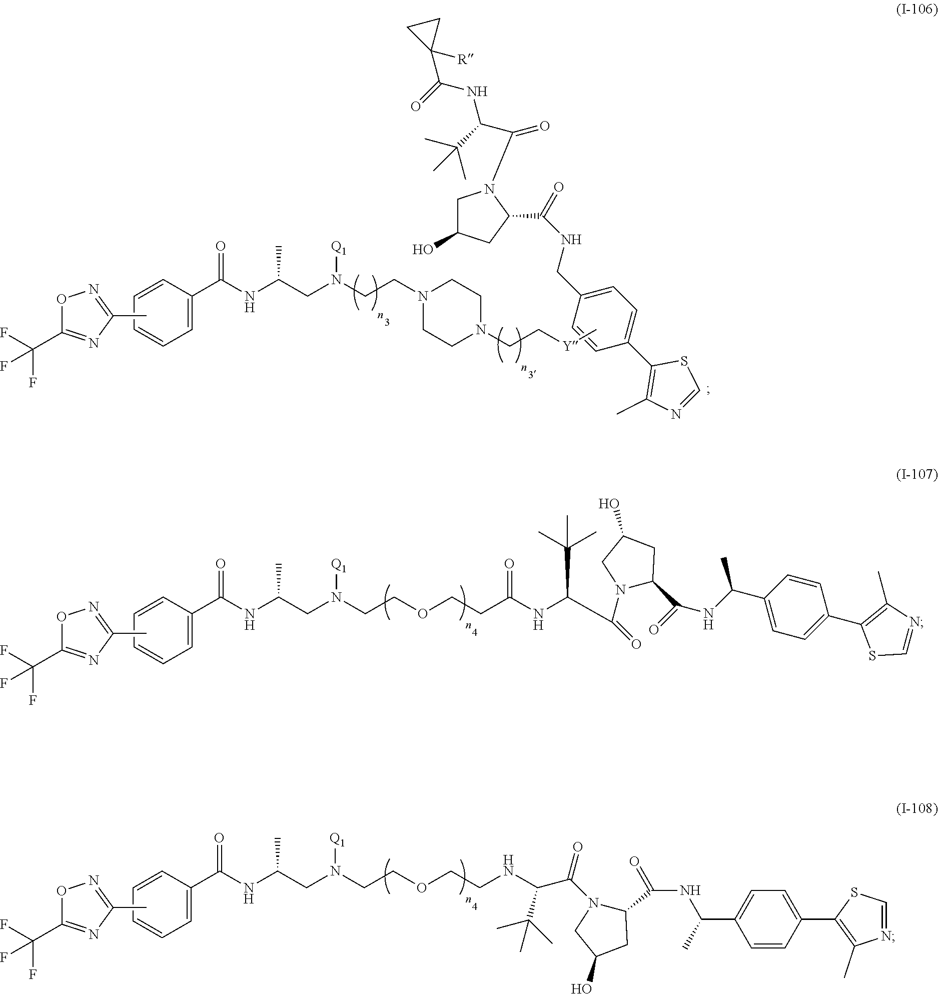

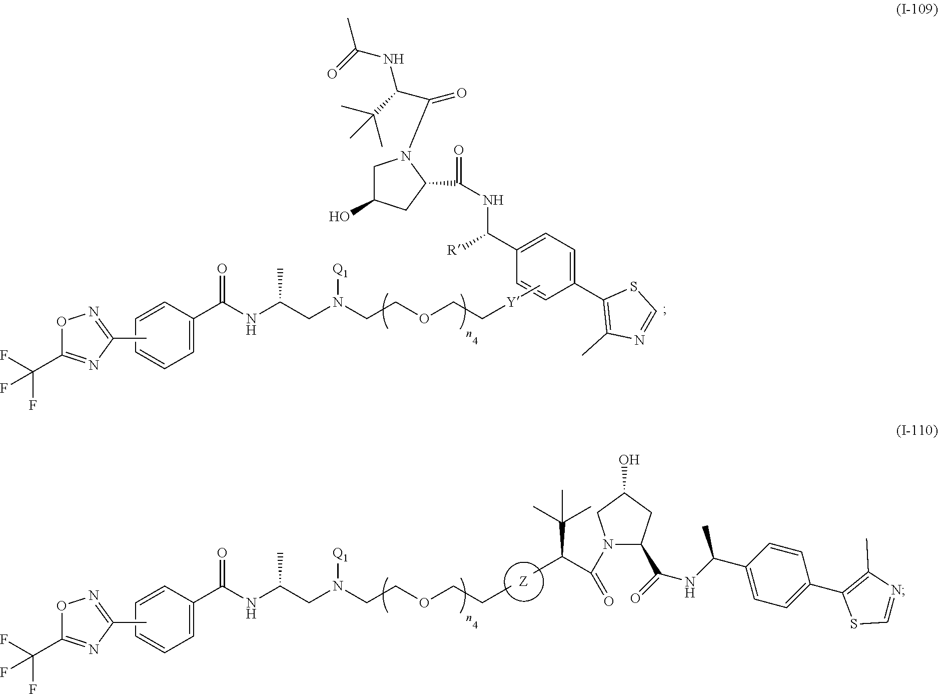

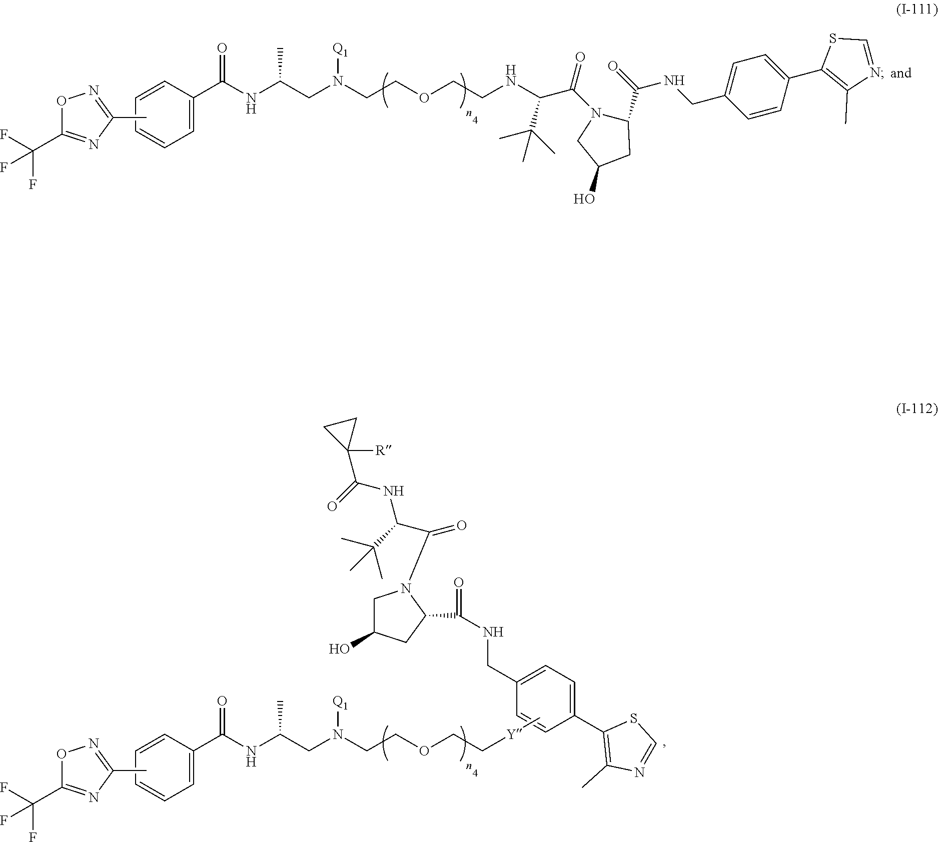

or a pharmaceutically acceptable salt or stereoisomer thereof.

In some embodiments, the degron binds an inhibitor of apoptosis protein (IAP), and is represented by any one of the following structures:

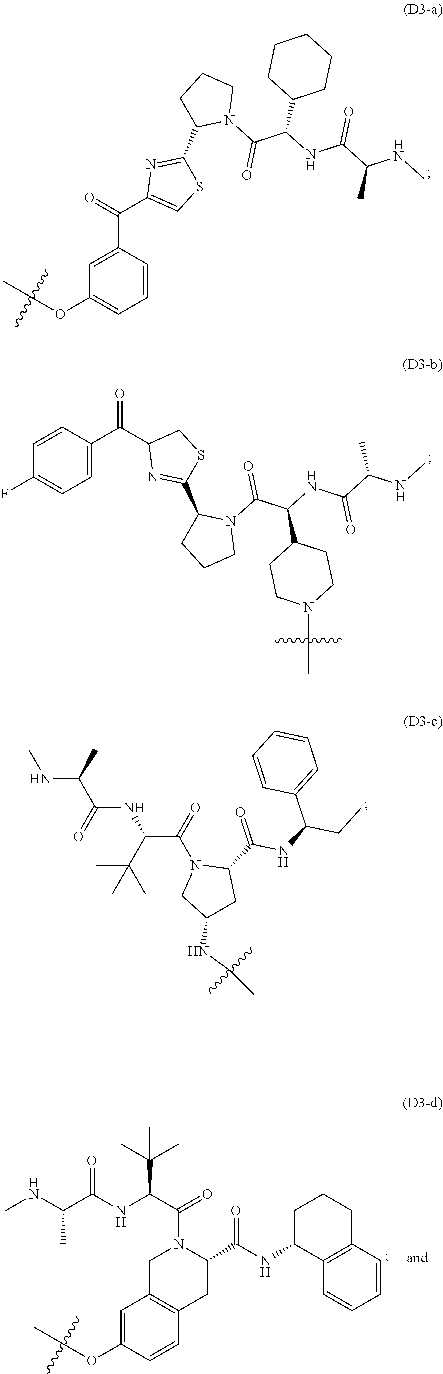

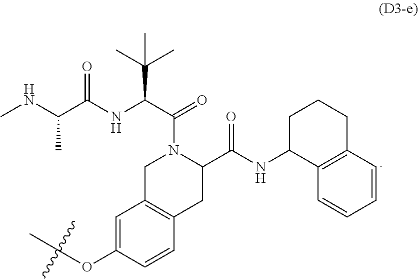 [SMILES: *Oc1ccc2c(c1)CN(C(=O)[C@@H](NC(=O)[C@H](C)NC)C(C)(C)C)C(C(=O)NC1CCCc3ccccc31)C2]

Yet other degrons that bind IAPs and which may be suitable for use as degrons in the present disclosure are disclosed in International Patent Application Publications WO 2008128171. WO 2008/016893, WO 2014/060768, WO 2014/060767, and WO 15092420. IAPs are known in the art to function as ubiquitin-E3 ligases.

Therefore, in some embodiments, the compounds of the present disclosure may be represented by any of structures (I-113) to (I-162):

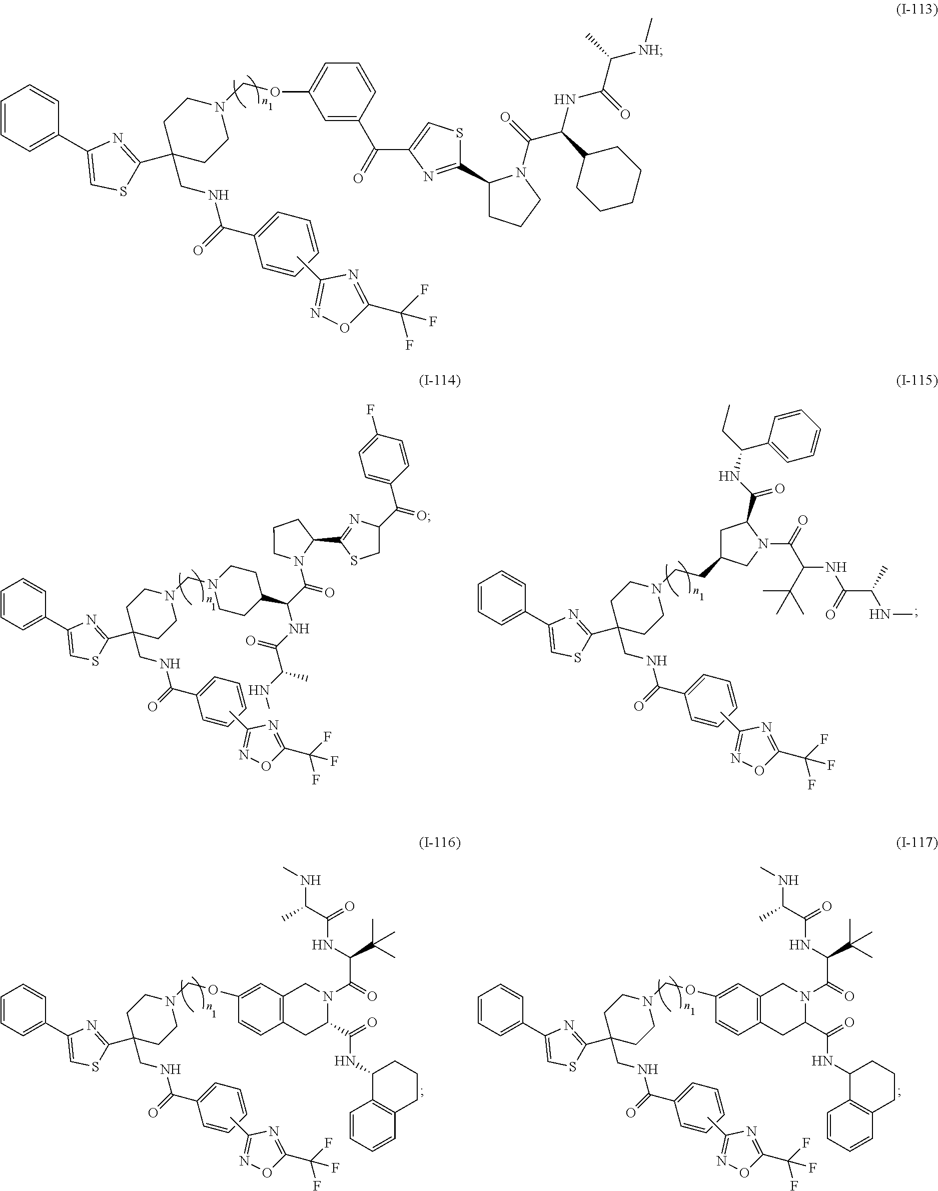

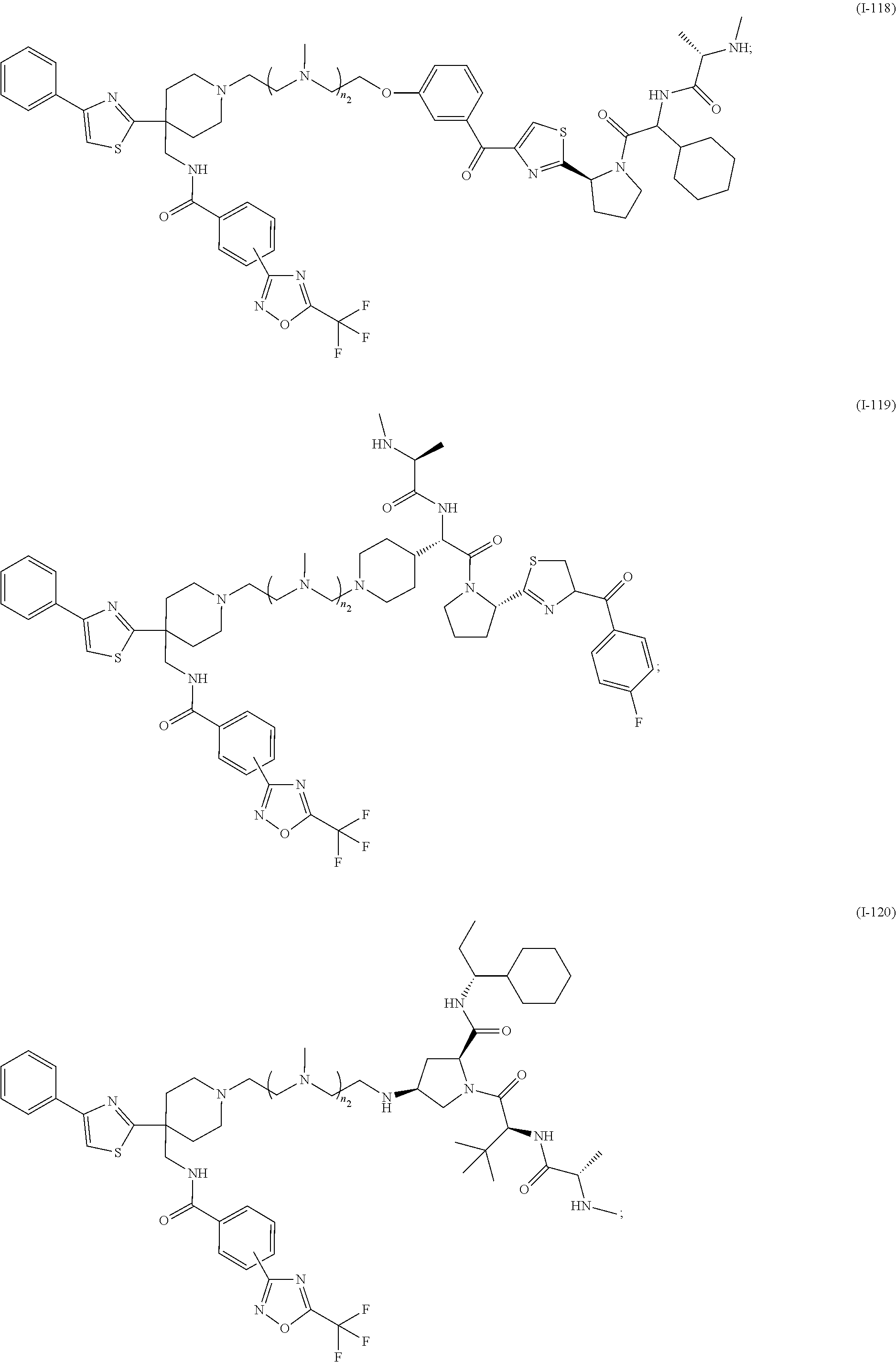

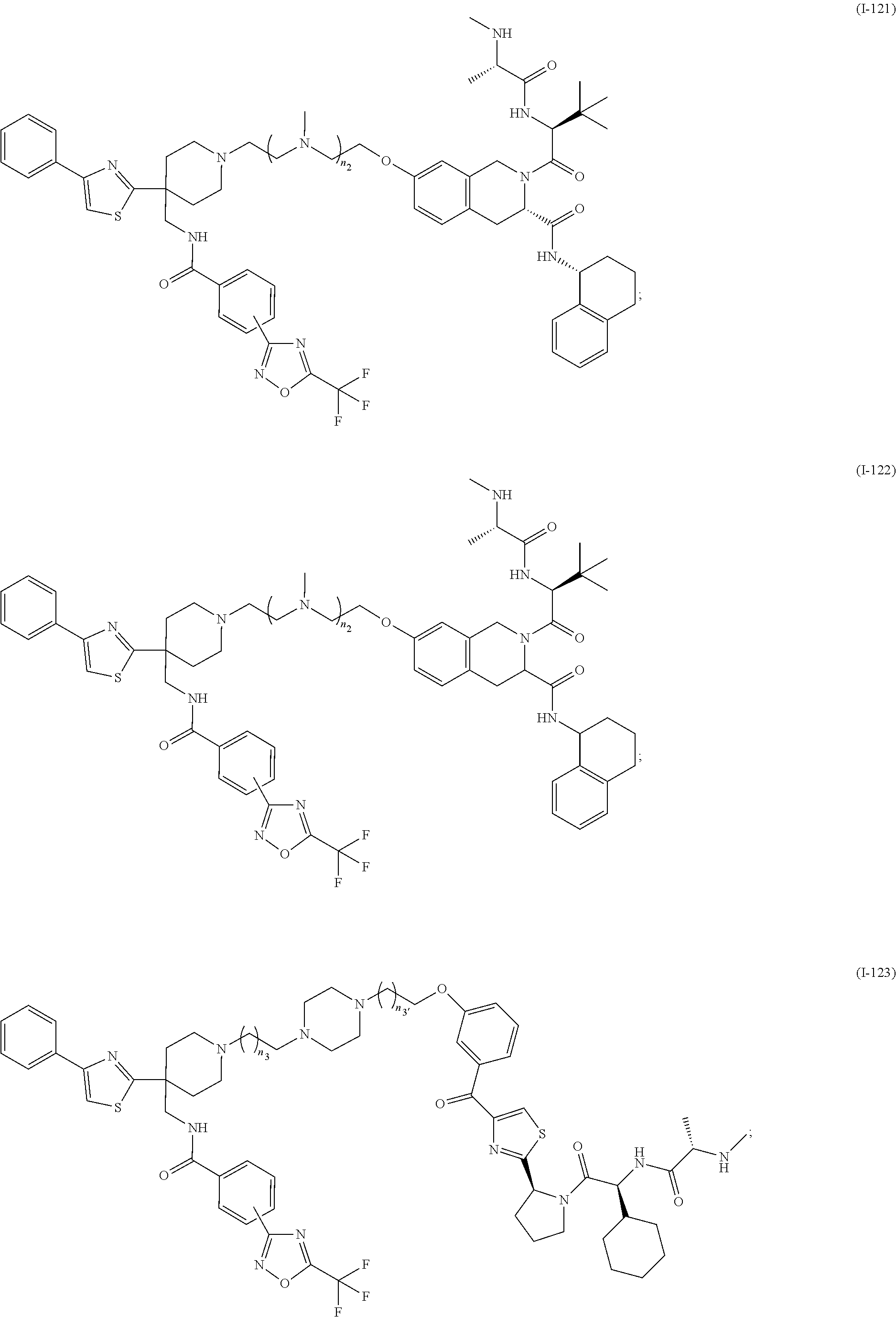

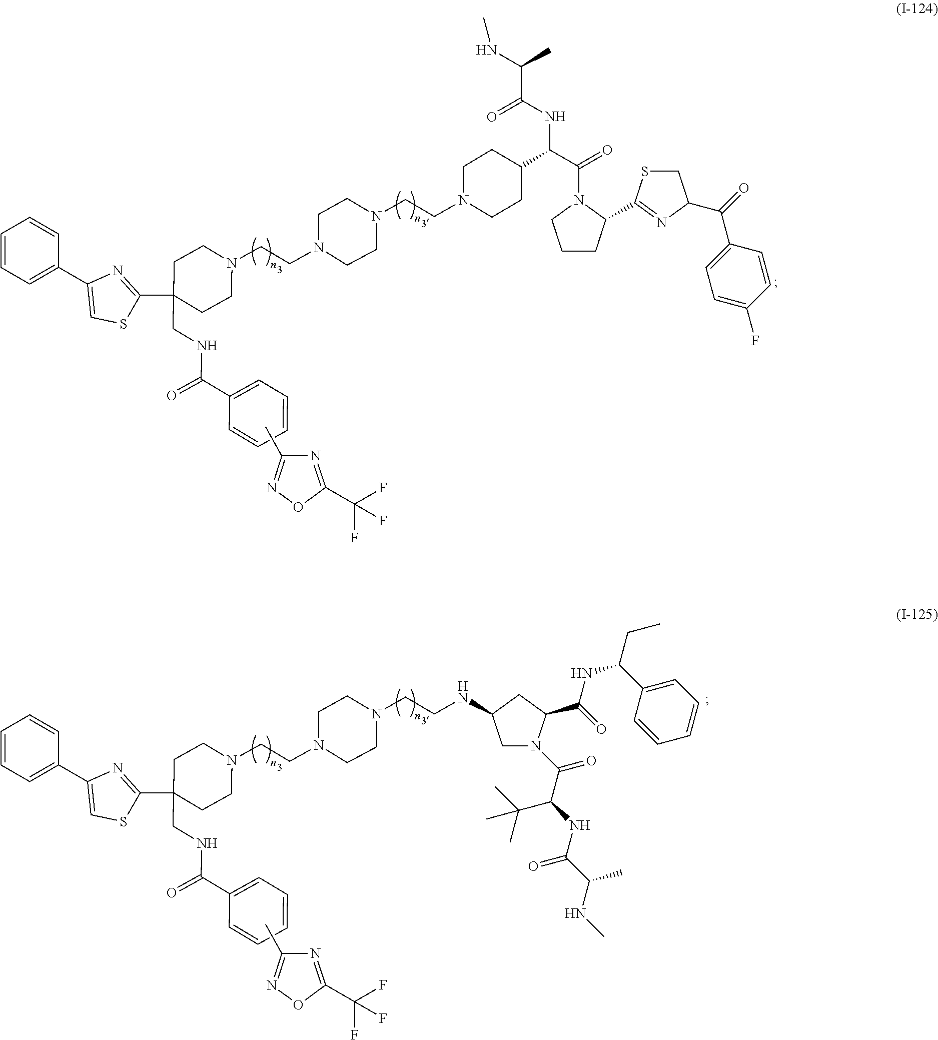

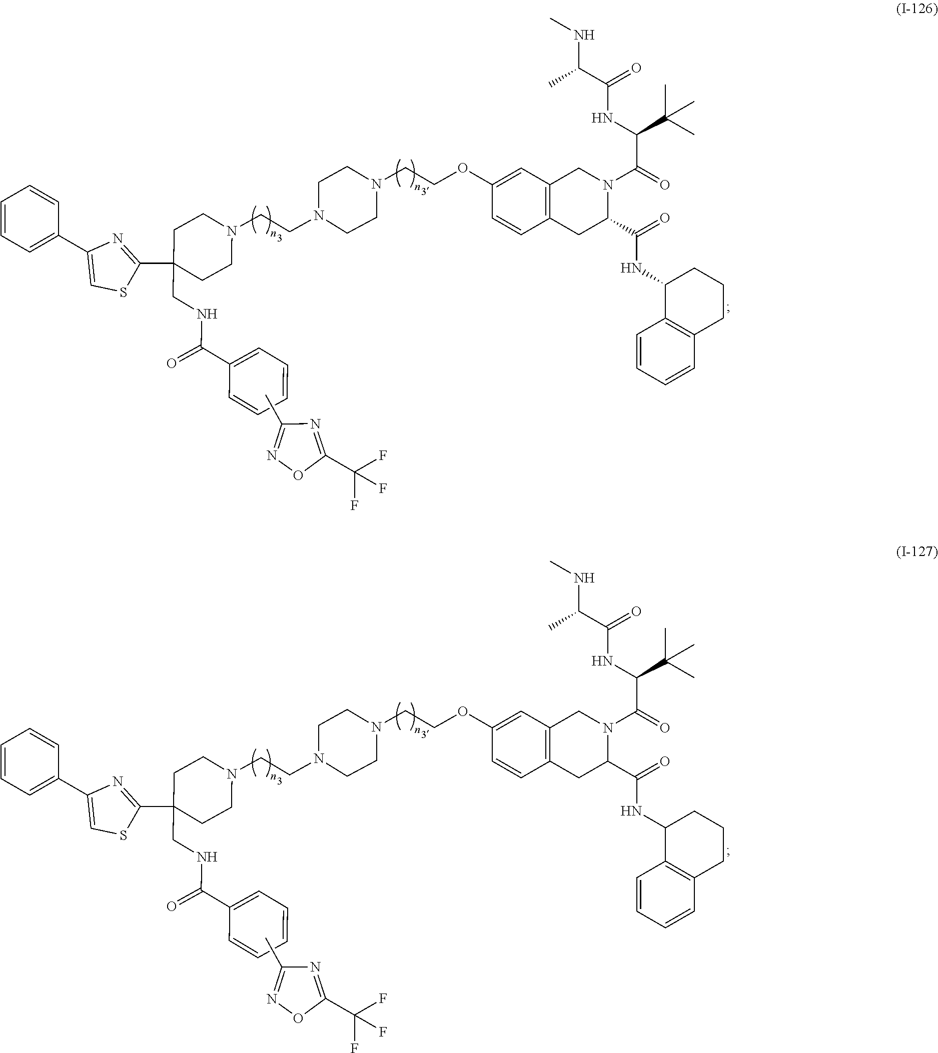

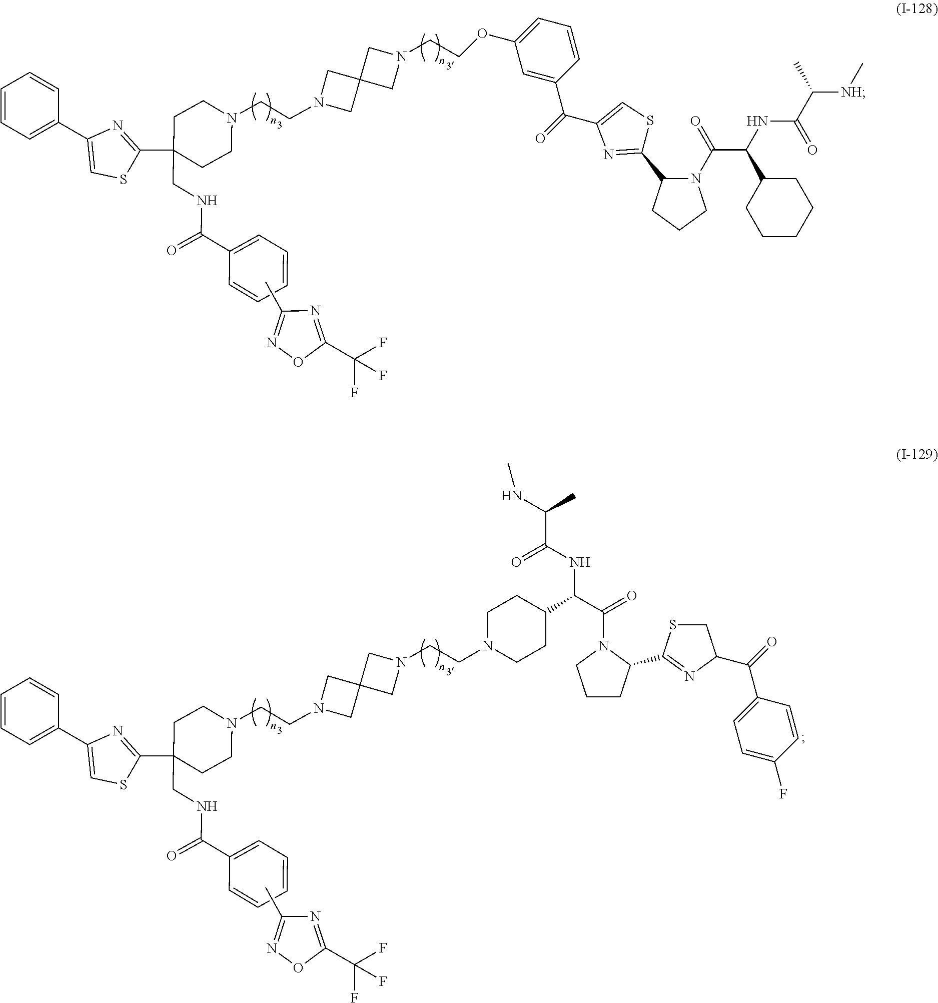

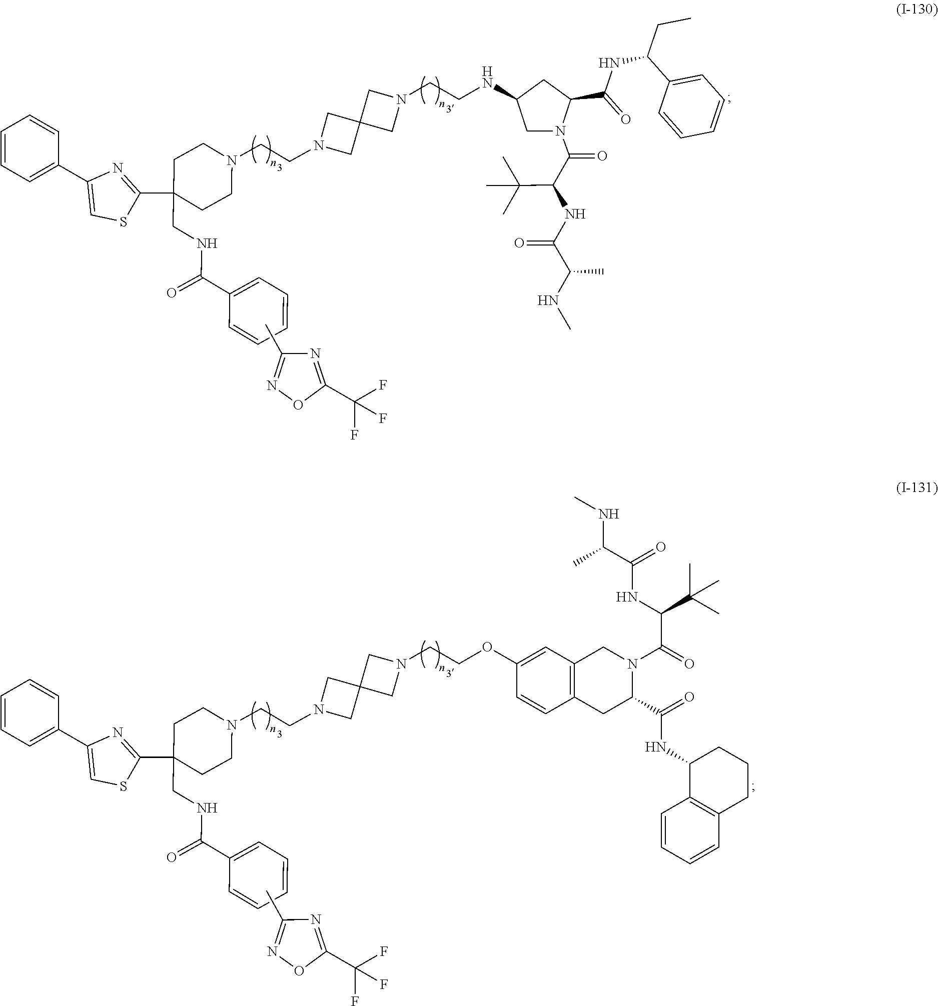

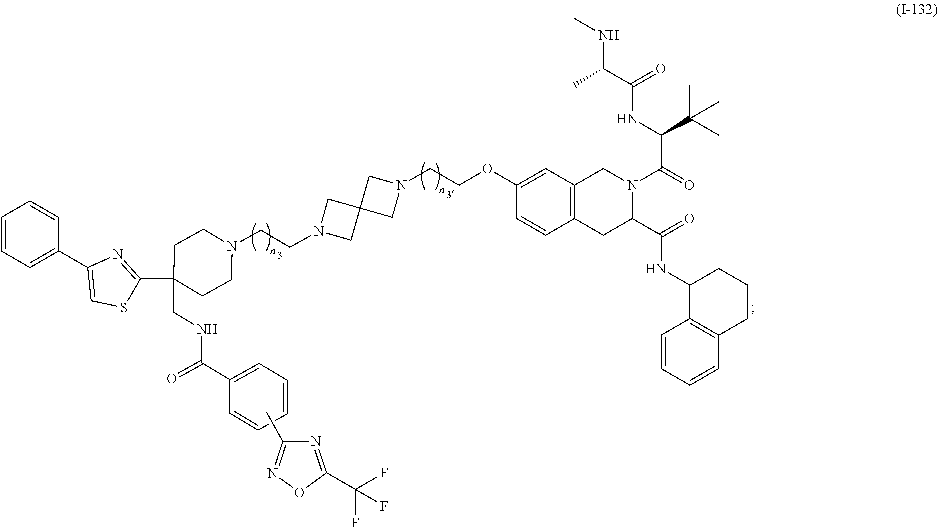

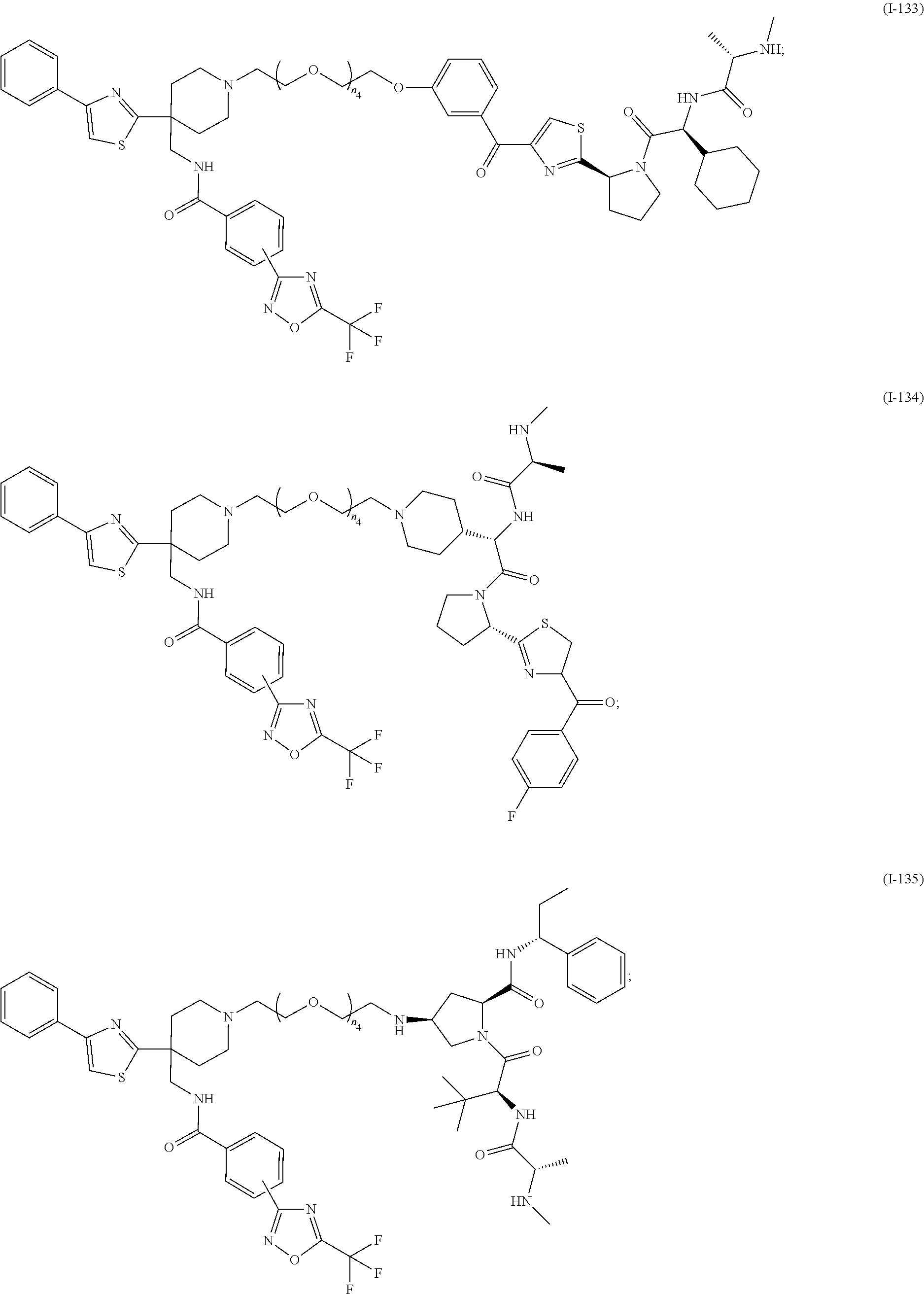

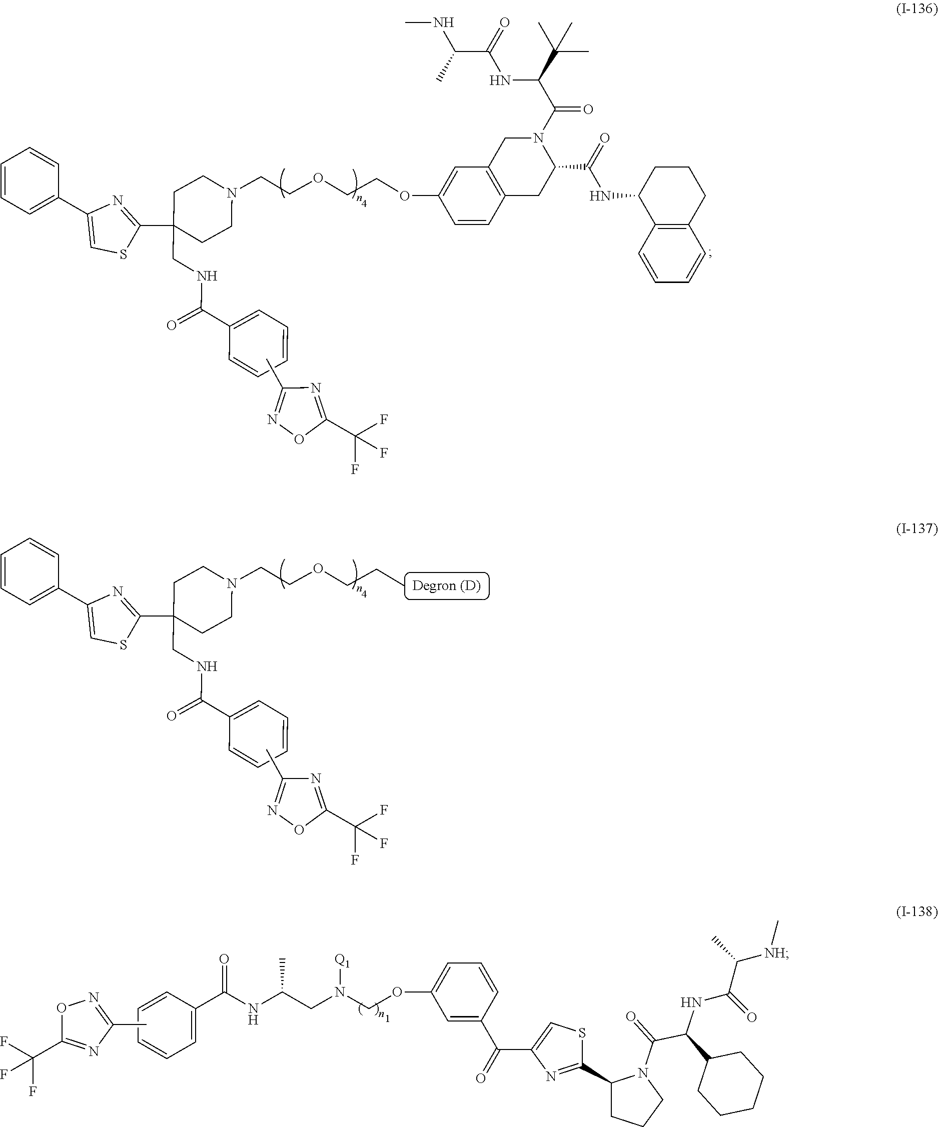

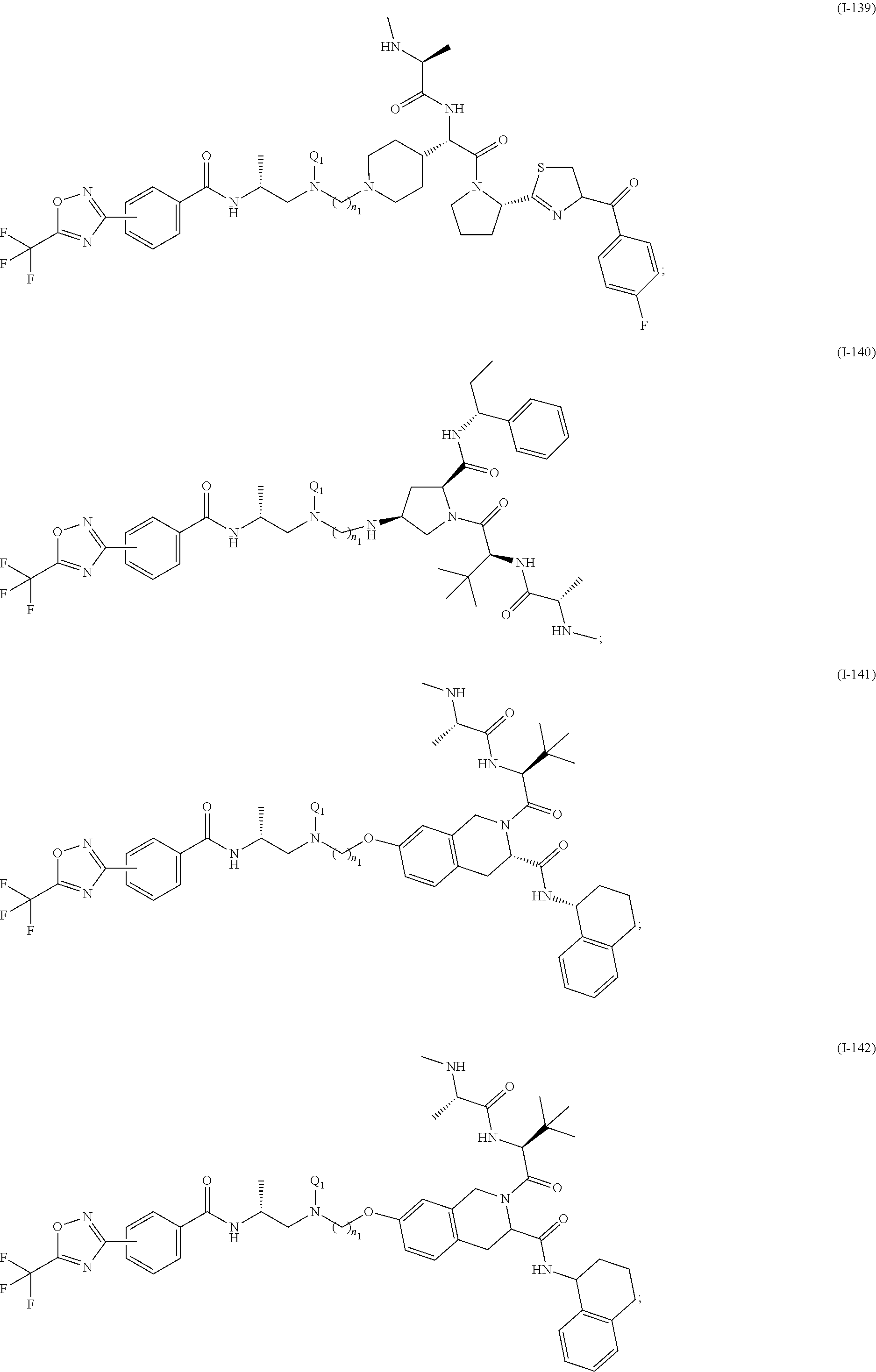

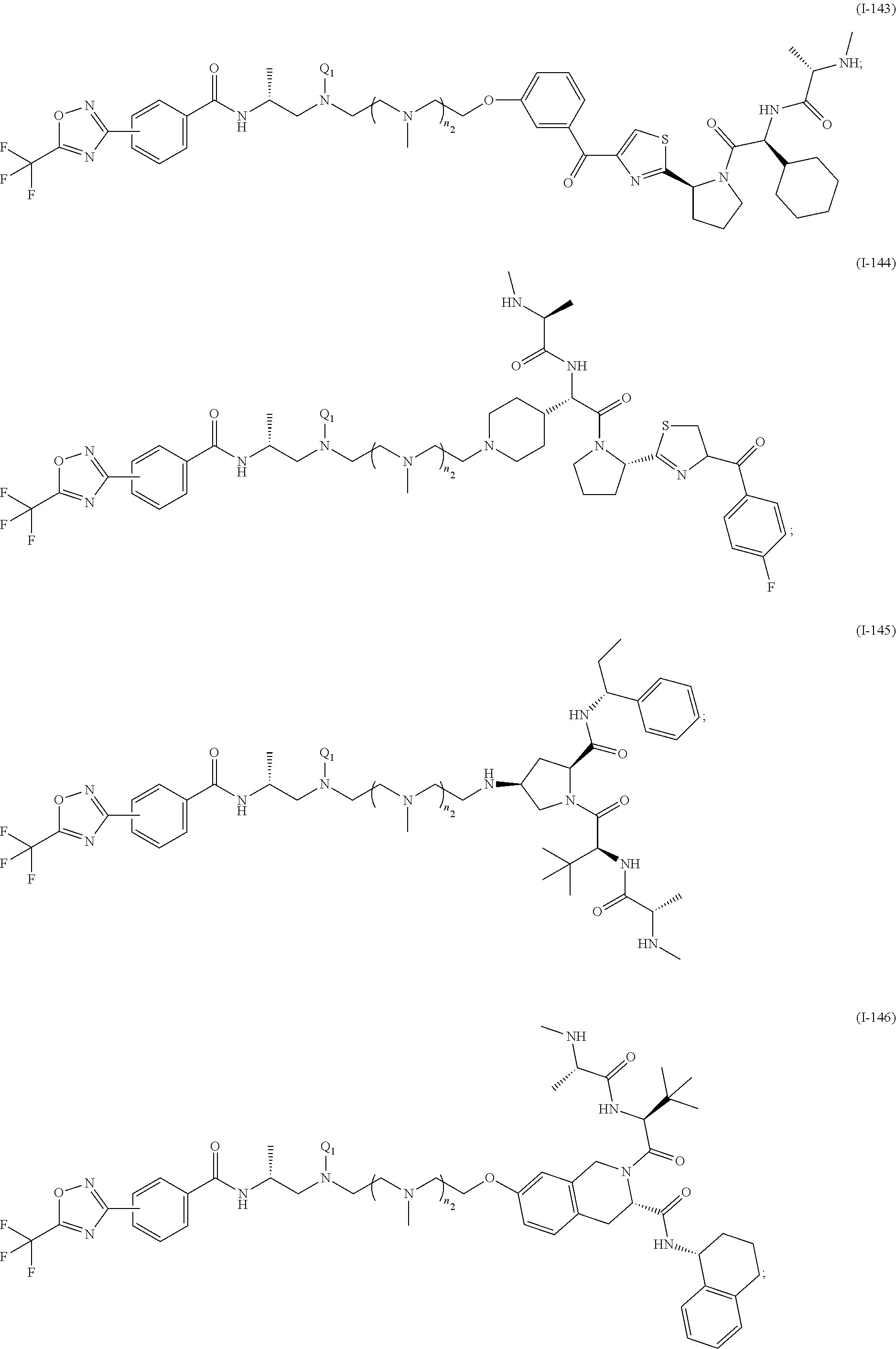

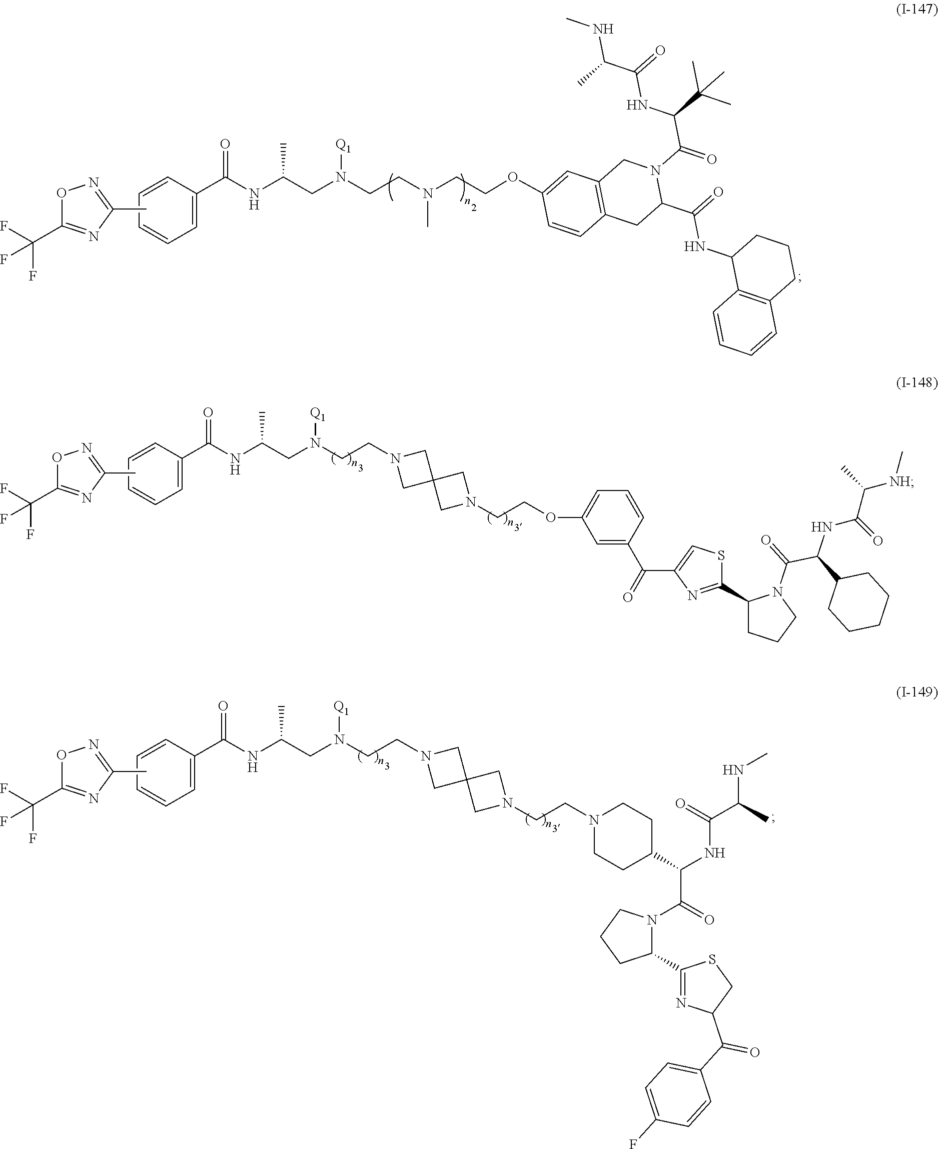

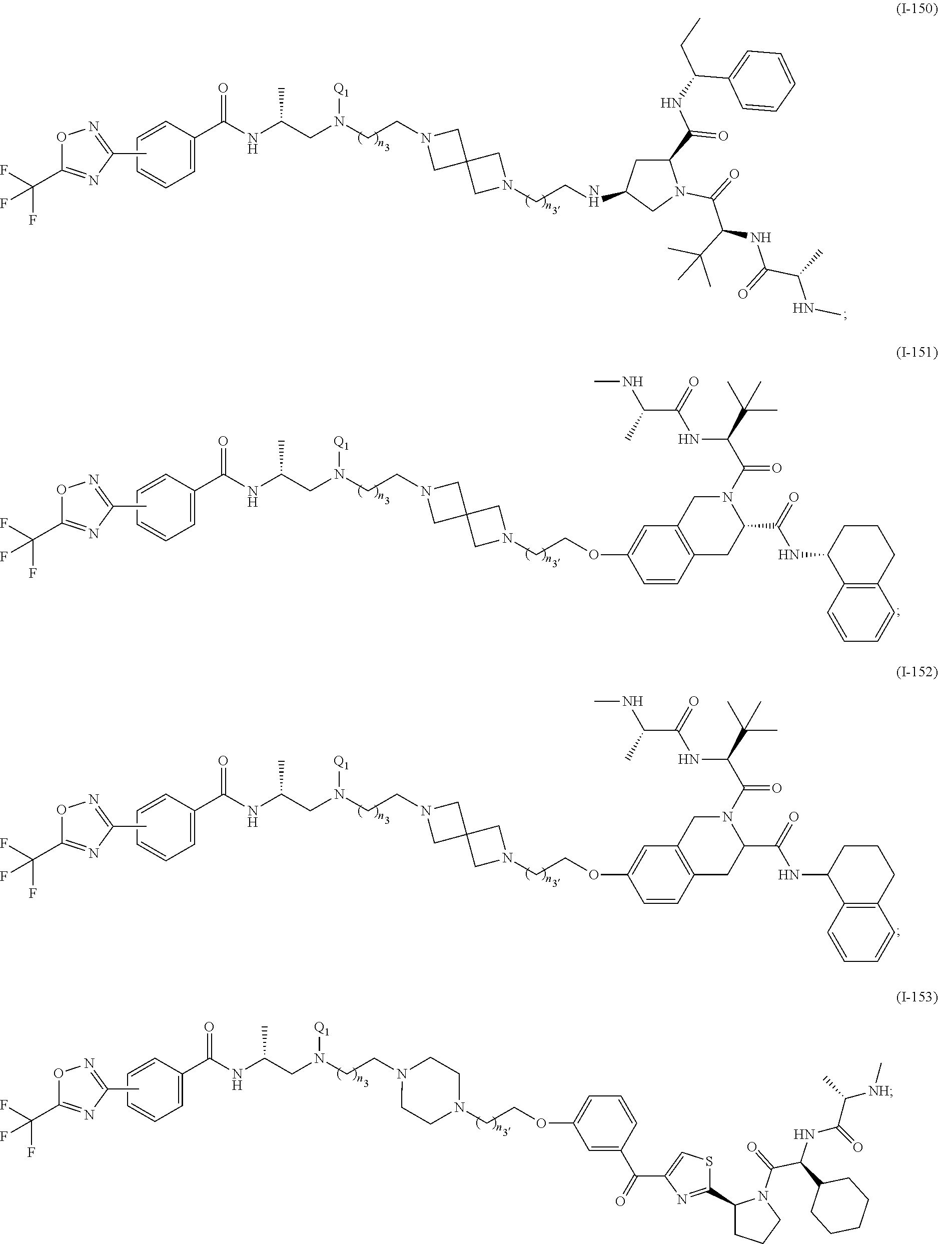

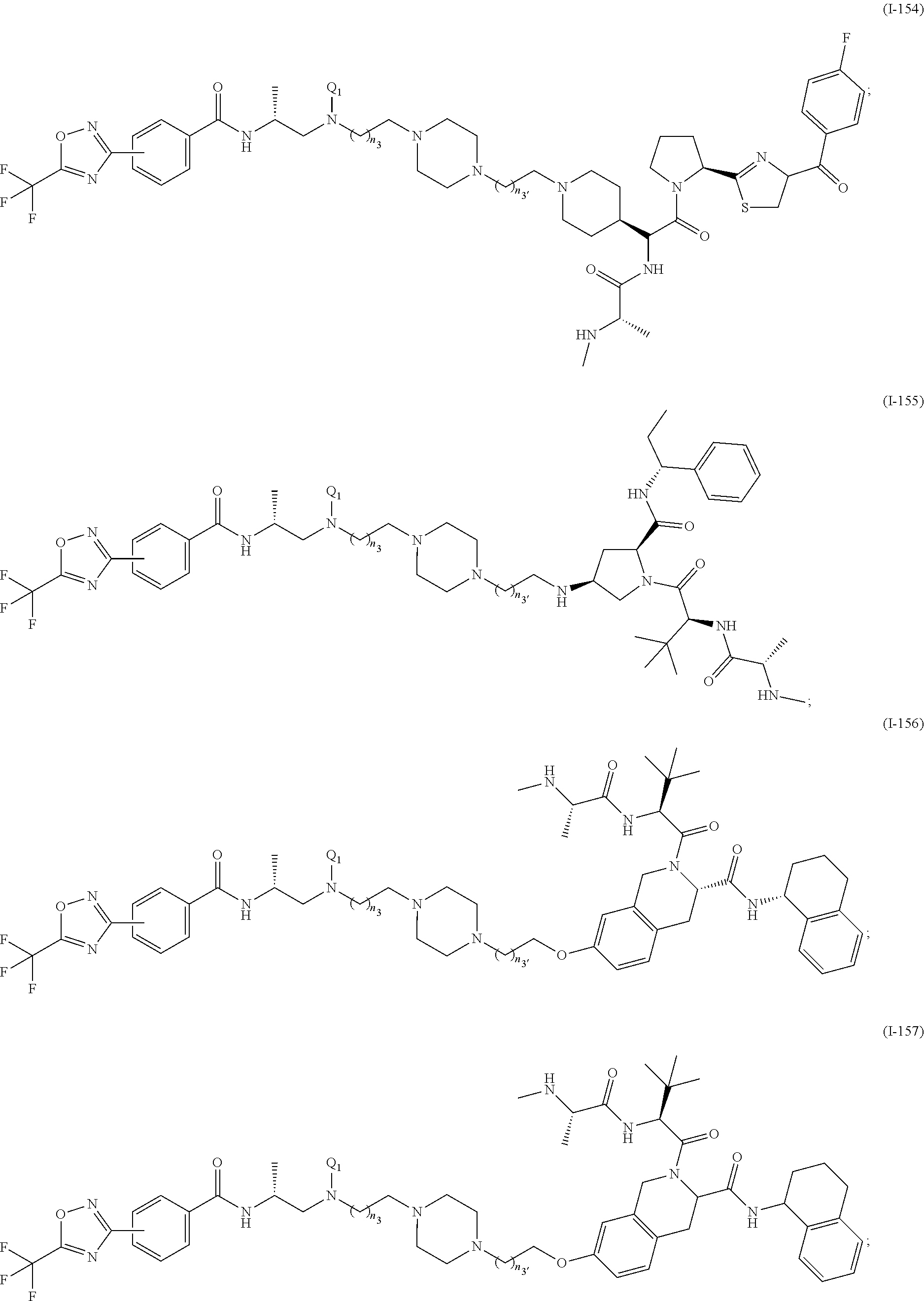

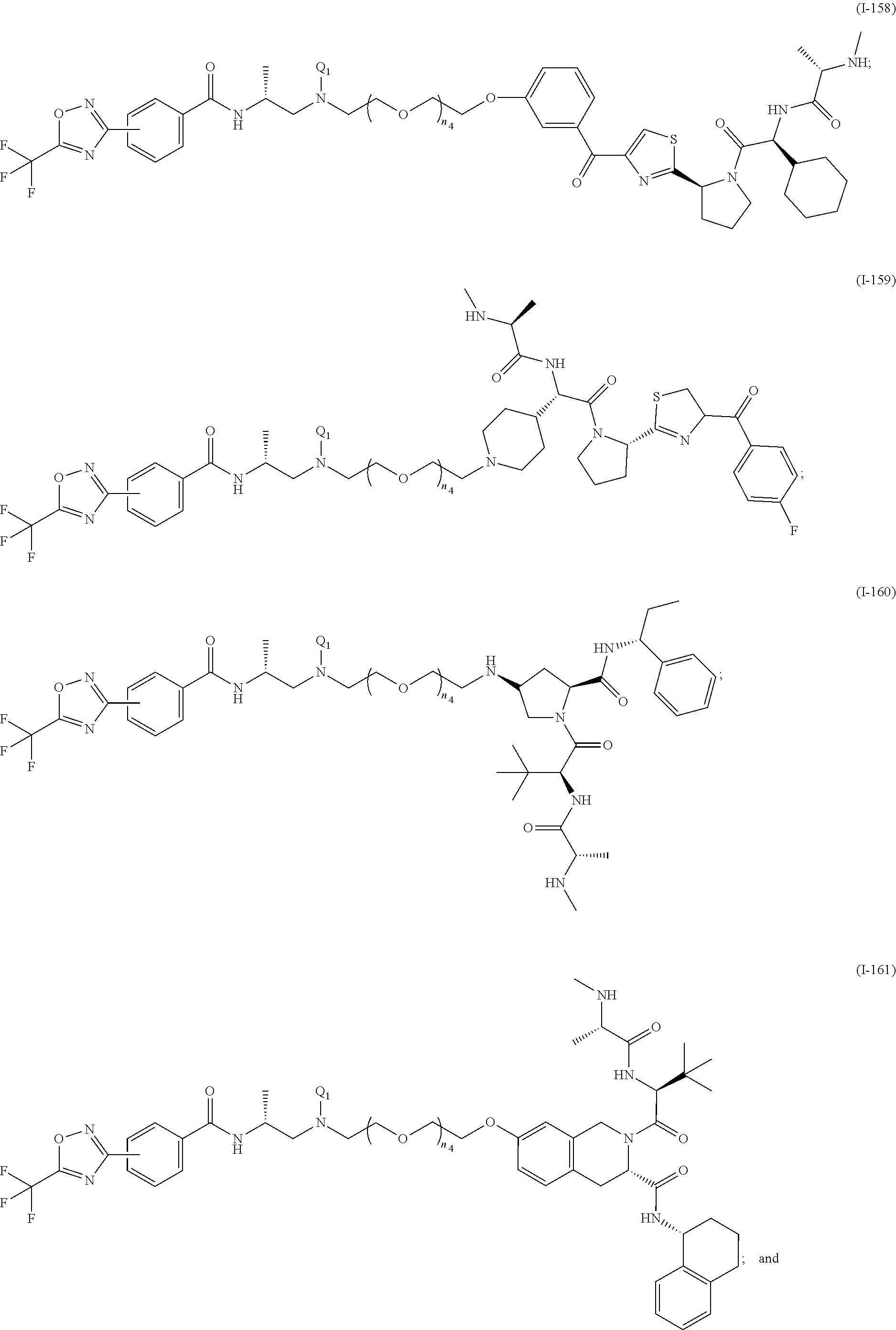

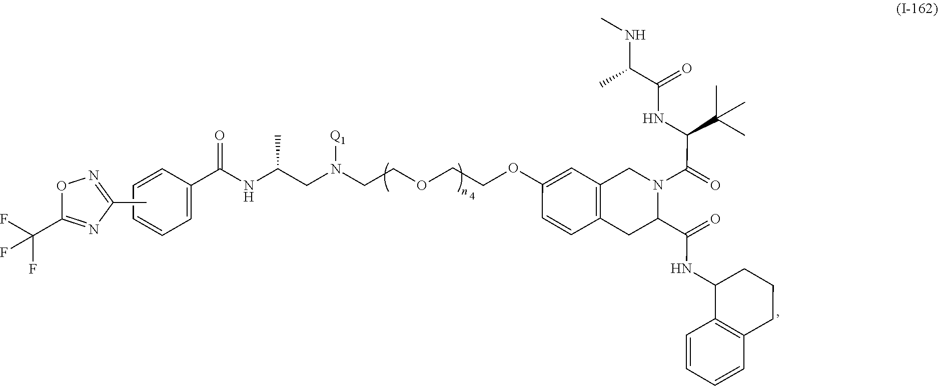

or a pharmaceutically acceptable salt or stereoisomer thereof.

In some embodiments, compounds of the present disclosure may be represented by any one of the following structures:

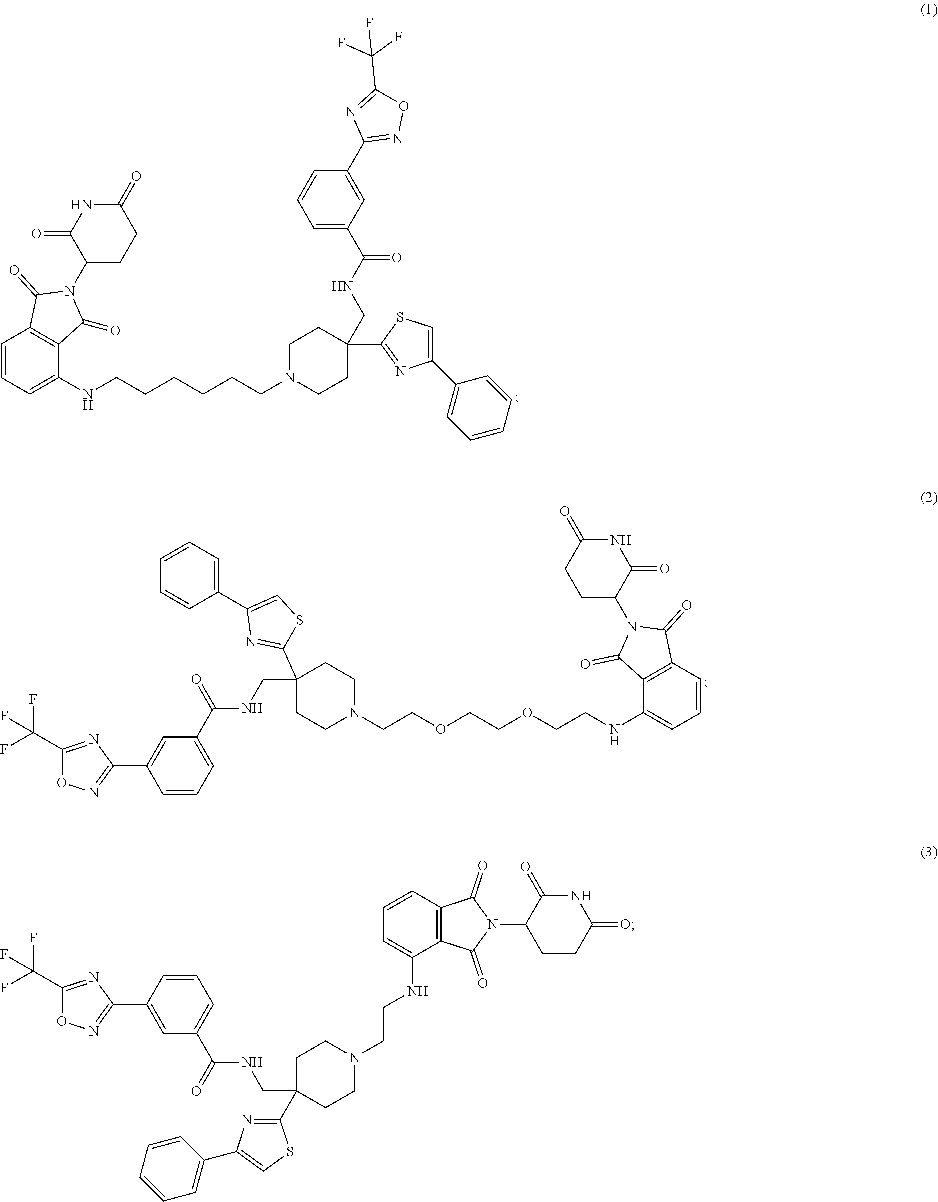 [SMILES: O=C1CCC(N2C(=O)c3cccc(NCCCCCCN4CCC(CNC(=O)c5cccc(-c6noc(C(F)(F)F)n6)c5)(c5nc(-c6ccccc6)cs5)CC4)c3C2=O)C(=O)N1 ; O=C1CCC(N2C(=O)c3cccc(NCCOCCOCCN4CCC(CNC(=O)c5cccc(-c6noc(C(F)(F)F)n6)c5)(c5nc(-c6ccccc6)cs5)CC4)c3C2=O)C(=O)N1 ; O=C1CCC(N2C(=O)c3cccc(NCCN4CCC(CNC(=O)c5cccc(-c6noc(C(F)(F)F)n6)c5)(c5nc(-c6ccccc6)cs5)CC4)c3C2=O)C(=O)N1]

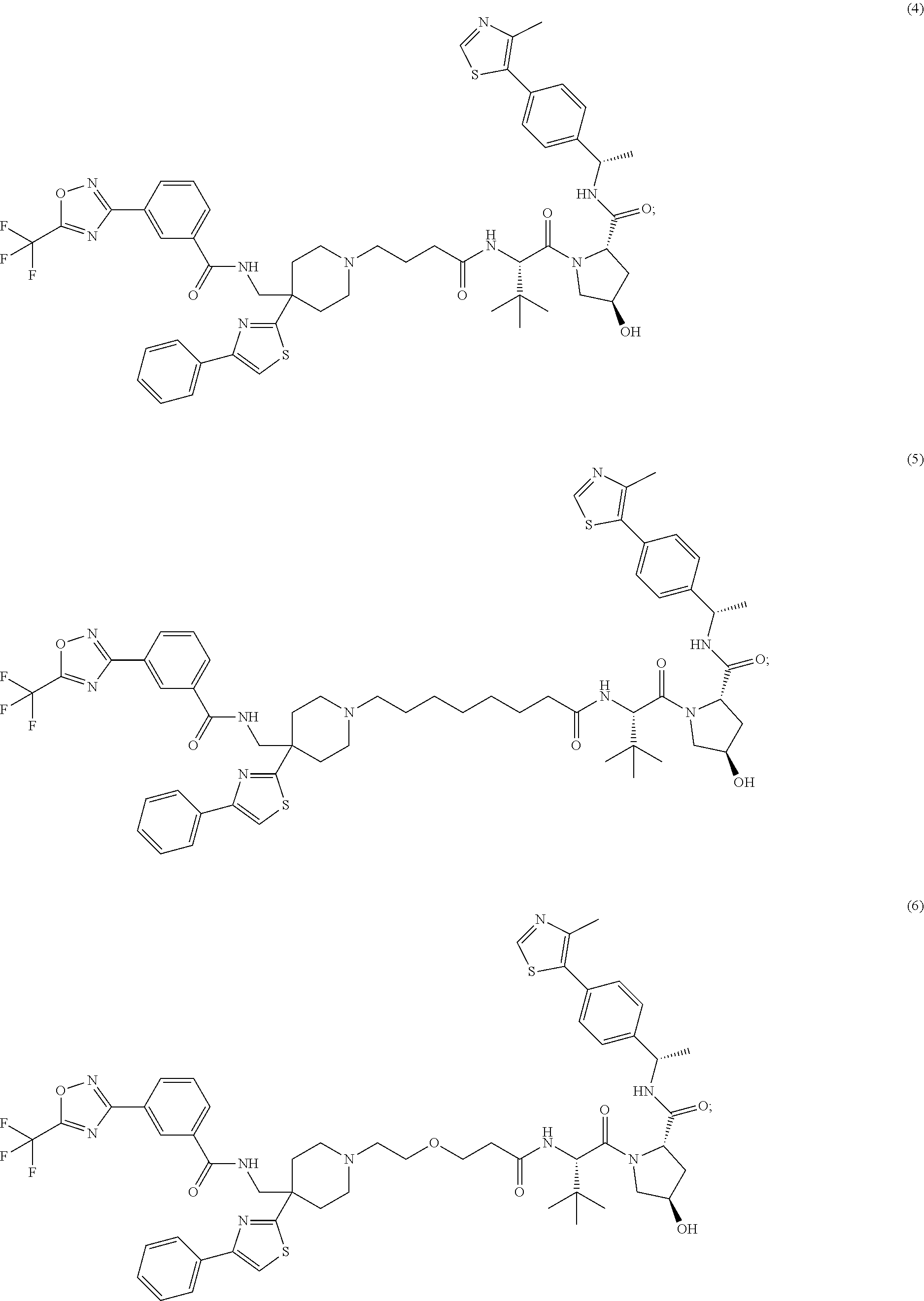 [SMILES: Cc1ncsc1-c1ccc([C@H](C)NC(=O)[C@@H]2C[C@@H](O)CN2C(=O)[C@@H](NC(=O)CCCN2CCC(CNC(=O)c3cccc(-c4noc(C(F)(F)F)n4)c3)(c3nc(-c4ccccc4)cs3)CC2)C(C)(C)C)cc1 ; Cc1ncsc1-c1ccc([C@H](C)NC(=O)[C@@H]2C[C@@H](O)CN2C(=O)[C@@H](NC(=O)CCCCCCCN2CCC(CNC(=O)c3cccc(-c4noc(C(F)(F)F)n4)c3)(c3nc(-c4ccccc4)cs3)CC2)C(C)(C)C)cc1 ; Cc1ncsc1-c1ccc([C@H](C)NC(=O)[C@@H]2C[C@@H](O)CN2C(=O)[C@@H](NC(=O)CCOCCN2CCC(CNC(=O)c3cccc(-c4noc(C(F)(F)F)n4)c3)(c3nc(-c4ccccc4)cs3)CC2)C(C)(C)C)cc1]

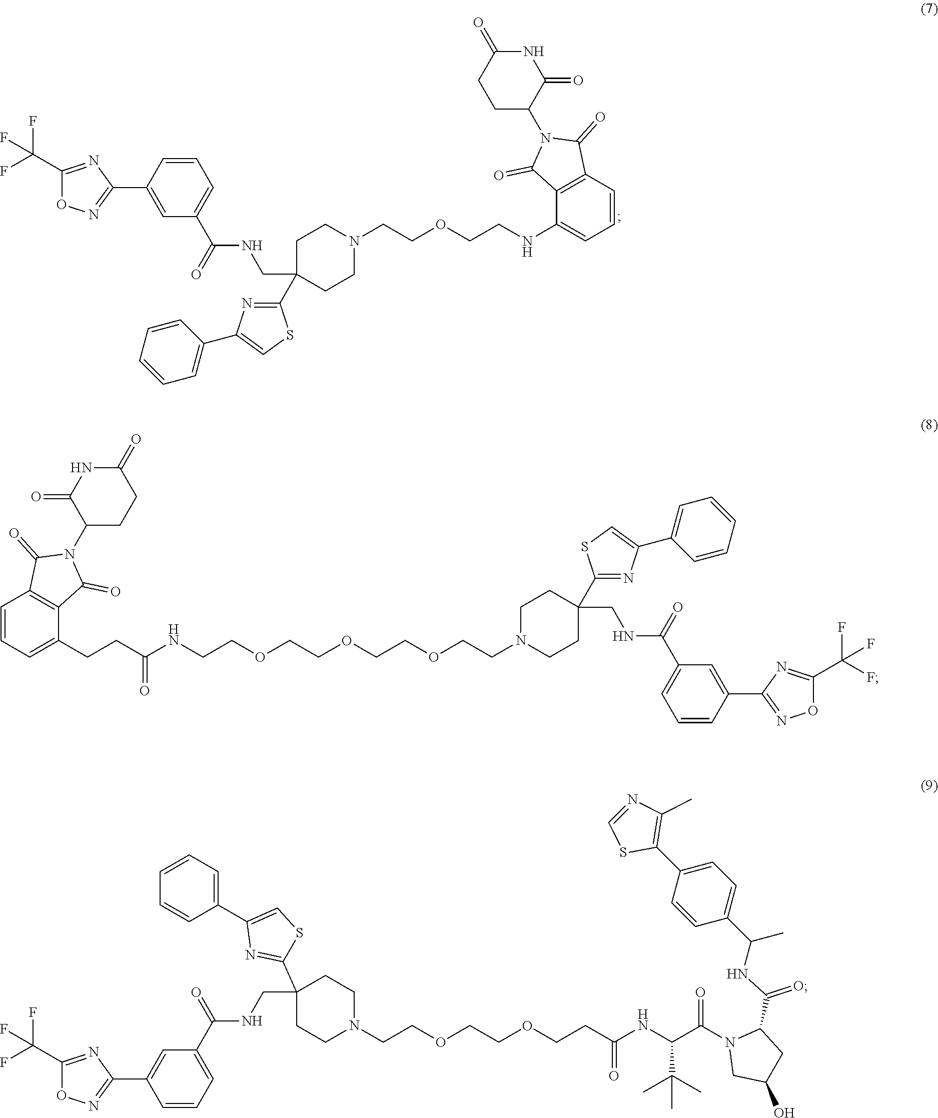 [SMILES: O=C1CCC(N2C(=O)c3cccc(NCCOCCN4CCC(CNC(=O)c5cccc(-c6noc(C(F)(F)F)n6)c5)(c5nc(-c6ccccc6)cs5)CC4)c3C2=O)C(=O)N1 ; O=C(CCc1cccc2c1C(=O)N(C1CCC(=O)NC1=O)C2=O)NCCOCCOCCOCCN1CCC(CNC(=O)c2cccc(-c3noc(C(F)(F)F)n3)c2)(c2nc(-c3ccccc3)cs2)CC1 ; Cc1ncsc1-c1ccc(C(C)NC(=O)[C@@H]2C[C@@H](O)CN2C(=O)[C@@H](NC(=O)CCOCCOCCN2CCC(CNC(=O)c3cccc(-c4noc(C(F)(F)F)n4)c3)(c3nc(-c4ccccc4)cs3)CC2)C(C)(C)C)cc1]

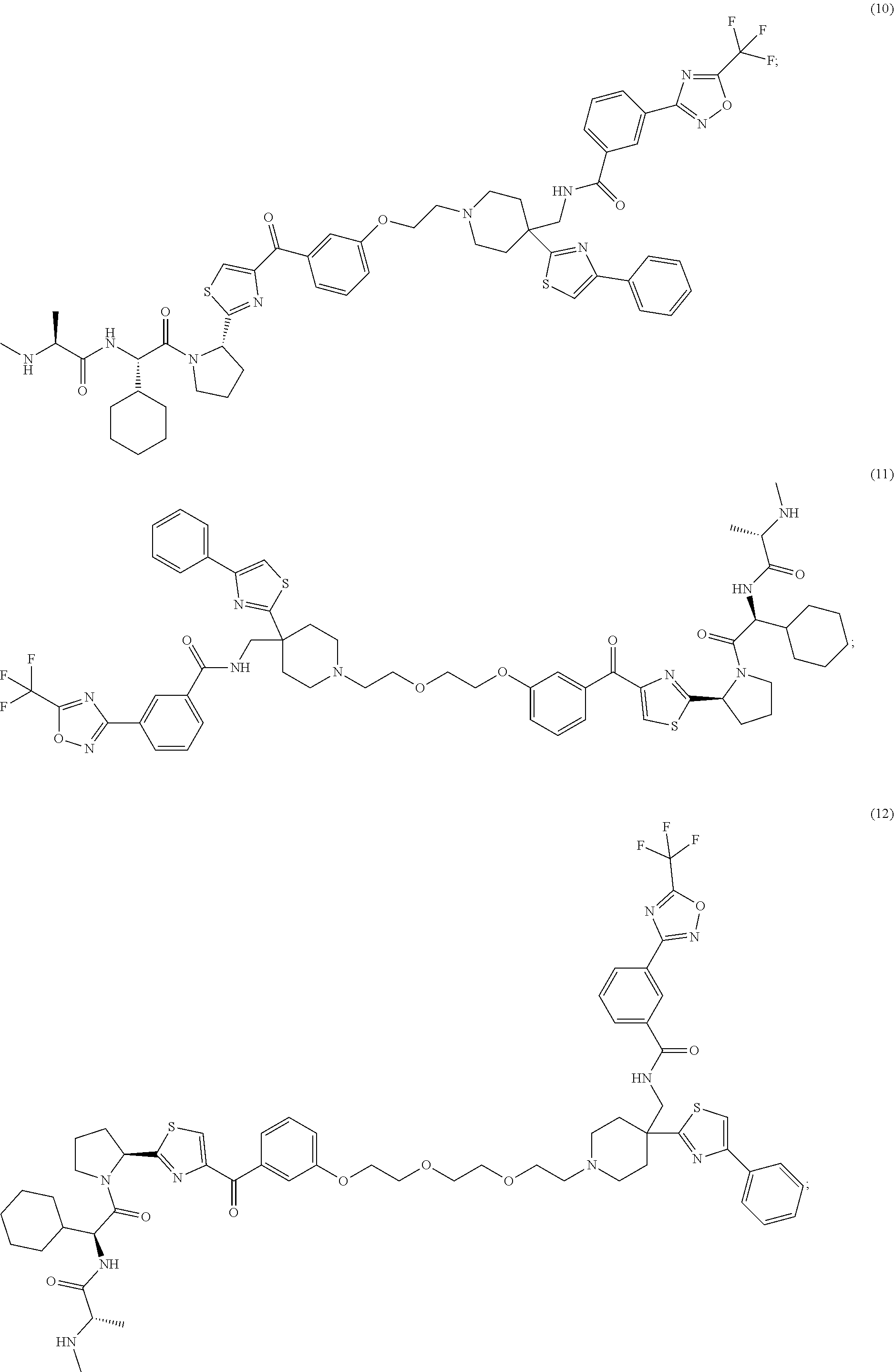 [SMILES: CN[C@@H](C)C(=O)N[C@H](C(=O)N1CCC[C@H]1c1nc(C(=O)c2cccc(OCCN3CCC(CNC(=O)c4cccc(-c5noc(C(F)(F)F)n5)c4)(c4nc(-c5ccccc5)cs4)CC3)c2)cs1)C1CCCCC1 ; CN[C@@H](C)C(=O)N[C@H](C(=O)N1CCC[C@H]1c1nc(C(=O)c2cccc(OCCOCCN3CCC(CNC(=O)c4cccc(-c5noc(C(F)(F)F)n5)c4)(c4nc(-c5ccccc5)cs4)CC3)c2)cs1)C1CCCCC1 ; CN[C@@H](C)C(=O)N[C@H](C(=O)N1CCC[C@H]1c1nc(C(=O)c2cccc(OCCOCCOCCN3CCC(CNC(=O)c4cccc(-c5noc(C(F)(F)F)n5)c4)(c4nc(-c5ccccc5)cs4)CC3)c2)cs1)C1CCCCC1]

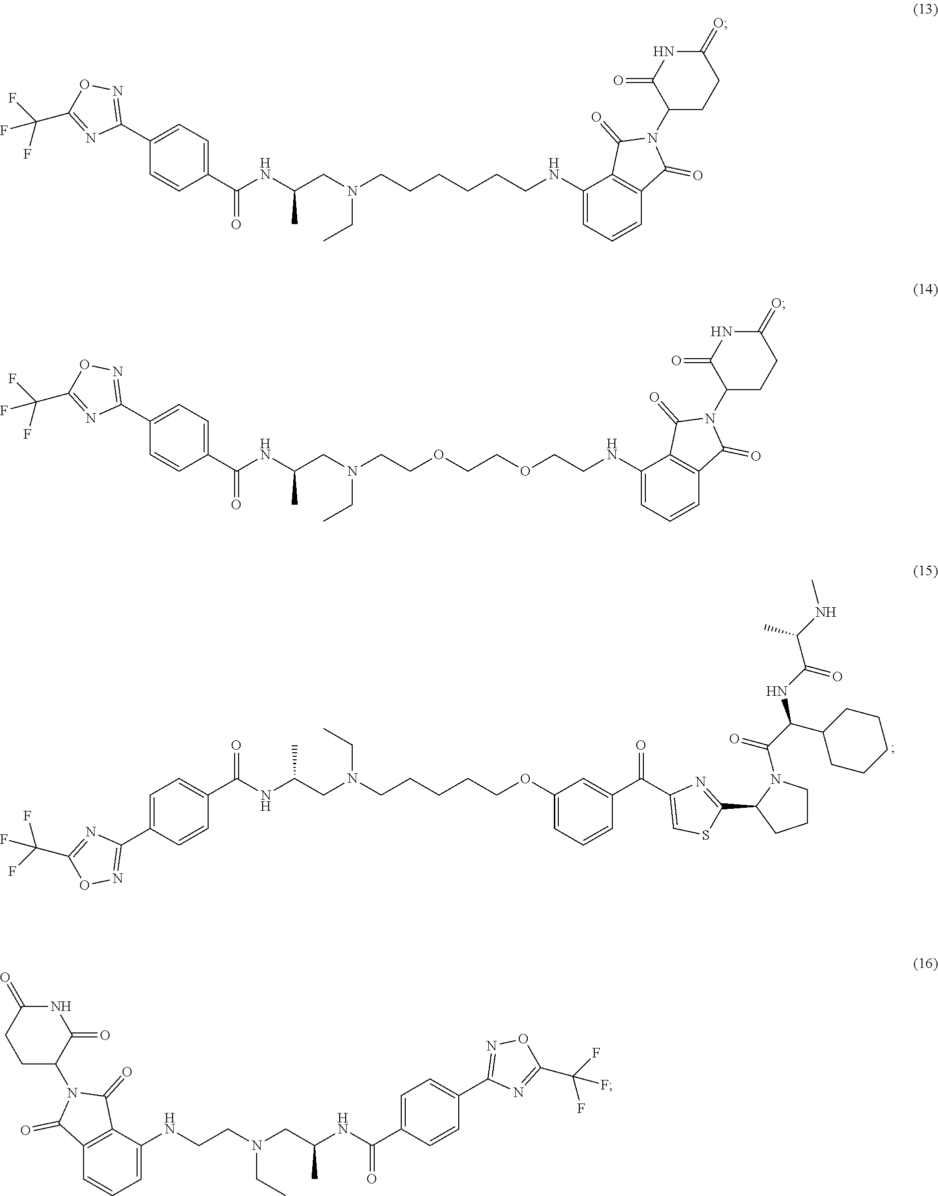 [SMILES: CCN(CCCCCCNc1cccc2c1C(=O)N(C1CCC(=O)NC1=O)C2=O)C[C@@H](C)NC(=O)c1ccc(-c2noc(C(F)(F)F)n2)cc1 ; CCN(CCOCCOCCNc1cccc2c1C(=O)N(C1CCC(=O)NC1=O)C2=O)C[C@@H](C)NC(=O)c1ccc(-c2noc(C(F)(F)F)n2)cc1 ; CCN(CCCCCOc1cccc(C(=O)c2csc([C@@H]3CCCN3C(=O)[C@@H](NC(=O)[C@H](C)NC)C3CCCCC3)n2)c1)C[C@@H](C)NC(=O)c1ccc(-c2noc(C(F)(F)F)n2)cc1 ; CCN(CCNc1cccc2c1C(=O)N(C1CCC(=O)NC1=O)C2=O)C[C@H](C)NC(=O)c1ccc(-c2noc(C(F)(F)F)n2)cc1]

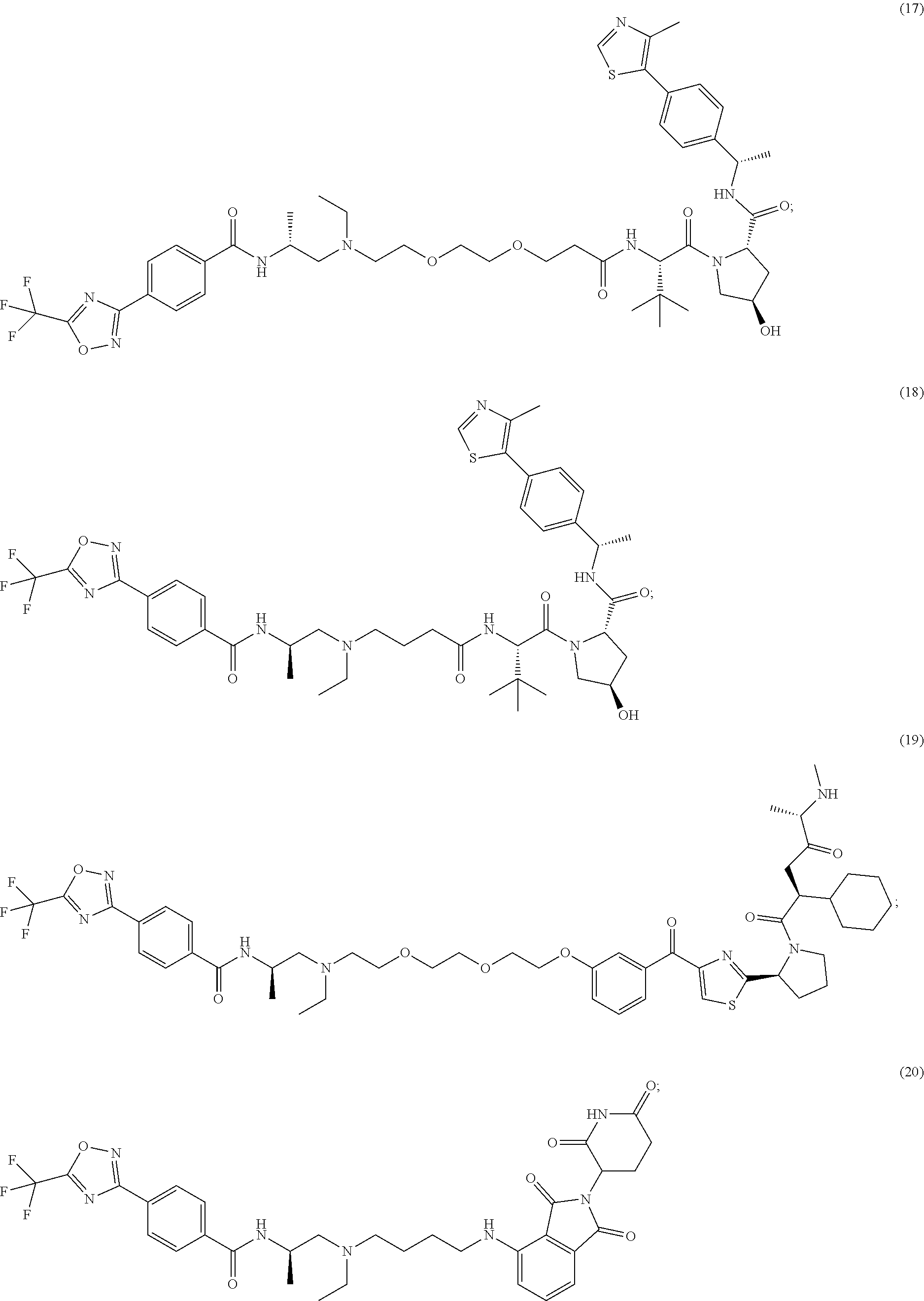 [SMILES: CCN(CCOCCOCCC(=O)N[C@H](C(=O)N1C[C@H](O)C[C@H]1C(=O)N[C@@H](C)c1ccc(-c2scnc2C)cc1)C(C)(C)C)C[C@@H](C)NC(=O)c1ccc(-c2noc(C(F)(F)F)n2)cc1 ; CCN(CCCC(=O)N[C@H](C(=O)N1C[C@H](O)C[C@H]1C(=O)N[C@@H](C)c1ccc(-c2scnc2C)cc1)C(C)(C)C)C[C@@H](C)NC(=O)c1ccc(-c2noc(C(F)(F)F)n2)cc1 ; CCN(CCOCCOCCOc1cccc(C(=O)c2csc([C@@H]3CCCN3C(=O)[C@@H](CC(=O)[C@H](C)NC)C3CCCCC3)n2)c1)C[C@@H](C)NC(=O)c1ccc(-c2noc(C(F)(F)F)n2)cc1 ; CCN(CCCCNc1cccc2c1C(=O)N(C1CCC(=O)NC1=O)C2=O)C[C@@H](C)NC(=O)c1ccc(-c2noc(C(F)(F)F)n2)cc1]

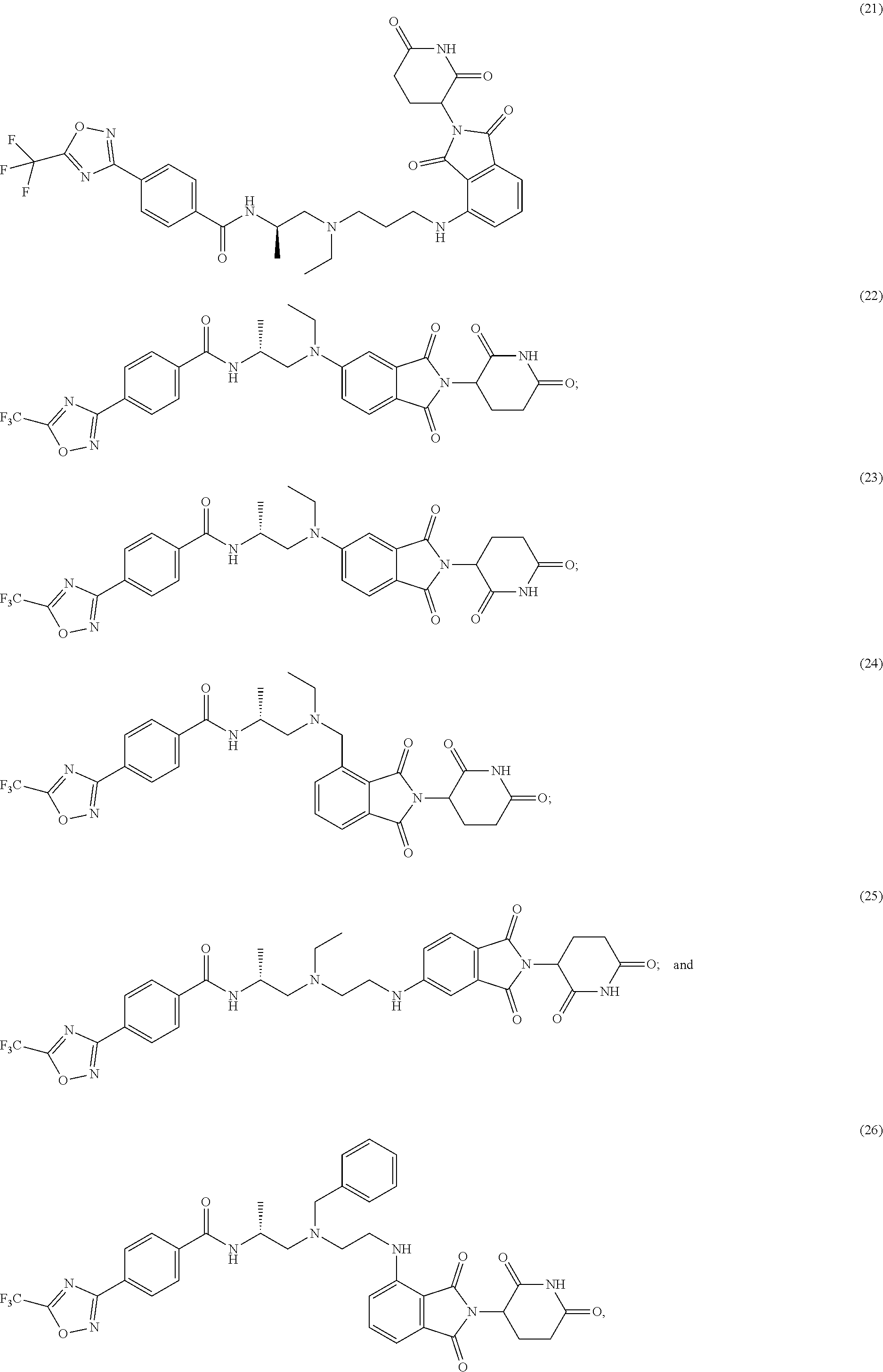

or a pharmaceutically acceptable salt or stereoisomer thereof.

Compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present disclosure with a suitable acid or a base. Examples of pharmaceutically acceptable salts of the compounds of this disclosure include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the disclosure can form pharmaceutically acceptable salts with various organic bases such as lysine, arginine, guanidine, diethanolamine or metformin.

Compounds of formula (I) may have at least one chiral center and thus may be in the form of a stereoisomer, which, as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z. R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms.

In addition, compounds of formula (I) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In some embodiments, the present disclosure is directed to a method for making a compound of formula (I) or a pharmaceutically acceptable salt or stereoisomer thereof. Broadly, the compounds or pharmaceutically acceptable salts or stereoisomers thereof, may be prepared by any process known to be applicable to the preparation of chemically related compounds. The compounds of the present disclosure will be better understood in connection with the synthetic schemes that described in various working examples that illustrate non-limiting methods by which the compounds of the disclosure may be prepared.

### Pharmaceutical Compositions

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro. Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrastemal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of the present disclosure may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs), semi-solid compositions (e.g., gels, suspensions and creams), and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the present disclosure may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compounds may be formulated for buccal or sublingual administration, examples of which include tablets, lozenges and gels.

The compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton. Fla. (1995), which surveys the use and testing of various skin penetration enhancers, and Buvuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents.

Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof, or a composition including a compound of formula (I) or a pharmaceutically acceptable salt or a stereoisomer thereof, effective in producing the desired therapeutic response in a particular patient suffering from a disease or disorder characterized or mediated by aberrant activity of at least one class IIa HDAC. The term “therapeutically effective amount” thus includes the amount of a compound of the disclosure or a pharmaceutically acceptable salt or a stereoisomer thereof, that when administered, induces a positive modification in the disease or disorder to be treated, or is sufficient to inhibit or even prevent development or progression of the disease or disorder, or alleviate to some extent, one or more of the symptoms of the disease or disorder being treated in a subject, or which simply kills or inhibits the growth of diseased (e.g., neurodegenerative diseases, alopecia, glucose homeostasis, muscular dystrophy, autoimmunity, and ischemic stroke) cells, or reduces the amount of at least one class IIa HDAC in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed: the duration of the treatment: drugs used in combination or coincidental with the compound: and like factors well known in the medical arts (see, for example, Goodman and Gilman's, The Pharmacological Basis of Therapeutics, 10th Edition, A Gilman, J. Hardman and L. Limbird, eds., McGraw-Hill Press, 155-173, 2001).

Compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

### Methods of Use

In some aspects, the present disclosure is directed to methods of treating diseases or disorders involving aberrant (e.g., dysfunctional or dysregulated) activity of at least one class IIa HDAC, that entails administration of a therapeutically effective amount of a compound formula (I) or a pharmaceutically acceptable salt or stereoisomer thereof, to a subject in need thereof.

The diseases or disorders are characterized or mediated by aberrant activity of at least one class IIa HDAC (e.g., elevated levels of at least one class IIa HDAC or one or more otherwise functionally abnormal class IIa HDACs relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal. e.g., a human or a non-human mammal. The methods are also applicable to companion animals such as dogs and cats. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject is suffering from the disease or disorder. Thus, subjects suffering from, and suspected of suffering from, a specific disease or disorder are not necessarily two distinct groups.

In some embodiments, compounds of formula (I) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

Exemplary types of non-cancerous (e.g., cell proliferative) diseases or disorders that may be amenable to treatment with the compounds of the present disclosure include inflammatory diseases and conditions, autoimmune diseases, neurodegenerative diseases, heart diseases, viral diseases, chronic and acute kidney diseases or injuries, metabolic diseases, and allergic and genetic diseases.

In some embodiments, the compounds may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, prion disease, motor neuron diseases (MND), Huntington's disease (HD). Pick's syndrome, spinocerebellar ataxia (SCA), spinal muscular atrophy (SMA), primary progressive aphasia (PPA), amyotrophic lateral sclerosis (ALS), traumatic brain injury (TBI), multiple sclerosis (MS), dementias (e.g., vascular dementia (VaD), Lewy body dementia (LBD), semantic dementia, and frontotemporal lobar dementia (FTD).

In some embodiments, the neurodegenerative disease is Parkinson's disease, Alzheimer's disease, or Huntington's disease.

In some embodiments, the compounds may be useful in the treatment of autoimmune diseases and disorders (autoimmunity). As used herein, the term “autoimmune disease” refers to conditions where the immune system produces antibodies that attack normal body tissues. Representative examples of such diseases include autoimmune hematological disorders (e.g., hemolytic anemia, aplastic anemia, anhidrotic ectodermal dysplasia, pure red cell anemia and idiopathic thrombocytopenia), Sjogren's syndrome. Hashimoto thyroiditis, rheumatoid arthritis, juvenile (type 1) diabetes, polymyositis, scleroderma, Addison's disease, lupus, including systemic lupus erythematosus, vitiligo, pernicious anemia, glomerulonephritis, pulmonary fibrosis, celiac disease, polymyalgia rheumatica, multiple sclerosis, ankylosing spondylitis, alopecia areata, vasculitis, autoimmune uveoretinitis, lichen planus, bullous pemphigus, pemphigus vulgaris, pemphigus foliaceus, paraneoplastic pemphigus, myasthenia gravis, immunoglobulin A nephropathy, Wegener granulomatosis, autoimmune oophoritis, sarcoidosis, rheumatic carditis, ankylosing spondylitis, Grave's disease, autoimmune thrombocytopenic purpura, psoriasis, psoriatic arthritis, dermatitis herpetiformis, ulcerative colitis, and temporal arteritis.

In some embodiments, the compounds may be useful in the treatment of alopecia, glucose homeostasis, muscular dystrophy, autoimmunity, and ischemic stroke.

In other embodiments, the methods are directed to treating subjects having cancer.

Broadly, the compounds of the present disclosure may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers include adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the compounds of the present disclosure include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomvosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present disclosure entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, agnogenic mveloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g. acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present disclosure may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome. Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

The compounds of formula (I) may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present disclosure may entail administration of compounds of formula (I) or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

Compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) in combination with one or more additional therapeutics known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 0th ed., McGraw-Hill, New York, 2001; Physician's Desk Reference 60th ed., 2006. For example, anti-cancer agents that may be suitable for use in combination with the compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, a compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, the compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present compounds and/or compositions containing them may be assembled into kits or pharmaceutical systems. Kits or pharmaceutical systems according to this aspect of the disclosure include a carrier or package such as a box, carton, tube or the like, having in close confinement therein one or more containers, such as vials, tubes, ampoules, or bottles, which contain a compound of formula (I) or a pharmaceutical composition thereof. The kits or pharmaceutical systems of the disclosure may also include printed instructions for using the compounds and compositions.

These and other aspects of the present disclosure will be further appreciated upon consideration of the following Examples, which are intended to illustrate certain particular embodiments of the disclosure but are not intended to limit its scope, as defined by the claims.

### Examples

These and other aspects of the present disclosure will be further appreciated upon consideration of the following Examples, which are intended to illustrate certain particular embodiments of the disclosure but are not intended to limit its scope, as defined by the claims.

### General Methods

Unless otherwise noted, reagents and solvents were used as received from commercial suppliers. All reactions were monitored using a Waters® Acquity ultra performance liquid chromatography/mass spectrometry (UPLC/MS) system using Acquity UPLC® BEH C18 column (2.1×50 mm, 1.7 μm particle size). UPLC method A: solvent gradient=80% A at 0 min, 5% A at 1.8 min; method B: solvent gradient=100% A at 0 min, 5% A at 1.8 min; solvent A=0.1% formic acid in H₂O: solvent B=0.1% formic acid in acetonitrile: flow rate: 0.6 mL/min; or an Agilent LC/MS system (Agilent 1200LC/G6130A MS) using SunFire™ C18 column (4.6×50 mm, 3.5 μm particle size), LC method: solvent gradient=95% A to 5% A: solvent A=0.01% TFA in H₂O; solvent B=0.01% TFA in acetonitrile (ACN); flow rate: 2.0 mL/min, column temperature 50° C. Purification of reaction products was carried out by flash chromatography using CombiFlash®Rf with Teledyne Isco RediSep® normal-phase silica flash columns (ISCO); or Waters® high performance liquid chromatography (HPLC) system using SunFire™ C18 column (19×100 mm, 5 μm particle size): solvent gradient 0% to 100% acetonitrile or MeOH in H₂O (0.035% TFA as additive); flow rate: 20 mL/min, or SunFire™ C18 column (30×250 mm, 5 μm particle size): solvent gradient 0% to 100% acetonitrile or MeOH in H₂O (0.035% trifluoroacetic acid (TFA) as additive); flow rate: 40 mL/min. The purity of all compounds was over 95% and was analyzed with Waters® UPLC system. ¹H NMR and ³C NMR spectra were obtained using Bruker Avance III™ spectrometers (400 MHz or 500 MHz for ¹H, and 125 MHz for ¹³C). Chemical shifts are reported relative to deuterated methanol (δ=3.31) or dimethyl sulfoxide (DMSO) (δ=2.50) for ¹H NMR Spectra are given in ppm (δ) and as br=broad, s=singlet, d=doublet, t=triplet, q=quartet, m=multiplet and coupling constants (J) are reported in Hertz.

### Example 1: Synthesis of Degron-Linker Intermediates

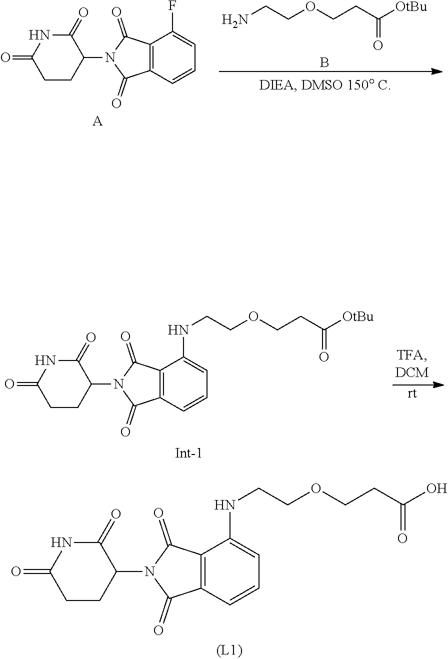

### 3-(2-((2-(2,6-Dioxopiperidin-3-yl)-1,3-dioxoisoindolin-4-yl)amino)ethoxy)propanoic acid (L1)

To solution of compound A (405 mg, 1.47 mmol, 1.0 eq.) and N,N-diisopropylethylamine (DIEA) (510 μL, 2.0 eq.) in DMSO (7 mL) in a reaction tube, tert-butyl 3-(2-aminoethoxy)propanoate (B)(250 mg, 0.9 eq.) was added in one batch, and the reaction tube was sealed and immediately heated to 150° C. After 30 min, the reaction mixture was cooled to room temperature (rt), and H₂O was added before extraction with ethyl acetate (three times). The combined organic layer was washed with H₂O and brine, dried over anhydrous Na₂SO₄, and concentrated in vacuo. The residue was purified using ISCO (dichloromethane/methanol, 0%-10%) to yield int-1 as a yellow oil (506 mg, 86% yield).

UPLC-MS RT: 1.32 min (Method A), Mass m/z: 389.87 [M-tBu+H]⁺.

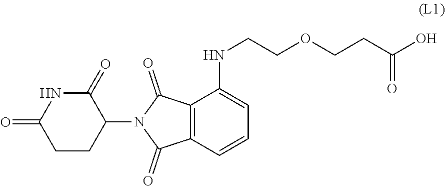 [SMILES: O=C(O)CCOCCNc1cccc2c1C(=O)N(C1CCC(=O)NC1=O)C2=O]

To a solution of int-1 (44 mg, 0.10 mmol, 1.0 eq.) in dichloromethane (3 mL), TFA (0.5 mL) was added, and the reaction was monitored by UPLC. Upon consumption of the starting material, the mixture was concentrated in vacuo and purified with HPLC (H₂O/MeOH, 0%-100%) to yield compound L1 as a yellow oil (30 mg, quant, yield).

UPLC-MS RT: 0.82 min (Method A), Mass m/z: 389.87 [M+H]⁺.

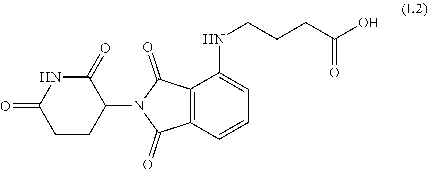 [SMILES: O=C(O)CCCNc1cccc2c1C(=O)N(C1CCC(=O)NC1=O)C2=O]

### 4-((2-(2,6-Dioxopiperidin-3-yl)-1,3-dioxoisoindolin-4-yl)amino)butanoic acid (L2)

Compound L2 was synthesized in an analogous manner to compound L1 from compounds A and tert-butyl 4-aminobutanoate using the above procedure, and was isolated as a yellow oil.

UPLC-MS RT: 0.85 min (Method A), Mass m/z: 360.27 [M+H]⁺.

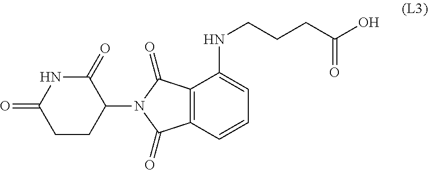 [SMILES: O=C(O)CCCNc1cccc2c1C(=O)N(C1CCC(=O)NC1=O)C2=O]

### 6-((2-(2,6-Dioxopiperidin-3-yl)-1,3-dioxoisoindolin-4-yl)amino)hexanoic acid (L3)

Compound L3 was synthesized in an analogous manner to compound L1 from compounds A and 6-aminohexanoic acid, and was isolated as a yellow oil.

UPLC-MS RT: 1.00 min Method A), Mass m/z: 387.97 [M+H]⁺.

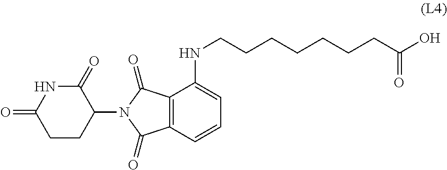 [SMILES: O=C(O)CCCCCCCNc1cccc2c1C(=O)N(C1CCC(=O)NC1=O)C2=O]

### 8-((2-(2,6-Dioxopiperidin-3-yl)-1,3-dioxoisoindolin-4-yl)amino)octanoic acid (L4)

Compound L4 was synthesized in an analogous manner to compound L1 from compounds A and 8-aminooctanoic acid, and was isolated as a yellow oil.

UPLC-MS RT: 1.15 min (Method A), Mass m/z: 415.97 [M+H]⁺.

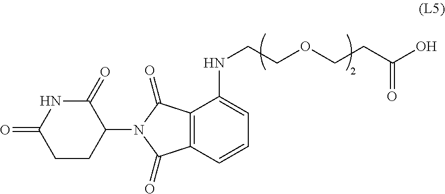

### 3-(2-(2-((2-(2,6-Dioxopiperidin-3-yl)-1,3-dioxoisoindolin-4-yl)amino)ethoxy)ethoxy) propanoic acid (LS)

Compound L5 was synthesized in an analogous manner to compound L1 from compounds A and tert-butyl 3-(2-aminoethoxy)-4-methoxybutanoate, and was isolated as a yellow oil.

UPLC-MS RT: 1.32 min (Method A), Mass m/z: 433.87 [M+H]⁺.

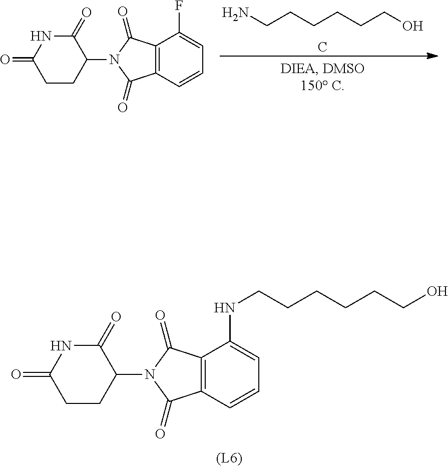

### 2-(2,6-Dioxopiperidin-3-yl)-4-((6-hydroxyhexyl)amino)isoindoline-1,3-dione (L6)

To solution of compound A (400 mg, 1.45 mmol, 1.0 eq.) and DIEA (378 μL, 1.5 eq.) in DMSO (6 mL) in a reaction tube, 6-aminohexan-1-ol (C) (204 mg, 1.2 eq.) was added in one batch, and the reaction tube was sealed and immediately heated to 150° C. After 1 h, the reaction mixture was cooled to room temperature, and H₂O was added before extraction with ethyl acetate (three times). The combined organic layer was washed with H₂O and brine, dried over anhydrous Na₂SO₄, and concentrated in vacuo. The residue was purified using HPLC (H₂O/acetonitrile, 0%-100%) to yield L6 as a yellow oil (409 mg, 76% yield).

UPLC-MS RT: 1.04 min (Method A), Mass m/z: 374.17 [M+H]⁺.

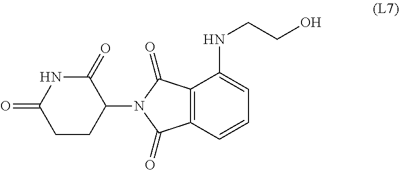 [SMILES: O=C1CCC(N2C(=O)c3cccc(NCCO)c3C2=O)C(=O)N1]

### 2-(2,6-Dioxopiperidin-3-yl)-4-((2-hydroxyethyl)amino)isoindoline-1,3-dione (L7)

Compound L7 was synthesized in an analogous manner to compound L6 from compounds A and 2-aminoethan-1-ol, and was isolated as a yellow oil.

UPLC-MS RT: 0.75 min (Method A), Mass m/z: 318.17 [M+H]⁺.

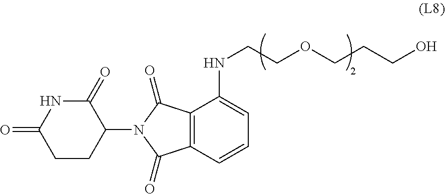

### 2-(2,6-dioxopiperidin-3-yl)-4-((2-(2-(2-hydroxyethoxy)ethoxy)ethyl)amino) isoindoline-1,3-dione (LS)

Compound L8 was synthesized in an analogous manner to compound L6 from compounds A and 2-(2-(2-aminoethoxy)ethoxy)ethan-1-ol, and was isolated as a yellow oil.

UPLC-MS RT: 0.82 min (Method A), Mass m/z: 406.27 [M+H]⁺.

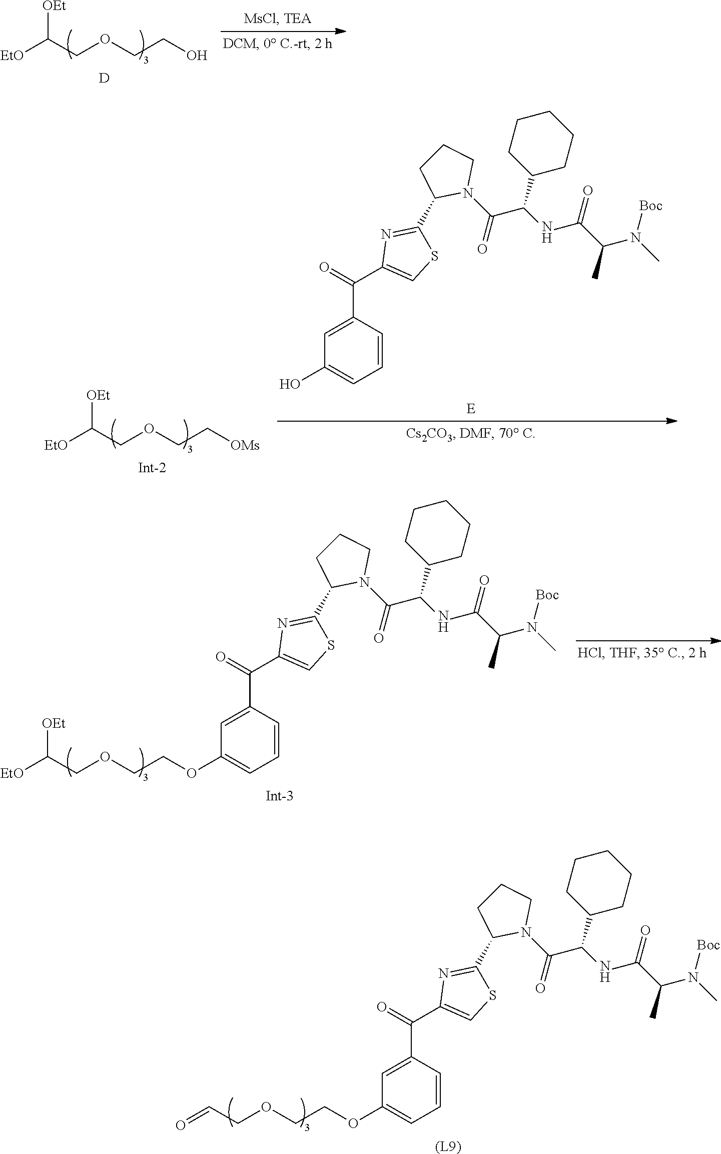

### tert-Butyl ((S)-1-(((S)-1-cyclohexyl-2-oxo-2-((S)-2-(4-(3-(2-(2-(2-(2-oxoethoxy)ethoxy)ethoxy)ethoxy)benzoyl)thiazol-2-yl)pyrrolidin-1-yl)ethyl)amino)-1-oxopropan-2-yl)(methyl)carbamate (L9)

To a solution of acetal D (133 mg, 0.5 mmol, 1.0 eq.) in dichloromethane (3 mL), MsCl (94 μL, 2.4 eq.) and NEt₃ (209 μL, 3 eq.) were added at 0° C. The reaction was stirred for 30 min and monitored by UPLC-MS. Upon consumption of the starting material, the reaction was quenched with H₂O, extracted with dichloromethane (three times), dried over Na₂SO₄, filtered, and concentrated in vacuo to yield compound int-2, which was used in the next step without further purification.

UPLC-MS RT: 0.96 min (Method A), Mass m/z: 367.27 [M+Na]⁺.

A mixture of compounds E (Shibata et al., J Med Chem 61:543-575 (2018)) (200 mg, 0.33 mmol, 1.0 eq.) and int-2 (1.5 eq., crude from last step) in DMF (3 mL) was treated with Cs₂CO₃ (82 mg, 2 eq.). The reaction mixture was heated at 70° C. for 12 h and monitored by UPLC-MS. Upon consumption of the starting material, the reaction was filtered, extracted with ethyl acetate (three times), dried over Na₂SO₄, filtered, concentrated in vacuo, and purified using ISCO (dichloromethane/methanol, 0%-10%) to yield compound int-3.

UPLC-MS RT: 1.76 min (Method A), Mass m/z: 869.52 [M+Na]⁺.

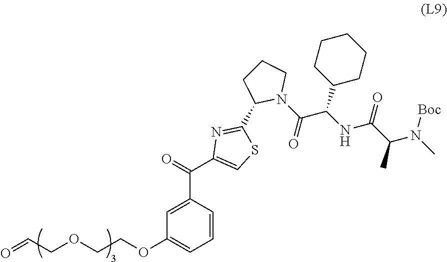

A solution of int-3 (40 mg, 0.047 mmol, 1.0 eq.) in THF (0.5 mL) was treated with 2N aqueous HCl (250 μL, 10 eq.). The reaction was stirred at 35° C. for 2 h and monitored by UPLC-MS. Upon consumption of the starting material, the reaction was quenched with aqueous NaHCO₃, extracted with iPrOH/CHC₃ (three times), dried over Na₂SO₄, filtered, and concentrated in vacuo to yield L9, which was used without further purification.

UPLC-MS RT: 1.46 min (Method A), Mass m/z: 795.41 [M+Na]⁺.

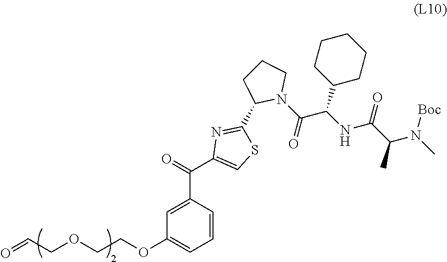

### tert-Butyl ((S)-1-(((S)-1-cyclohexyl-2-oxo-2-((S)-2-(4-(3-(2-(2-(2-oxoethoxy)ethoxy)ethoxy)benzoyl)thiazol-2-yl)pyrrolidin-1-yl)ethyl)amino)-1-oxopropan-2-yl)(methyl)carbamate (L10)

Compound L10 was synthesized in an analogous manner to compound L9 from 2-(2-(2,2-diethoxyethoxy)ethoxy)ethan-1-ol.

UPLC-MS RT: 1.41 min (Method A), Mass m/z: 728.71 [M+H]⁺.

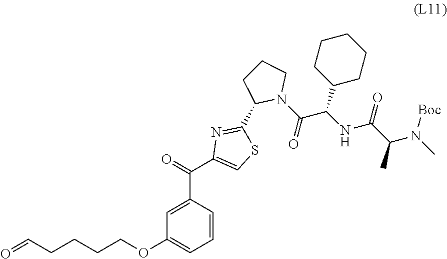 [SMILES: C[C@@H](C(=O)N[C@H](C(=O)N1CCC[C@H]1c1nc(C(=O)c2cccc(OCCCCC=O)c2)cs1)C1CCCCC1)N(C)C(=O)OC(C)(C)C]

### tert-Butyl ((S)-1-(((S)-1-cyclohexyl-2-oxo-2-((S)-2-(4-(3-((5-oxopentyl)oxy)benzoyl)thiazol-2-yl)pyrrolidin-1-yl)ethyl)amino)-1-oxopropan-2-yl)(methyl)carbamate (L11)

Compound L11 was synthesized in an analogous manner to compound L9 from 2-(4-bromobutyl)-1,3-dioxolane.

UPLC-MS RT: 1.69 min (Method A), Mass m/z: 683.60 [M+H]⁺.

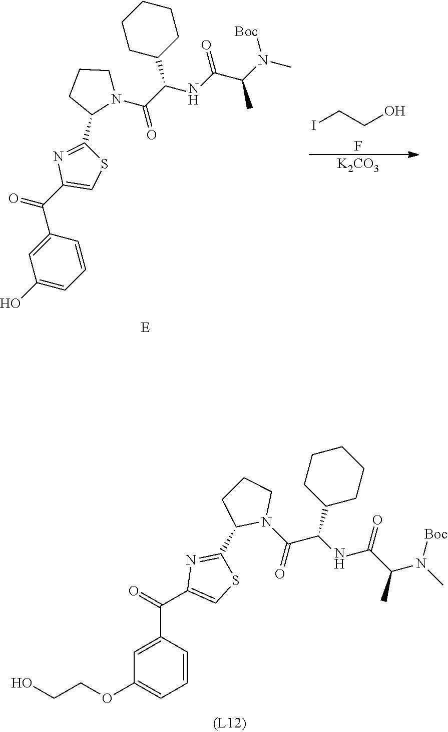

### tert-Butyl ((S)-1-(((S)-1-cyclohexyl-2-((S)-2-(4-(3-(2-hydroxyethoxy)benzoyl)thiazol-2-yl) pyrrolidin-1-yl)-2-oxoethyl)amino)-1-oxopropan-2-yl)(methyl)carbamate (12)

A mixture of compound E (Shibata et al., J Med Chem 61:543-575 (2018)) (50 mg, 0.083 mmol, 1.0 eq.) and 2-iodoethan-1-ol (F) (15.4 μL, 2.4 eq.) in DMF (1 mL) was treated with K₂CO₃ (17 mg, 1.5 eq.). The reaction mixture was heated at 70° C. for 2 days and monitored by UPLC-MS. Upon consumption of the starting material, the reaction was filtered, extracted with ethyl acetate three times, dried over Na₂SO₄, filtered, concentrated in vacuo, and purified using column chromatography (silica gel, dichloromethane/MeOH) to yield compound L12.

UPLC-MS RT: 1.44 min (Method A), Mass m/z: 643.00 [M+H]⁺.

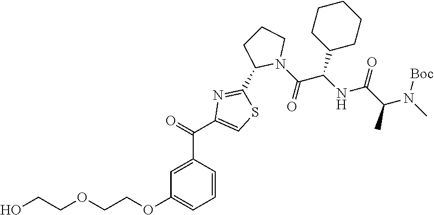 [SMILES: C[C@@H](C(=O)N[C@H](C(=O)N1CCC[C@H]1c1nc(C(=O)c2cccc(OCCOCCO)c2)cs1)C1CCCCC1)N(C)C(=O)OC(C)(C)C]

### tert-Butyl ((S)-1-(((S)-1-cyclohexyl-2-((S)-2-(4-(3-(2-(2-hydroxyethoxy)ethoxy)benzoyl)thiazol-2-yl)pyrrolidin-1-yl)-2-oxoethyl)amino)-1-oxopropan-2-yl)(methyl)carbamate (L13)

Compound L13 was synthesized in an analogous manner to compound L12 from compound E and 2-(2-bromoethoxy)ethan-1-ol.

UPLC-MS RT: 1.44 min (Method A), Mass m/z: 687.00 [M+H]⁺.

### Example 2: Synthesis of N-((1-(6-((2-(2,6-dioxopiperidin-3-yl)-1,3-dioxoisoindolin-4-yl)amino)hexyl)-4-(4-phenylthiazol-2-ylpiperidin-4-yl)methyl)-3-(5-(trifluoromethyl)-1,2,4-oxadiazol-3-yl)benzamide (1)

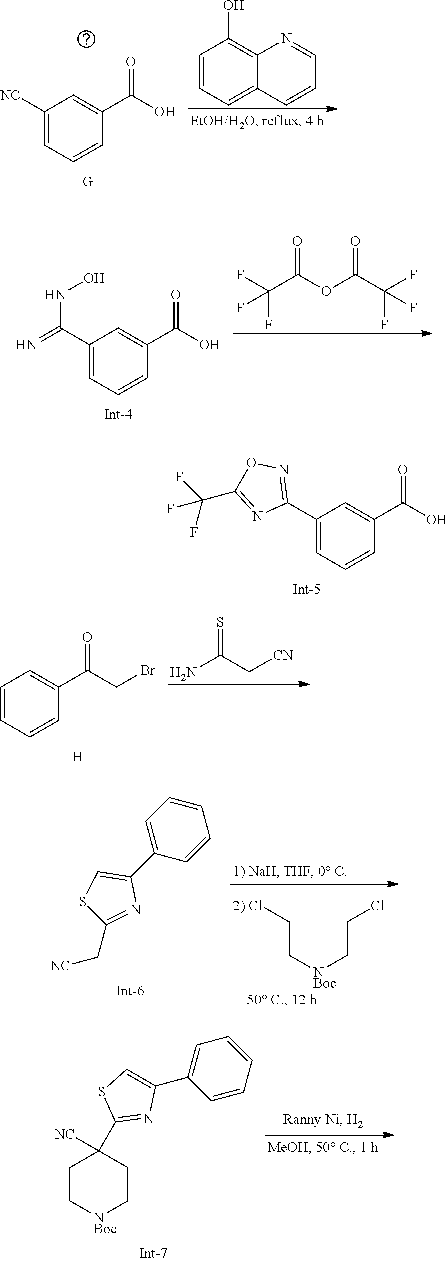

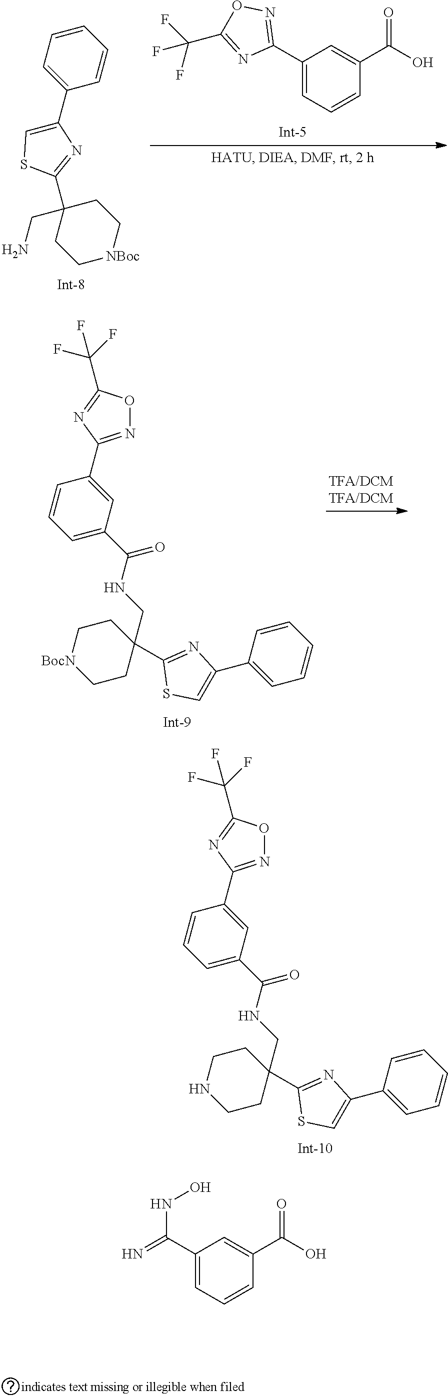 [SMILES: CC(C)(C)OC(=O)N1CCC(CN)(c2nc(-c3ccccc3)cs2)CC1 ; O=C(O)c1cccc(-c2noc(C(F)(F)F)n2)c1 ; CC(C)(C)OC(=O)N1CCC(CNC(=O)c2cccc(-c3noc(C(F)(F)F)n3)c2)(c2nc(-c3ccccc3)cs2)CC1 ; O=C(NCC1(c2nc(-c3ccccc3)cs2)CCNCC1)c1cccc(-c2noc(C(F)(F)F)n2)c1 ; N=C(NO)c1cccc(C(=O)O)c1]

### 3-(N-Hydroxycarbamimidoyl)benzoic acid (Int-4)

8-hydroxyquinoline (11.8 mg, 0.3 mol) was added to a solution of 3-cyanobenzoic acid (G)(4 g, 27.2 mmol, 1.0 eq.) in ethanol (204 mL). To the reaction mixture was added a solution of hydroxylamine hydrochloride (4.04 g, 2.1 eq.) in water (30 mL), followed by the addition of a solution of sodium carbonate (4.67 g, 1.6 eq.) in water (31 mL). The mixture was heated to reflux for 4 h. After removal of ethanol under reduced pressure, the residue was diluted with water (100 mL), and the aqueous solution was acidified to pH-3 with 2N HCl solution. The resulting white precipitate was filtered, washed with water, and dried under reduced pressure to yield int-4 as a white solid (6 g, crude, 100% yield).

¹H-NMR (DMSO-d₆, 400 MHz): δ (ppm) 9.75 (s, 1H), 8.27 (s, 1H), 7.91 (dd, J=15.2, 8.1 Hz, 2H), 7.50 (t, J=7.8 Hz, 1H), 5.92 (s, 2H).

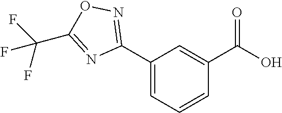 [SMILES: O=C(O)c1cccc(-c2noc(C(F)(F)F)n2)c1]

### 3-(5-(Trifluoromethyl)-1,2,4-oxadiazol-3-yl)benzoic acid (Int-5)

A solution of compound int-4 (3 g, 16.7 mmol, 1.0 eq.) in anhydrous pyridine (45 mL) was cooled to 0° C. and then trifluoroacetic anhydride (10.52 g, 3.0 eq.) was added dropwise. The reaction mixture was allowed to warm to room temperature and then heated at 50° C. for 3 h. The reaction mixture was poured into ice water, adjusted to pH-4 with 1.5 N HCl solution, and extracted with ethyl acetate (60 mL×3). The combined organic layers were washed with brine (50 mL), dried over anhydrous Na₂SO₄, filtered, and concentrated under reduced pressure. The resulting residue was purified by flash column chromatography on silica gel (ethyl acetate in petroleum ether (24% v/v), with 1% v/v of trifluoroacetic acid) to yield int-5 as a white solid (2 g, 46.5% yield).

LC-MS: Mass m/z: 259 [M+H]⁺.

¹H NMR (400 MHz, CDCl₃) δ (ppm) 8.79 (s, 1H), 8.31 (d, J=7.7 Hz, 1H), 8.24 (d, J=7.8 Hz, 1H), 7.61 (t, J=7.9 Hz, 1H).

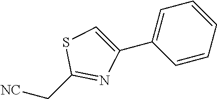 [SMILES: N#CCc1nc(-c2ccccc2)cs1]

### 2-(4-Phenylthiazol-2-yl)acetonitrile (Int-6)

A mixture of 2-bromoacetophenone (H) (6 g, 30.3 mmol, 1.0 eq.) and 2-cyanothioacetamide (3.03 g, 1.0 eq.) in ethanol (75 mL) was heated at 80° C. for 4 h. The reaction mixture was cooled to room temperature and then poured into an aqueous ammonia solution (final pH>7). The mixture was then extracted with ethyl acetate (30 mL×3). The combined organic layers were washed with brine (30 mL×2), dried over anhydrous Na₂SO₄, and concentrated under reduced pressure. The resulting residue was purified by flash column chromatography on silica gel (ethyl acetate in petroleum ether, 20% v/v) to yield int-6 as a solid (4.5 g, 74.2% yield).

LC-MS: Mass m/z: 201 [M+H]⁺.

¹H NMR (400 MHz, CDCl₃) δ (ppm) 7.88 (dd, J=5.3, 3.3 Hz, 2H), 7.48 (d, J=4.2 Hz, 1H), 7.47-7.40 (m, 2H), 7.37 (dtd, J=7.3, 4.8, 2.3 Hz, 1H), 4.18 (d, J=4.9 Hz, 2H).

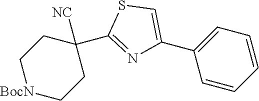 [SMILES: CC(C)(C)OC(=O)N1CCC(C#N)(c2nc(-c3ccccc3)cs2)CC1]

### tert-butyl 4-cyano-4-(4-phenylthiazol-2-yl)piperidine-1-carboxylate (Int-7)

To a solution of compound int-6 (3.4 g, 17 mmol, 1.0 eq.) in anhydrous THF (102 mL) at 0° C., NaH (2.04 g, 60% dispersion in oil, 3.0 eq.) was added portionwise over 10 minutes. The resulting mixture was allowed to warm to room temperature and then stirred for 30 minutes before the dropwise addition of N-Boc-N,N-bis(2-chloroethyl)amine (12.3 g, 3.0 eq.). The reaction mixture was stirred at 50° C. overnight. The resulting mixture was quenched with saturated NH₄Cl solution (50 mL) and extracted with ethyl acetate (50 mL×3). The combined organic layers were washed with brine (50 mL), dried over anhydrous Na₂SO₄, and concentrated under reduced pressure. The resulting residue was purified by flash column chromatography on silica gel (ethyl acetate in petroleum ether, 18% v/v) to int-7 as a solid (2.0 g, 31.8% yield).

LC-MS: Mass m/z: 314 [M-tBu+H]⁺.

¹H NMR (400 MHz, CDCl₃) δ 7.95-7.83 (m, 2H), 7.50 (s, 1H), 7.47-7.40 (m, 2H), 7.39-7.30 (m, 1H), 4.23 (s, 2H), 3.27 (s, 2H), 2.37 (d, J=13.1 Hz, 2H), 2.29-2.18 (m, 2H), 1.49 (s, 9H).

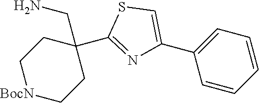 [SMILES: CC(C)(C)OC(=O)N1CCC(CN)(c2nc(-c3ccccc3)cs2)CC1]

### tert-Butyl 4-(aminomethyl)-4-(4-phenylthiazol-2-yl)piperidine-1-carboxylate (Int-8)

A mixture of compound int-7 (0.8 g, 2.2 mmol, 1.0 eq.), Raney®-Nickel (0.8 g, slurry in water), and ammonia (4 mL) in methanol (80 mL) was stirred at 50° C. under hydrogen (1 atm) for 1 h. The resulting mixture was filtered through a pad of Diatomite®, and the cake was washed with a solution of methanol (5 mL) and dichloromethane (50 mL). The filtrate was concentrated and purified by flash column chromatography on silica gel (methanol in dichloromethane, 16% v/v) to yield int-8 as an oil (0.7 g, 85.6% yield).

LC-MS: Mass m/z: 374 [M+H].

¹H NMR (400 MHz, DMSO-d₆) δ 8.06 (s, 1H), 7.96 (d. J=7.9 Hz, 2H), 7.44 (t, J=7.6 Hz, 2H), 7.33 (t, J=7.2 Hz, 1H), 3.72 (d, J=13.4 Hz, 2H), 3.03 (s, 2H), 2.77 (s, 2H), 2.11 (d, J=13.9 Hz, 2H), 1.86-1.70 (m, 2H), 1.39 (s, 9H).

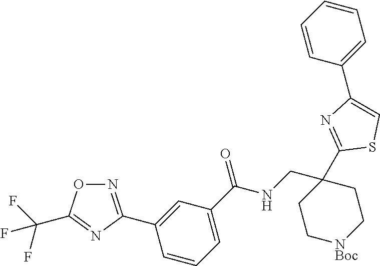 [SMILES: CC(C)(C)OC(=O)N1CCC(CNC(=O)c2cccc(-c3noc(C(F)(F)F)n3)c2)(c2nc(-c3ccccc3)cs2)CC1]

### tert-Butyl 4-(4-phenylthiazol-2-yl)-4-((3-(5-(trifluoromethyl)-1,2,4-oxadiazol-3-yl)benzamido)methyl)piperidine-1-carboxylate (Int-9)

To a solution of compounds int-9 (0.4 g, 1.07 mmol, 1.0 eq.) and int-5 (0.304 g, 1.1 eq.) in anhydrous dimethylformide (DMF) (7 mL) at 0° C. was added hexafluorophosphate azabenzotriazole tetramethyl uronium (HATU) (0.448 g, 1.1 eq.) and N,N-diisopropylethylamine (DIPEA) (0.277 g, 2.0 eq.). The reaction mixture was stirred at room temperature for 2 h under nitrogen atmosphere. The mixture was quenched with water (30 mL) and extracted with ethyl acetate (30 mL×3). The combined organic layers were washed with brine (30 mL), dried over anhydrous Na₂SO₄, and concentrated under vacuum. The resulting residue was purified by flash column chromatography on silica gel (ethyl acetate in petroleum ether, 35% v/v) to yield int-9 as a white solid (0.38 g, 57.8% yield).

LC-MS: Mass m/z: 614 [M+H]⁺.

¹H NMR (400 MHz, DMSO-d₆) δ 8.81 (t, J=6.2 Hz, 1H), 8.45 (s, 1H), 8.21 (t. J=10.4 Hz, 1H), 8.12-8.02 (m, 2H), 7.94 (d, J=7.5 Hz, 2H), 7.69 (t, J=7.8 Hz, 1H), 7.40 (t, J=7.6 Hz, 2H), 7.31 (t, J=7.3 Hz, 1H), 3.85 (d, J=13.3 Hz, 2H), 3.58 (d, J=6.1 Hz, 2H), 2.96 (s, 2H), 2.28 (d, J=13.7 Hz, 2H), 1.88 (t, J=10.4 Hz, 2H), 1.39 (s, 9H).

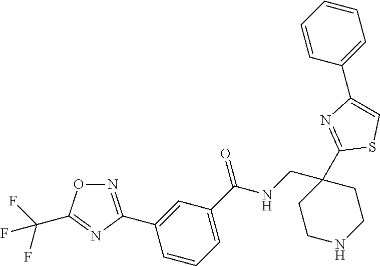 [SMILES: O=C(NCC1(c2nc(-c3ccccc3)cs2)CCNCC1)c1cccc(-c2noc(C(F)(F)F)n2)c1]

### N-((4-(4-Phenylthiazol-2-yl)piperidin-4-yl)methyl)-3-(5-(trifluoromethyl)-1,2,4-oxadiazol-3-yl)benzamide (Int-10)

Compound int-9 (21 mg, 0.034 mmol, 1.0 eq.) was dissolved in dichloromethane (3 mL) and treated with TFA (0.5 mL). The reaction was monitored by UPLC and, upon consumption of the starting material, the mixture was concentrated in vacuo and used in the next step without further purification.

UPLC-MS RT: 1.22 min (Method A), Mass m/z: 514.28 [M+H]⁺.

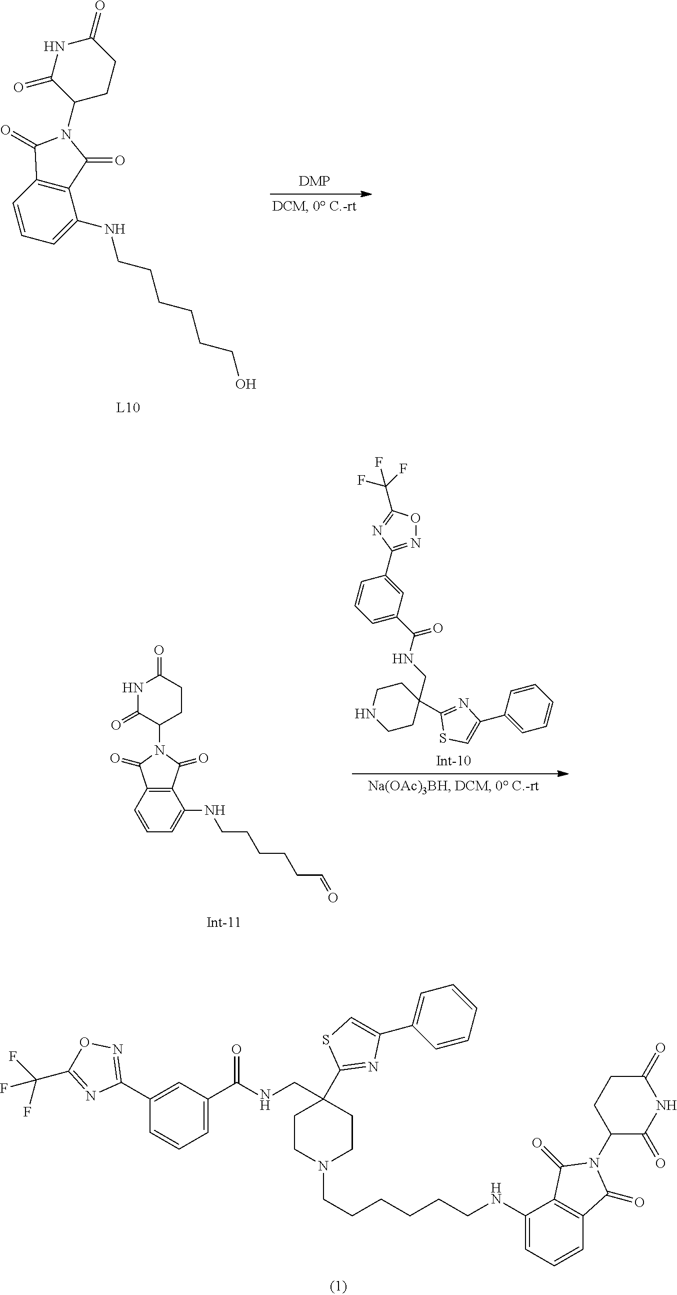 [SMILES: O=C1CCC(N2C(=O)c3cccc(NCCCCCCO)c3C2=O)C(=O)N1 ; O=CCCCCCNc1cccc2c1C(=O)N(C1CCC(=O)NC1=O)C2=O ; O=C(NCC1(c2nc(-c3ccccc3)cs2)CCNCC1)c1cccc(-c2noc(C(F)(F)F)n2)c1 ; O=C1CCC(N2C(=O)c3cccc(NCCCCCCN4CCC(CNC(=O)c5cccc(-c6noc(C(F)(F)F)n6)c5)(c5nc(-c6ccccc6)cs5)CC4)c3C2=O)C(=O)N1]

To a solution of 2-(2,6-dioxopiperidin-3-yl)-4-((6-hydroxyhexyl)amino)isoindoline-1,3-dione (L6)(25 mg, 0.067 mmol, 1.0 eq.) in dichloromethane (1 mL) was treated with Dess-Martin periodinane (30 mg, 1.05 eq.) at 0° C. The reaction mixture was allowed to warm to room temperature and stirred for 3 h. Upon consumption of the starting material, the reaction mixture was quenched with aqueous NaHCO₃ and extracted three times with ethyl acetate. The combined organic layers were washed with brine, dried over Na₂SO₄, filtered, and concentrated in vacuo. The resulting residue (int-11) was used in the next step without further purification.

UPLC-MS RT: 1.14 min (Method A), Mass m/z: 354.17 [M−H₂O+H]⁺.

To a solution of compounds Int-11 (25 mg, 0.067 mmol, 1.0 eq.) and int-10 (34 mg, 1.0 eq., crude from deprotection step) in dichloromethane (1 mL) was added NaBH(OAc)₃ (21 mg, 1.5 eq.) at room temperature, and the reaction mixture was stirred for 18 h. Upon consumption of the starting material, the reaction was quenched with aqueous NaHCO₃ and extracted three times with dichloromethane. The combined organic layers were washed with brine, dried over Na₂SO₄, filtered, and concentrated in vacuo. The resulting residue was purified using ISCO (dichloromethane/methanol, 0%-10%), followed by HPLC (H₂O/acetonitrile, 0%-100%) to yield compound 1 as a yellow powder (5.9 mg, 10% yield over 3 steps).

UPLC-MS RT: 1.59 min (Method A), Mass m/z: 869.42 [M+H]⁺.

¹H NMR (500 MHz, DMSO-d₆) δ 11.08 (s, 1H), 8.72 (t, J=6.4 Hz, 1H), 8.43 (t, J=1.8 Hz, 1H), 8.18 (dt, J=7.8, 1.5 Hz, 1H), 8.07-8.02 (m, 2H), 7.92 (d, J=7.0 Hz, 2H), 7.68 (t, J=7.8 Hz, 1H), 7.56 (dd, J=8.6, 7.1 Hz, 1H), 7.38 (t, J=7.7 Hz, 2H), 7.29 (t, J=7.4 Hz, 1H), 7.07 (d, J=8.6 Hz, 1H), 7.00 (d, J=7.0 Hz, 1H), 6.51 (t, J=6.0 Hz, 1H), 5.04 (dd, J=12.8, 5.5 Hz, 1H), 3.53 (d, J=6.3 Hz, 2H), 3.26 (q, J=6.8 Hz, 2H), 2.87 (ddd, J=16.8, 13.7, 5.4 Hz, 1H), 2.75 (br s, 2H), 2.61-2.45 (m, 2H), 2.31 (s, 1H), 2.28 (s, 1H), 2.19 (br s, 2H), 2.12-1.89 (m, 5H), 1.55 (p, J=7.1 Hz, 2H), 1.45-1.35 (m, 2H), 1.35-1.22 (m, 4H).

¹³C NMR (126 MHz, DMSO) δ 172.81, 170.09, 168.94, 168.10, 167.30, 165.64, 165.32, 164.97, 153.71, 146.43, 136.27, 135.77, 134.38, 132.19, 131.25, 129.84, 129.62 (2C), 128.61 (2C), 127.75, 126.31, 125.97 (2C), 124.55, 117.18, 114.24, 110.36, 108.99, 57.90, 49.71 (3C), 48.53, 44.76, 41.79, 33.65, 33.65, 30.97, 28.61, 26.66, 26.23, 26.18, 22.16.

### Example 3: Synthesis of N-((1-(2-(2-(2-((2-(2,6-dioxopiperidin-3-yl)-1,3-dioxoisoindolin-4-yl)amino)ethoxy)ethyl)-4-(4-phenylthiazol-2-yl)piperidin-4-yl)methyl)-3-(5-(trifluoromethyl)-1,2,4-oxadiazol-3-yl)benzamide (2)

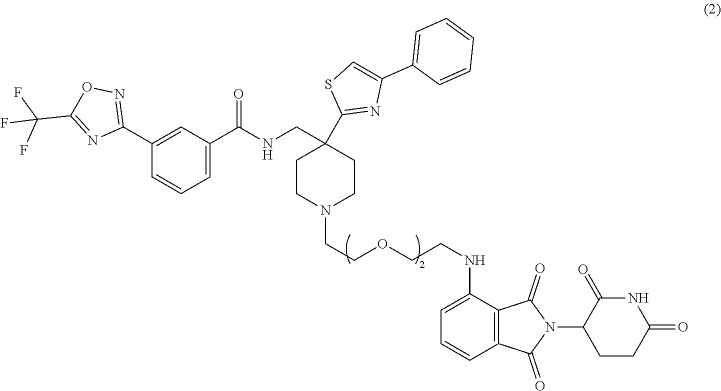

Compound 2 was synthesized in an analogous manner to compound 1 in Example 2 from compounds L8 and int-10, and isolated as a yellow powder.

UPLC-MS RT: 1.44 min (Method A), Mass m/z: 900.72 [M+H]⁺.

### Example 4: Synthesis of N-((1-(2-((2-(2,6-dioxopiperidin-3-yl)-1,3-dioxoisoindolin-4-yl)amino)ethyl)-4-(4-phenylthiazol-2-yl)piperidin-4-yl)methyl)-3-(5-(trifluoromethyl)-1,2,4-oxadiazol-3-yl)benzamide (3)

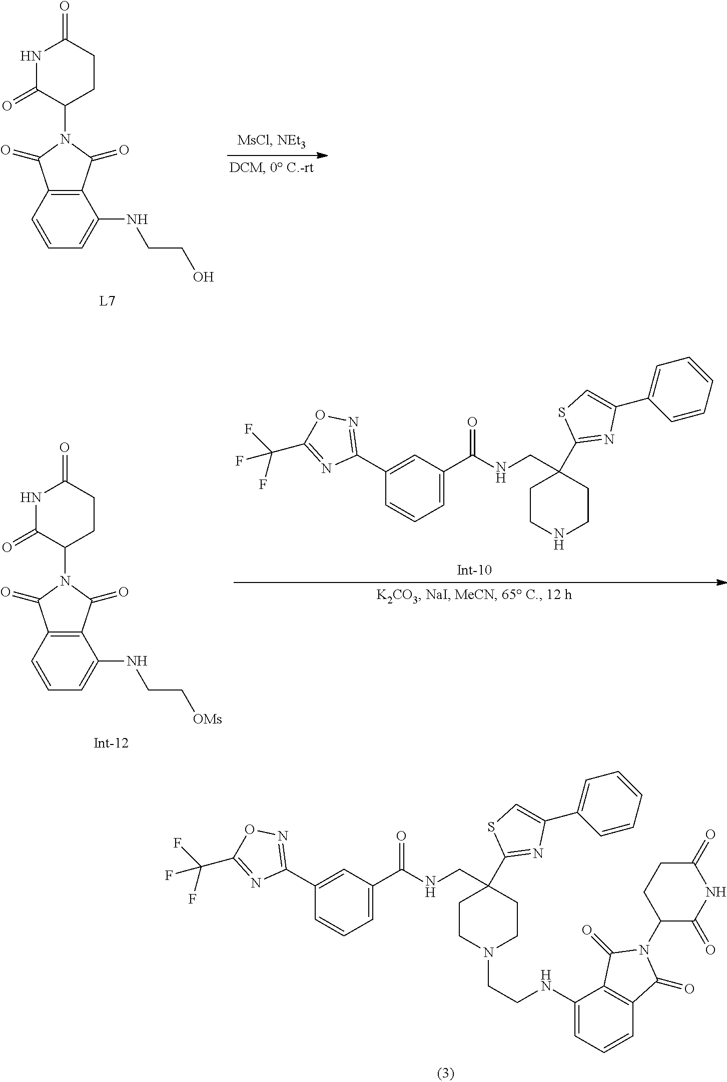 [SMILES: O=C1CCC(N2C(=O)c3cccc(NCCO)c3C2=O)C(=O)N1 ; CS(=O)(=O)OCCNc1cccc2c1C(=O)N(C1CCC(=O)NC1=O)C2=O ; O=C(NCC1(c2nc(-c3ccccc3)cs2)CCNCC1)c1cccc(-c2noc(C(F)(F)F)n2)c1 ; O=C1CCC(N2C(=O)c3cccc(NCCN4CCC(CNC(=O)c5cccc(-c6noc(C(F)(F)F)n6)c5)(c5nc(-c6ccccc6)cs5)CC4)c3C2=O)C(=O)N1]

To a solution of compound L7 (20 mg, 0.063 mmol, 1.0 eq.) in dichloromethane (1 mL), MsCl (7.3 μL, 1.5 eq.) and NEt₃ (1.8 μL, 1.8 eq.) at 0° C. were added. The reaction was allowed to warm to room temperature, stirred for 2 h, and monitored by UPLC-MS. Upon consumption of the starting material, the reaction was quenched with H₂O, extracted three times with dichloromethane three times. The combined organic layers were dried over Na₂SO₄, filtered, and concentrated in vacuo to yield 2-((2-(2,6-dioxopiperidin-3-yl)-1,3-dioxoisoindolin-4-yl)amino)ethyl methanesulfonate (int-12), which was used in the next step without further purification.

UPLC-MS RT: 0.85 min (Method A), Mass m/z: 395.87 [M+H]⁺.

To a solution of compounds int-10 (24 mg, 0.046 mmol, 1.0 eq. crude from deprotection step) and int-12 (25 mg, 1.0 eq. crude from last step) in acetonitrile (1 mL), K₂CO₃ (17 mg, 2.5 eq.) and NaI (0.73 mg, 0.1 eq.) were added in one portion. The reaction mixture was heated to 65° C. and stirred for 12 h. Upon consumption of the starting material, the mixture was filtered through a pad of Celitet, concentrated in vacuo, and the resulting residue was purified using ISCO (dichloromethane/methanol, 0%-10%) to yield compound 3 as a yellow powder (13.5 mg, 35% yield over three steps).

UPLC-MS RT: 1.51 min (Method A), Mass m/z: 812.71 [M+H]⁺.

### Example 5: Synthesis of (2S,4R)-1-((S)-3,3-dimethyl-2-(4-(4-(4-phenylthiazol-2-yl)-4-((3-(5-(trifluoromethyl)-1,2,4-oxadiazol-3-yl)benzamido)methyl)piperidin-1-yl)butanamido)butanoyl)-4-hydroxy-N-((S)-1-(4-(4-methylthiazol-5-yl)phenyl)ethyl)pyrrolidine-2-carboxamide (4)

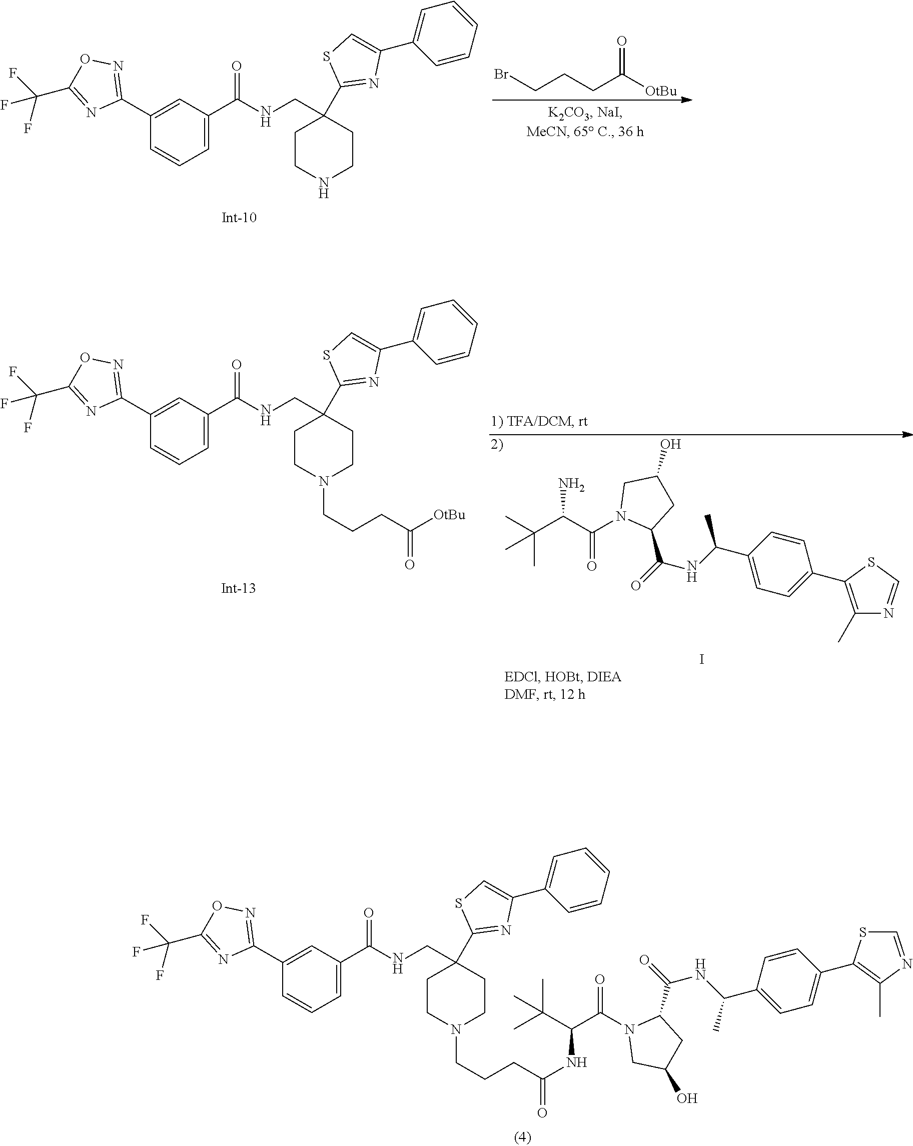

To a solution of compound int-10 (25 mg, 0.049 mmol, 1.0 eq. crude from deprotection step) and tert-butyl 4-bromobutanoate (16 mg, 1.5 eq.) in acetonitrile (1 mL), K₂CO₃ (17 mg, 2.5 eq.) and NaI (0.73 mg, 0.1 eq.) were added in one portion. The reaction mixture was heated to 65° C. and stirred for 36 h. Upon consumption of the starting material, the mixture was filtered through a pad of Celitet, concentrated in vacuo, and the residue was purified using ISCO (dichloromethane/methanol, 0%-10%) to yield tert-butyl 4-(4-(4-phenylthiazol-2-yl)-4-((3-(5-(trifluoromethyl)-1,2,4-oxadiazol-3-yl)benzamido)methyl)piperidin-1-yl)butanoate (int-13) (30 mg, 94% yield over 2 steps).

UPLC-MS RT: 1.37 min (Method A), Mass m/z: 655.80 [M+H]⁺.

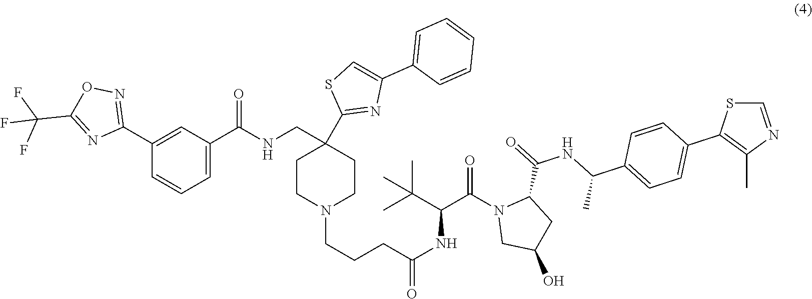 [SMILES: Cc1ncsc1-c1ccc([C@H](C)NC(=O)[C@@H]2C[C@@H](O)CN2C(=O)[C@@H](NC(=O)CCCN2CCC(CNC(=O)c3cccc(-c4noc(C(F)(F)F)n4)c3)(c3nc(-c4ccccc4)cs3)CC2)C(C)(C)C)cc1]

Compound int-13 (30 mg, 0.046 mmol, 1.0 eq.) was treated with a mixture of TFA/dichloromethane (1:5) at room temperature. The reaction was stirred for 3 h. Upon consumption of the starting material, the solvent was removed in vacuo, and the residue was used without further purification.

UPLC-MS RT: 1.34 min (Method A), Mass m/z: 599.79 [M+H]⁺.

A solution of the crude residue from last step (1.0 eq.) and compound I (VHL ligand) (2.0 mg, 0.041 mmol, 0.9 eq.) in DMF (0.5 mL) was treated with 1-ethyl-3-(3-dimethylaminopropyl)carbodiimide (EDCI) (9.7 mg, 1.1 eq.), hydroxybenzotriazole (HOBt) (6.8 mg, 0.051 mmol, 1.1 eq.), and DIEA (28 μL, 2.0 eq.). The reaction mixture was stirred at room temperature for 12 h and monitored by UPLC-MS. Upon completion of the reaction, the mixture was quenched with H₂O and extracted three times with ethyl acetate. The combined organic layers were washed with brine, dried over Na₂SO₄, filtered, and concentrated in vacuo. The resulting residue was purified using HPLC (H₂O/acetonitrile, 0%-100%) to yield compound 4 as a white powder (13.5 mg, 32% yield over 2 steps).

UPLC-MS RT: 1.60 min (Method A), Mass m/z: 1025.64 [M+H]⁺.

¹H NMR (500 MHz, DMSO-d₆) δ 8.98 (s, 1H), 8.77 (br s, 1H), 8.44 (s, 1H), 8.36 (d, J=7.8 Hz, 1H), 8.19 (dd, J=7.7, 1.6 Hz, 1H), 8.11-8.02 (m, 2H), 7.93 (d, J=6.8 Hz, 2H), 7.82 (br s, 1H), 7.69 (t, J=7.9 Hz, 1H), 7.45-7.34 (m, 6H), 7.30 (t, J=7.3 Hz, 1H), 5.09 (d, J=3.5 Hz, 1H), 4.91 (p, J=7.1 Hz, 1H), 4.49 (d, J=9.3 Hz, 1H), 4.41 (t, 0.1=8.0 Hz, 1H), 4.27 (s, 1H), 3.66-3.47 (m, 4H), 3.01-2.57 (m, 2H), 2.45 (s, 3H), 2.42-1.92 (m, 11H), 1.78 (ddd, J=12.9, 8.5, 4.7 Hz, 1H), 1.74-1.57 (m, 2H), 1.37 (d, J=7.0 Hz, 3H), 0.92 (s, 9H).

¹³C NMR (126 MHz, DMSO) δ 171.81, 170.60, 169.51, 168.09, 165.73, 165.33, 164.99, 153.77, 151.49, 147.76, 144.64, 135.67, 134.33, 131.29, 131.11, 129.91, 129.70, 129.65, 129.50, 128.82 (2C), 128.63 (2C), 127.82, 126.38 (2C), 126.32, 126.0) (2C), 124.59, 114.67, 68.76, 58.55, 57.98, 56.46, 56.25, 49.46, 47.70, 44.76, 37.73, 35.21, 26.44 (3C), 22.41, 15.98. Four CH₂ carbons of the piperidine ring, and three CH₂ carbons of the propyl linker are not observed in ¹³C NMR.

### Example 6: Synthesis of (2S,4R)-1-((S)-3,3-dimethyl-2-(8-(4-(4-phenylthiazol-2-yl)-4-((3-(5-(trifluoromethyl)-1,2,4-oxadiazol-3-yl)benzamido)methyl)piperidin-1-yl)octanamido)butanoyl)-4-hydroxy-N-((S)-1-(4-(4-methylthiazol-5-yl)phenyl)ethyl)pyrrolidine-2-carboxamide (5)

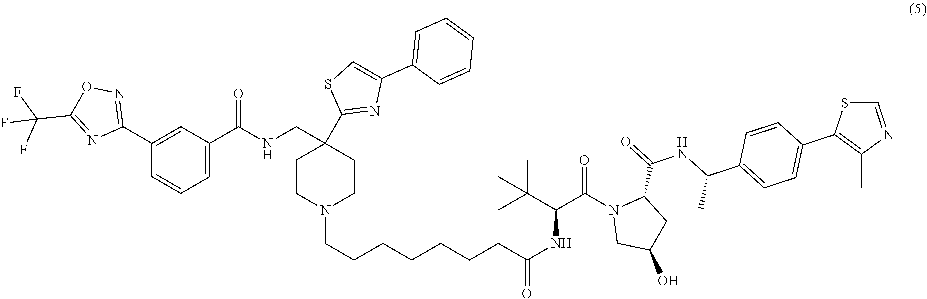 [SMILES: Cc1ncsc1-c1ccc([C@H](C)NC(=O)[C@@H]2C[C@@H](O)CN2C(=O)[C@@H](NC(=O)CCCCCCCN2CCC(CNC(=O)c3cccc(-c4noc(C(F)(F)F)n4)c3)(c3nc(-c4ccccc4)cs3)CC2)C(C)(C)C)cc1]

Compound 5 was synthesized in an analogous manner to compound 4 in Example 5 from compounds int-10 and tert-butyl 8-bromooctanoate, and isolated as a white powder.

UPLC-MS RT: 1.52 min (Method A), Mass m/z: 1081.75 [M+H]⁺.

### Example 7: Synthesis of (2S,4R)-1-((S)-3,3-dimethyl-2-(3-(2-(4-(4-phenylthiazol-2-yl)-4-((3-(5-(trifluoromethyl)-1,2,4-oxadiazol-3-yl)benzamido)methyl)piperidin-1-yl)ethoxy)propanamido)butanoyl)-4-hydroxy-N-((S)-1-(4-(4-methylthiazol-5-yl)phenyl)ethyl)pyrrolidine-2-carboxamide (6)

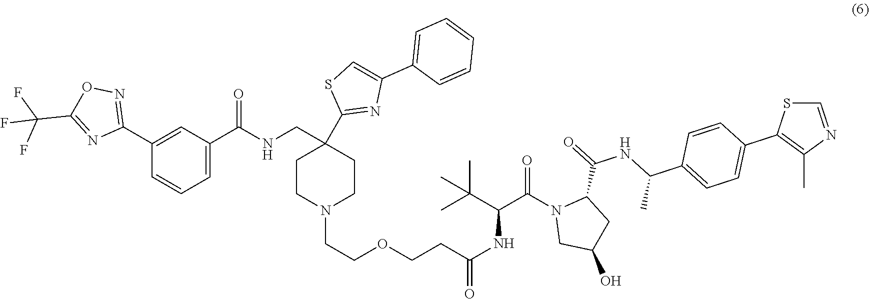 [SMILES: Cc1ncsc1-c1ccc([C@H](C)NC(=O)[C@@H]2C[C@@H](O)CN2C(=O)[C@@H](NC(=O)CCOCCN2CCC(CNC(=O)c3cccc(-c4noc(C(F)(F)F)n4)c3)(c3nc(-c4ccccc4)cs3)CC2)C(C)(C)C)cc1]

Compound 6 was synthesized in an analogous manner to compound 4 in Example 5 from compounds int-10 and tert-butyl 3-(2-bromoethoxy)propanoate, and isolated as a white powder.

UPLC-MS RT: 1.56 min (Method A), Mass m/z: 1055.74 [M+H]⁺.

### Example 8: Synthesis of N-((1-(2-(2-(2-((2-(2,6-dioxopiperidin-3-yl)-1,3-dioxoisoindolin-4-yl)amino)ethoxy)ethoxy)ethyl)-4-(4-phenylthiazol-2-yl)piperidin-4-yl)methyl)-3-(5-(trifluoromethyl)-1,2,4-oxadiazol-3-yl)benzamide (7)

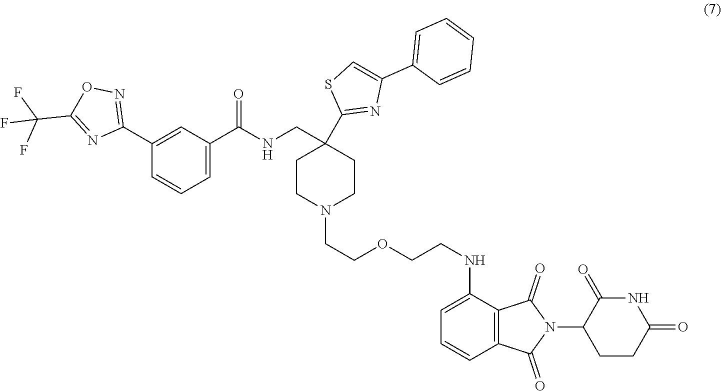 [SMILES: O=C1CCC(N2C(=O)c3cccc(NCCOCCN4CCC(CNC(=O)c5cccc(-c6noc(C(F)(F)F)n6)c5)(c5nc(-c6ccccc6)cs5)CC4)c3C2=O)C(=O)N1]

Compound 7 was synthesized in an analogous manner to compound 2 in Example 5 from compound int-10.

### Example 9: Synthesis of N-((1-(1-((2-(2,6-dioxopiperidin-3-yl)-1,3-dioxoisoindolin-4-yl)oxy)-2-oxo-5,8,11-trioxa-3-azatridecan-13-yl)-4-phenylthiazol-2-yl)piperidin-4-yl)methyl)-3-(5-(trifluoromethyl)-1,2,4-oxadiazol-3-yl)benzamide (8)

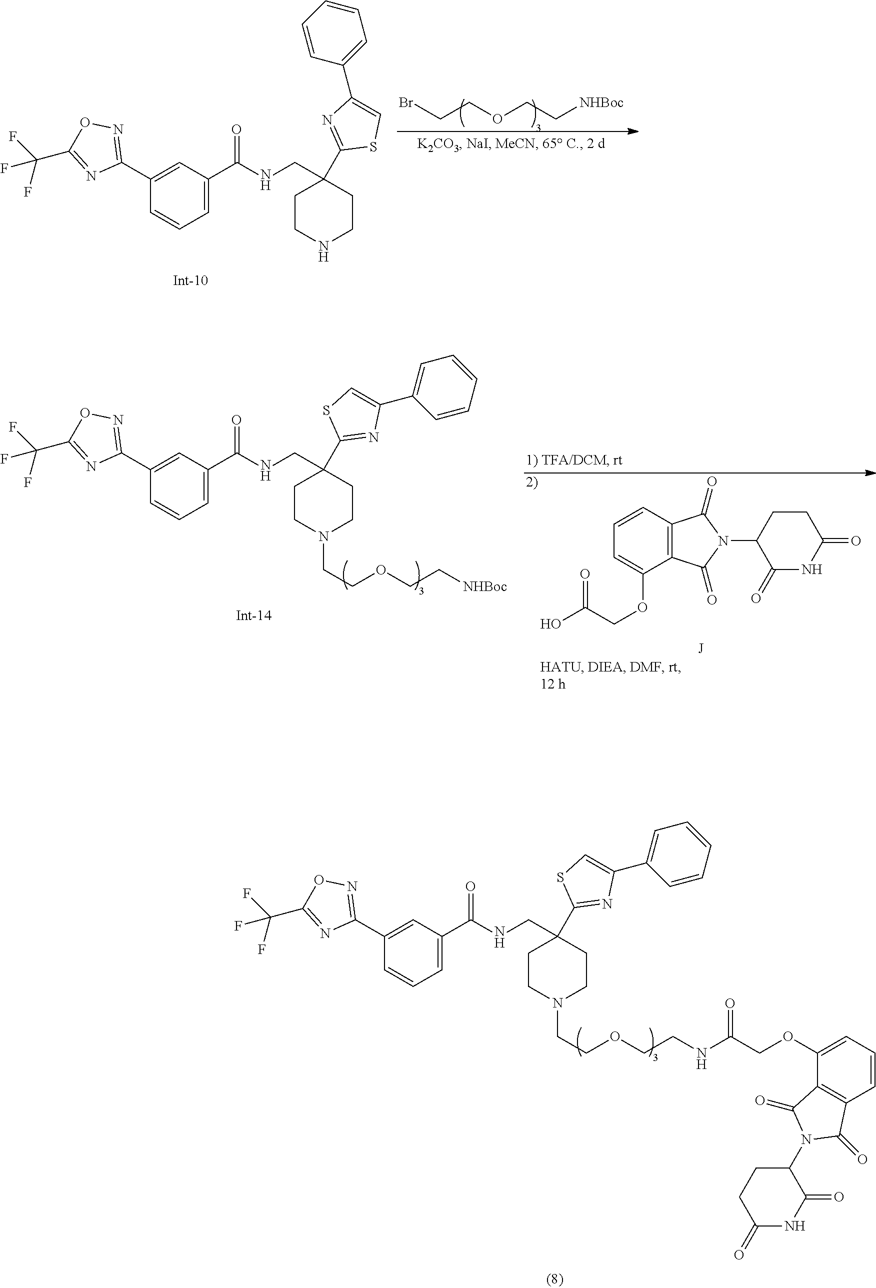

To a solution of compound int-10 (25 mg, 0.049 mmol, 1.0 eq. crude from deprotection step) and tert-butyl (2-(2-(2-(2-bromoethoxy)ethoxy)ethoxy)ethyl)carbamate (33 mg, 1.9 eq.) in acetonitrile (0.5 mL), K₂CO₃ (17 mg, 2.5 eq.) and NaI (0.73 mg, 0.1 eq.) were added in one portion. The reaction mixture was heated to 65° C. and stirred for 2 days. Upon consumption of the starting material, the mixture was filtered through a pad of Celite®, concentrated in vacuo, and the residue was purified using ISCO (dichloromethane/methanol, 0%-10%) to yield tert-butyl (2-(2-(2-(2-(4-(4-phenylthiazol-2-yl)-4-((3-(5-(trifluoromethyl)-1,2,4-oxadiazol-3-yl)benzamido)methyl)piperidin-1-yl)ethoxy)ethoxy)ethoxy)ethyl)carbamate (int-14).

UPLC-MS RT: 1.30 min (Method A), Mass m/z: 788.81 [M+H]⁺.

Compound int-14 (38 mg, 0.049 mmol, 1.0 eq.) was treated with a mixture of TFA/dichloromethane (1:5) at room temperature, and the reaction was stirred for 2 h. Upon consumption of the starting material, the solvent was removed in vacuo, and the resulting residue was used in the next step without further purification.

UPLC-MS RT: 1.16 min (Method A), Mass m/z: 688.80 [M+H]⁺.

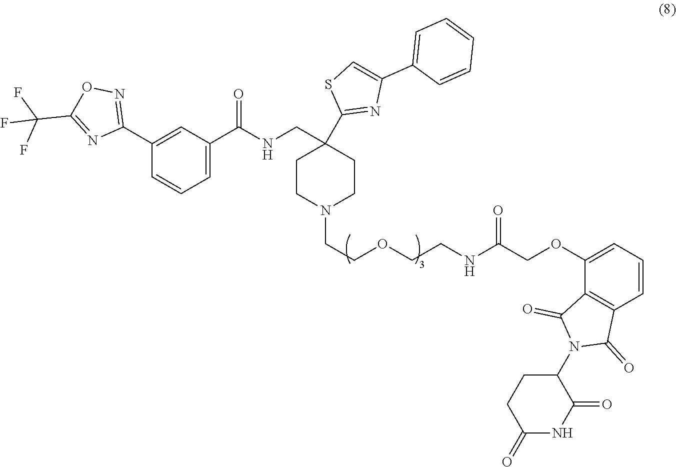

The crude residue from last step (1.0 eq.) and 2-((2-(2,6-dioxopiperidin-3-yl)-1,3-dioxoisoindolin-4-yl)oxy)acetic acid (J) (6.5 mg, 0.6 eq.) was dissolved in DMF (1 mL). The mixture was treated with HATU (14.6 mg, 1.2 eq.) and DIEA (14 μL, 2.5 eq.), and the reaction mixture was stirred at room temperature for 12 h. The reaction was monitored by UPLC-MS. Once the reaction was completed, the mixture was quenched with H₂O, and extracted three times with ethyl acetate. The organic layer was combined and washed with brine, dried over Na₂SO₄, filtered and concentrated in vacuo. The residue was purified using HPLC (H₂O/acetonitrile, 0%-100%) to yield the title compound 8 as a white powder (3.3 mg, 6.8% yield over 3 steps).

UPLC-MS RT: 1.33 min (Method A), Mass m/z: 1002.73 [M+H]⁺.

### Example 10: Synthesis of (2S,4R)-1-((S)-3,3-dimethyl-2-(3-(2-(2-(44-(4-phenylthiazol-2-yl)-4-((3-(5-(trifluoromethyl)-1,2,4-oxadiazol-3-yl)benzamido)methyl)piperidin-1-yl)ethoxy)ethoxy)propanamido)butanoyl)-4-hydroxy-N-((S)-1-(4-(4-methylthiazol-5-yl)phenyl)ethyl)pyrrolidine-2-carboxamide (9)

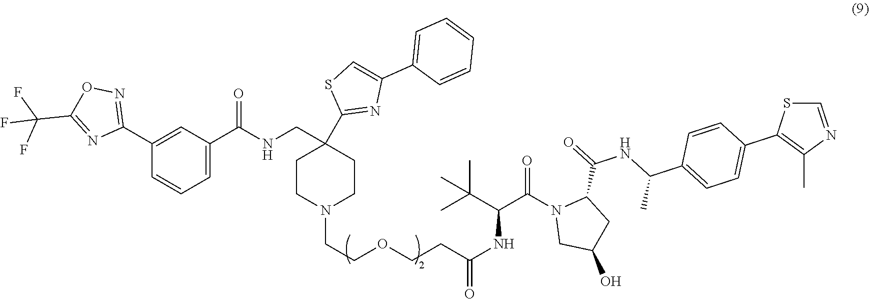

Compound 6 was synthesized in an analogous manner to compound 4 in Example 5 from compounds int-10 and tert-butyl 3-(2-(2-bromoethoxy)ethoxy)propanoate, and isolated as a white powder.

UPLC-MS RT: 1.37 min (Method A), Mass m/z: 1099.65 [M+H]⁺.

### Example 11: Synthesis of N-((1-(2-(3-(2-((S)-1-((S)-2-cyclohexyl-2-((S)-2-(methylamino)propanamido)acetylpyrrolidin-2-yl)thiazole-4-carbonyl)phenoxy)ethyl)-4-(4-phenylthiazol-2-yl)piperidin-4-yl)methyl)-3-(5-(trifluoromethyl)-1,2,4-oxadiazol-3-yl)benzamide (10)

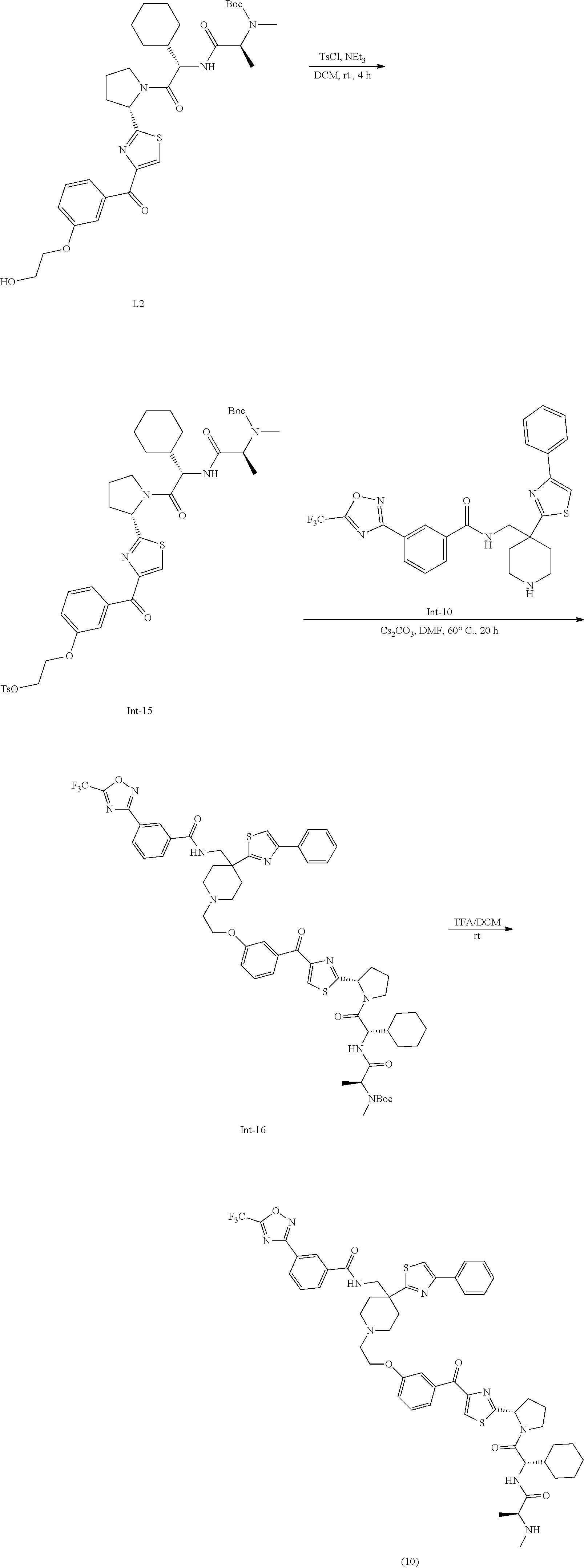

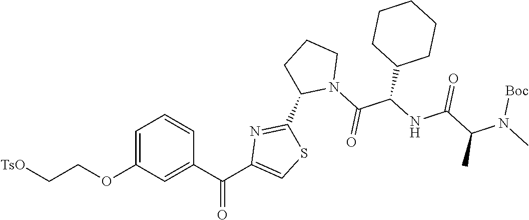 [SMILES: Cc1ccc(S(=O)(=O)OCCOc2cccc(C(=O)c3csc([C@@H]4CCCN4C(=O)[C@@H](NC(=O)[C@H](C)N(C)C(=O)OC(C)(C)C)C4CCCCC4)n3)c2)cc1]

### 2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethyl-4-methylbenzenesulfonate (Int-15)

To a solution of compound L12 (54 mg, 1.0 eq.) in dichloromethane (1 mL), TsCl (32 μL, 2.0 eq.) and NEt₃ (35 μL, 3.0 eq.) were added at (PC. The reaction was allowed to warm to room temperature, stirred for 4 h, and monitored by UPLC-MS. Upon consumption of the starting material, the reaction was quenched with H₂O and extracted three times with dichloromethane. The combined organic layers were dried over Na₂SO₄, filtered, concentrated in vacuo, and purified using ISCO (dichloromethane/methanol, 0%-10%) to yield int-15.

UPLC-MS RT: 1.77 min (Method A), Mass m/z: 796.81 [M+H]⁺.

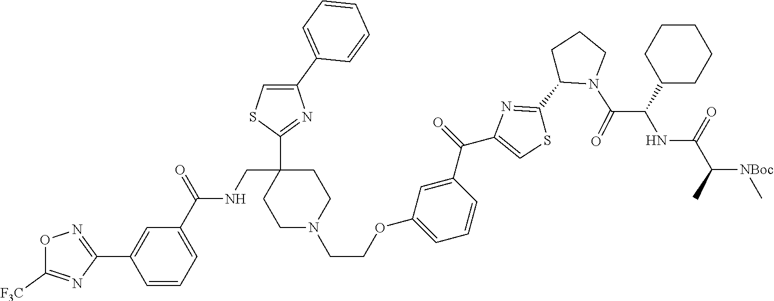 [SMILES: C[C@@H](C(=O)N[C@H](C(=O)N1CCC[C@H]1c1nc(C(=O)c2cccc(OCCN3CCC(CNC(=O)c4cccc(-c5noc(C(F)(F)F)n5)c4)(c4nc(-c5ccccc5)cs4)CC3)c2)cs1)C1CCCCC1)N(C)C(=O)OC(C)(C)C]

### tert-butyl ((S)-1-(((S)-1-cyclohexyl-2-oxo-2-((S)-2-(4-(3-(2-(4-(4-phenylthiazol-2-yl)-4-((3-(5-(trifluoromethyl)-1,2,4-oxadiazol-3-yl)benzamido)methyl)piperidin-1-yl)ethoxy)benzoyl)thiazol-2-yl)pyrrolidin-1-yl)ethyl)amino)-1-oxopropan-2-yl)(methyl)carbamate (Int-16)

To a solution of compound int-10 (77 mg, 0.15 mmol, 1.5 eq.) and compound int-S (1.0 eq., from last step) in DMF (1 mL) was added Cs₂CO₃ (41 mg, 1.5 eq.) in one portion. The reaction mixture was heated to 60° C. and stirred for 20 h. Upon consumption of the starting material, the mixture was filtered through a pad of CeliteQ®, concentrated in vacuo, and the resulting residue was purified using ISCO (dichloromethane/methanol, 0%-10%) to yield int-16 (56 mg, 49% yield over 3 steps).

UPLC-MS RT: 1.37 min (Method A), Mass m/z: 1137.56 [M+H]⁺.

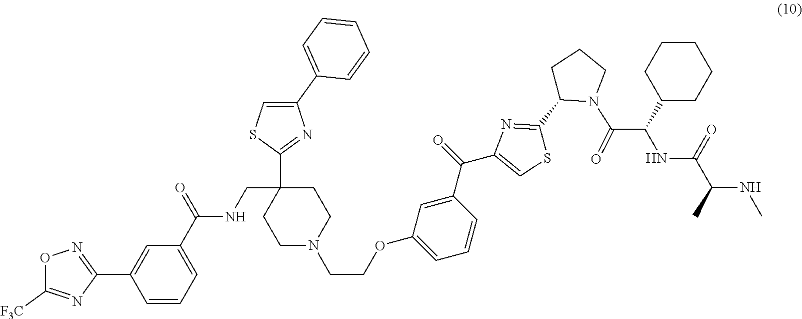 [SMILES: CN[C@@H](C)C(=O)N[C@H](C(=O)N1CCC[C@H]1c1nc(C(=O)c2cccc(OCCN3CCC(CNC(=O)c4cccc(-c5noc(C(F)(F)F)n5)c4)(c4nc(-c5ccccc5)cs4)CC3)c2)cs1)C1CCCCC1]

Compound int-16 (28 mg, 0.025 mmol, 1.0 eq.) was treated with a mixture of TFA/dichloromethane (1:5) at room temperature, and the reaction was monitored by UPLC-MS. Upon consumption of the starting material, the solvent was removed in vacuo, and the resulting residue was purified using HPLC (H₂O/acetonitrile, 0%-100%) to yield compound 10 as a white powder (9.2 mg, 36% yield).

UPLC-MS RT: 1.62 min (Method A), Mass m/z: 1037.74 [M+H]⁺.

¹H NMR (500 MHz, DMSO-d₆) δ 9.97-9.73 (m, 1H, tertiary NH*), 8.97 (t, J=6.6 Hz, 1H), 8.97-8.76 (m, 1H), 8.72 (d, J=8.1 Hz, 1H), 8.53-8.46 (m, 2H), 8.22 (d, J=7.7 Hz, 11H), 8.18 (s, 11H), 8.13-8.07 (m, 11H), 7.95 (d, J=7.2 Hz, 2H), 7.75 (d, J=7.7 Hz, 1H), 7.72 (t. J=7.8 Hz, 111), 7.60 (s, 1H), 7.47 (t, J=7.9 Hz, 11H), 7.40 (t, J=7.6 Hz, 2H), 7.32 (t, J=7.3 Hz, 1H), 7.26 (dd. J=8.1, 2.6 Hz, 1H), 5.38 (dd, J=7.7, 3.4 Hz, 1H), 4.48 (t, J=7.6 Hz, 1H), 4.38 (s, 2H), 3.88 (q, J=6.4 Hz, 1H), 3.84-3.73 (m, 2H), 3.72-3.62 (m, 2H), 3.57-3.48 (m, 4H), 3.12-2.98 (m, 2H), 2.56 (d, J=14.3 Hz, 2H), 2.53-2.48 (m, 3H), 2.38-2.26 (m, 2H), 2.25-2.14 (m, 2H), 2.11-1.96 (m, 2H), 1.78-1.50 (m, 5H), 1.33 (d, J=6.9 Hz, 3H), 1.20-0.94 (m, 6H).

### Example 12: Synthesis of N-((1-(2-(2-(3-(2-((S)-1-((S)-2-cyclohexyl-2-((S)-2-(methylamino)propanamido)acetyl)pyrrolidin-2-yl)thiazole-4-carbonyl)phenoxy)ethoxy)ethyl)-4-(4-phenylthiazol-2-yl)piperidin-4-ylmethyl)-3-(5-(trifluoromethyl)-1,2,4-oxadiazol-3-yl)benzamide (11)

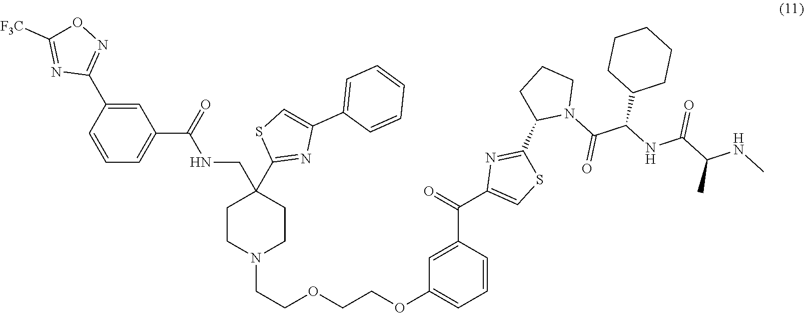 [SMILES: CN[C@@H](C)C(=O)N[C@H](C(=O)N1CCC[C@H]1c1nc(C(=O)c2cccc(OCCOCCN3CCC(CNC(=O)c4cccc(-c5noc(C(F)(F)F)n5)c4)(c4nc(-c5ccccc5)cs4)CC3)c2)cs1)C1CCCCC1]

Compound 11 was synthesized in an analogous manner to compound 10 in Example 11 from compounds int-10 and L13, and isolated as a white powder.

### Example 13: Synthesis of N-((1-(2-(2-(2-(3-(2-((S)-1-((S)-2-cyclohexyl-2-((S)-2-(methylamino)propanamido)acetyl) pyrrolidin-2-yl)thiazole-4-carbonyl)phenoxy)ethoxy)ethoxy)ethyl)-4-(4-phenylthiazol-2-yl)piperidin-4-yl)methyl)-3-(5-(trifluoromethyl)-1,2,4-oxadiazol-3-yl)benzamide (12)

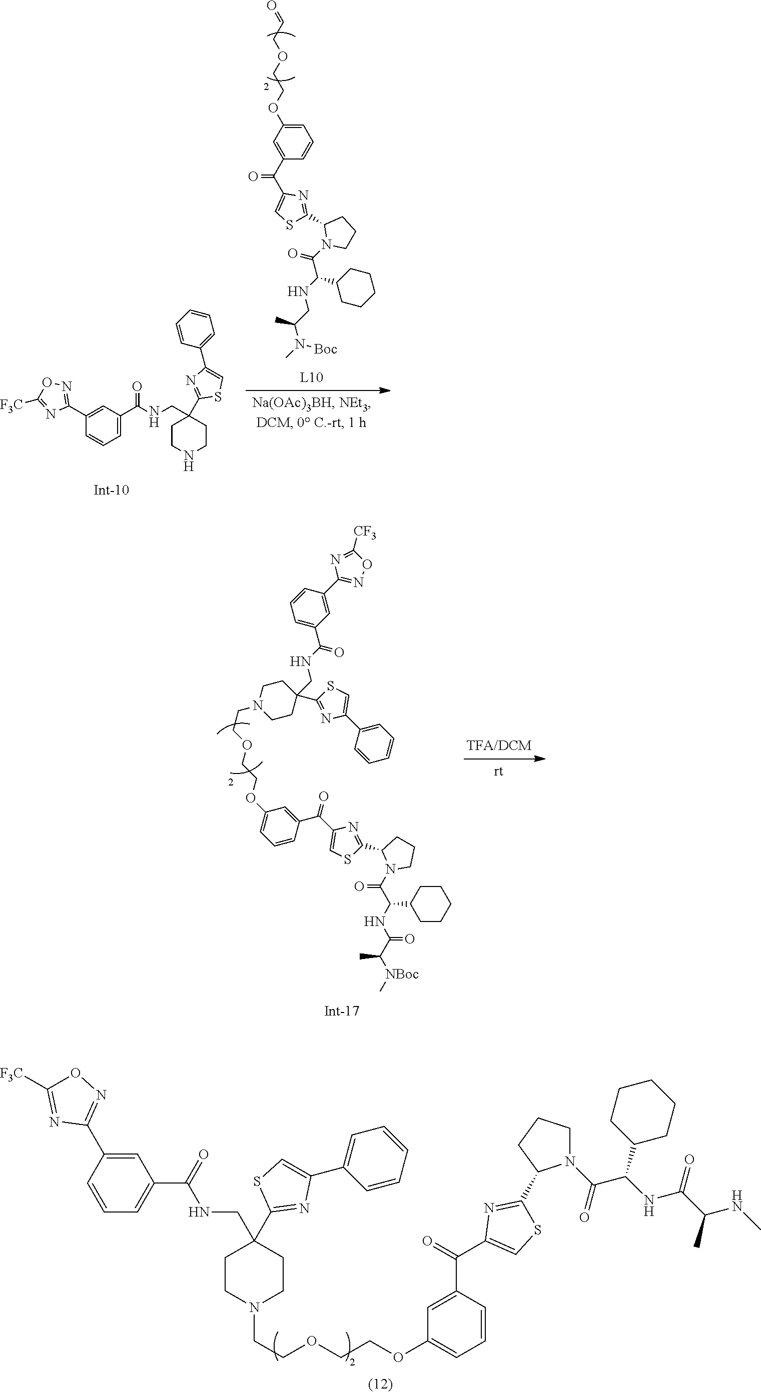

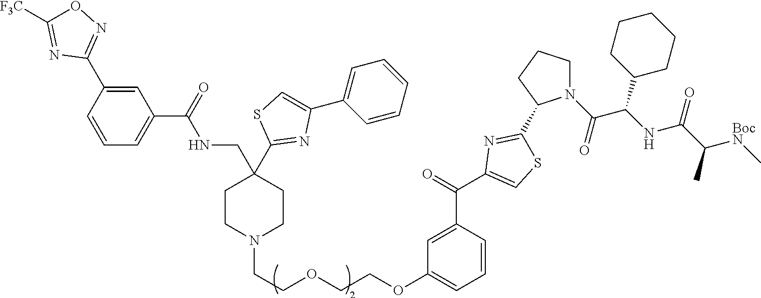

### tert-butyl ((S)-1-(((S)-1-cyclohexyl-2-oxo-2-((S)-2-(4-(3-(2-(2-(2-(4-(4-phenylthiazol-2-yl)-4-((3-(5(trifluoromethyl)-1,2,4-oxadiazol-3-yl)benzamido)methyl)piperidin-1-yl)ethoxy) ethoxy)ethoxy)benzoyl)thiazol-2-yl)pyrrolidin-1-yl)ethyl)amino)-1-oxopropan-2-yl)(methyl)carbamate (int-17)

To a solution of compound int-10 (23 mg, 0.033 mmol, 1.0 eq., crude from the deprotection step) in dichloromethane (1 mL), compound L10 (12 mg, 0.7 eq., crude from deprotection step of 25 mg corresponding acetal in Example 1) and NEt₃(4.6 μL, 2.0 eq.) were added at room temperature, followed by NaBH(OAc)₃ (8.4 mg, 1.2 eq.). The reaction mixture was stirred at room temperature for 1 h. Upon consumption of the starting material, the reaction was quenched with aqueous NaHCO₃ and extracted three times with dichloromethane. The combine organic layers were washed with brine, dried over Na₂SO₄, filtered, and concentrated in vacuo. The resulting residue was purified using ISCO (dichloromethane/methanol, 0%-10%), followed by HPLC (H₂O/acetonitrile, 0%-100%) to yield int-17.

UPLC-MS RT: 2.16 min (Method A), Mass m/z: 1227.50 [M+H]⁺.

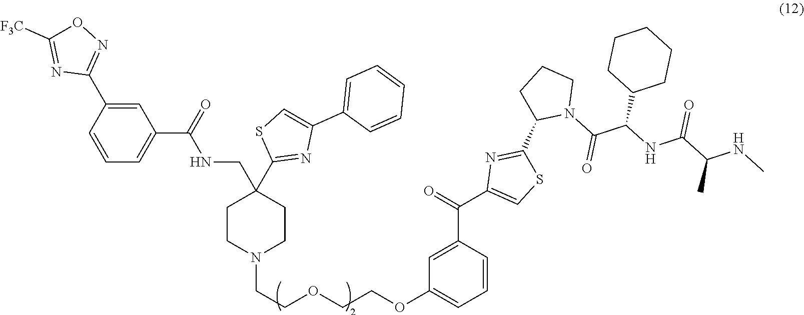

Compound int-17 (1.0 eq. from last step) was treated with a mixture of TFA/dichloromethane (1:5) at room temperature for 2 h, and the reaction was monitored by UPLC-MS. When the starting material was consumed, solvent was removed in vacuo, and the residue using HPLC (H₂O/acetonitrile, 0%-100%) to yield compound 12 as a white powder (10.1 mg, 29% yield over 3 steps).

UPLC-MS RT: 1.41 min (Method A), Mass m/z: 1127.36 [M+H]⁺.

### Example 14: Synthesis of N-((2R)-1-((6-((2-(2,6-dioxopiperidin-3-yl)-1,3-dioxoisoindolin-4-yl)amino)hexyl)(ethyl)amino)propan-2-yl)-4-(5-(trifluoromethyl)-1,2,4-oxadiazol-3-yl)benzamide (13)

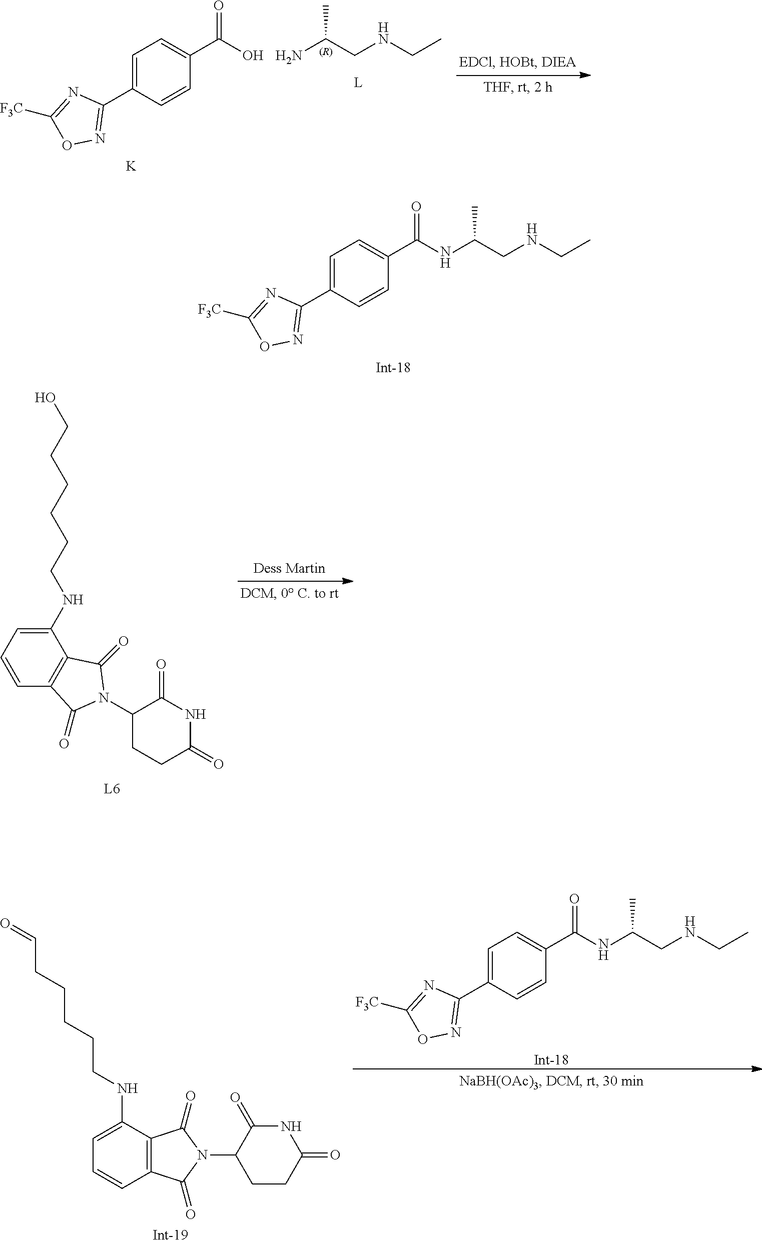

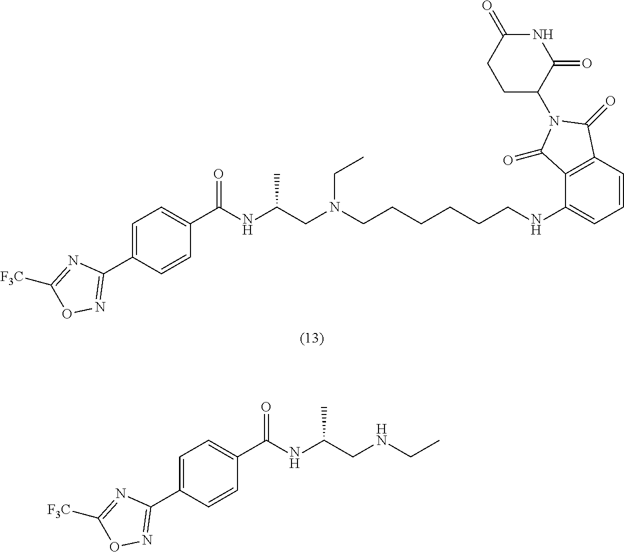 [SMILES: CCN(CCCCCCNc1cccc2c1C(=O)N(C1CCC(=O)NC1=O)C2=O)C[C@@H](C)NC(=O)c1ccc(-c2noc(C(F)(F)F)n2)cc1 ; CCNC[C@@H](C)NC(=O)c1ccc(-c2noc(C(F)(F)F)n2)cc1]

### (R)-N-(1-(Ethylamino)propan-2-yl)-4-(5-(trifluoromethyl)-1,2,4-oxadiazol-3-yl)benzamide (Int-18)

A solution of 4-(5-(trifluoromethyl)-1,2,4-oxadiazol-3-yl)benzoic acid (K) (632 mg, 2.45 mmol, 1.0 eq.) and (R)-N¹-ethylpropane-1,2-diamine (L) (250 mg, 1.0 eq.) in THF (9.8 mL) was treated with EDCI (706 mg, 1.5 eq.), HOBt (430 mg, 1.3 eq.), and DIEA (1.278 mL, 3.0 eq.) at room temperature. The reaction mixture was stirred for 2 h and monitored by UPLC-MS. Upon consumption of the starting material, the reaction was quenched with aqueous NaHCO₃ and extracted three times with ethyl acetate. The combined organic layers were washed with brine, dried over Na:2SO₄, filtered, and concentrated in vacuo. The resulting residue was purified using ISCO (dichloromethane/methanol, 0%-10%) to yield int-18 as a white powder (440 mg, 52.5% yield).

UPLC-MS RT: 0.99 min (Method A), Mass m/z: 343.07 [M+H]⁺.

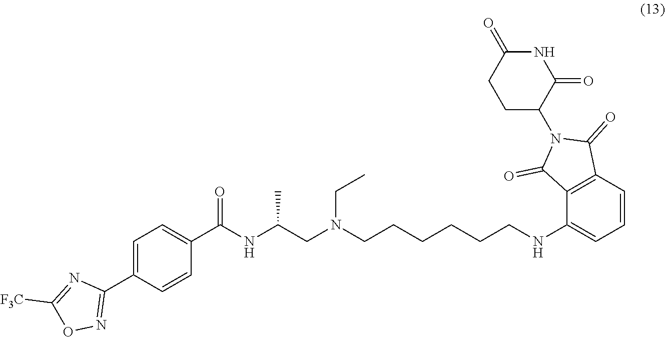 [SMILES: CCN(CCCCCCNc1cccc2c1C(=O)N(C1CCC(=O)NC1=O)C2=O)C[C@@H](C)NC(=O)c1ccc(-c2noc(C(F)(F)F)n2)cc1]

A solution of 2-(2,6-dioxopiperidin-3-yl)-4-((6-hydroxyhexyl)amino)isoindoline-1,3-dione (L6) (25 mg, 0.067 mmol, 1.0 eq.) in dichloromethane (1 mL) was treated with Dess-Martin periodinane (30 mg, 1.05 eq.) at 0° C. The reaction mixture was allowed to warm to room temperature and stirred for 4 h. Upon consumption of the starting material, the reaction mixture was quenched with aqueous NaHCO₃ and extracted three times with ethyl acetate. The combined organic layers were washed with brine, dried over Na₂SO₄, filtered, and concentrated in vacuo. The resulting residue was used in the next step without further purification.

UPLC-MS RT: 1.09 min (Method A), Mass m/z: 354.17 [M−H₂O+H]⁺.

To a solution of crude residue from the above step (1.0 eq.) in dichloromethane (1 mL), compound 70 (23 mg, 1.0 eq.) was added at room temperature, followed by NaBH(OAc)₃ (21 mg, 1.5 eq.). The reaction mixture was stirred at room temperature for 30 minutes. Upon concentration of the starting material, the reaction was quenched with aqueous NaHCO₃ and extracted three times with ethyl acetate. The combined organic layers were washed with brine, dried over Na₂SO₄, filtered, and concentrated in vacuo. The resulting residue was purified using HPLC (H₂O/acetonitrile, 0%-100%) to yield compound 13 as a yellow powder (3.1 mg, 7% over two steps).

UPLC-MS RT: 1.31 min (Method A), Mass m/z: 698.50 [M+H]⁺.

### Example 15: Synthesis of N-((2R)-1-((2-(2-(2-((2-(2,6-dioxopiperidin-3-yl)-1,3-dioxoisoindolin-4-yl)amino)ethoxy)ethoxy)ethyl)(ethyl)amino)propan-2-yl)-4-(5-(trifluoromethyl)-1,2,4-oxadiazol-3-yl)benzamide (14)

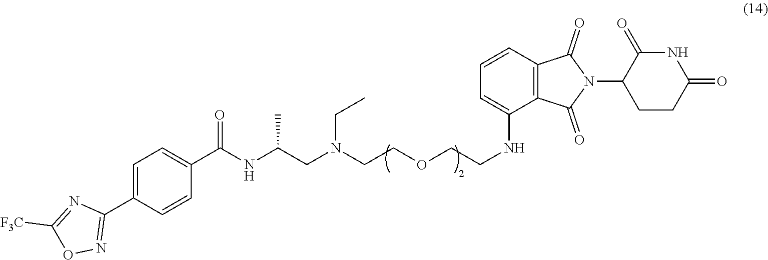

Compound 14 was synthesized in an analogous manner to compound 13 in Example 14 from compounds int-18 and L8, and isolated as a yellow powder.

UPLC-MS RT: 1.29 min (Method A), Mass m/z: 730.41 [M+H]⁺.

### Example 16: Synthesis of N-((R)-1-((5-(3-(2-((S)-1-((S)-2-cyclohexyl-2-((S)-2-(methylamino)propanamido)acetyl)pyrrolidin-2-yl)thiazole-4-carbonyl)phenoxy)pentyl)(ethyl)amino)propan-2-yl)-4-(5-(trifluoromethyl)-1,2,4-oxadiazol-3-yl)benzamide (15)

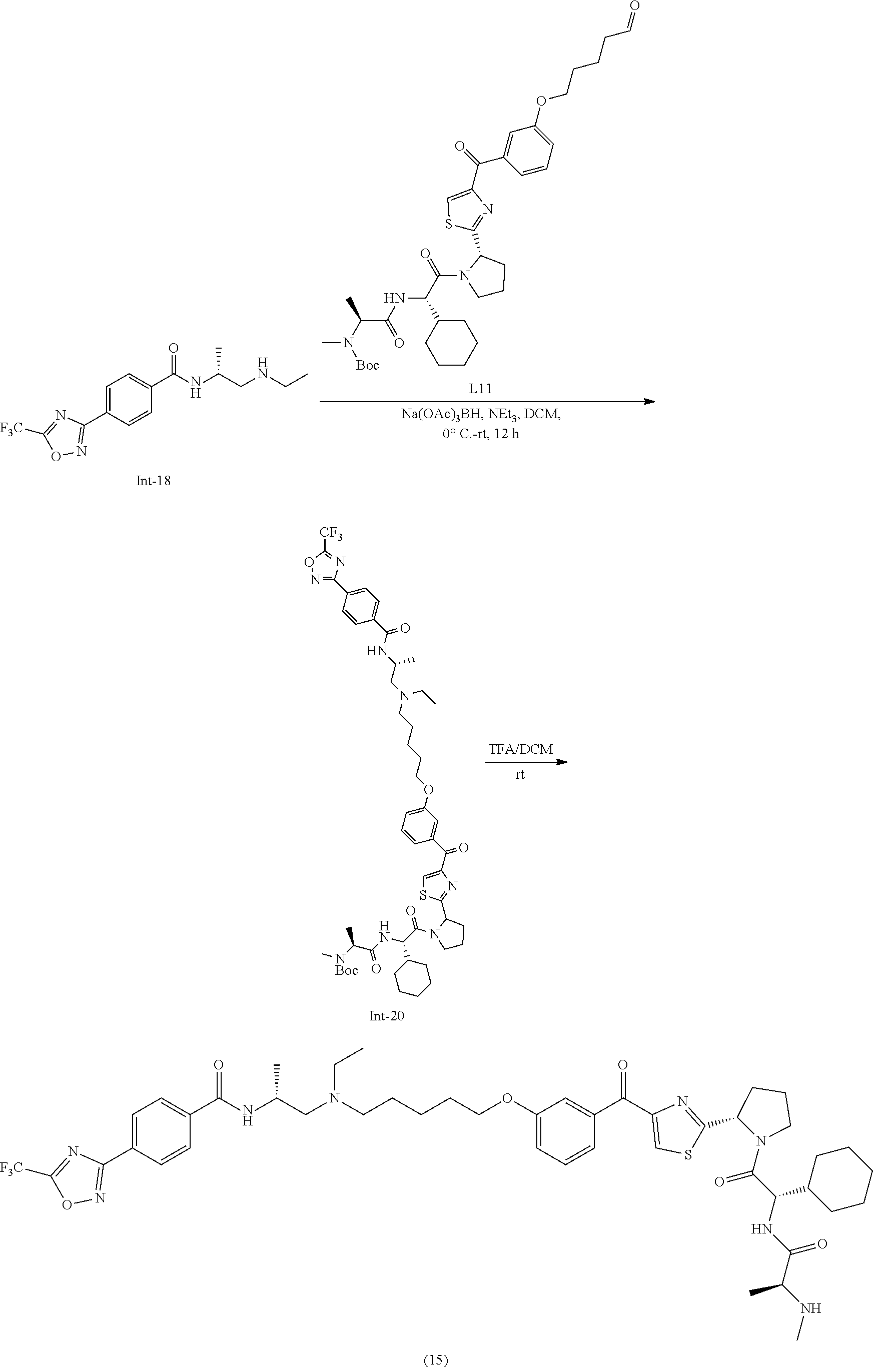

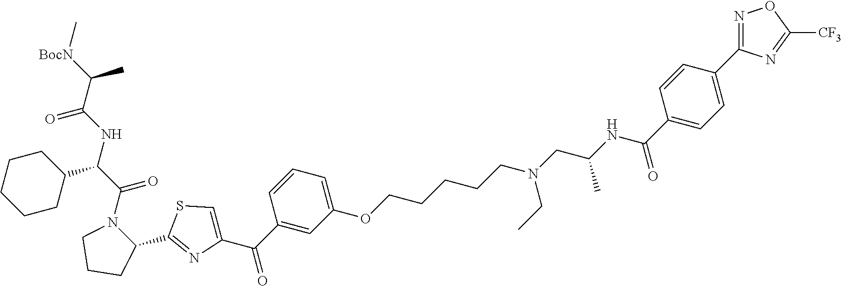 [SMILES: CCN(CCCCCOc1cccc(C(=O)c2csc([C@@H]3CCCN3C(=O)[C@@H](NC(=O)[C@H](C)N(C)C(=O)OC(C)(C)C)C3CCCCC3)n2)c1)C[C@@H](C)NC(=O)c1ccc(-c2noc(C(F)(F)F)n2)cc1]

### tert-Butyl ((S)-1-(((S)-1-cyclohexyl-2-((S)-2-(4-(3-((5-(ethyl((R)-2-(4-(5-(trifluoromethyl)-1,2,4-oxadiazol-3-yl)benzamido)propyl)amino)pentyl)oxy)benzoyl)thiazol-2-yl)pyrrolidin-1-yl)-2-oxoethyl)amino)-1-oxopropan-2-yl)(methyl)carbamate (Int-20)

To a solution of compound int-18 (13.2 mg, 0.039 mmol, 0.8 eq.) in dichloromethane (0.5 mL), compound L11 (33 mg, 1.0 eq., crude from deprotection step of 35 mg corresponding acetal) and NEt₃ (13 μL, 2.0 eq.) were added at 0° C., followed by NaBH(OAc)₃ (10 mg, 1.0 eq.). The reaction mixture was stirred at room temperature for 1 h. Upon consumption of the starting material, the reaction was quenched with aqueous NaHCO₃ and extracted three times with ethyl acetate. The combined organic layers were washed with brine, dried over Na₂SO₄, filtered, and concentrated in vacuo. The resulting residue was purified using ISCO (dichloromethane/methanol, 0%-10) to yield int-20.

UPLC-MS RT: 1.83 min (Method A), Mass m/z: 1009.63 [M+H]⁺.

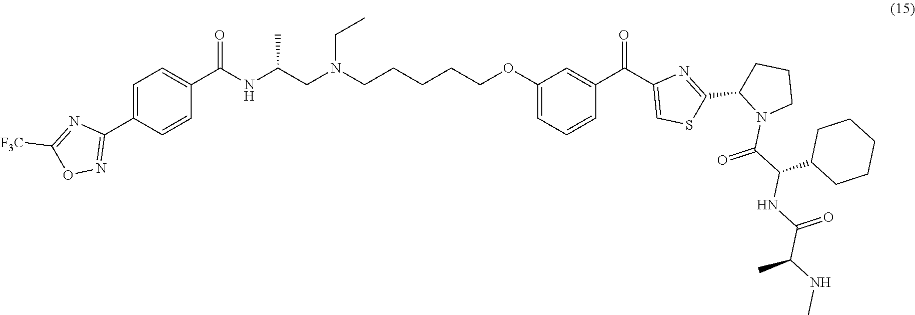 [SMILES: CCN(CCCCCOc1cccc(C(=O)c2csc([C@@H]3CCCN3C(=O)[C@@H](NC(=O)[C@H](C)NC)C3CCCCC3)n2)c1)C[C@@H](C)NC(=O)c1ccc(-c2noc(C(F)(F)F)n2)cc1]

The residue from the above step (1.0 eq.) was treated with a mixture of TFA/dichloromethane (1:5) at room temperature for 90 minutes, and the reaction was monitored by UPLC-MS. Upon consumption of the starting material, the solvent was removed in vacuo, and the resulting residue was purified using HPLC (H₂O/acetonitrile, 0%-100%) to yield compound 15 as a white powder (12.6 mg, 36% yield over 3 steps).

UPLC-MS RT: 1.61 min (Method A), Mass m/z: 908.82 [M+H]⁺.

¹H NMR (500 MHz, DMSO-d₆) δ 9.02 (br s, 1H, tertiary NH*), 8.94-8.78 (m, 1H), 8.76 (t, J=7.6 Hz, 1H), 8.72 (d, J=8.1 Hz, 1H), 8.48 (d, J=3.4 Hz, 1H), 8.19 (dd, J=8.4, 2.0 Hz, 2H), 8.10 (dd, J=8.5, 2.5 Hz, 2H), 7.70 (d, J=7.6 Hz, 1H), 7.56 (dt, J=13.8, 2.2 Hz, 1H), 7.45 (td, J=8.0, 4.6 Hz, 1H), 7.26-7.18 (m, 1H), 5.44-5.32 (m, 1H), 4.60-4.40 (m, 2H), 4.04 (dt, J=12.9, 6.3 Hz, 2H), 3.91-3.75 (m, 3H), 3.38-3.05 (m, 6H), 2.54-2.44 (m, 3H), 2.30-2.12 (m, 2H), 2.04 (d, J=7.6 Hz, 2H), 1.87-1.39 (m, 11H), 1.33 (d, J=6.9 Hz, 3H), 1.30-1.18 (m, 6H), 1.18-0.93 (m, 6H).

### Example 17: Synthesis of N-((2R)-1-((2-((2-(2,6-dioxopiperidin-3-yl)-1,3-dioxoisoindolin-4-yl)amino)ethyl)(ethyl)amino)propan-2-yl)-4-(5-(trifluoromethyl)-1,2,4-oxadiazol-3-yl)benzamide (16)

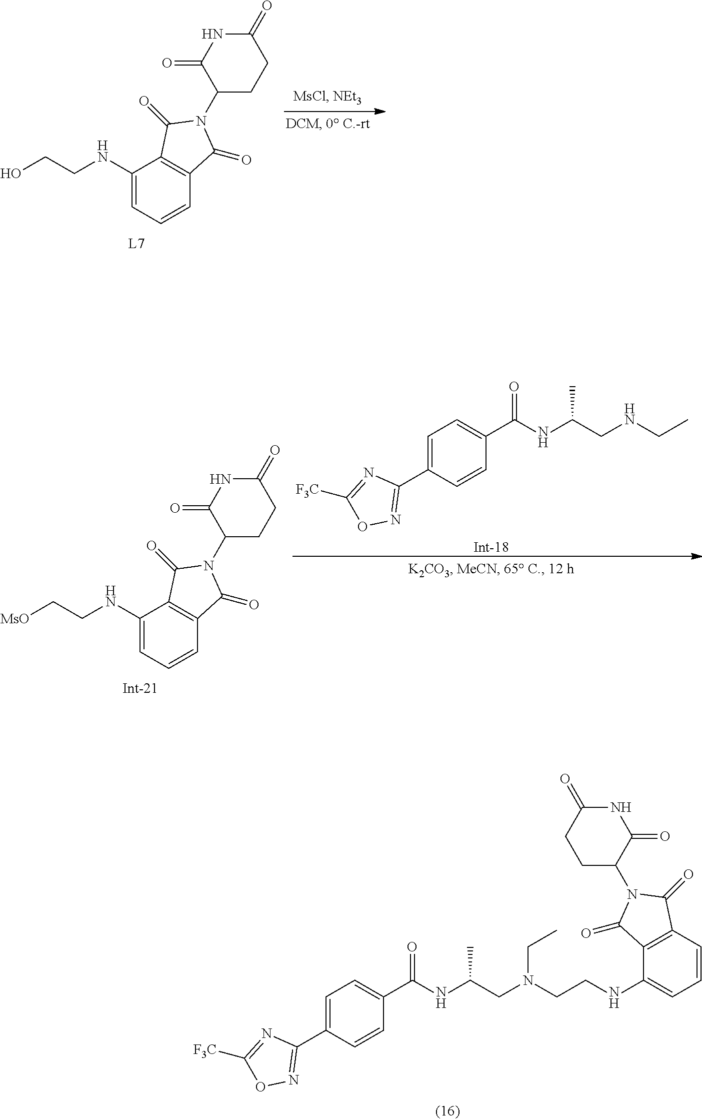

To a solution of compound L7 (25 mg, 0.079 mmol, 1.0 eq.) in dichloromethane (1 mL) was added MsCl (36.8 μL, 6.0 eq.) and NEt₃ (79 μL, 7.2 eq.) at 0° C. The reaction was allowed to warm to room temperature, stirred for 3 h, and monitored by UPLC-MS. Upon consumption of the starting material, the reaction was quenched with H₂O, extracted three times with dichloromethane, dried over Na₂SO₄, filtered, and concentrated in vacuo to yield compound int-21, which was used in the next step without further purification.

UPLC-MS RT: 0.85 min (Method A), Mass m/z: 395.87 [M+H]⁺.

To a solution of compounds int-18 (24 mg, 0.070 mmol, 0.9 eq.) and int-21 (1.0 eq. crude from last step) in acetonitrile (1 mL), K₂CO₃ (22 mg, 2.0 eq.) was added in one portion. The reaction mixture was heated to 65° C. and stirred for 12 h. Upon consumption of the starting material, the mixture was filtered through a pad of Celite® t and concentrated in vacuo. The resulting residue was purified using HPLC (H₂O/acetonitrile, 0%-100%) to yield compound 16 as a yellow powder (2.7 mg, 5.3% yield over two steps).

UPLC-MS RT: 1.13 min (Method A), Mass m/z: 641.90 [M+H]⁺.

### Example 18: Synthesis of (2S,4R)-1-((3R,16S)-16-(tert-butyl)-5-ethyl-3-methyl-1,14-dioxo-1-(4-(5-(trifluoromethyl)-1,2,4-oxadiazol-3-yl)phenyl)-8,11-dioxa-2,5,15-triazaheptadecan-17-oyl)-4-hydroxy-N-((S)-1-(4-(4-methylthiazol-5-yl)phenylethyl)pyrrolidine-2-carboxamide (17)

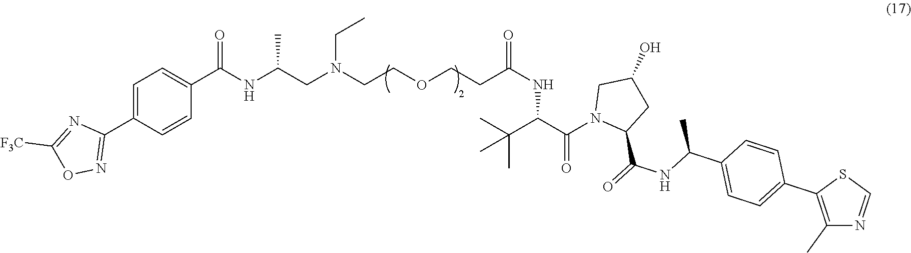

Compound 17 was synthesized in an analogous manner to compound 18 in Example 19, below, from compounds int-18 and tert-butyl 3-(2-(2-bromoethoxy)ethoxy)propanoate, and isolated as a white powder.

### Example 19: Synthesis of (2S,4R)-1-((S)-2-(4-(ethyl((R)-2-(4-(5-(trifluoromethyl)-1,2,4-oxadiazol-3-yl)benzamido)propyl)amino)butanamido)-3,3-dimethylbutanoyl)-4-hydroxy-N-((S)-1-(4-(4-methylthiazol-5-yl)phenyl)ethyl)pyrrolidine-2-carboxamide (18)

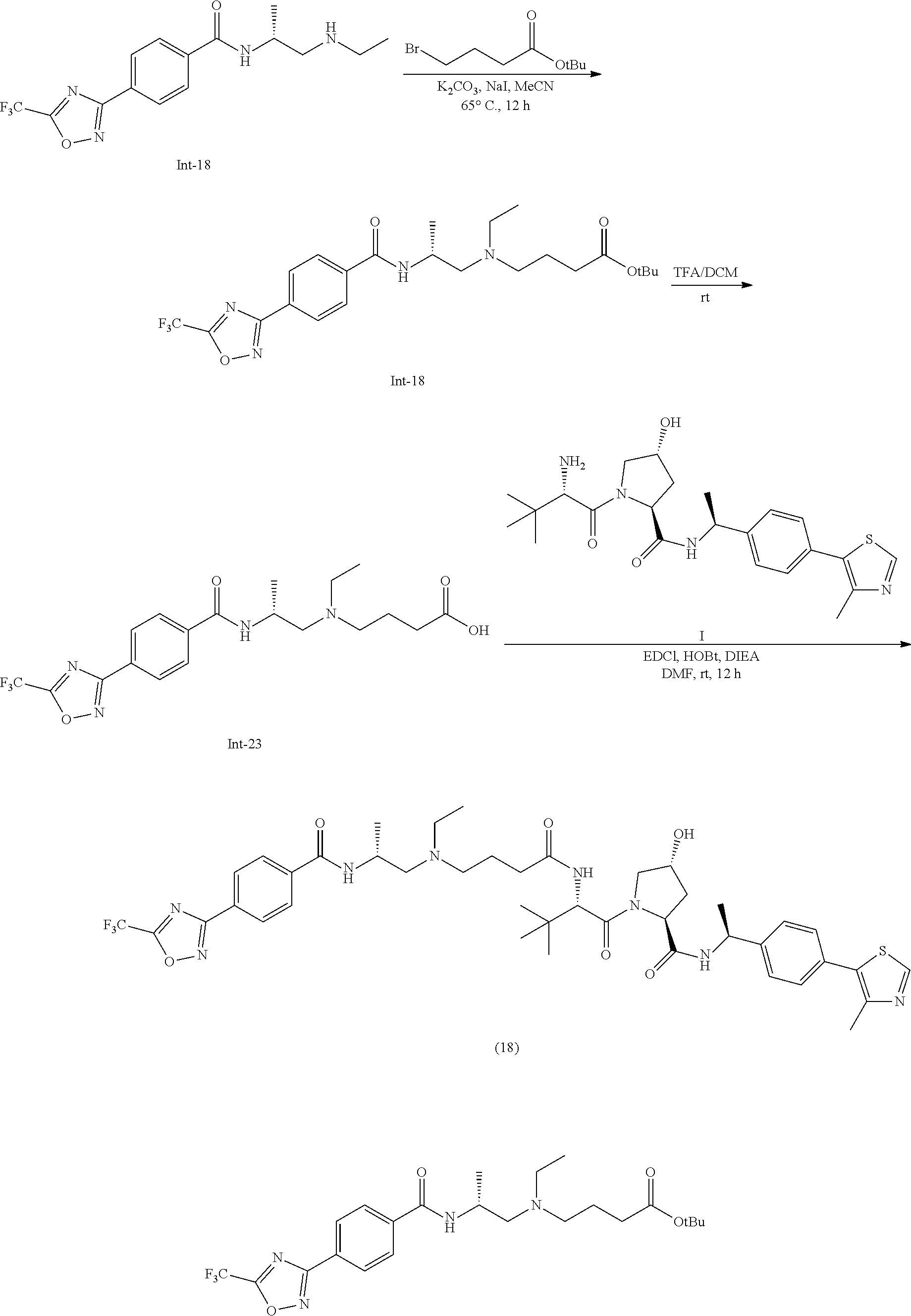

### tert-Butyl (R)-4-(ethyl(2-(4-(5-(trifluoromethyl)-1,24-oxadiazol-3-yl)benzamido)propyl) amino)butanoate (Int-2)

To a solution of compound int-18 (40 mg, 0.12 mmol, 1.0 eq.) and tert-butyl 4-bromobutanoate (21 μL, 1.2 eq.) in acetonitrile (1 mL). K₂CO₃ (32 mg, 2.0 eq.) and NaI (1.8 mg, 0.1 eq.) were added in one portion. The reaction mixture was heated to 65° C. and stirred for 12 h. Upon consumption of the starting material, the mixture was filtered through a pad of Celite® and concentrated in vacuo. The resulting residue was purified using ISCO (dichloromethane/methanol, 0%-10%) to yield int-22.

UPLC-MS RT: 1.28 min (Method A), Mass m/z: 484.98 [M+H]⁺.

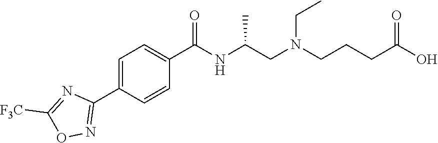 [SMILES: CCN(CCCC(=O)O)C[C@@H](C)NC(=O)c1ccc(-c2noc(C(F)(F)F)n2)cc1]

### (R)-4-(Ethyl(2-(4-(5-(trifluoromethyl)-1,24-oxadiazol-3-yl)benzamido)propyl)amino) butanoic acid (Int-23)

Compound int-22 (1.0 eq. from last step) was treated with a mixture of TFA/dichloromethane (1:5), and the reaction was stirred at room temperature for 4 h. Upon consumption of the starting material, the solvent was removed in vacuo, and the resulting residue (Int-23) was used in the next step without further purification.

UPLC-MS RT: 1.03 min (Method A), Mass m/z: 428.97 [M+H]⁺.

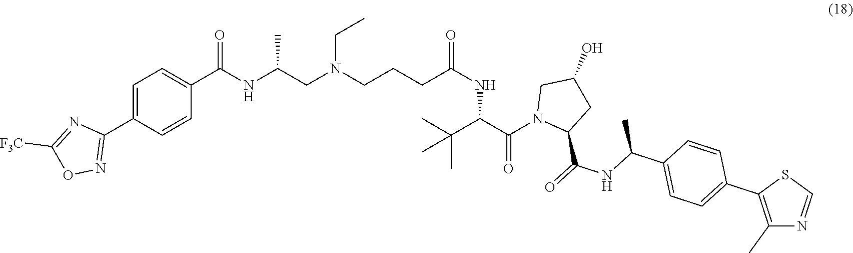 [SMILES: CCN(CCCC(=O)N[C@H](C(=O)N1C[C@H](O)C[C@H]1C(=O)N[C@@H](C)c1ccc(-c2scnc2C)cc1)C(C)(C)C)C[C@@H](C)NC(=O)c1ccc(-c2noc(C(F)(F)F)n2)cc1]

A solution of the crude residue from the above step (1.0 eq.) and L9 (28 mg, 0.5 eq.) in DMF (1 mL) was treated with EDCI (13.5 mg, 0.6 eq.), HOBt (9.5 mg, 0.6 eq.), and DIEA (20 μL, 1.0 eq.). The reaction mixture was stirred at room temperature for 12 h and was monitored by UPLC-MS. Upon completion of the reaction, the mixture was quenched with H₂O and extracted three times with ethyl acetate. The combined organic layers were washed with brine, dried over Na₂SO₄, filtered, and concentrated in vacuo. The resulting residue was purified using HPLC (H₂O/acetonitrile, 0%-100%) to yield compound 18 as a white powder (1.4 mg, 1.4% yield over 3 steps).

UPLC-MS RT: 1.33 min (Method A), Mass m/z: 854.82 [M+H]⁺.

### Example 20: Synthesis of N-((2R)-1-((2-((1-(3-(2-((S)-1-((S)-2-cyclohexyl-2-((S)-2-(methylamino)propanamido)acetyl)pyrrolidin-2-yl)thiazole-4-carbonyl)phenoxy)-3-methoxypropan-2-yl)oxy)ethyl)(ethyl)amino)propan-2-yl)-4-(5-(trifluoromethyl)-1,2,4-oxadiazol-3-yl)benzamide (19)

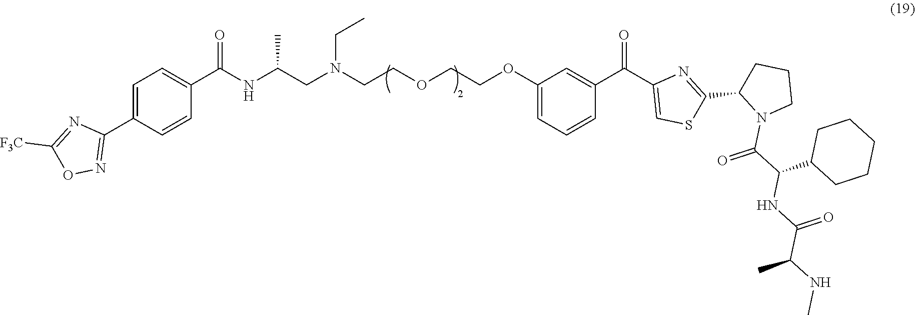

Compound 19 was synthesized in an analogous manner to compound 15 in Example 16 from compounds int-18 and L10, and isolated as a white powder.

UPLC-MS RT: 1.25 min (Method A), Mass m/z: 954.63 [M+H]⁺.

### Example 21: Synthesis of N-((2R)-1-((4-((2-(2,6-dioxopiperidin-3-yl)-1,3-dioxoisoindolin-4-yl)amino)butyl)(ethyl)amino)propan-2-yl)-4-(5-(trifluoromethyl)-1,2,4-oxadiazol-3-yl)benzamide (20)

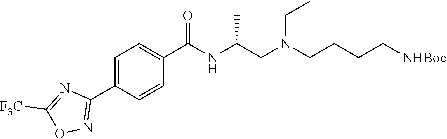 [SMILES: CCN(CCCCNC(=O)OC(C)(C)C)C[C@@H](C)NC(=O)c1ccc(-c2noc(C(F)(F)F)n2)cc1]

### tert-butyl (R)-(4-(ethyl(2-(4-(5-(trifluoromethyl)-1,2,4-oxadiazol-3-yl)benzamido)propyl) amino)butyl)carbamate (int-24)

To a solution of int-18 (50 mg, 0.5 mmol, 1.0 eq.) and tert-butyl (4-bromobutyl)carbamate (75 mg, 2.0 eq.) in acetonitrile (1 mL), K₂CO₃ (60 mg, 2.0 eq.) and NaI (2 mg, 0.1 eq.) were added in one portion. The reaction mixture was heated to 65° C. and stirred for 16 h. Upon consumption of the starting material, the mixture was filtered through a pad of Celite®, concentrated in vacuo, and the resulting residue was purified using ISCO (dichloromethane/methanol, 0%-10%) to yield compound int-24 (75 mg, quant, yield).

UPLC-MS RT: 1.19 min (Method A), Mass m/z: 513.78 [M+H]⁺.

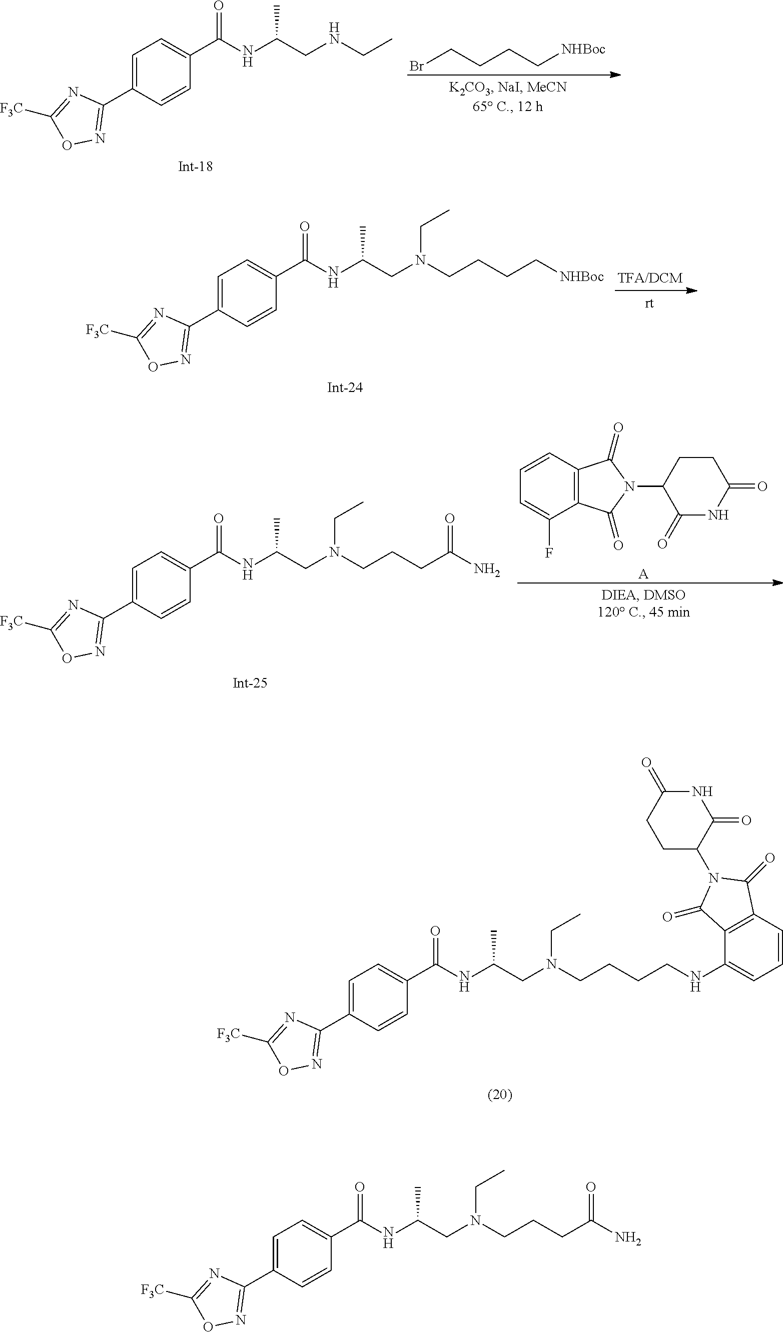

### (R)-N-(1-((4-Aminobutyl)(ethyl)amino)propan-2-yl)-4-(5-(trifluoromethyl)-1,2,4-oxadiazol-3-yl)benzamide (Int-25)

Compound int-24 (75 mg, 0.15 mmol, 1.0 eq.) was treated with a mixture of TFA/dichloromethane (1:5), and the reaction was stirred at room temperature for 2 h. Upon consumption of the starting material, the solvent was removed in vacuo, and the resulting residue (int-25) was used in the next step without further purification.

UPLC-MS RT: 0.90 min (Method A) Mass m/z: 414.07 [M+H]⁺.

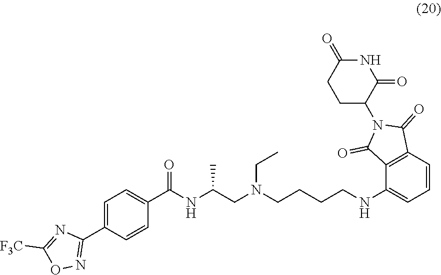 [SMILES: CCN(CCCCNc1cccc2c1C(=O)N(C1CCC(=O)NC1=O)C2=O)C[C@@H](C)NC(=O)c1ccc(-c2noc(C(F)(F)F)n2)cc1]

Compound int-25 (37.5 mg crude from the deprotection step, 1.0 eq.) and DIEA (32 μL, 2.5 eq.) were dissolved in DMSO (1 mL) in a sealed tube. Compound A (30 mg, 1.5 eq.) was added in one batch to the mixture, and the reaction tube was resealed immediately and heated at 120° C. for 45 minutes. The reaction mixture was allowed to cool to room temperature and concentrated in vacuo. The crude product was purified using HPLC (H₂O/acetonitrile, 0%-100%) to yield compound 20 as a yellow powder (8.7 mg, 14% yield over 2 steps).

UPLC-MS RT: 1.19 min (Method A), Mass m/z: 669.80 [M+H]⁺

### Example 22: Synthesis of N-((2R)-1-((3-((2-(2,6-dioxopiperidin-3-yl)-1,3-dioxoisoindolin-4-yl)amino)propyl)(ethyl)amino)propan-2-yl)-4-(5-(trifluoromethyl)-1,2,4-oxadiazol-3-yl)benzamide (21)

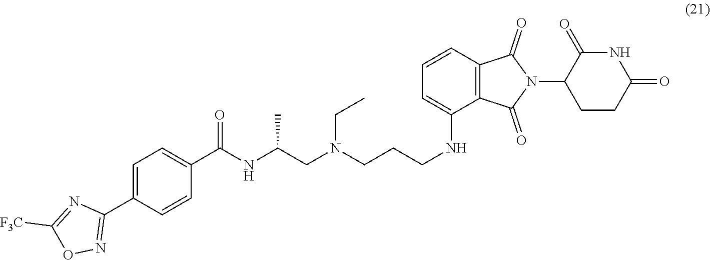 [SMILES: CCN(CCCNc1cccc2c1C(=O)N(C1CCC(=O)NC1=O)C2=O)C[C@@H](C)NC(=O)c1ccc(-c2noc(C(F)(F)F)n2)cc1]

Compound 21 was synthesized in an analogous manner to compound 15 in Example 16 from compounds int-18 and tert-butyl (3-bromopropyl)carbamate, and isolated as a white powder.

UPLC-MS RT: 1.19 min (Method A), Mass m/z: 656.50 [M+H]⁺.

### Example 23: Cellular CRBN Engagement Assay

BRD4_{BD2} was subcloned into mammalian pcDNA5/FRT Vector (Ampicillin and Hygromycin B resistant) modified to contain MCS-eGFP-P2A-mCherry. Stable cell lines expressing eGFP-protein fusion and mCherry reporter were generated using Flp-In™ 293 system. Plasmid (0.3 μg) and pOG44 (4.7 μg) DNA were preincubated in 100 μL of Opti-MEM™ I (Gibco™, Life Technologies™) media containing 0.05 mg/ml Lipofectamine 2000 (Invitrogen™) for 20 min and added to Flp-In™ 293 cells containing 1.9 ml of Dulbecco's Modified Eagle Medium (DMEM) media (Gibco™, Life Technologies™) per well in a 6-well plate format (Falcon, 353046). Cells were propagated after 48 h and transferred into a 10 cm² plate (Corning®, 430165) in DMEM media containing 50 μg/ml of Hygromycin B (Invitrogen™, REF 10687010) as a selection marker. Following 2-3 passage cycles, FACS (FACSAria™ II, BD) was used to enrich for cells expressing eGFP and mCherry.

Stable cells expressing the BRD4_{BD2}-eGFP protein fusion and the mCherry reporter were seeded at a density of 1000-4000 cells/well in a 384-well plate with 50 μL per well of FluoroBrite™ DMEM media (Thermo Fisher Scientific, A18967) supplemented with 10% FBS a day before compound treatment. Compounds and 100 nM dBET6 were dispensed using a D300e Digital Dispenser (HP), normalized to 0.5% DMSO, and incubated with the cells for 5 h. The assay plate was imaged immediately using an acument High Content Imager (TTP Labtech) with 488 nm and 561 nm lasers in a 2 μm×1 μm grid per well format. The resulting images were analyzed using CellProfiler™ (Carpenter et al., Genome Biol. 7(10):R100 (2006)). A series of image analysis steps (an ‘image analysis pipeline’) was constructed. First, the red and green channels were aligned and cropped to target the middle of each well (to avoid analysis of the heavily clumped cells at the edges). A background illumination function was calculated for both red and green channels of each well individually and subtracted to correct for illumination variations across the 384-well plate from various sources of error. An additional step was then applied to the green channel to suppress the analysis of large auto fluorescent artifacts and enhance the analysis of cell specific fluorescence by way of selecting for objects under a given size (30 A.U.) and with a given shape (speckles). mCherry-positive cells were then identified in the red channel by filtering for objects 8-60 pixels in diameter and by using intensity to distinguish between clumped objects. The green channel was then segmented into GFP positive and negative areas and objects were labeled as GFP positive if at least 40% of it overlapped with a GFP positive area. The fraction of GFP-positive cells/mCherry-positive cells in each well was then calculated, and the green and red images were rescaled for visualization. The values for the concentrations that lead to a 50% increase in BRD4_{BD2}-eGFP accumulation (EC₅₀) were calculated using the nonlinear fit variable slope model (GraphPad Software).

The cellular CRBN engagement assay measures the binding affinity by measuring the ability of thalidomide-based degrader molecules to compete with pan-BET bromodomain degrader dBET6 (Nowak et al., Nat. Chem. Biol. 14:706-714 (2018)) for CRBN binding in cells. If no degrader compound is present in the cell, BRD4_{BRD2-e}GFP is degraded by dBET6 via the proteasome system. Therefore, treatment with an increasing concentration of cell-permeable thalidomide-based degrader results in competition with dBET6 for CRBN occupancy, thereby recovering GRP signal and provides a measure of inhibition for deriving the IC₅₀.

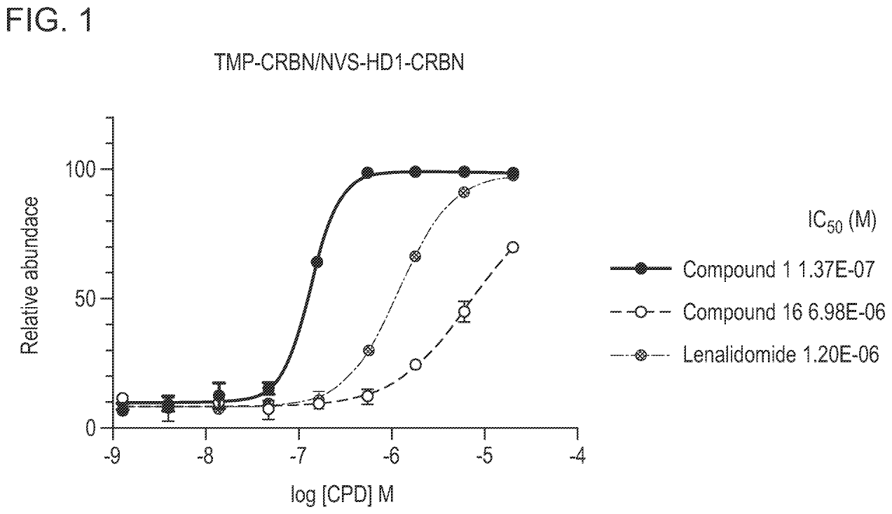

The results of the cellular CRBN engagement assay are illustrated in FIG. 1. They show that compounds 1 and 16 are cell permeable, with IC₅₀ values of 0.14 and 6.98 μM, respectively.

### Example 24: In Vitro Histone Deacetylase (HDAC) Enzymatic Assay

The in vitro HDAC enzymatic assays were performed by Reaction Biology (Devault, PA).

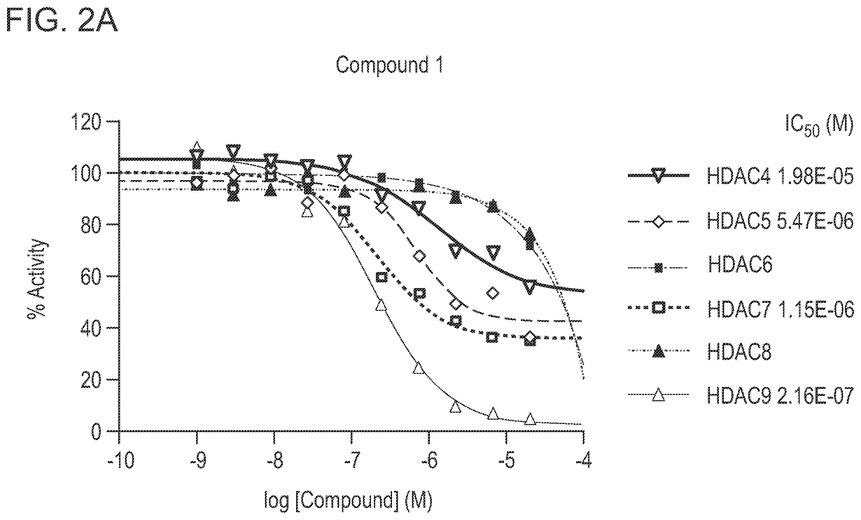

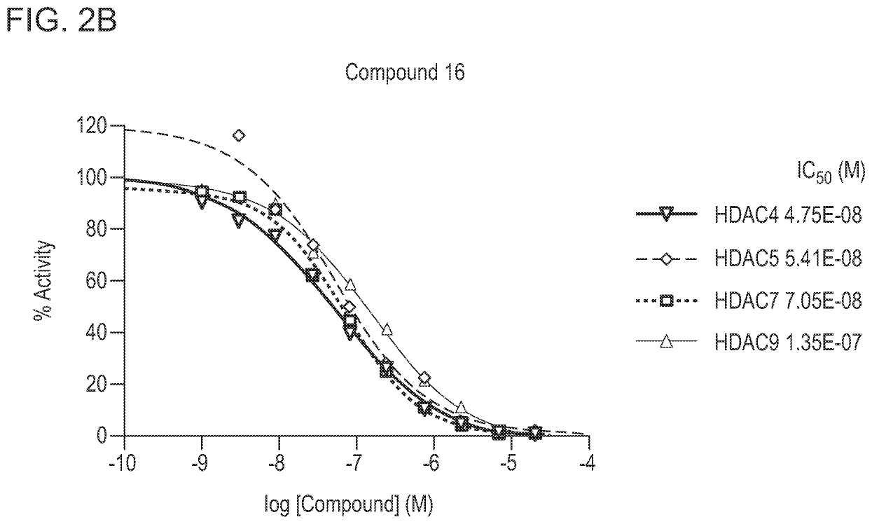

The results are illustrated in FIG. 2A-FIG. 2B. They show that compounds 1 (FIG. 2A) and 16 (FIG. 2B)inhibited HDAC4, 5, 7, and 9 in dose dependent manner. They also show that compound 1 did not inhibit HDAC6 and 8 (FIG. 2A).

### Example 24: Analysis of Change to Cellular Protein Abundance in Response to Treatment with Compounds

Kelly cells or MM.1S were treated with DMSO (biological triplicate) or compound 1 (1 μM), compound 3 (5 μM), compound 16 (1 μM) or compound 17 (1 μM) for 5 hours. Cells were washed once with phosphate-buffered saline (PBS), harvested with Cellstripper™ (Corning®), washed two additional times with PBS and snap frozen in liquid nitrogen. Lysis buffer (8 M Urea, 50 mM NaCl, 50 mM 4-(2hydroxyethyl)-1-piperazineethanesulfonic acid (EPPS) pH 8.5, protease and phosphatase inhibitors from Roche®) were added to the cell pellets and homogenized by 20 passes through a 21-gauge (1.25 in. long) needle to achieve a cell lysate with a protein concentration between 1-4 mg/mL. A micro-BCA assay (Pierce™) was used to determine the final protein concentration in the cell lysate. 200 μg of protein for each sample were reduced and alkylated as described in Donovan et al., Elife 7:e38430 (2018).

Proteins were precipitated using methanol/chloroform in Donovan et al., Elife 7:e38430 (2018). The precipitated protein was resuspended in 4 M Urea, 50 mM HEPES pH 7.4, followed by dilution to 1 M urea with the addition of 200 mM EPPS, pH 8. Proteins were first digested with LysC (1:50: enzyme:protein) for 12 hours at room temperature. The LysC digestion was diluted to 0.5 M Urea with 200 mM EPPS pH 8 followed by digestion with trypsin (1:50: enzyme:protein) for 6 hours at 37° C. Tandem mass tag (TMT) reagents (Thermo Fisher Scientific) were dissolved in anhydrous acetonitrile (ACN) according to manufacturer's instructions.

Anhydrous ACN was added to each peptide sample to a final concentration of 30% v/v, and labeling was induced with the addition of TMT reagent to each sample at a ratio of 1:4 peptide:TMT label. The 11-plex labeling reactions were performed for 1.5 hours at room temperature and the reaction quenched by the addition of hydroxylamine to a final concentration of 0.3% for 15 minutes at room temperature. The sample channels were combined at a 1:1 ratio, desalted using C18 solid phase extraction cartridges (Waters®) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®-, Waters®). Samples were then offline fractionated into 96 fractions by high pH reverse-phase HPLC (Agilent® LC1260) through an aeris peptide xb-c18 column (Phenomenex®) with mobile phase A containing 5% acetonitrile and 10 mM NH₄HCOs in LC-MS grade H₂O, and mobile phase B containing 90° % acetonitrile and 10 mM NH₄HCO₃ in LC-MS grade H₂O (both pH 8.0). The 96 resulting fractions were then pooled in a non-contiguous manner into 24 fractions and these fractions were used for subsequent mass spectrometry analysis.

Data were collected using an Orbitrap Fusion™ Lumos™ mass spectrometer (Thermo Fisher Scientific, San Jose, CA, USA) coupled with a Proxeon EASY-nLC™ 1200 LC pump (Thermo Fisher Scientific). Peptides were separated on an EasySpray™ ES803.rev2 75 μm inner diameter microcapillary column (ThermoFisher Scientific). Peptides were separated using a 190 min gradient of 6-27% acetonitrile in 1.0% formic acid with a flow rate of 300 nL/min.

Each analysis used an MS3-based TMT method as described in McAlister et al., Anal. Chem. 86(14):7150-7158 (2014). The data were acquired using a mass range of m/z 340-1350, resolution 120,000, automatic gain control (AGC) target 1×10⁶, maximum injection time 100 ms, dynamic exclusion of 120 seconds for the peptide measurements in the Orbitrap Fusion™ Lumos™ mass spectrometer. Data dependent MS2 spectra were acquired in the ion trap with a normalized collision energy (NCE) set at 55%, AGC target set to 1.5×10⁵ and a maximum injection time of 150 ms. MS3 scans were acquired in the Orbitrap Fusion™ Lumos™ mass spectrometer with a higher energy collision dissociation (HCD) set to 55%, AGC target set to 1.5×10⁵, maximum injection time of 150 ms, resolution at 50,000 and with a maximum synchronous precursor selection (SPS) precursors set to 10.

Proteome Discoverer 2.2 (Thermo Fisher Scientific) was used for .RAW file processing and controlling peptide and protein level false discovery rates, assembling proteins from peptides, and protein quantification from peptides. MS/MS spectra were searched against a Swissprot human database (December 2016) with both the forward and reverse sequences.

Database search criteria are as follows: tryptic with two missed cleavages, a precursor mass tolerance of 10 ppm, fragment ion mass tolerance of 0.6 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m/z for each reporter ion in the MS3 scan. Peptide spectral matches with poor quality MS3 spectra were excluded from quantitation (summed signal-to-noise across 11 channels <100 and precursor isolation specificity <0.5), and resulting data was filtered to only include proteins that had a minimum of 2 unique peptides identified. Reporter ion intensities were normalized and scaled using in-house scripts in the R framework. Statistical analysis was carried out using the limma package within the R framework as described in Ritchie et al., Nucleic Acids Res. 43(7):e47 (2015).

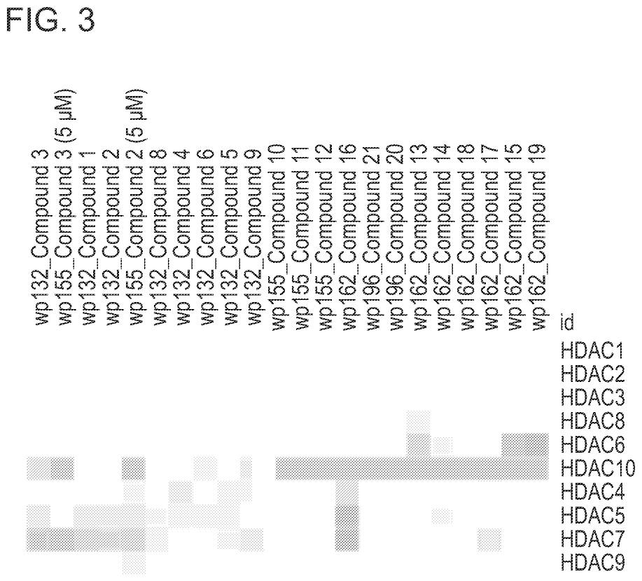

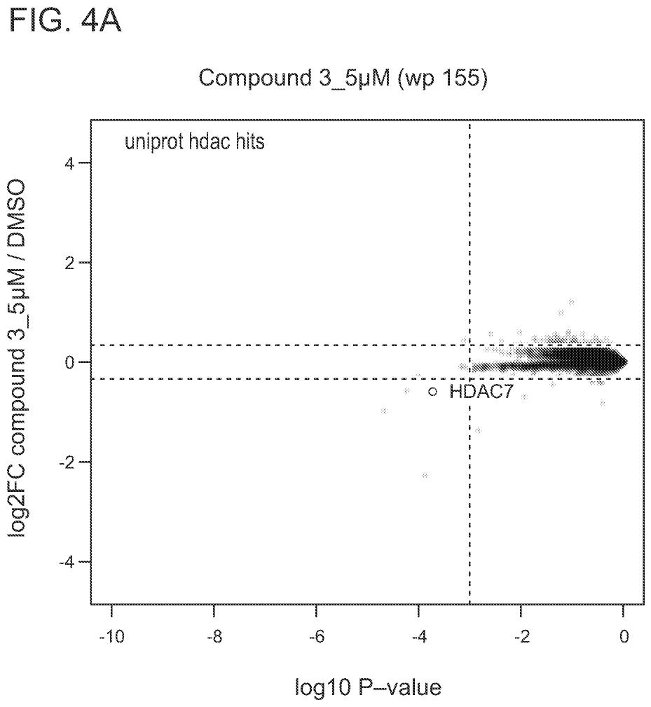

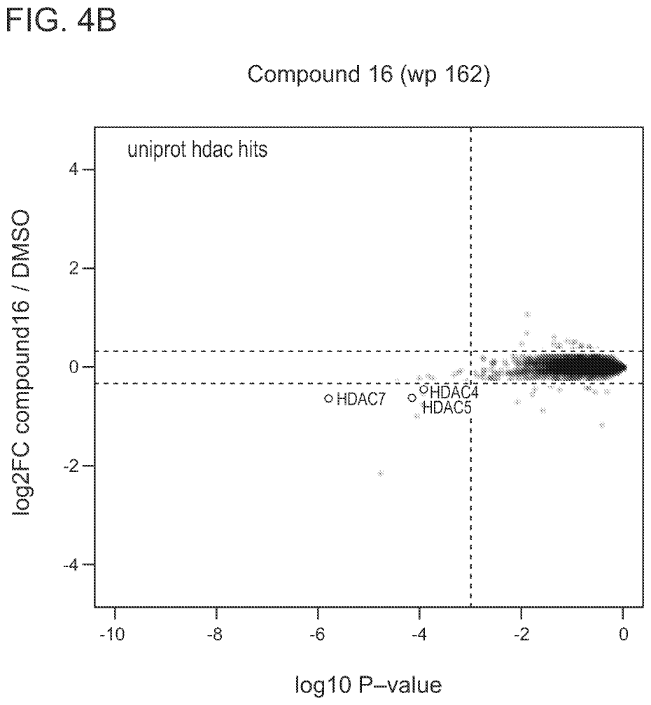

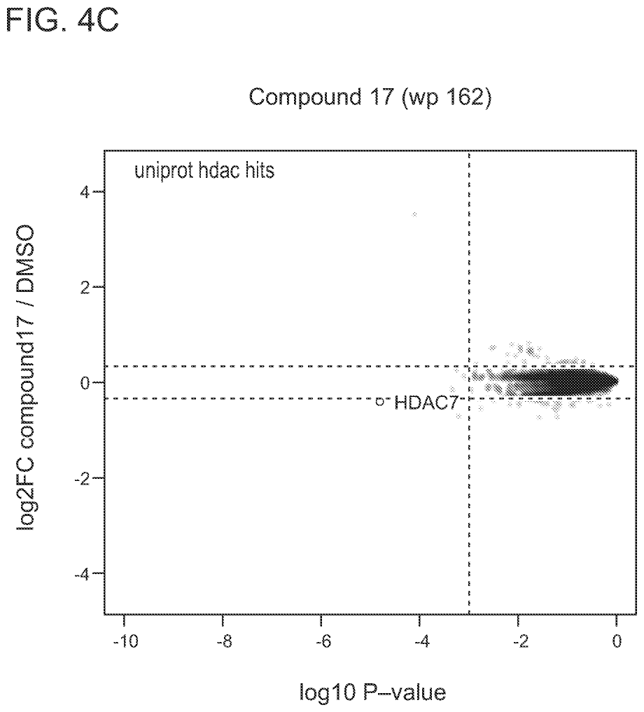

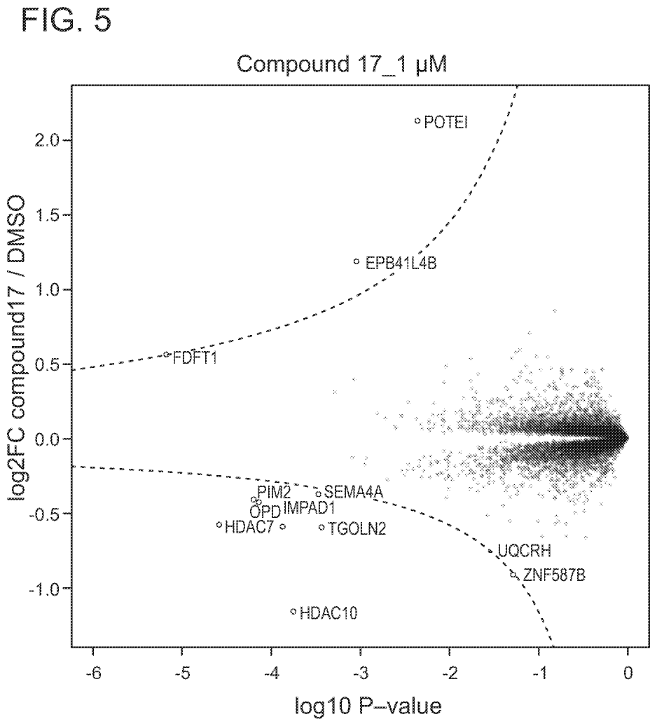

The results are summarized in FIG. 3, FIG. 4A-FIG. 4C, and FIG. 5.

The heatmap in FIG. 3 shows the degradation of class IIa HDACs by compounds 1-6 and 8-21. These data show that 5-hour treatment of Kelly cells with 1 μM of compounds 1 and 17 induced selective reduction in protein expression level of HDAC7, 5 μM treatment with compound 3 induced a reduction in protein expression level of HDAC5 and 7, and 1 μM treatment with compound 16 induced a reduction in protein expression level of HDAC4, 5 and 7.

The scatterplots in FIG. 4A-FIG. 4C show the change in relative protein abundance with treatment of Kelly cells with compounds 3 (FIG. 4A), 16 (FIG. 4B), and 17 (FIG. 4C), compared to dimethyl sulfoxide (DMSO) control. Significant changes were assessed by moderated t-test and displayed with log 2-fold change on the y-axis and negative log₁₀ P values on the x-axis for one independent biological replicate of the compound and three independent biological replicates of DMSO. As shown, treatment with each of compounds 3, 16, and 17 induced a significant reduction in class IIa HDAC levels when compared to the DMSO treated cells.

The scatterplot in FIG. 5 shows the change in relative protein abundance with treatment of MM.1S cells with compound 17, compared to dimethyl sulfoxide (DMSO) control. Significant changes were assessed by moderated t-test and displayed with log 2-fold change on the y-axis and negative log₁₀ P values on the x-axis for one independent biological replicate of compound and three independent biological replicates of DMSO. As shown, 5-hour treatment of MM.1S cells with 1 μM of compound 17 induced a significant reduction in class IIa HDAC levels when compared to the DMSO treated cells.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present invention as defined by the appended claims.

What is claimed is:

1. A compound comprising a moiety that binds at least one class IIa histone deacetylase (HDAC) and a degron covalently attached to each other by a linker that comprises an alkylene chain or a polyethylene glycol (PEG) chain, wherein the compound has a structure represented by formula (I): 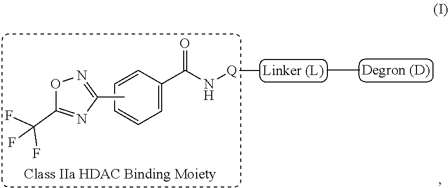
wherein:
Q represents 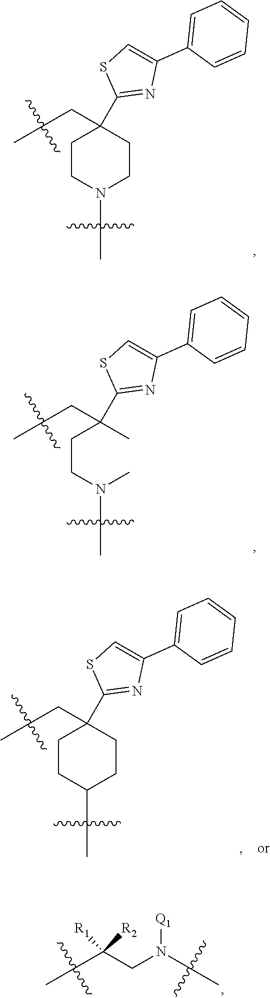
wherein R₁ and R₂ are independently H or C₁-C₄ alkyl and Q₁ is optionally C₁-C₄ alkyl;
and the degron represents a ligand that binds cereblon (CRBN), von Hippel Landau tumor suppressor (VHL), or inhibitor of apoptosis protein (IAP),
or a pharmaceutically acceptable salt or stereoisomer thereof.

2. The compound of claim 1, wherein Q is

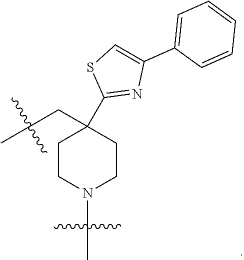 [SMILES: *CC1(c2nc(-c3ccccc3)cs2)CCN(*)CC1]

3. The compound of claim 1, wherein Q is

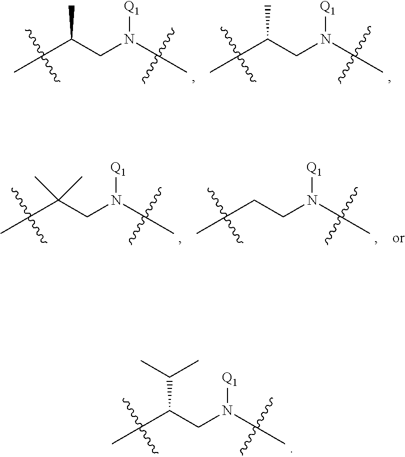

4. (canceled)

5. The compound of claim 3, wherein Q₁ is ethyl or benzyl.

6. The compound of claim 1, which has an one of structures I-1 and (I-2): 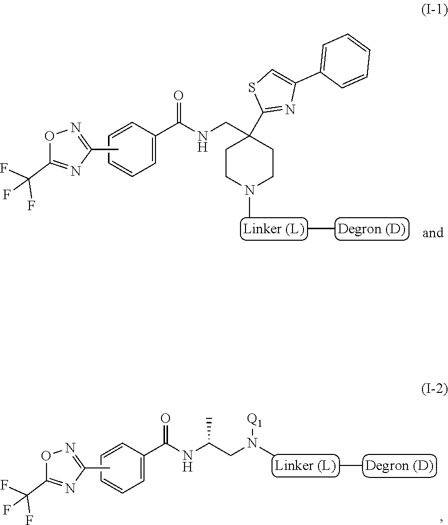
or a pharmaceutically acceptable salt or stereoisomer thereof.

7. The compound of claim 1, wherein the linker comprises an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—,—N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—,—S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

8. The compound of claim 1, wherein the alkylene chain comprises 1-12 alkylene units.

9. The compound of claim 1, wherein the linker comprises a polyethylene glycol chain that may be interrupted by, and/or terminate (at either or both termini) at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—,—OC(O)N(R′)—, —C(NR′)—, —N(R′)C(NR′)—, —C(NR′)N(R′)—, —N(R′)C(NR′)N(R′)—,—OB(Me)O—, —S(O)₂—, —OS(O)—, —S(O)O—, —S(O)—, —OS(O)₂—, —S(O)₂O—,—N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

10. The compound of claim 9, wherein the polyethylene glycol chain comprises 1-6 PEG units.

11. The compound of claim 1, wherein the linker is any one of structures:

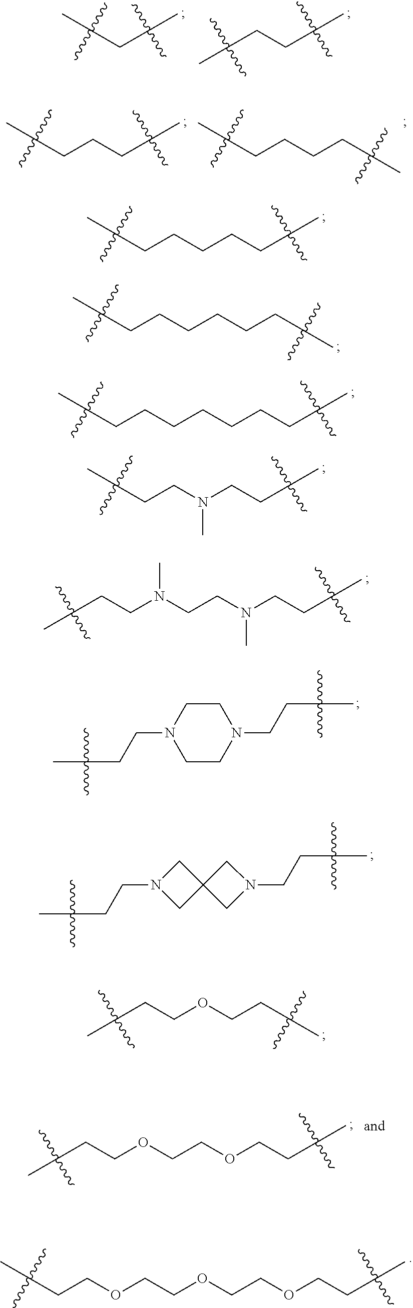

12. The compound of claim 1, which is represented by any one of structures (I-3) to (I-12): 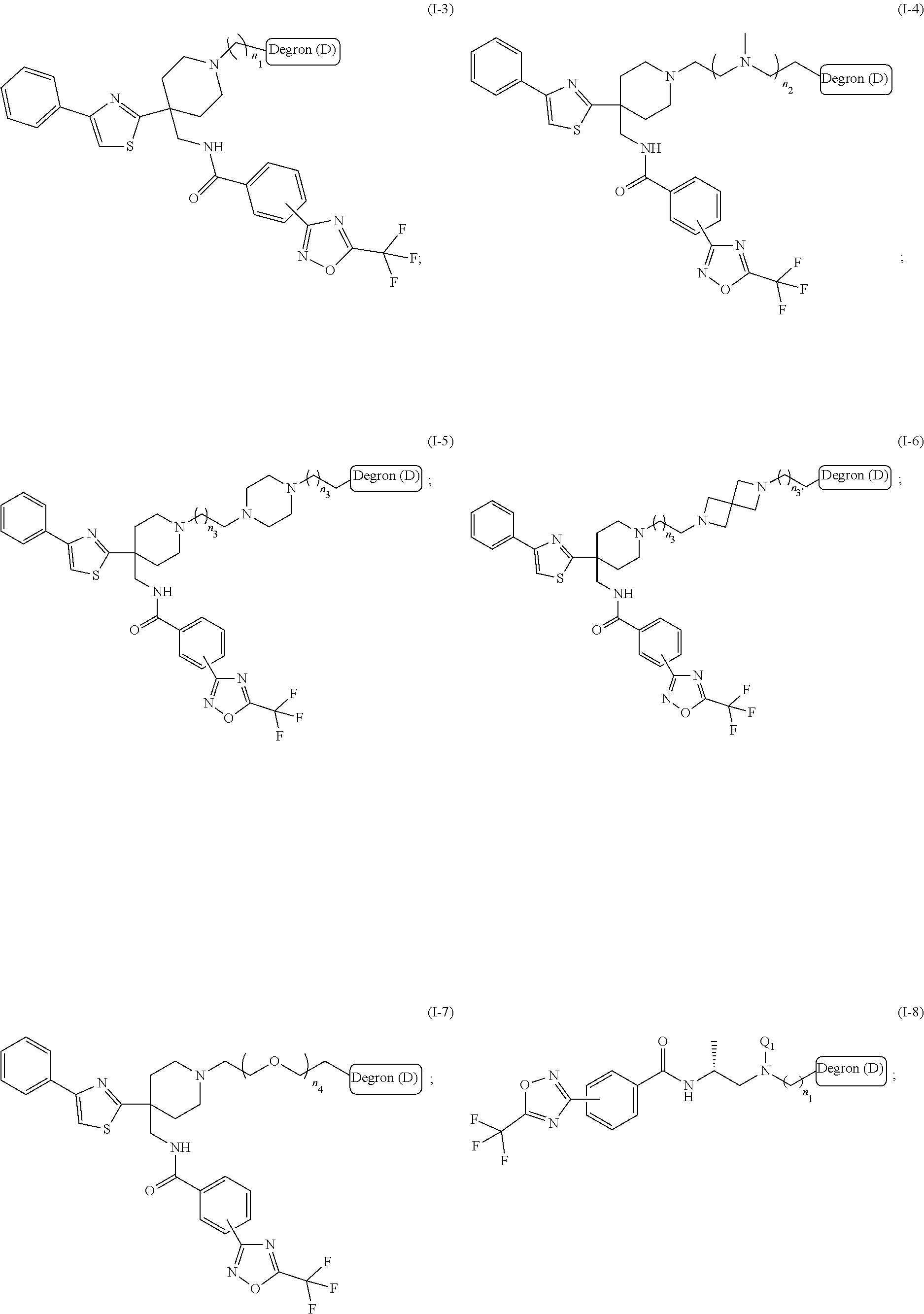 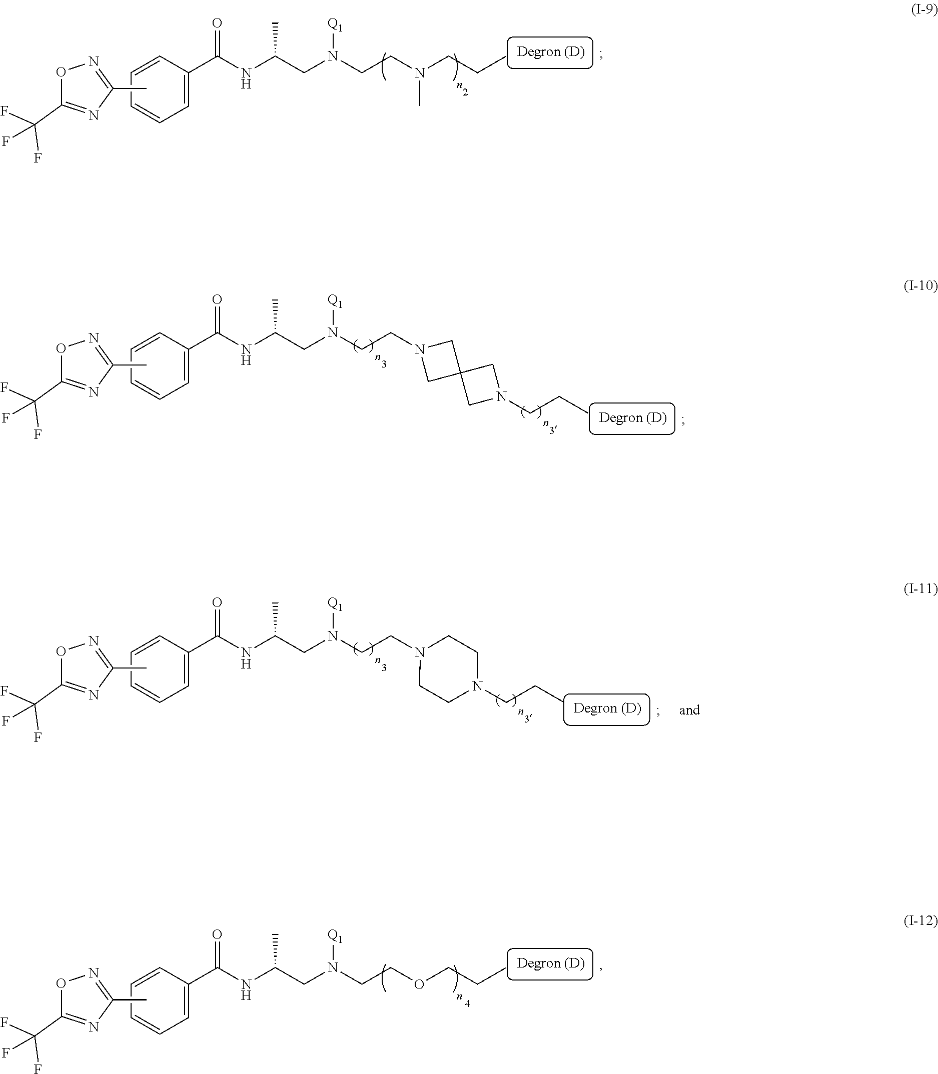
wherein n₁ is an integer from 0-12, n₂ is an integer from 1-2, n₃ and n_{3′} are independently an integer from 1-8, n₄ is an integer from 1-5, and Q₁ is optionally substituted C₁-C₃ alkyl, or a pharmaceutically acceptable salt or stereoisomer thereof.

13. The compound of claim 1, wherein the degron binds CRBN and is represented by any one of structures (D1a) to (D1d): 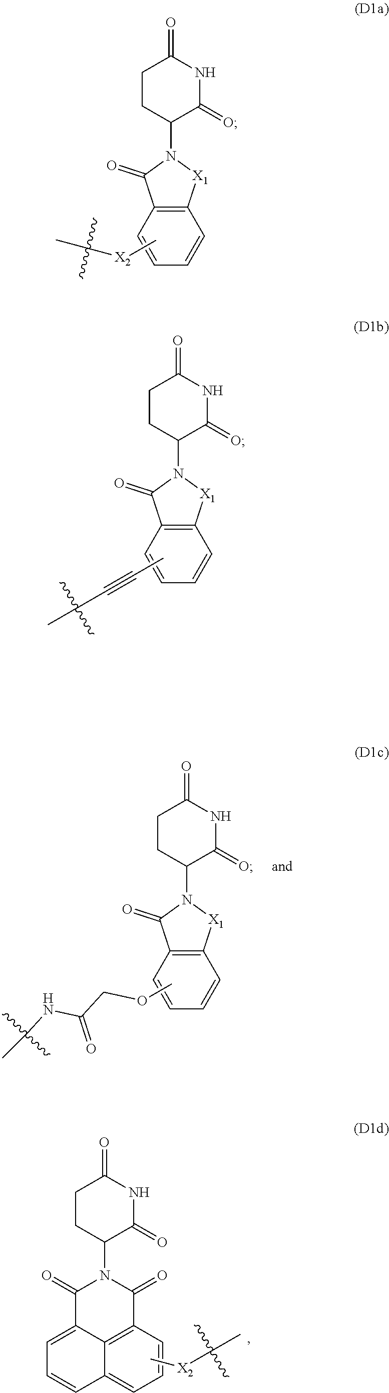
wherein X₁ is CH₂ or C(O) and X is a bond, CH₂, NH, or O.

14. (canceled)

15. The compound of claim 13, which is represented by any one of structures (I-13) to (I-52): 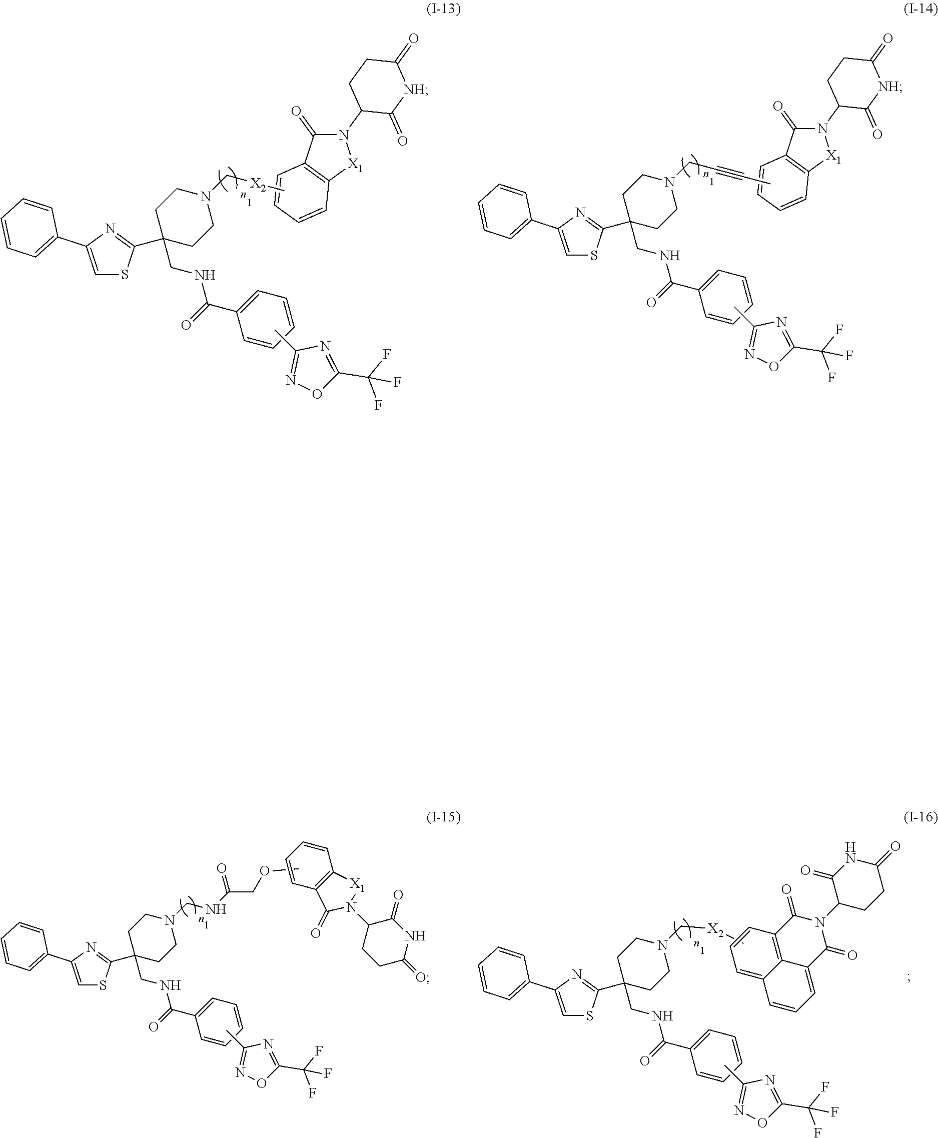 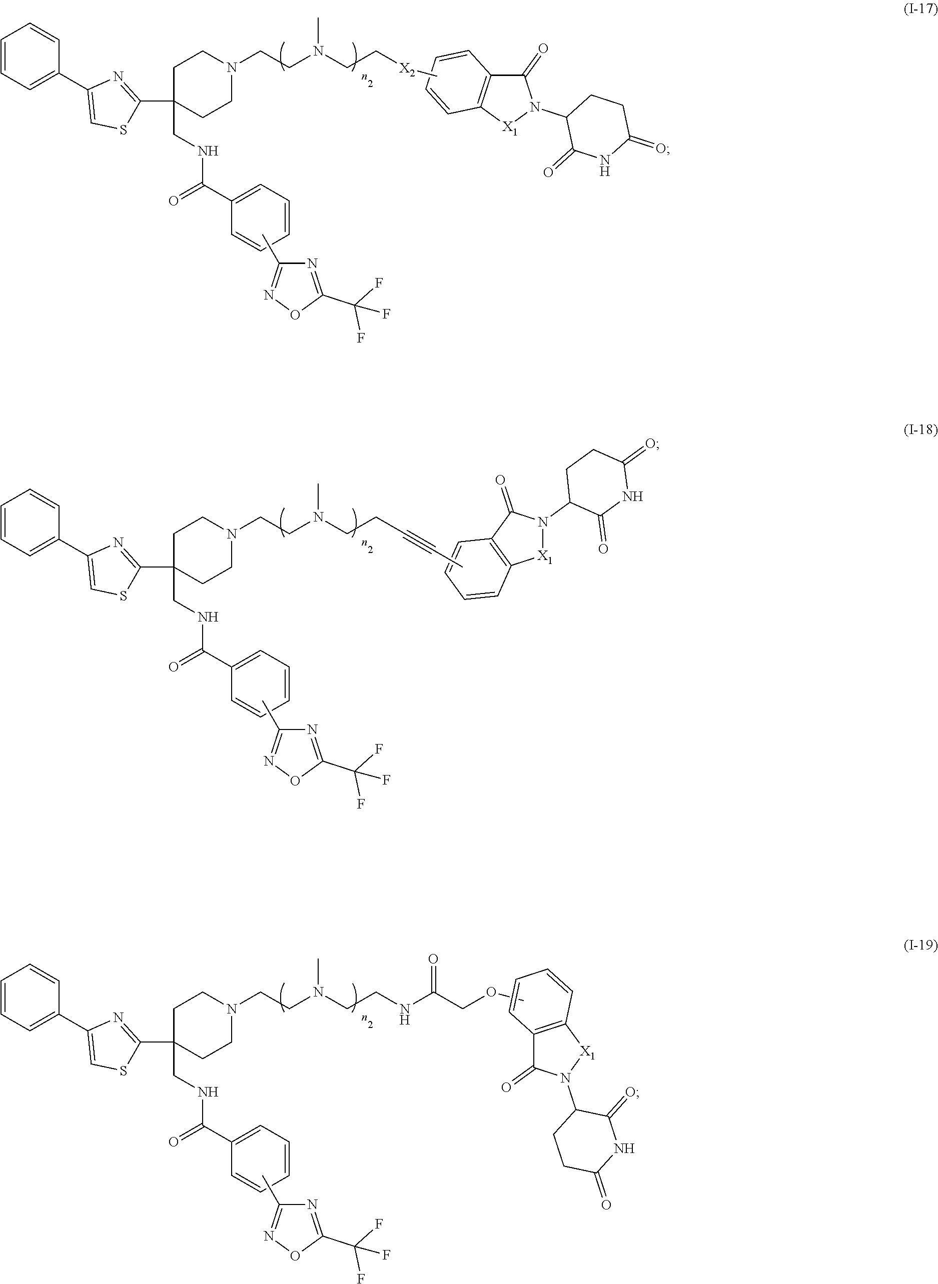 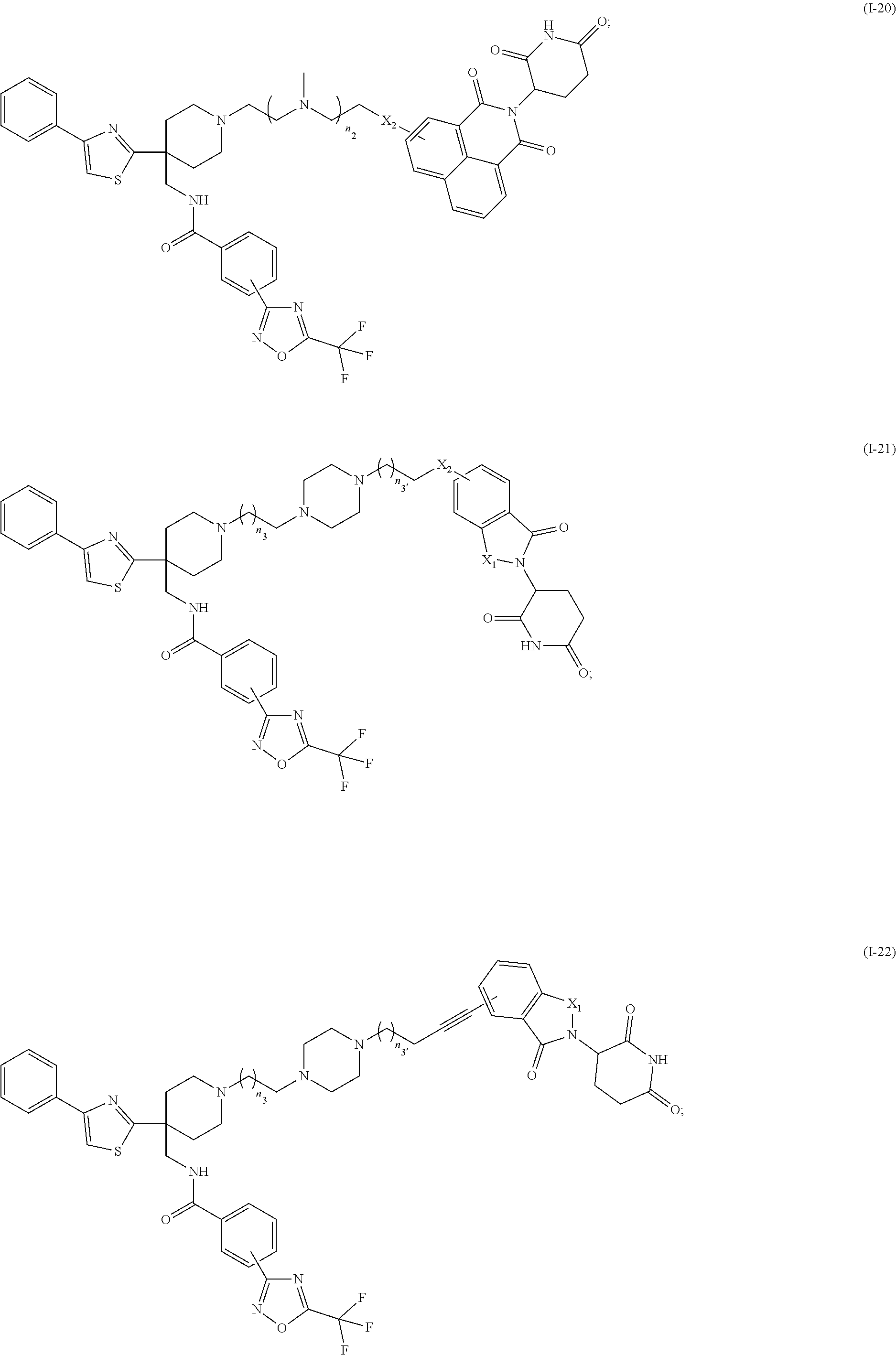 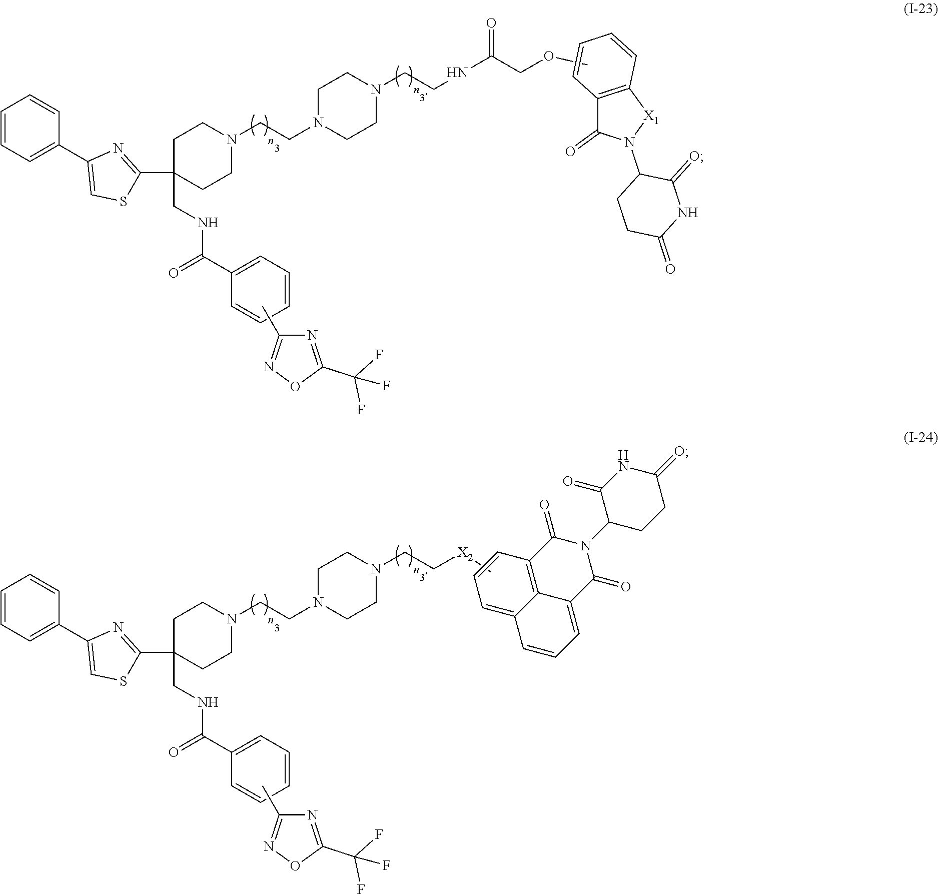 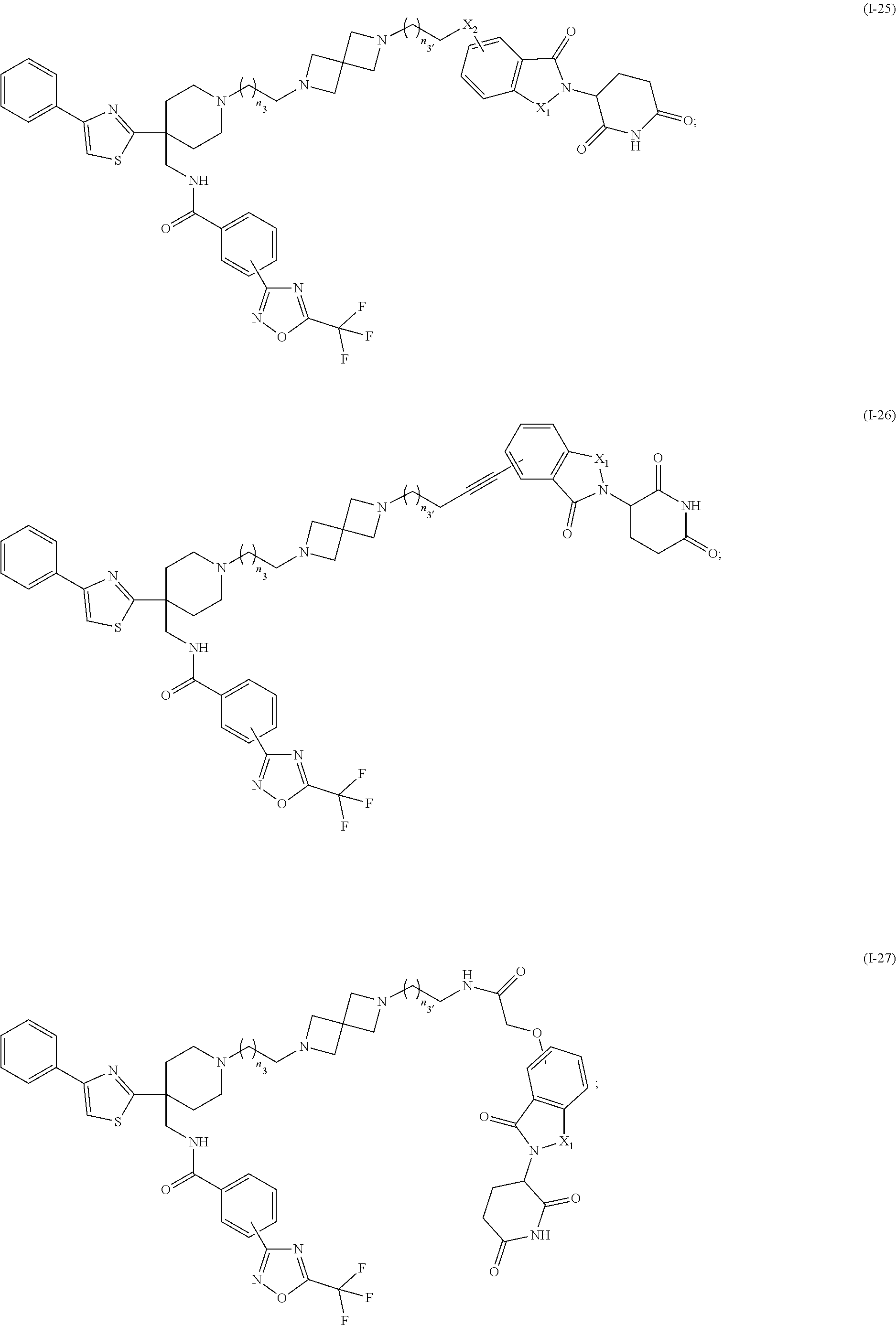 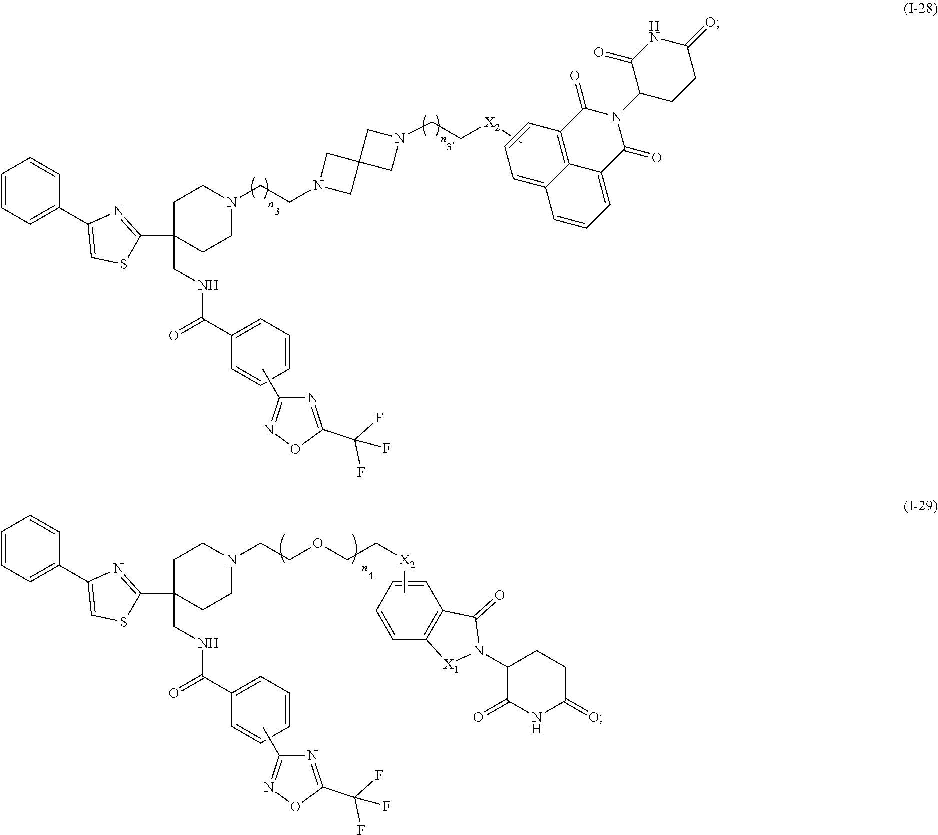 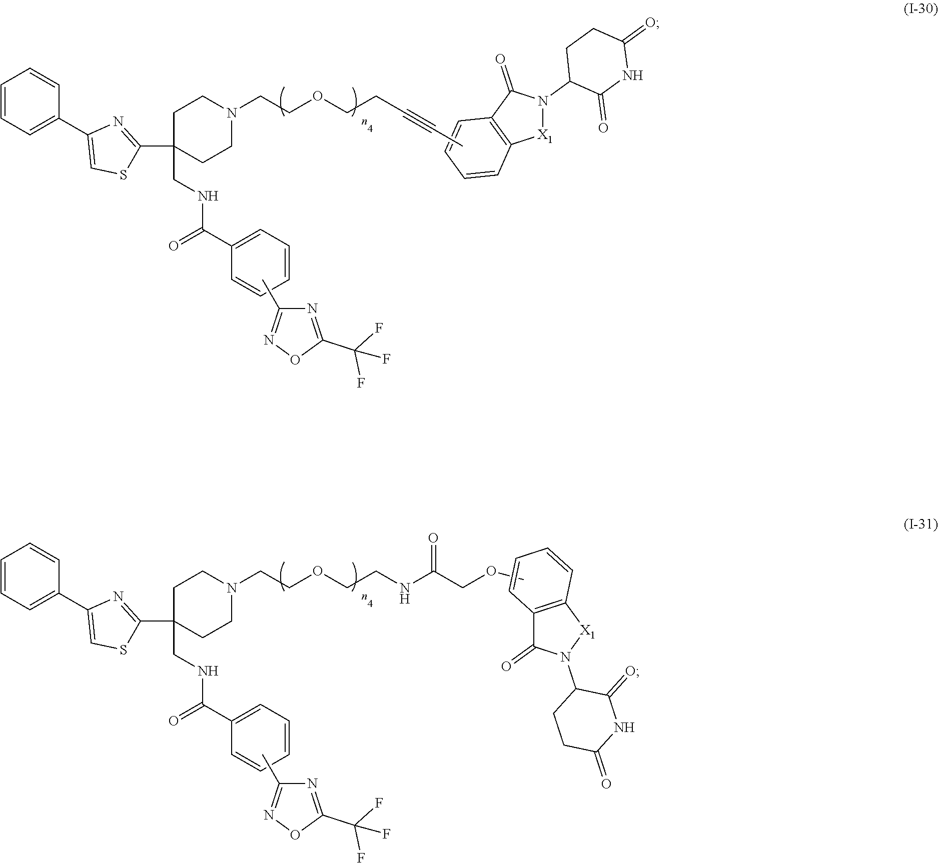 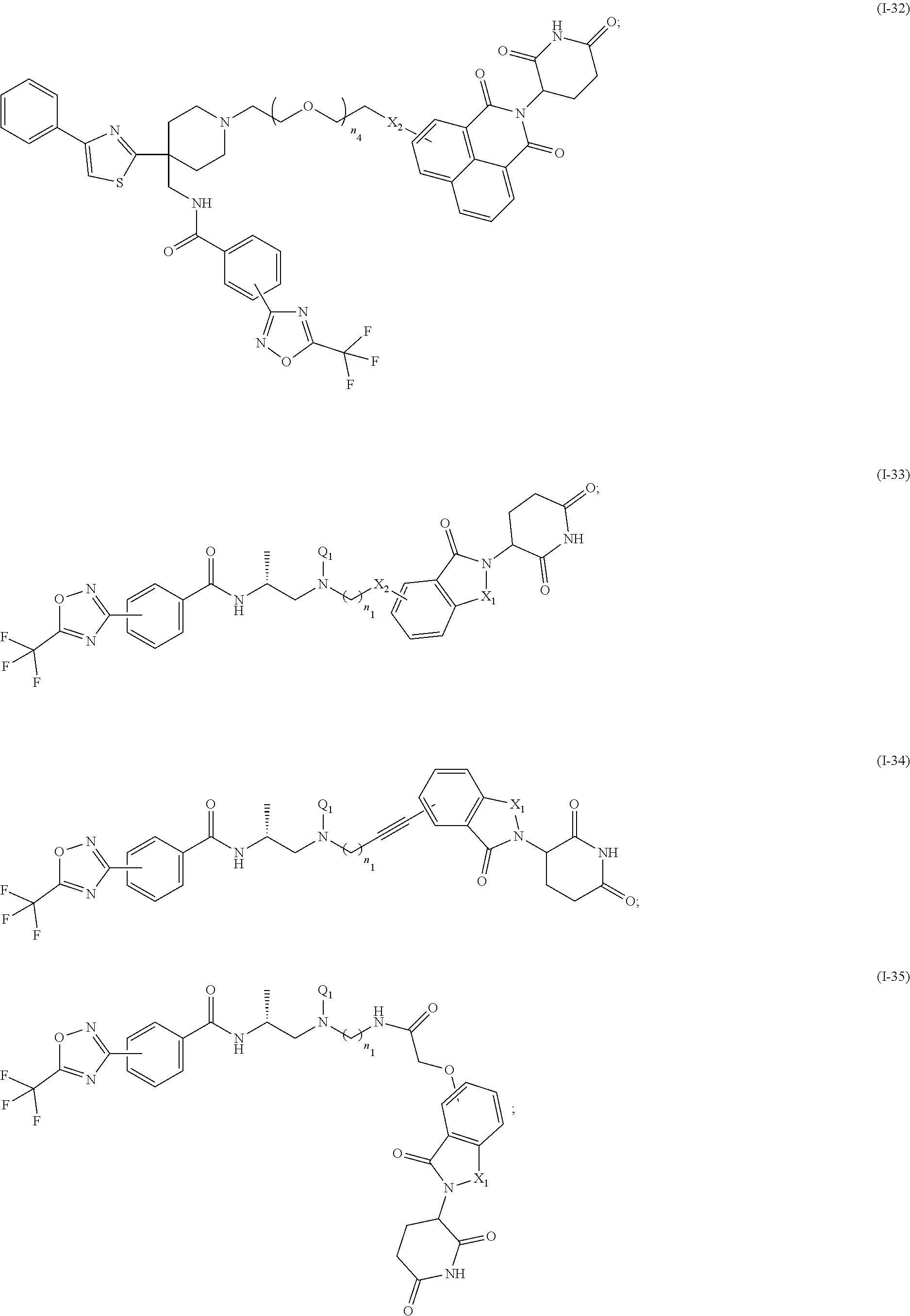 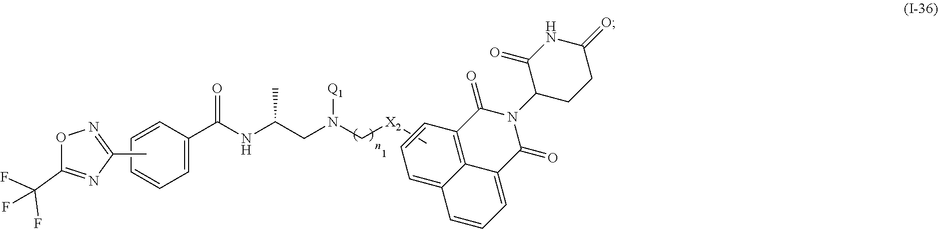 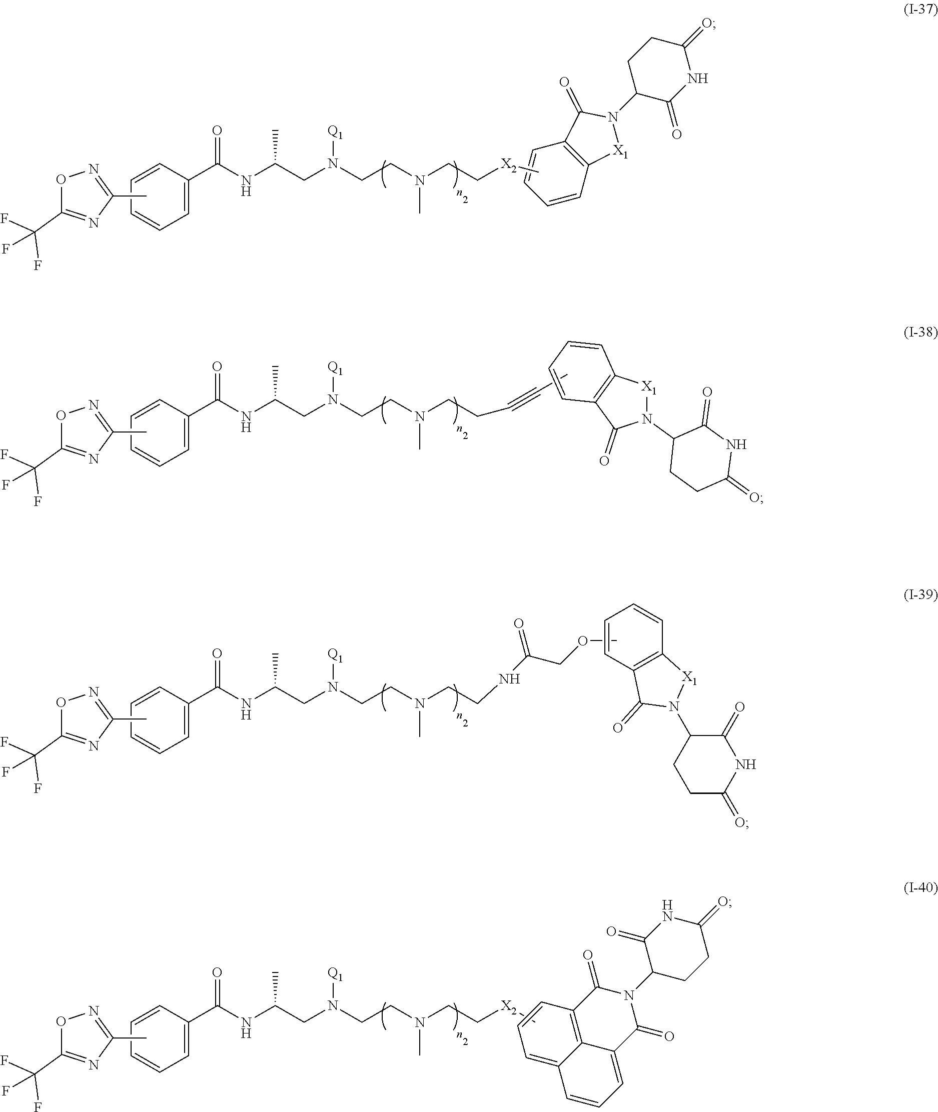 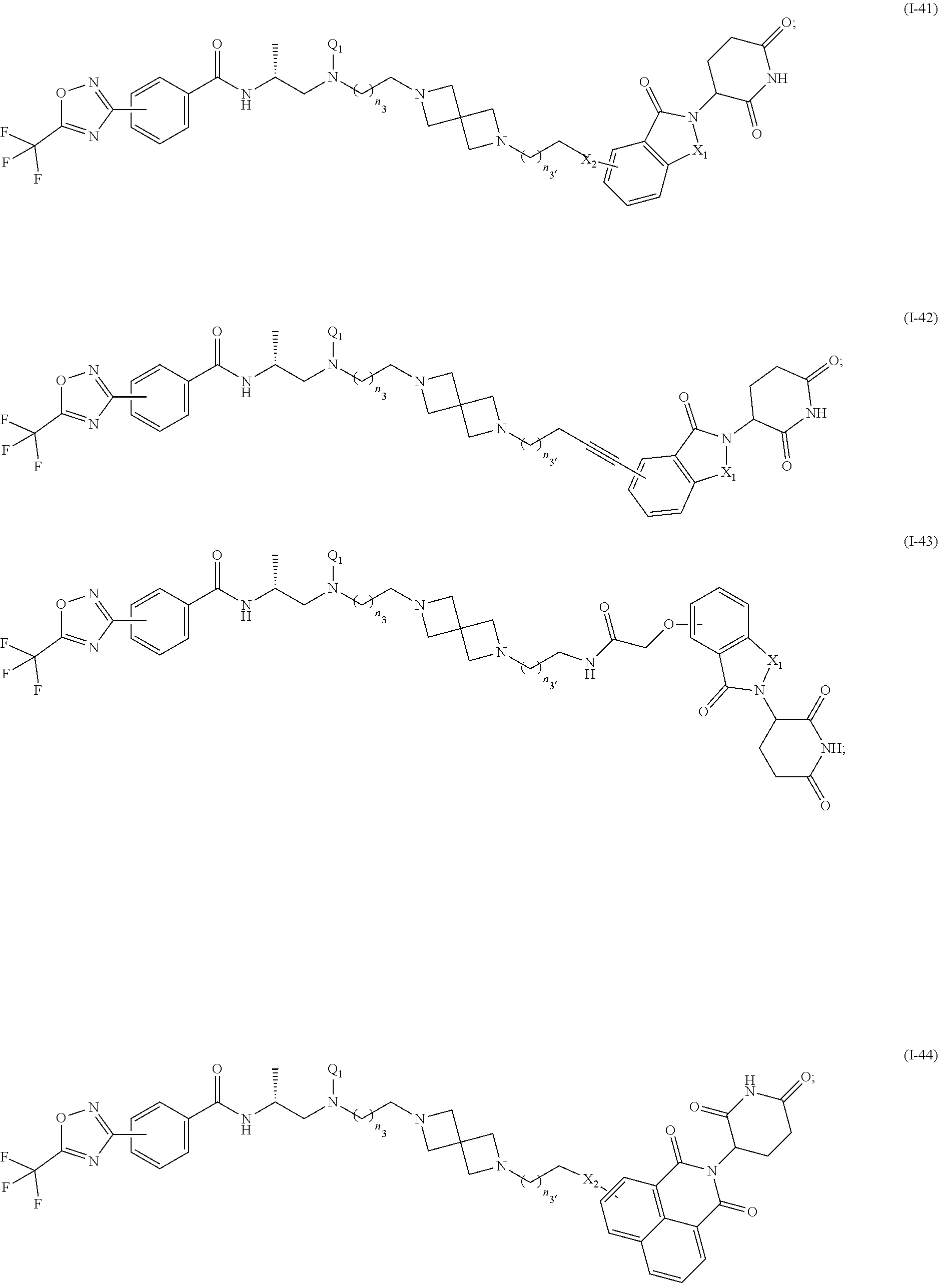 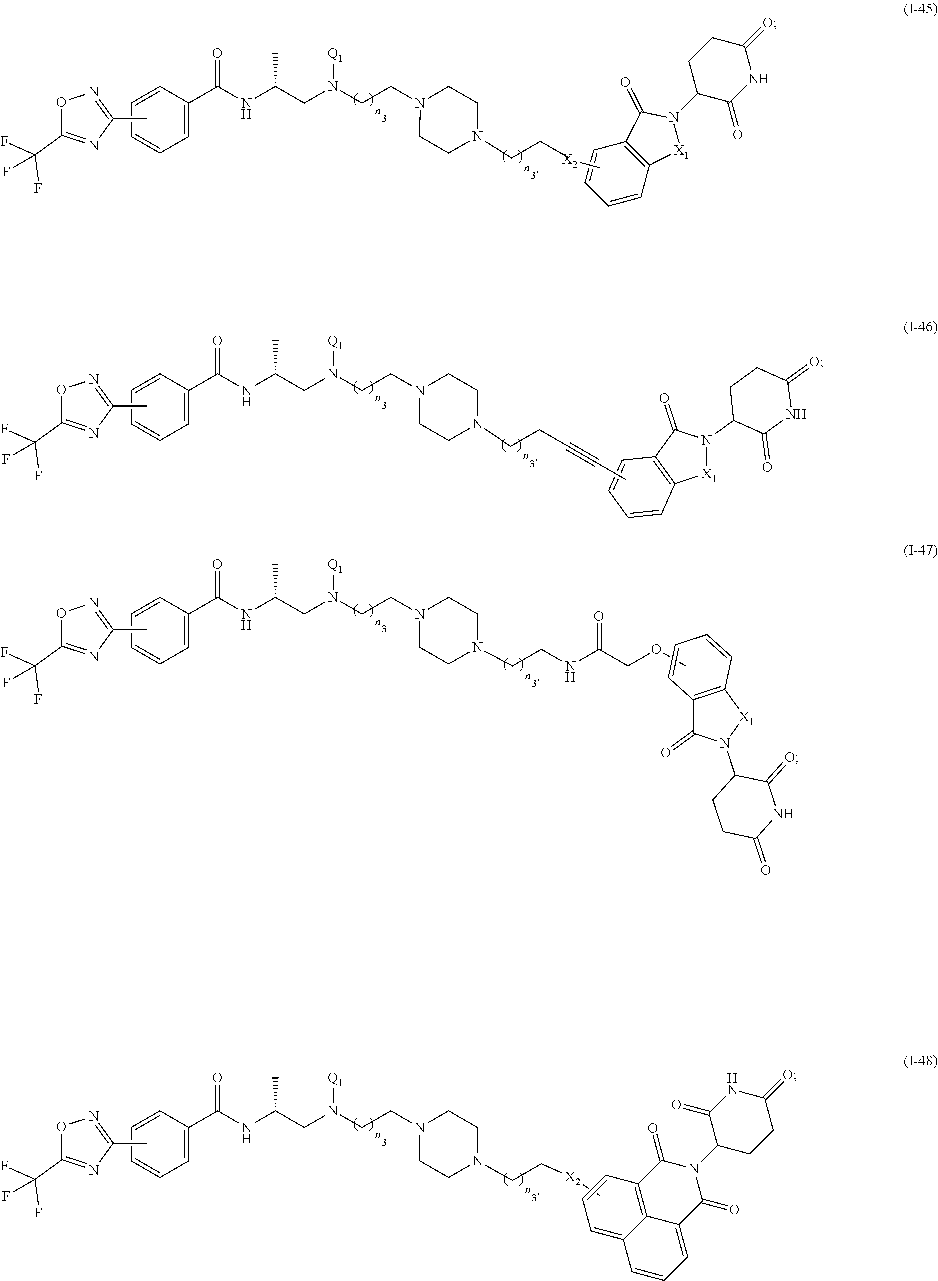 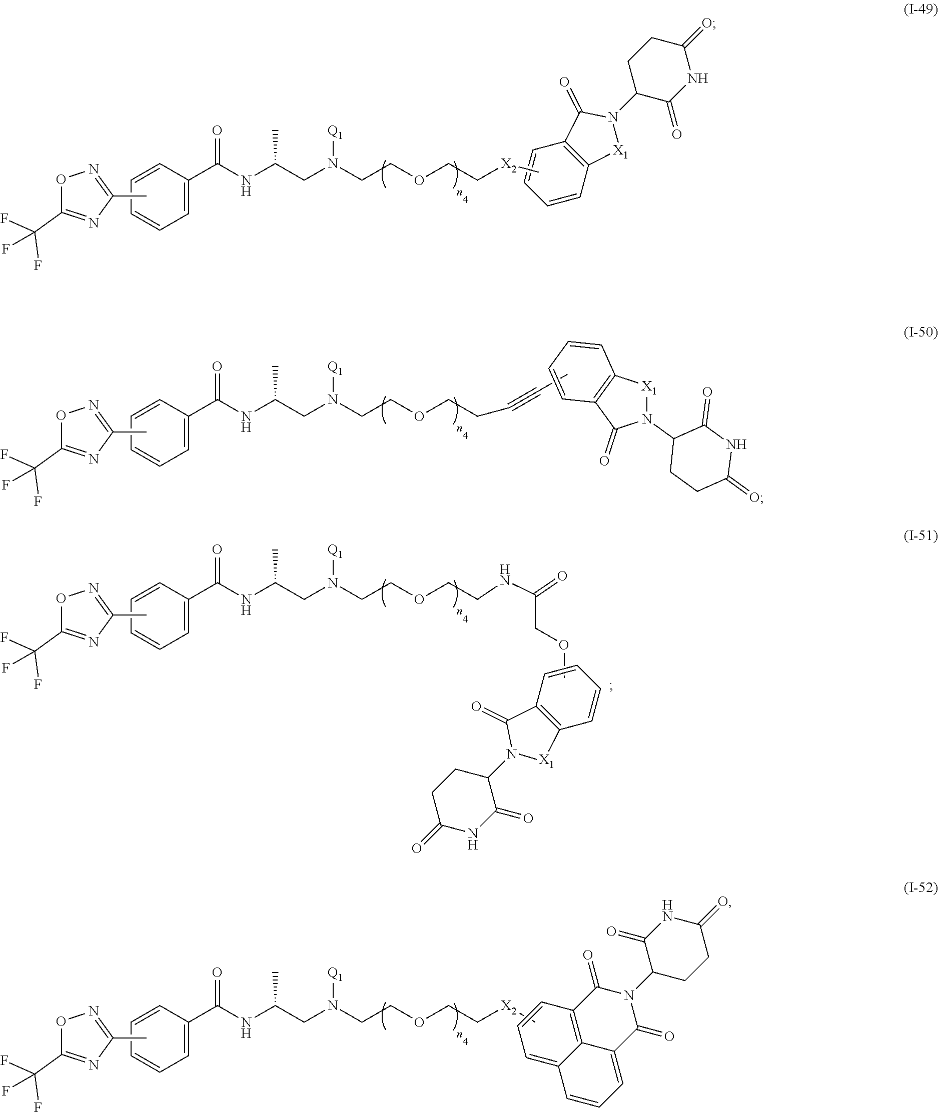
or a pharmaceutically acceptable salt or stereoisomer thereof.

16. The compound of claim 1, wherein the degron binds VHL and is represented by any one of structures (D2-a) to (D2-f): 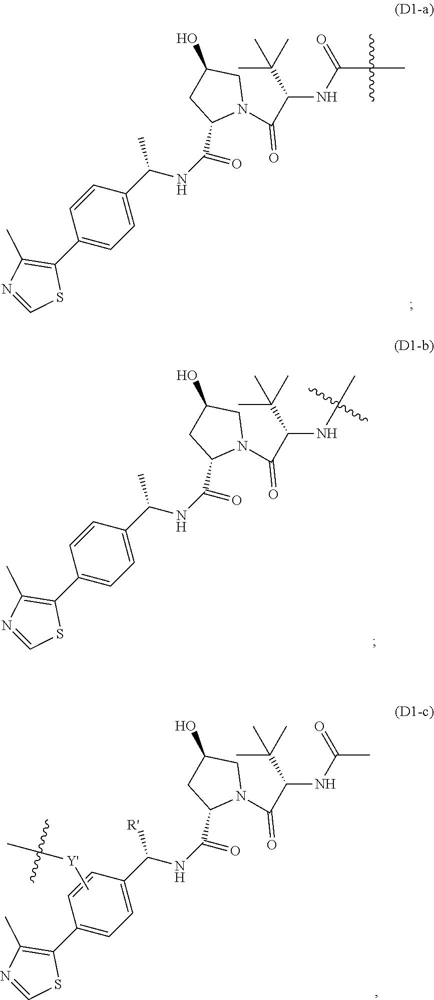
wherein Y′ is a bond, N, O or C and R′ is H or methyl: 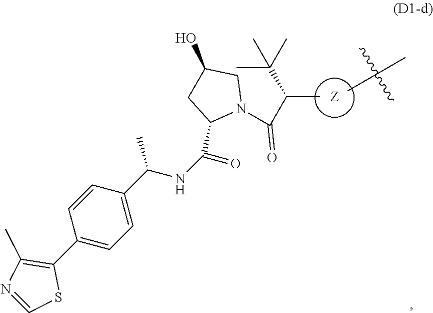
wherein Z is a C₅-C₆ carbocyclic or a C₅-C₆ heterocyclic group 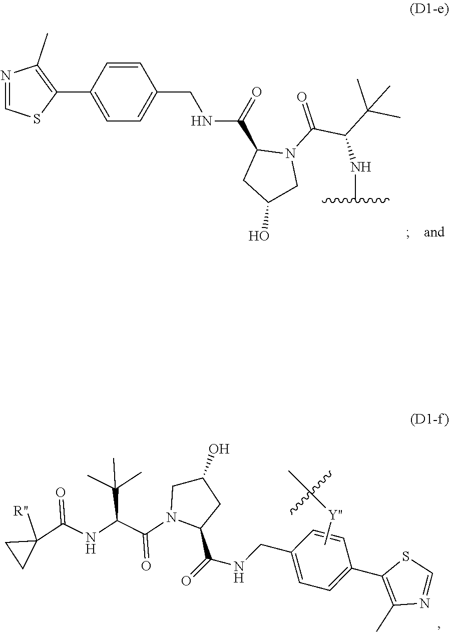
wherein Y″ is a bond, N, O or C and R″ is F or CN,
or a stereoisomer thereof.

17. (canceled)

18. The compound of claim 16, wherein Z is

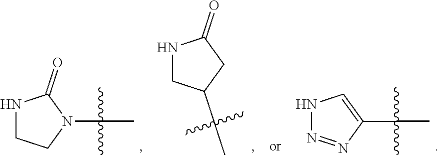 [SMILES: *N1CCNC1=O ; *C1CNC(=O)C1 ; *c1c[nH]nn1]

19. The compound of claim 16, which is represented by any one of structures (I-53) to (I-112): 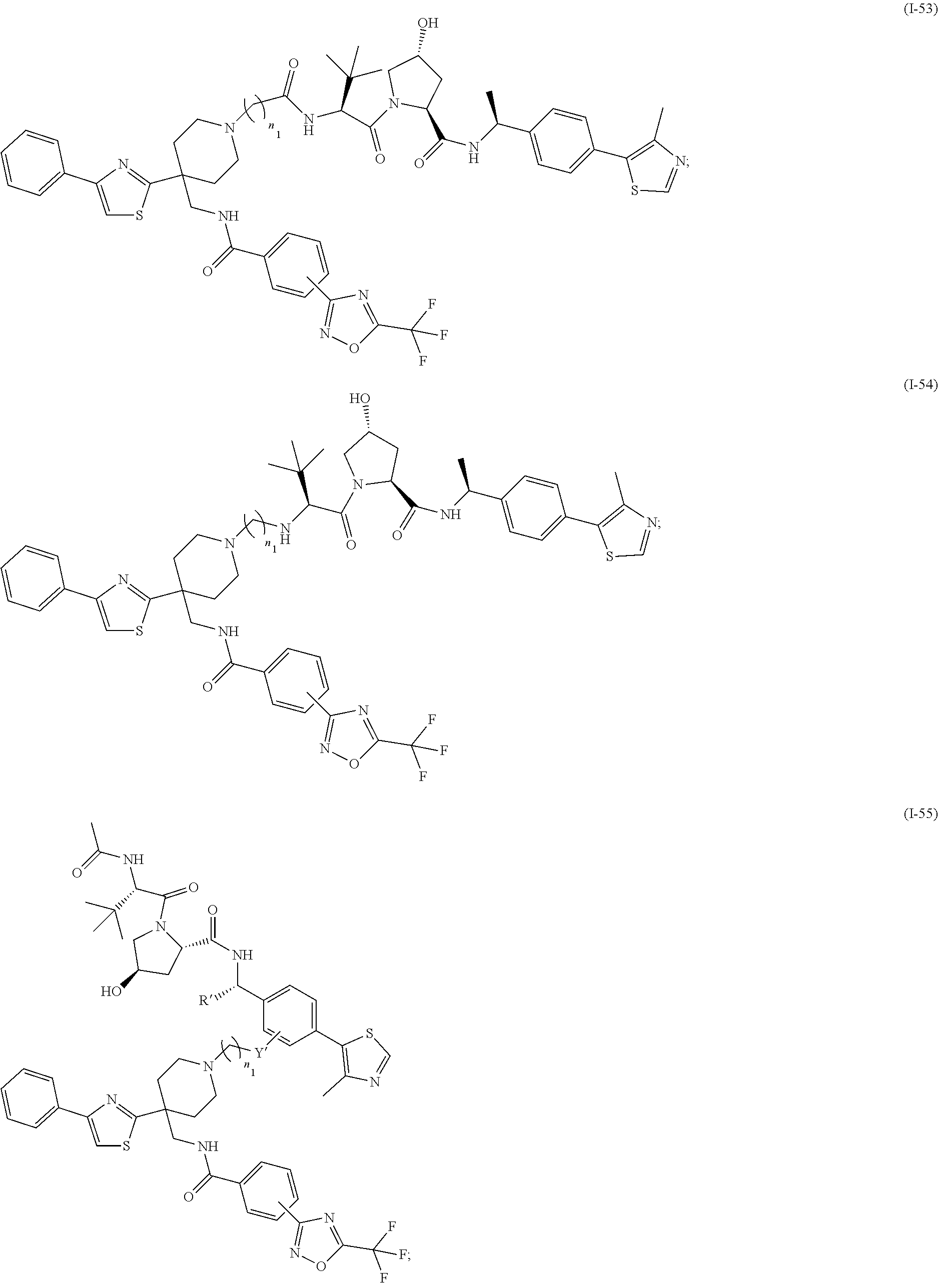 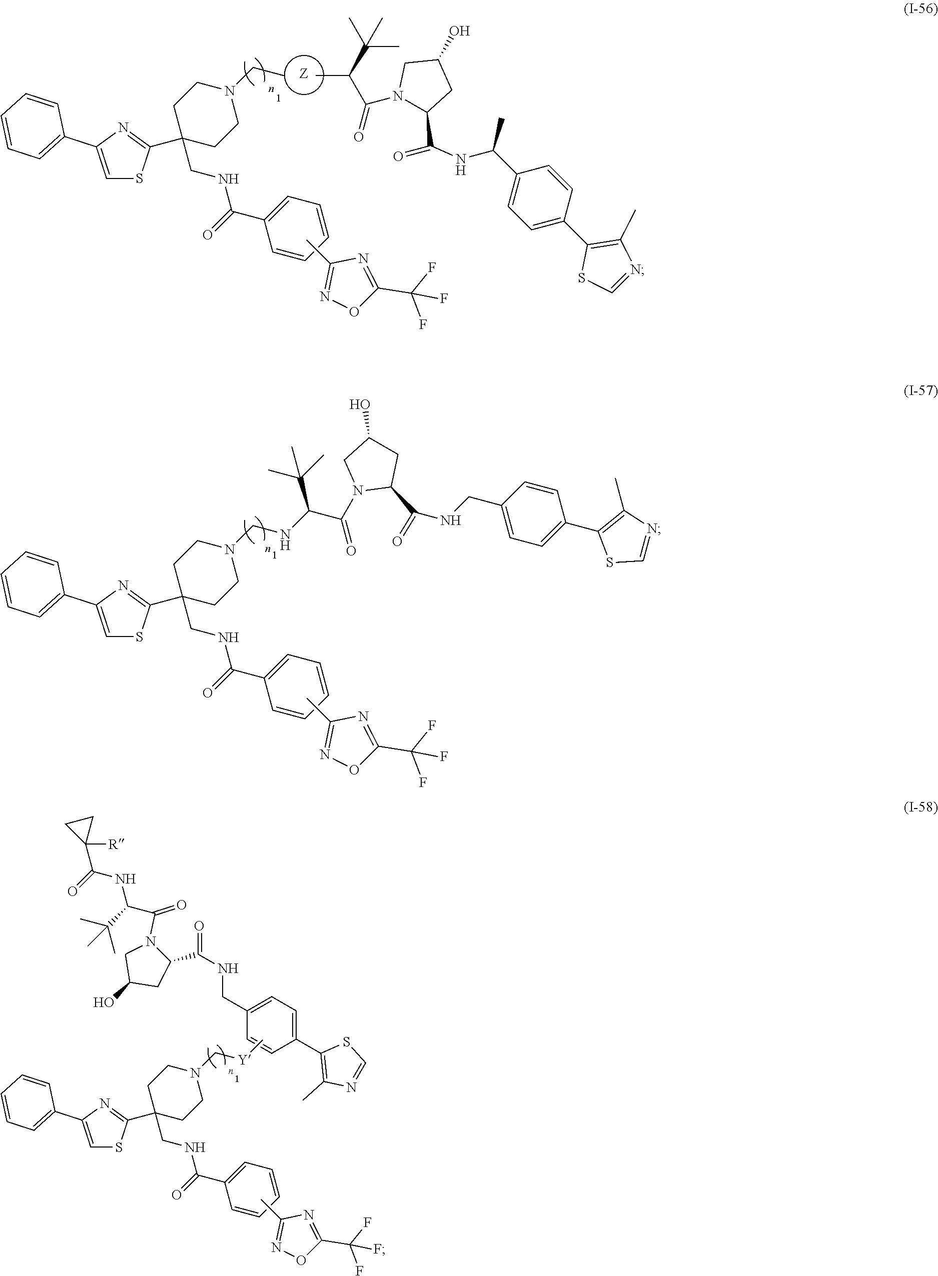 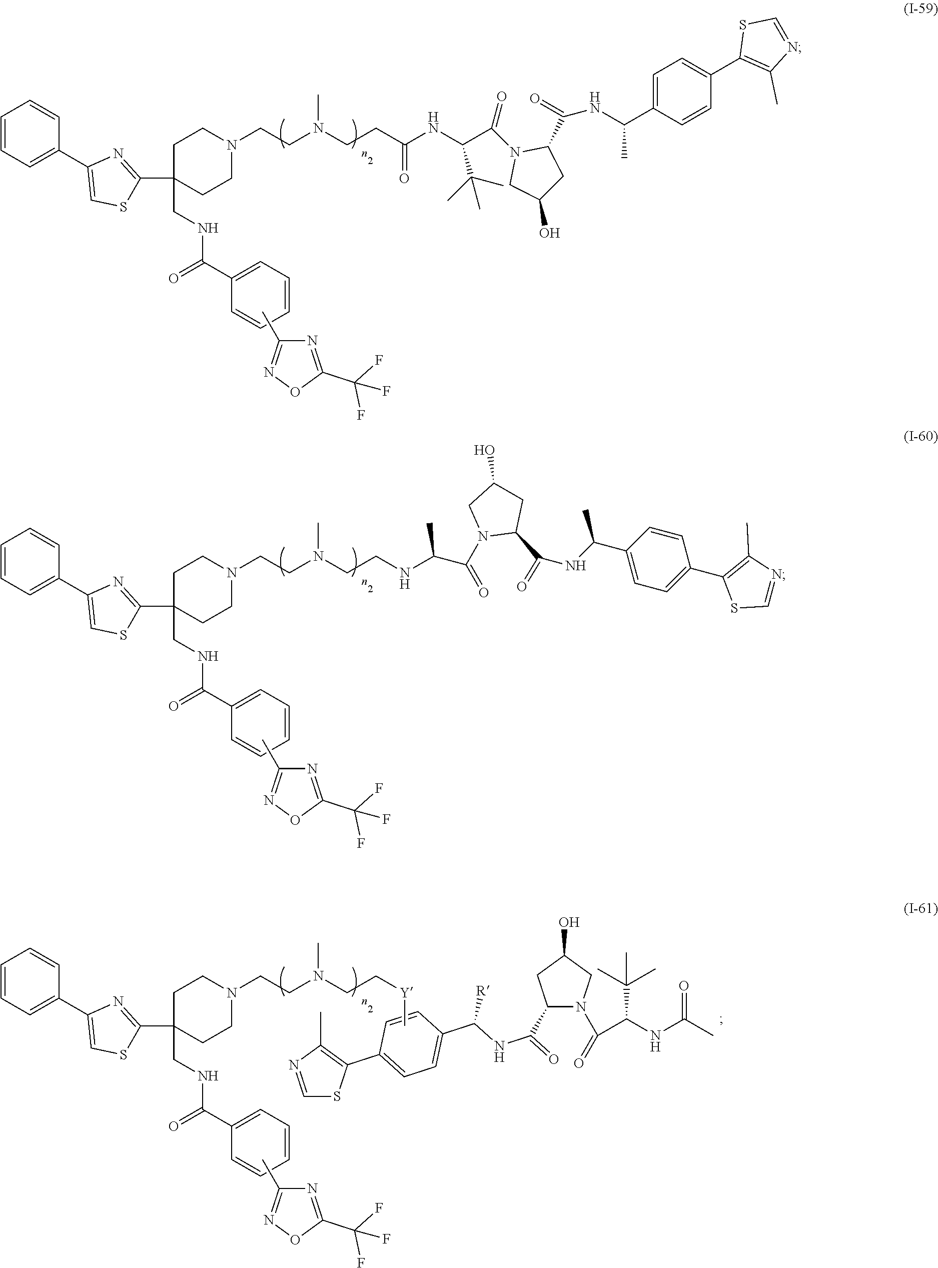 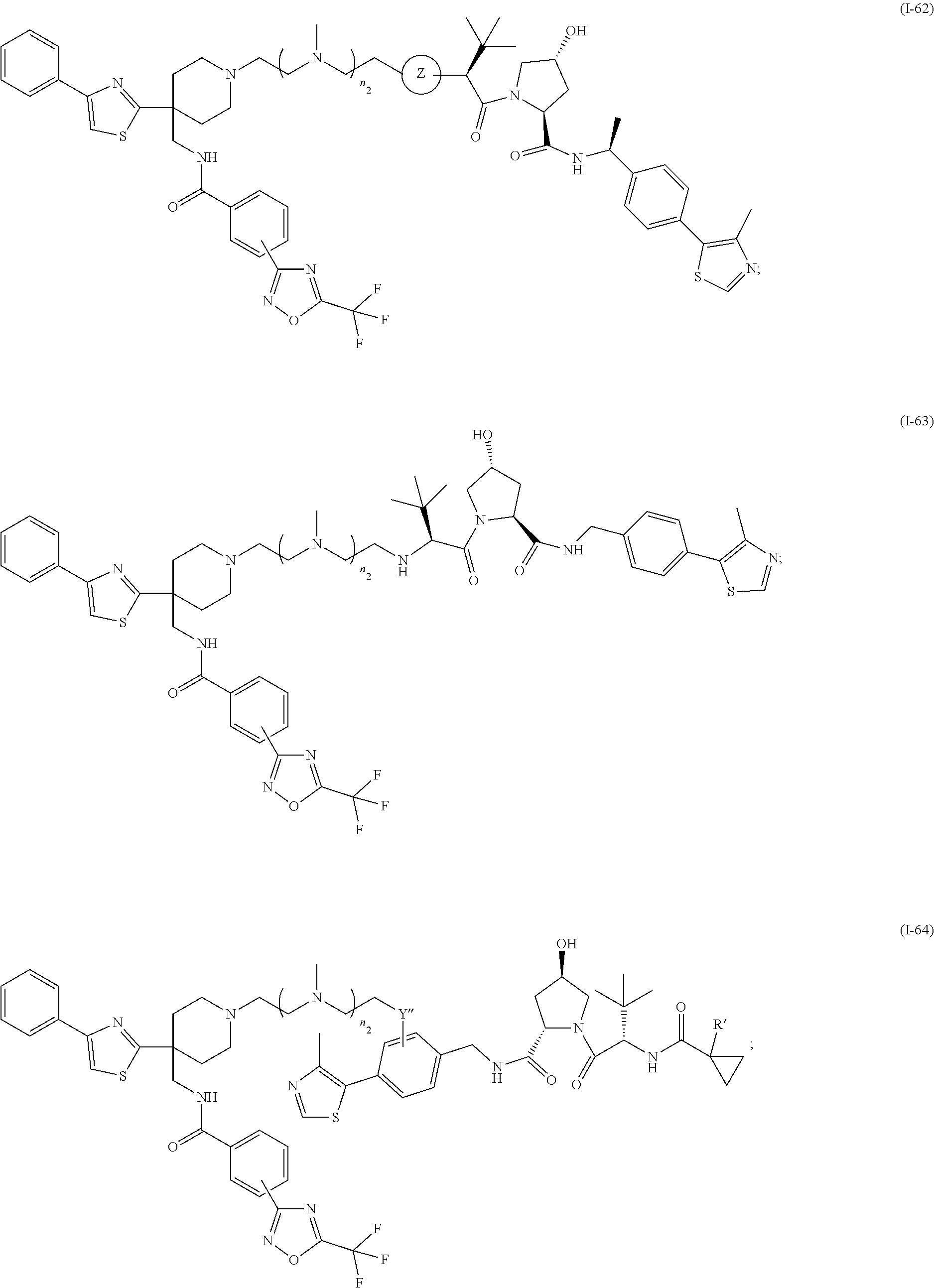 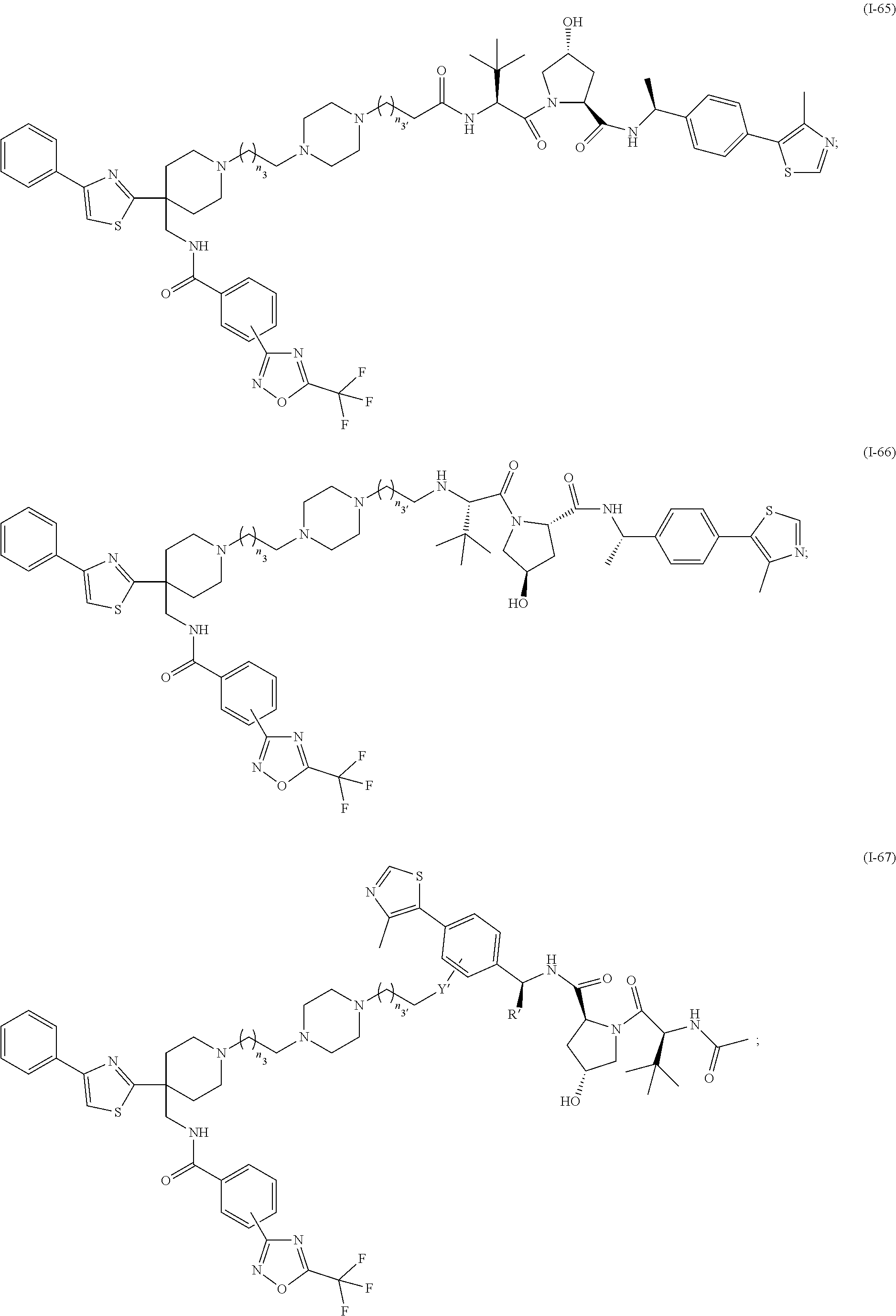 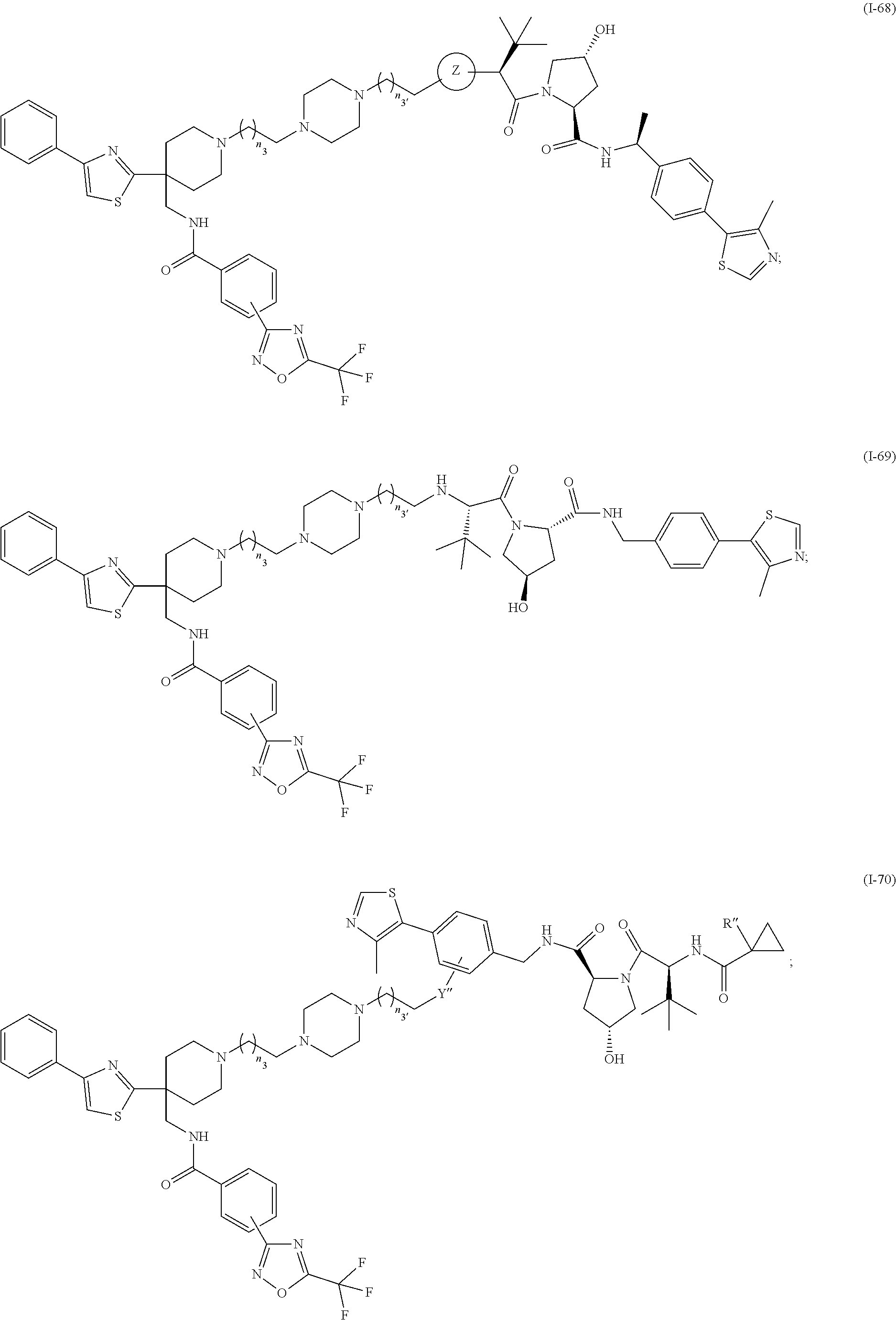 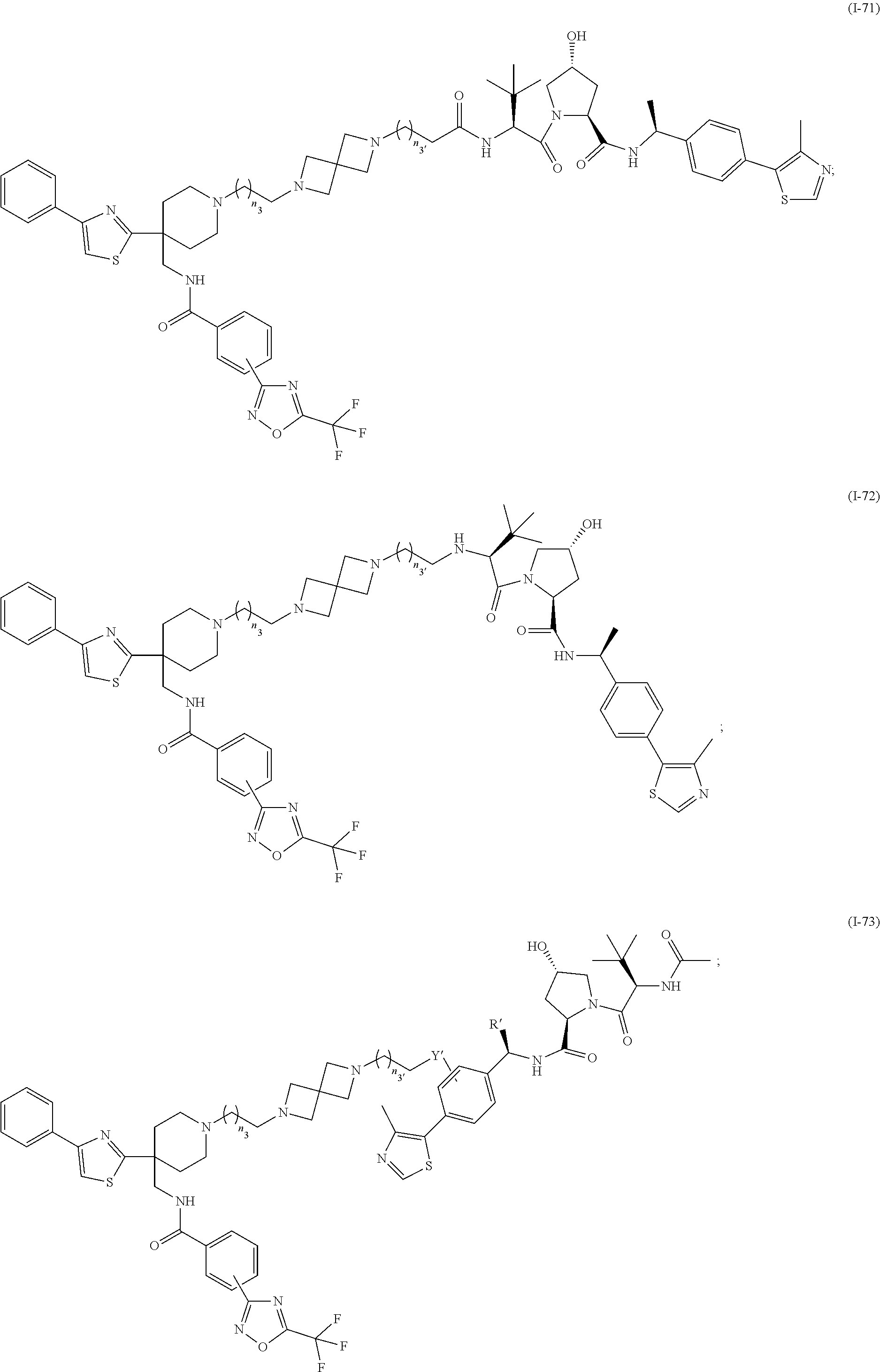 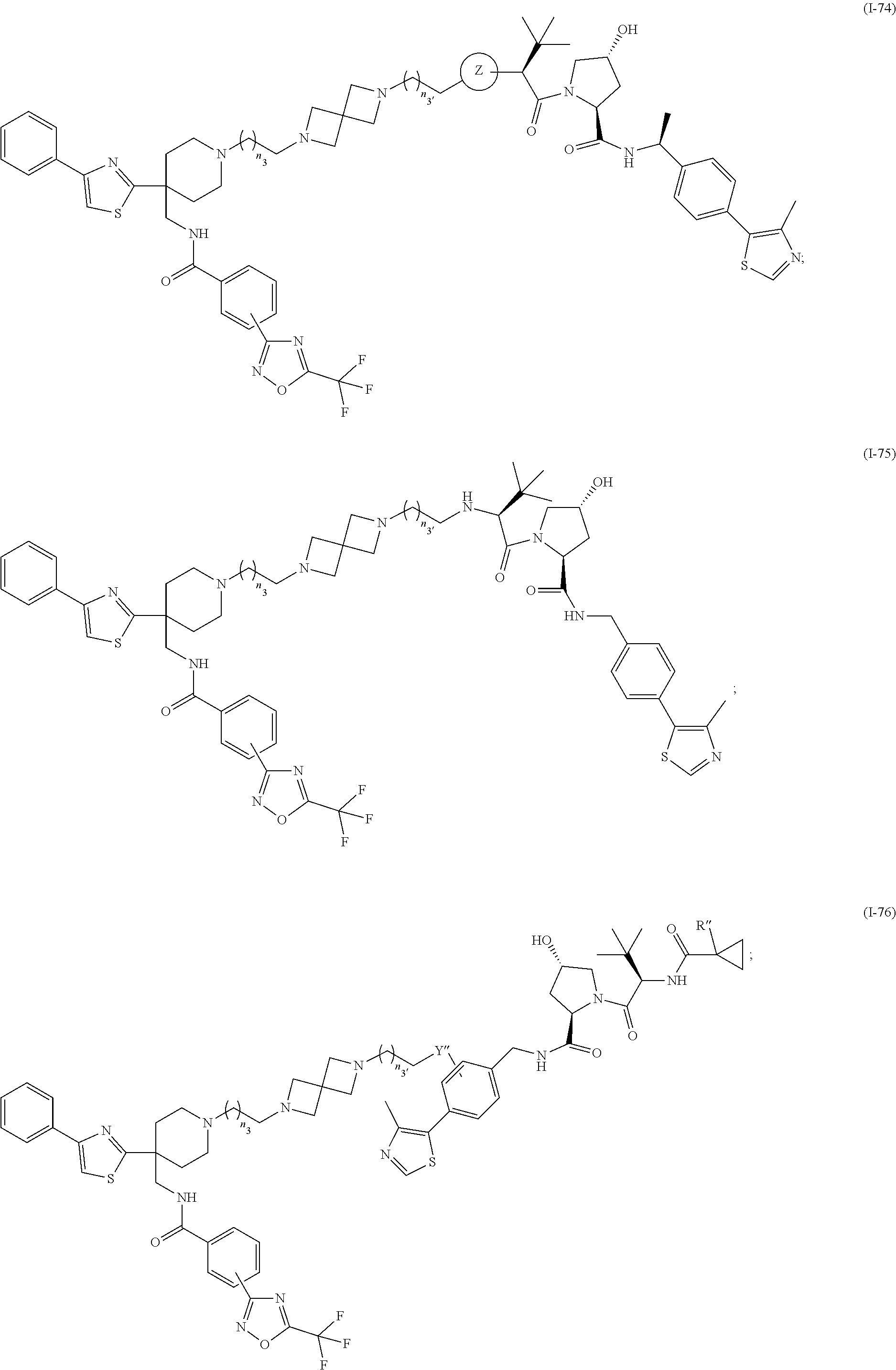 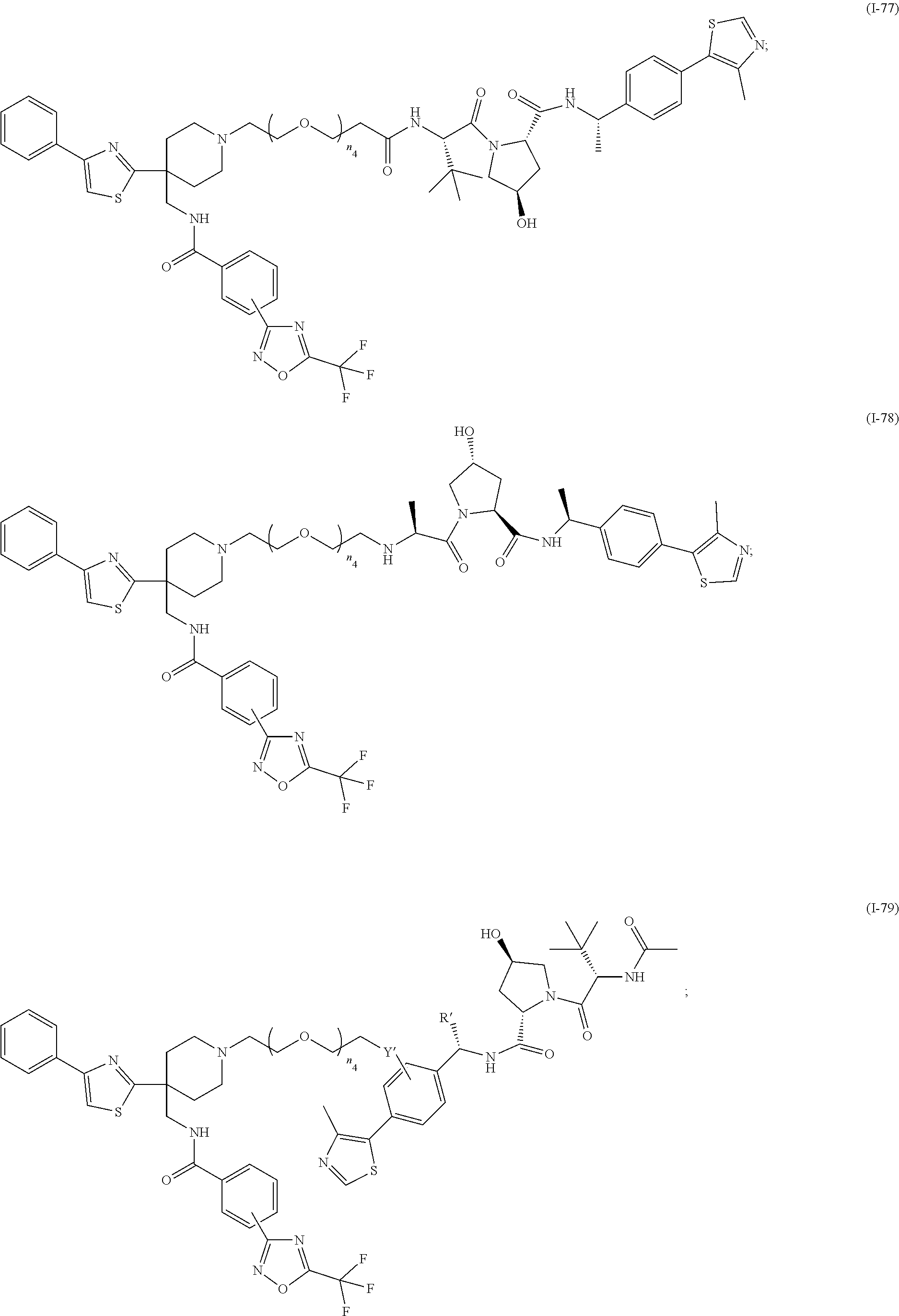 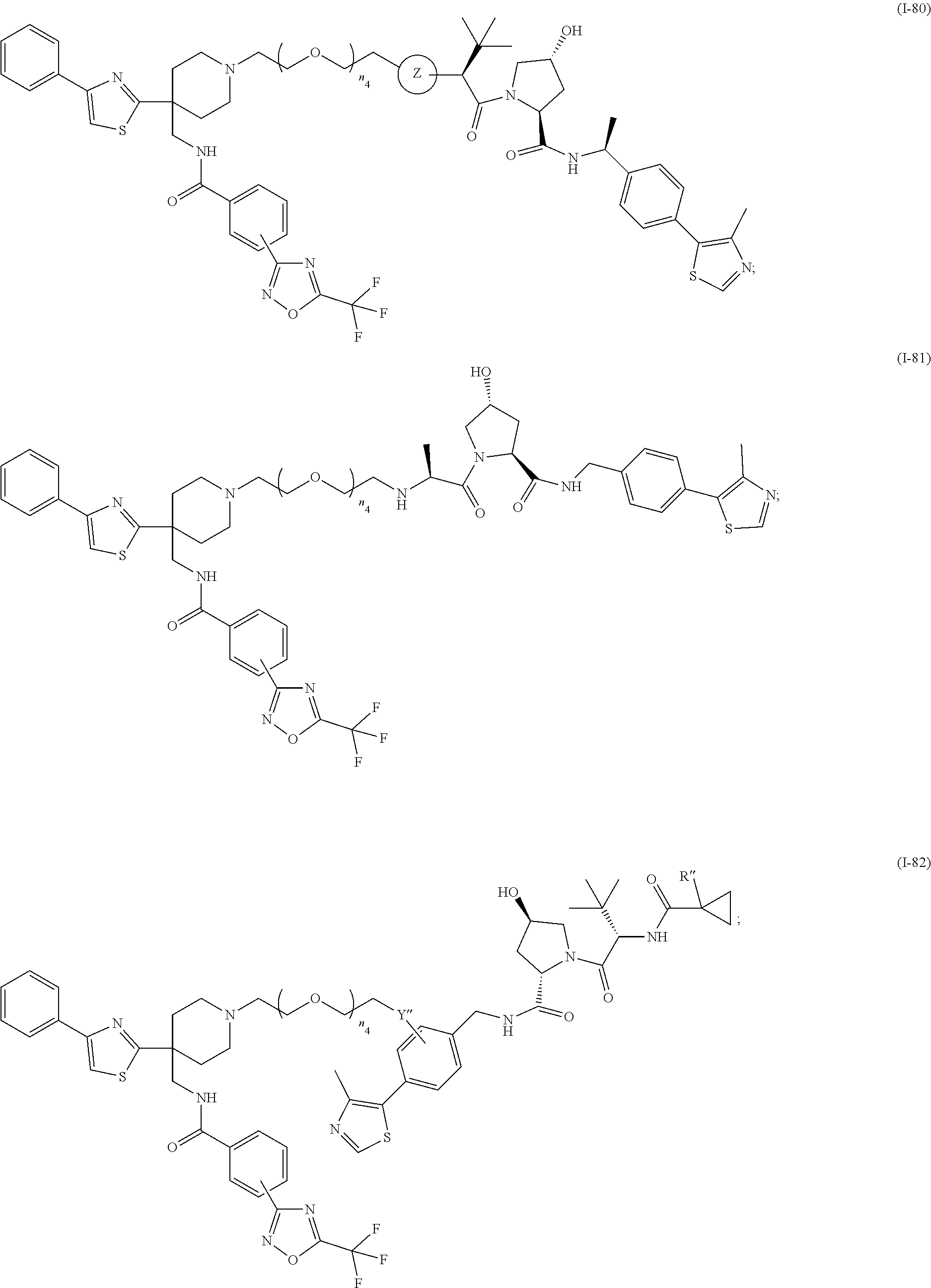 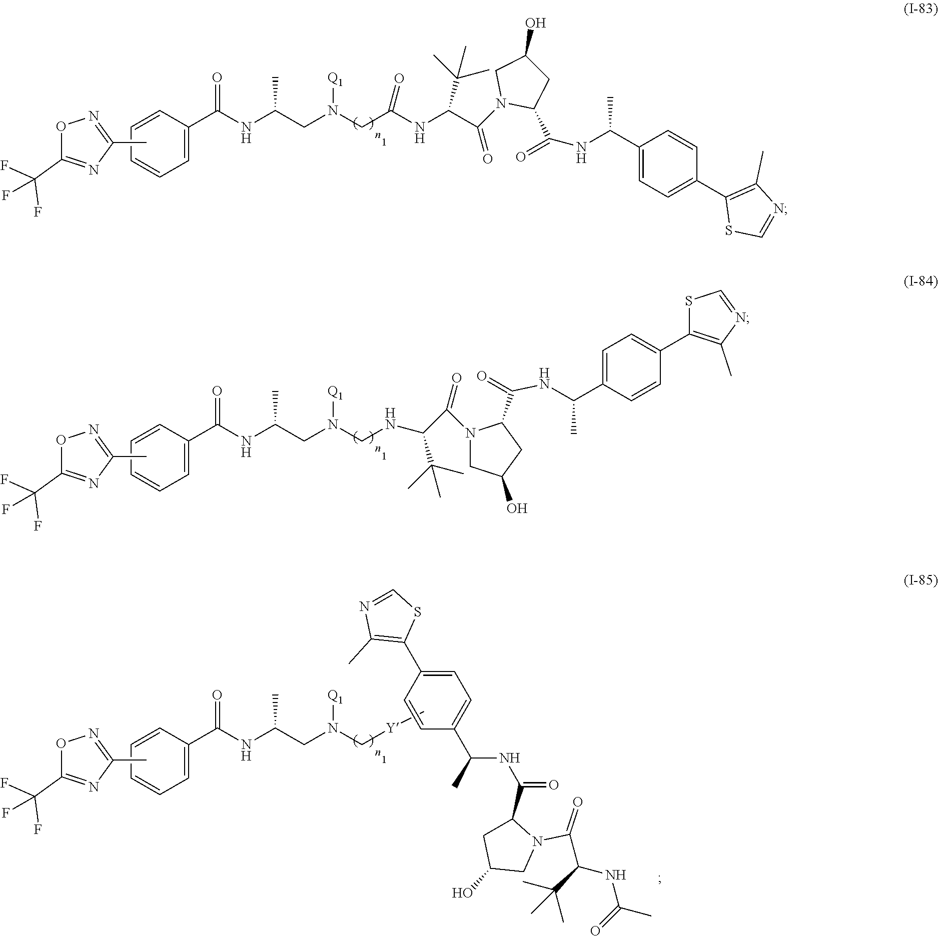 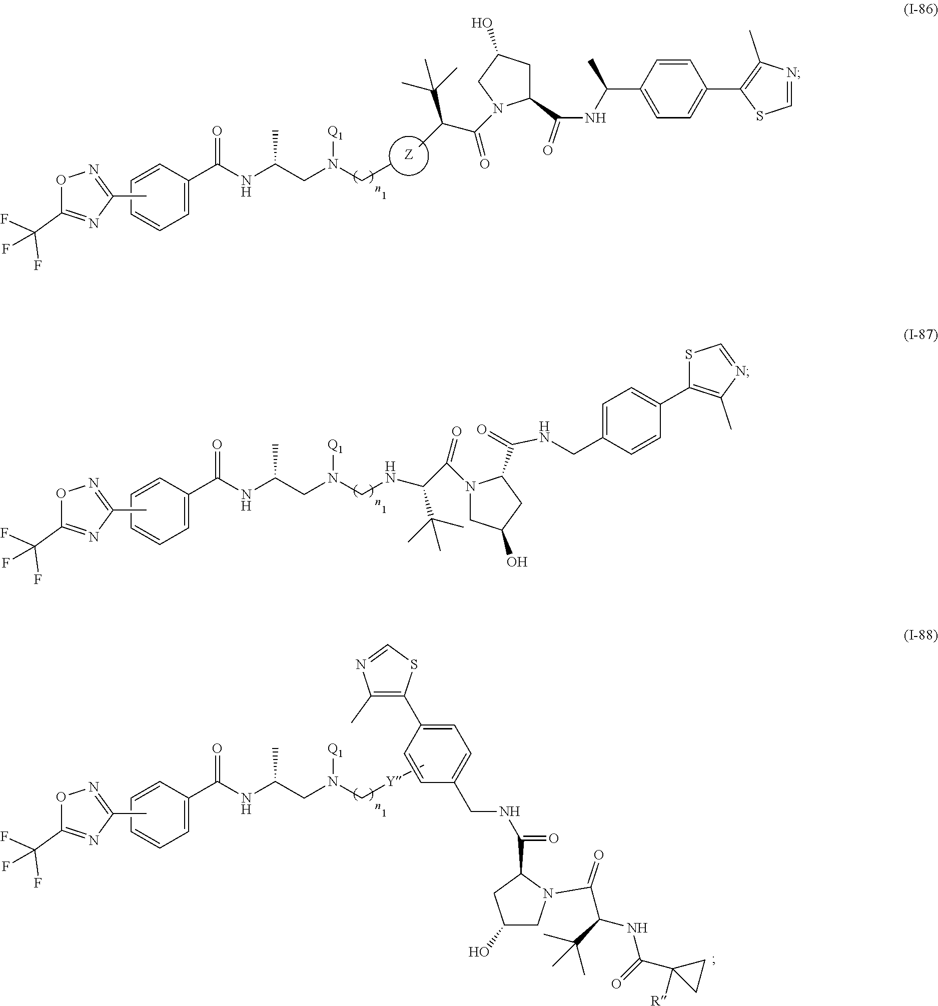 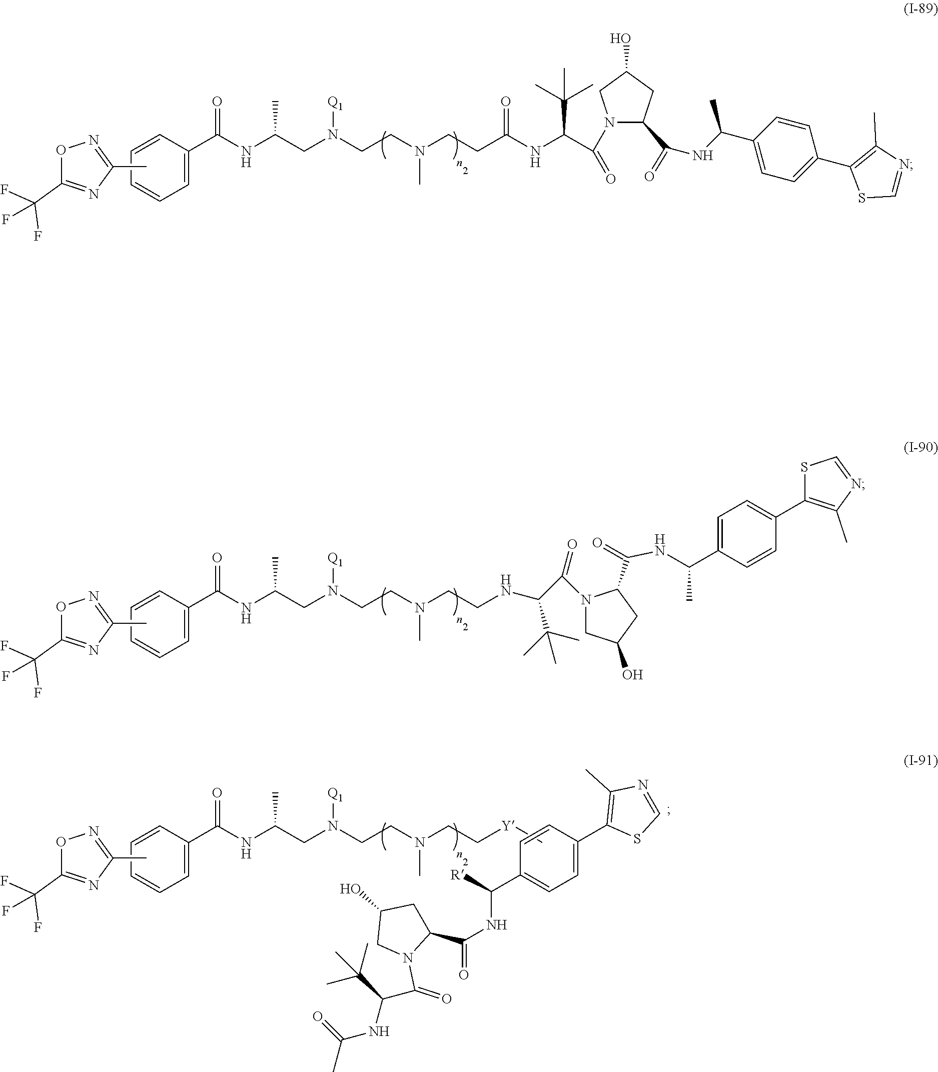 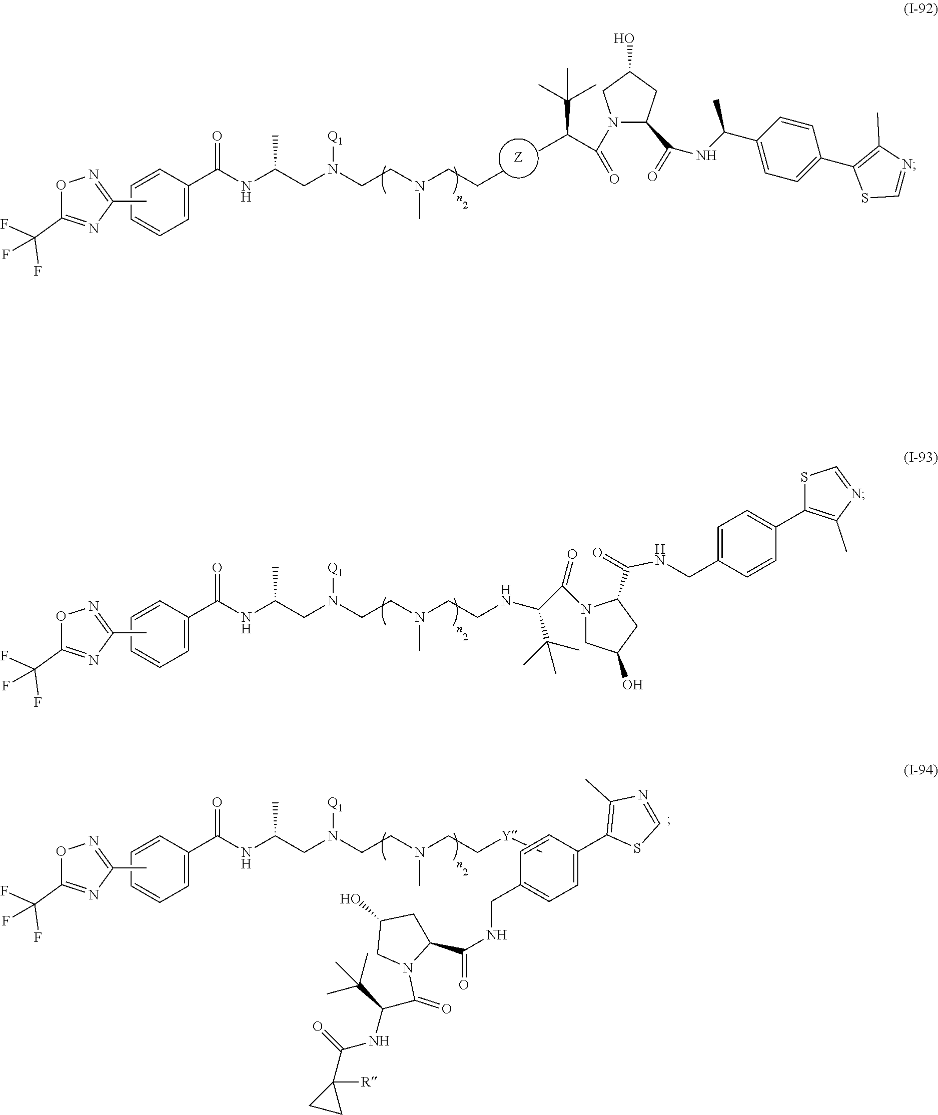 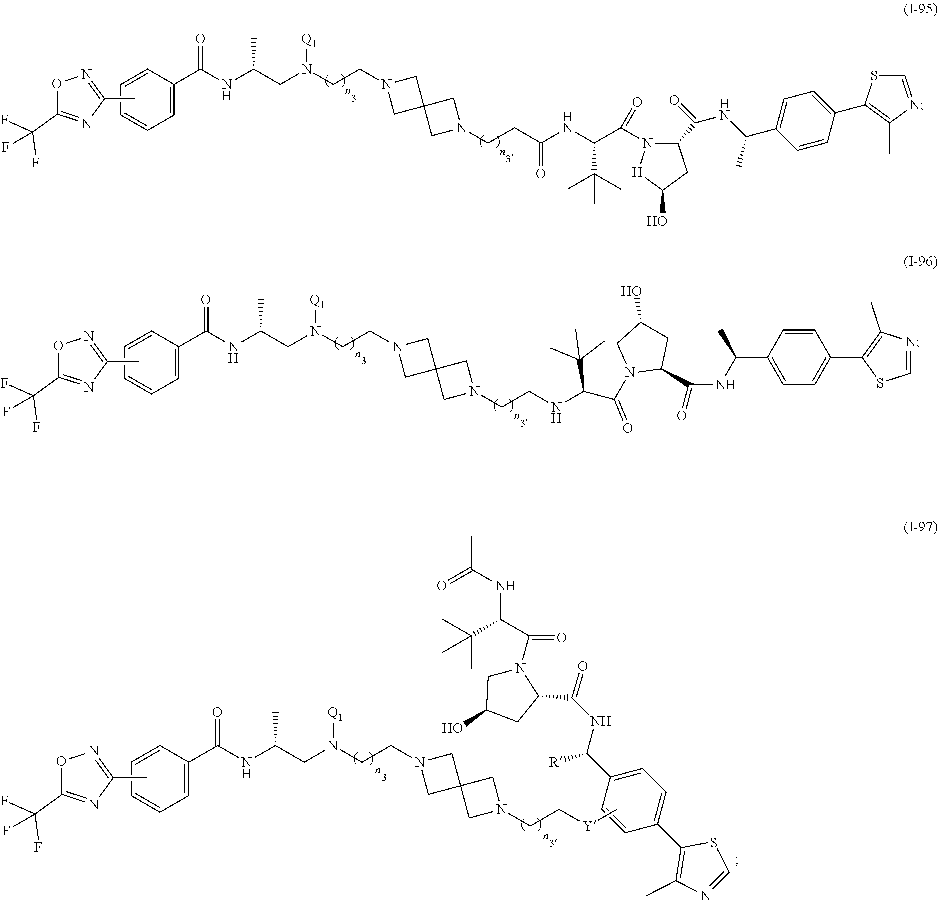 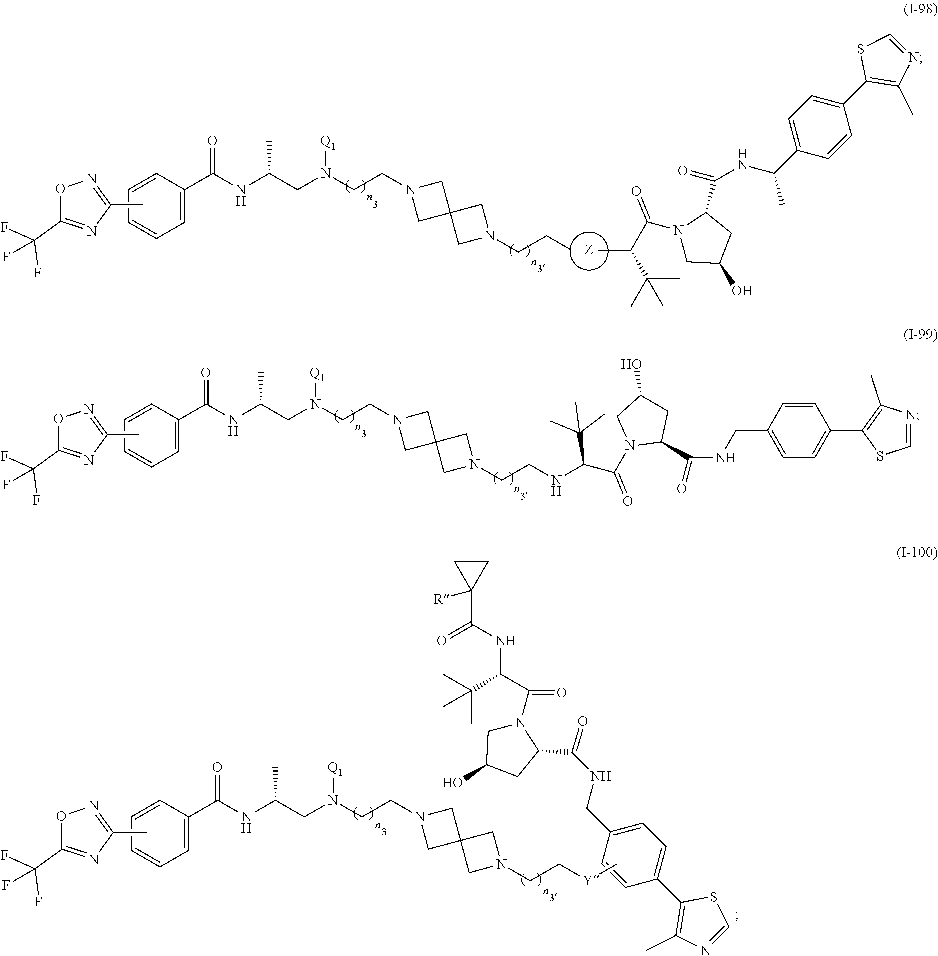 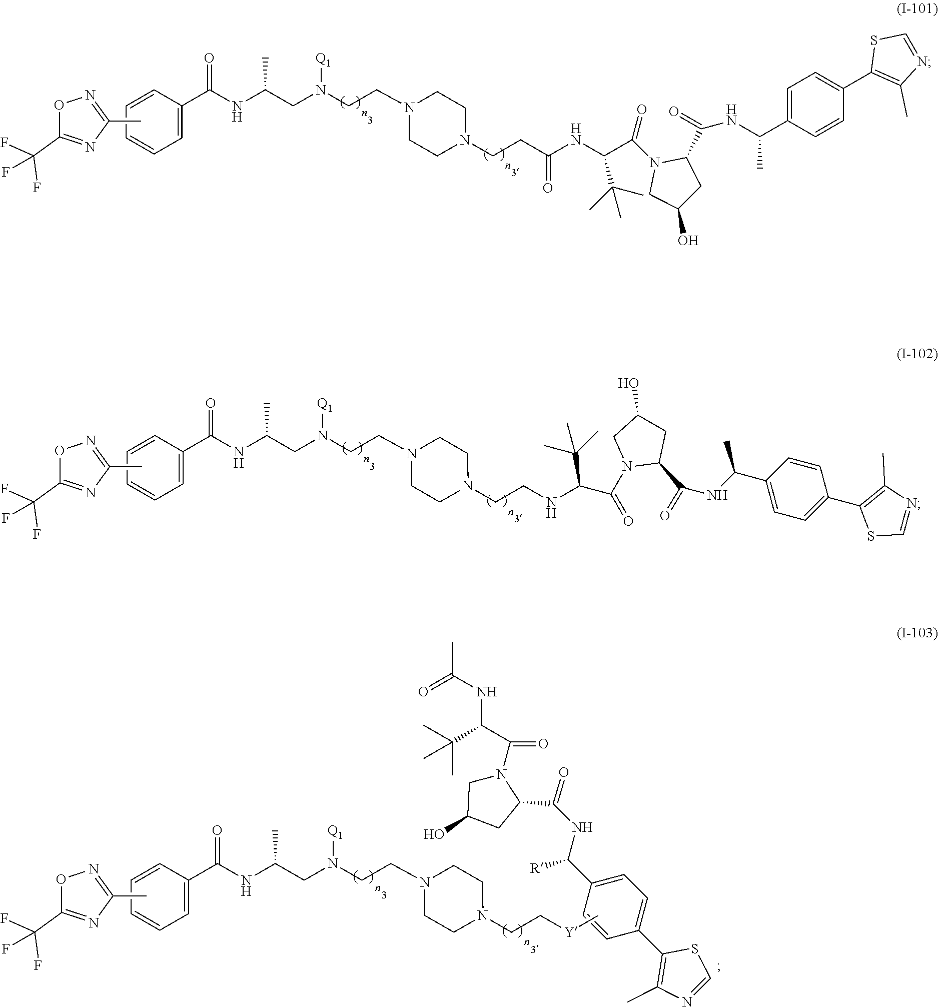 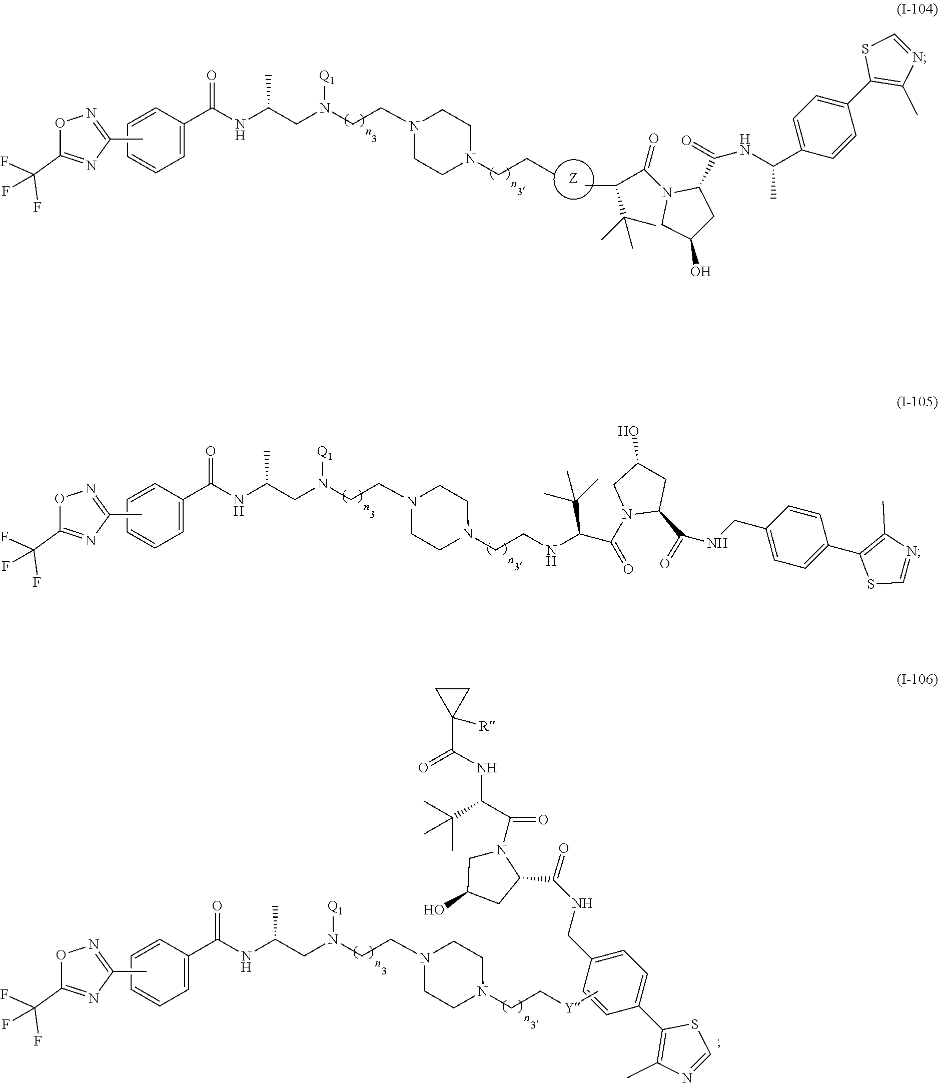 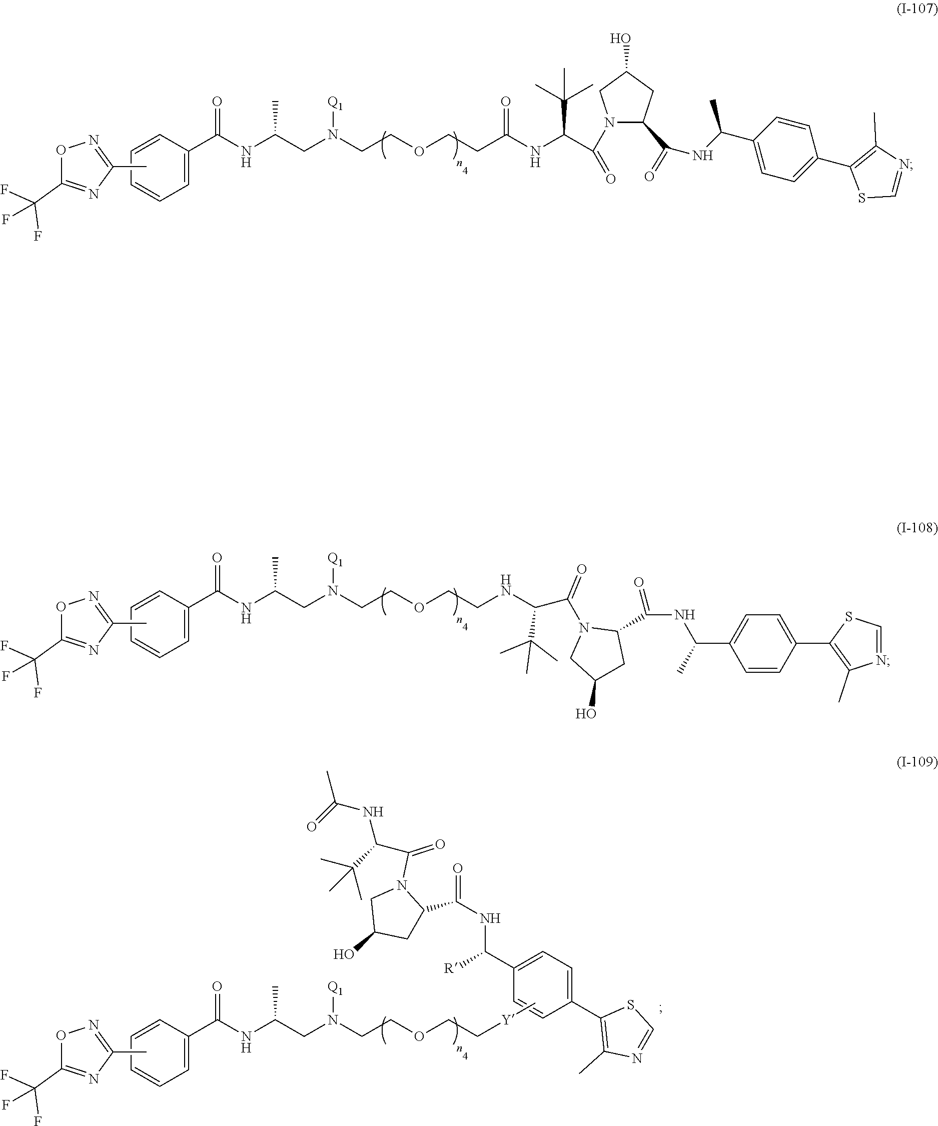 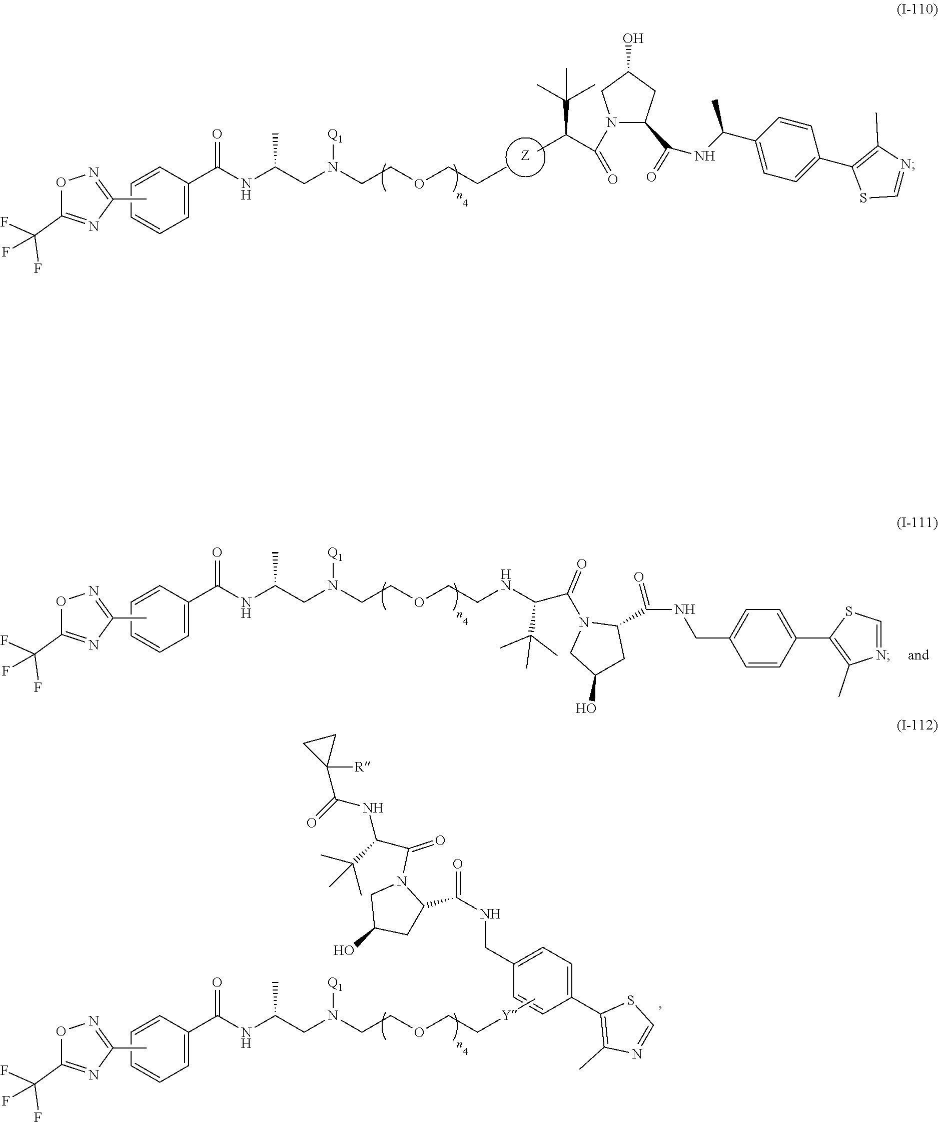
or a pharmaceutically acceptable salt or stereoisomer thereof.

20. The compound of claim 1, wherein the degron binds IAP and has a structure represented by any one of structures (D3-a) to (D3-e):

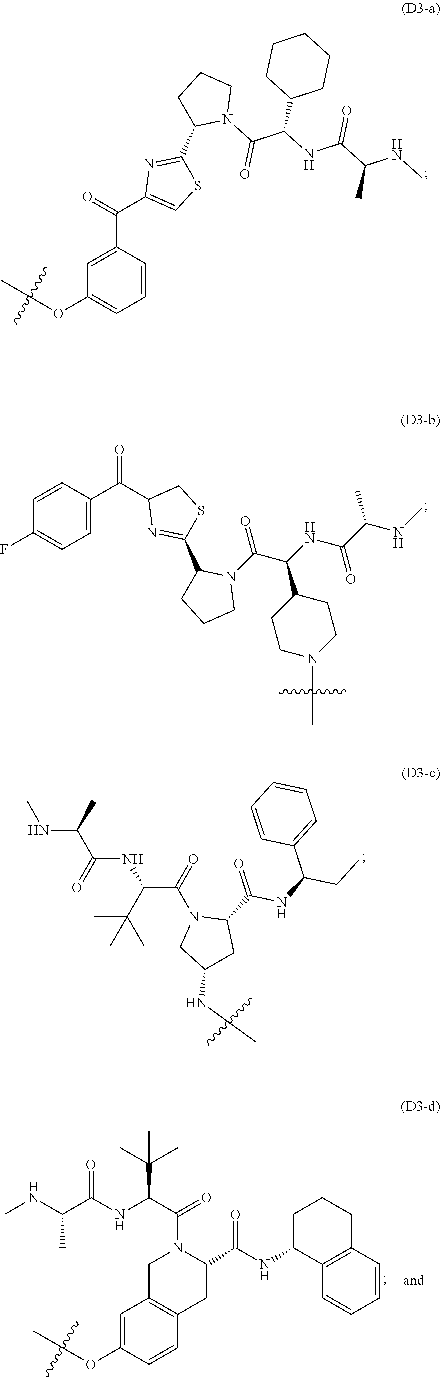

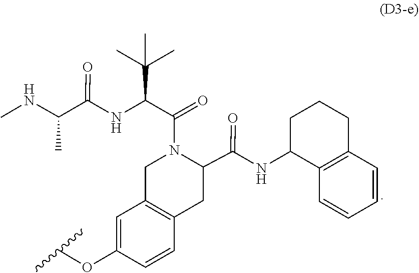 [SMILES: *Oc1ccc2c(c1)CN(C(=O)[C@@H](NC(=O)[C@H](C)NC)C(C)(C)C)C(C(=O)NC1CCCc3ccccc31)C2]

21. (canceled)

22. The compound of claim 20, which is represented by any one of structures (I-113) to (I-162): 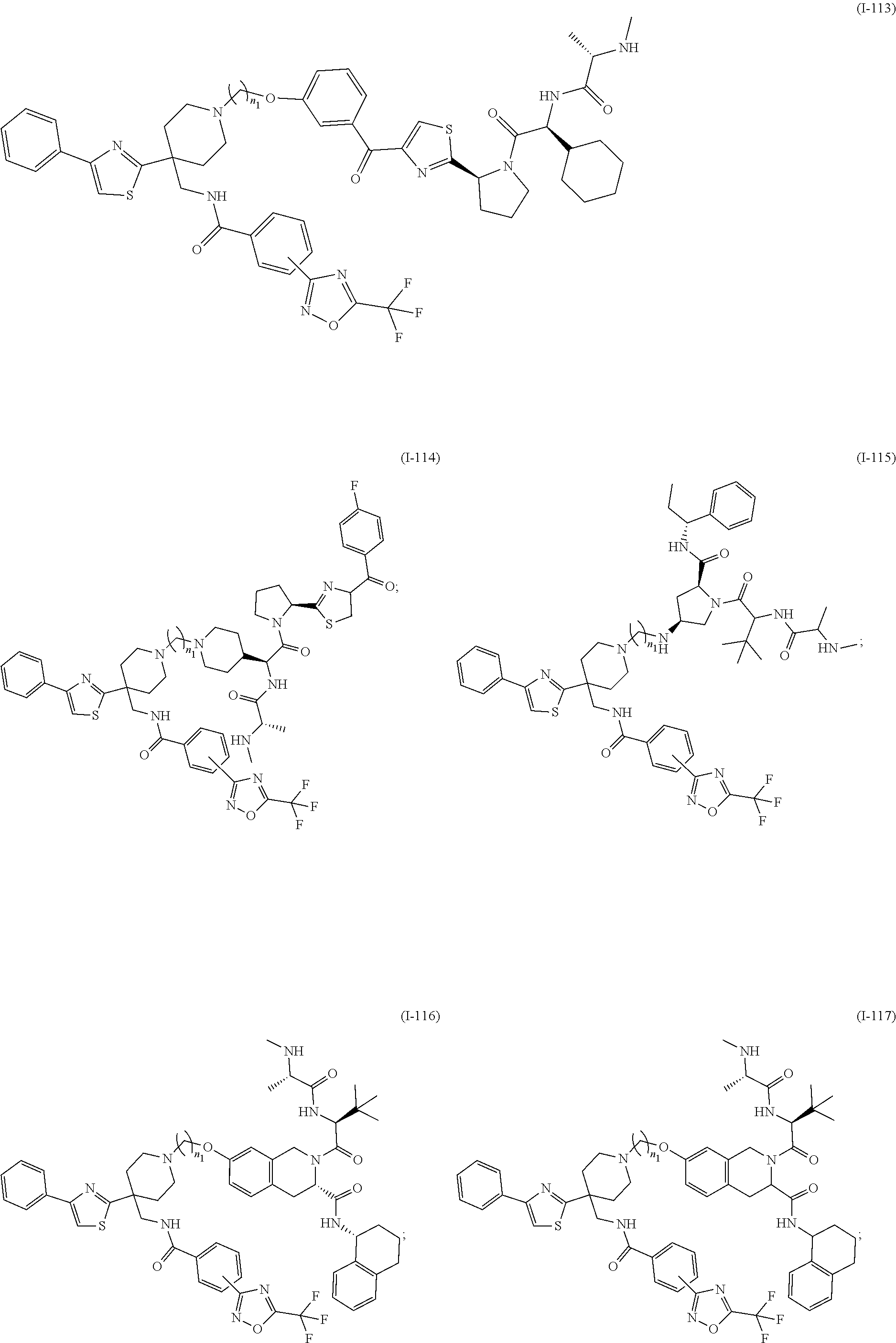 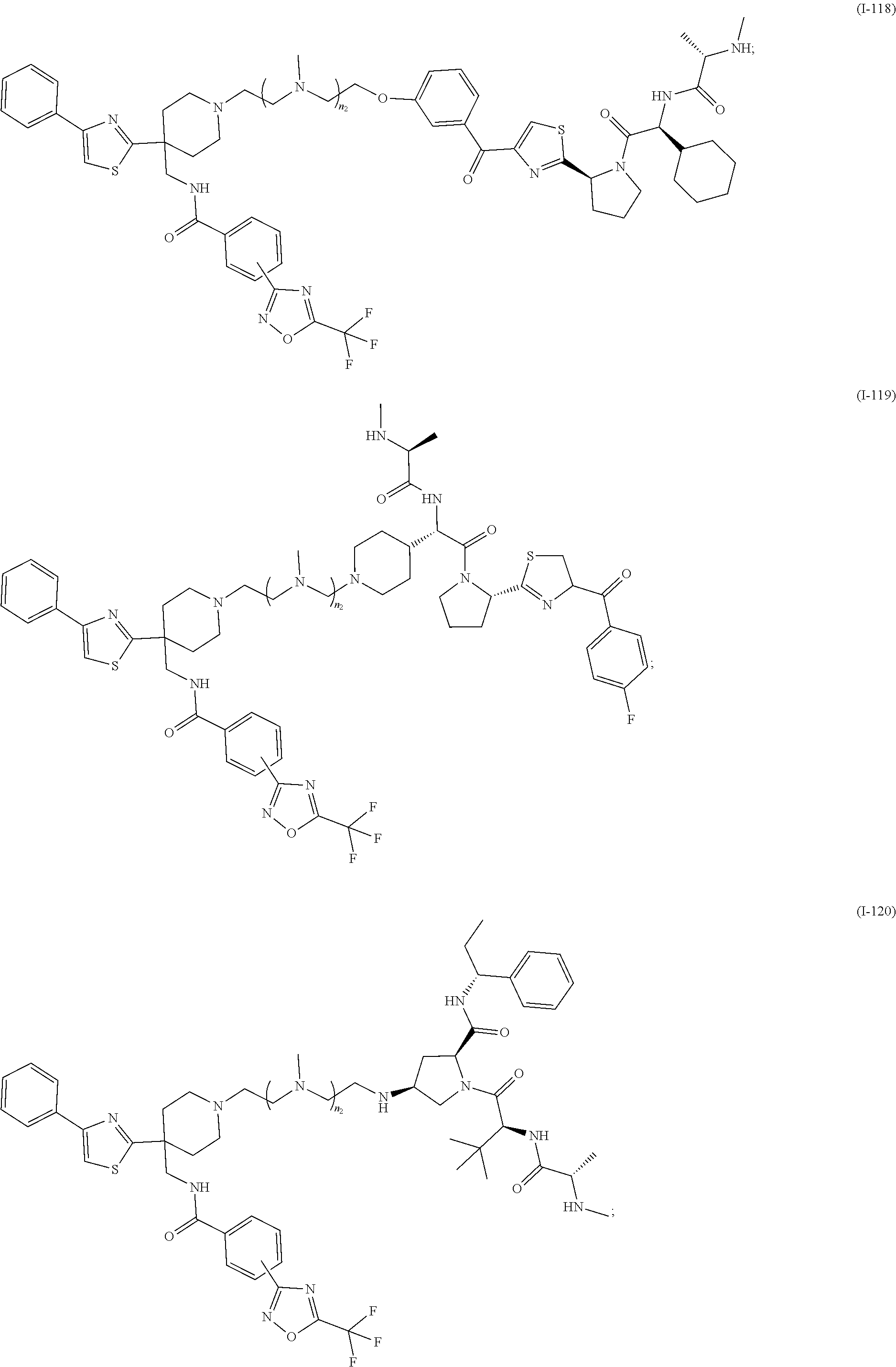 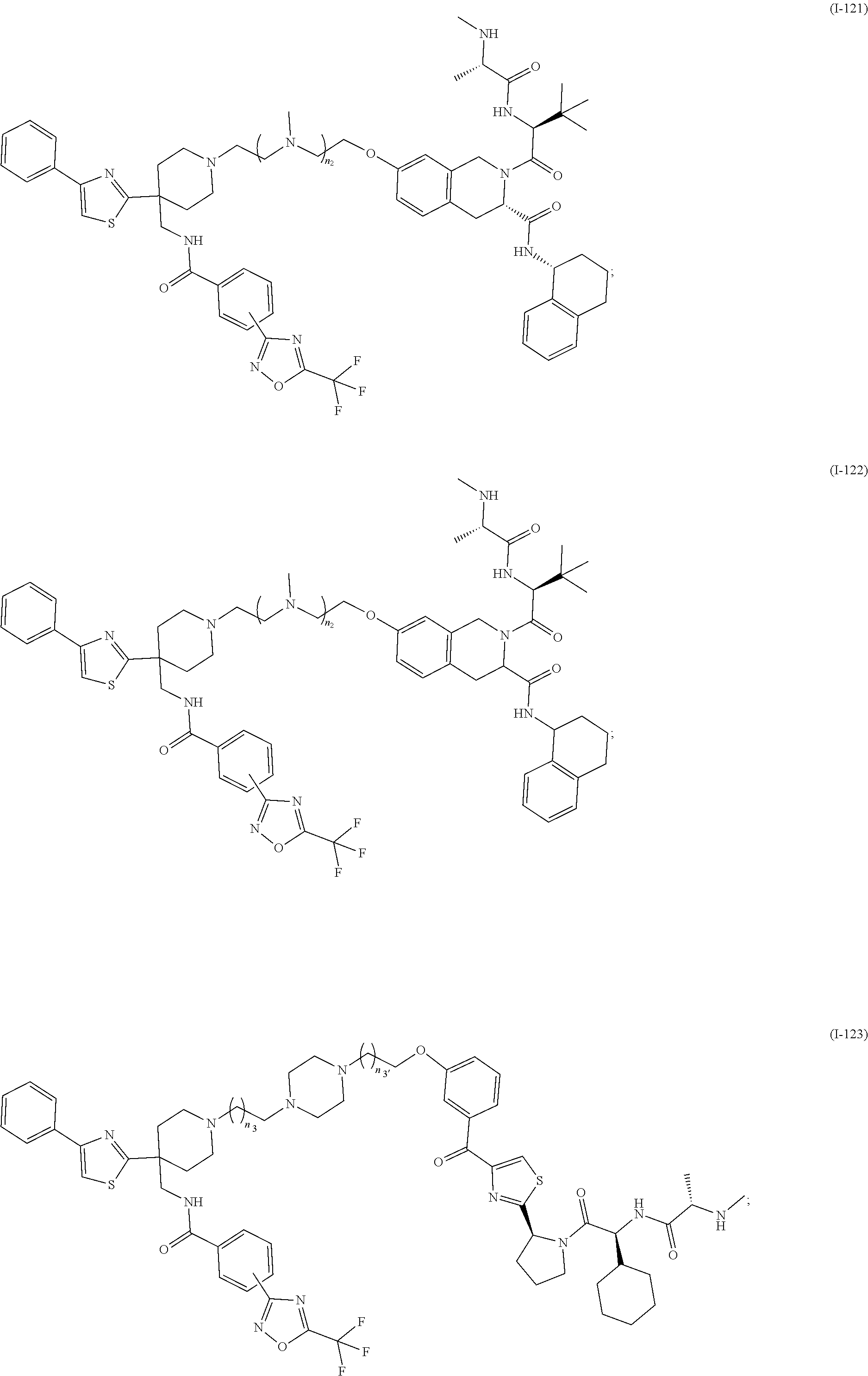 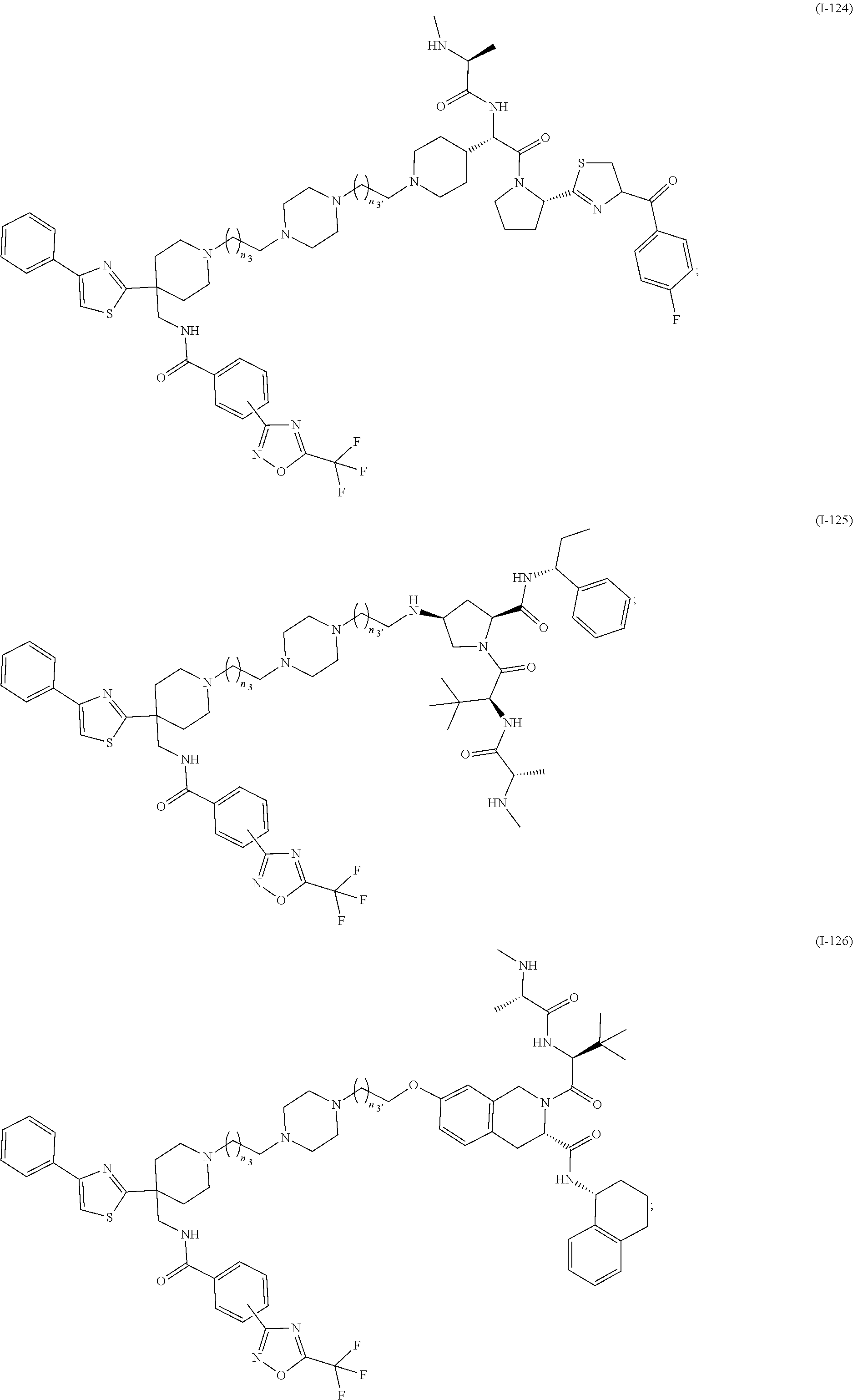 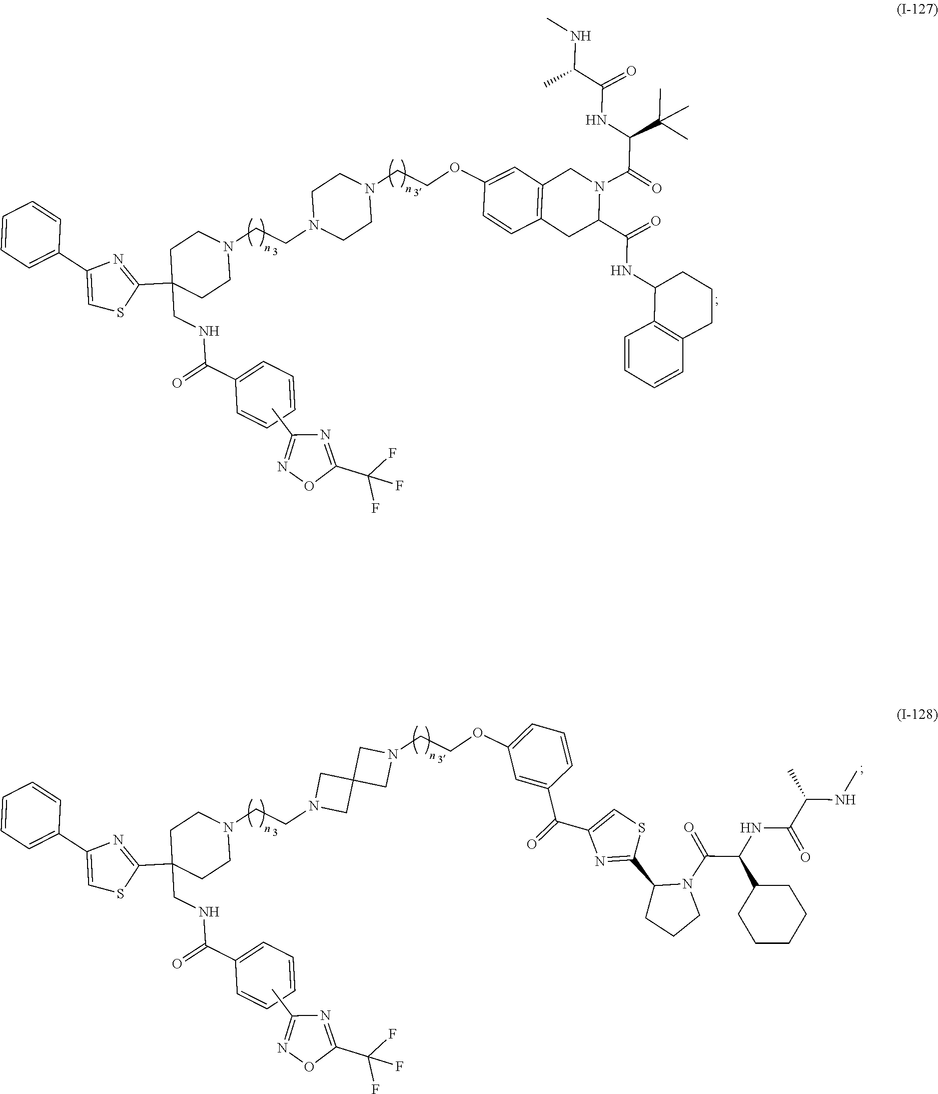 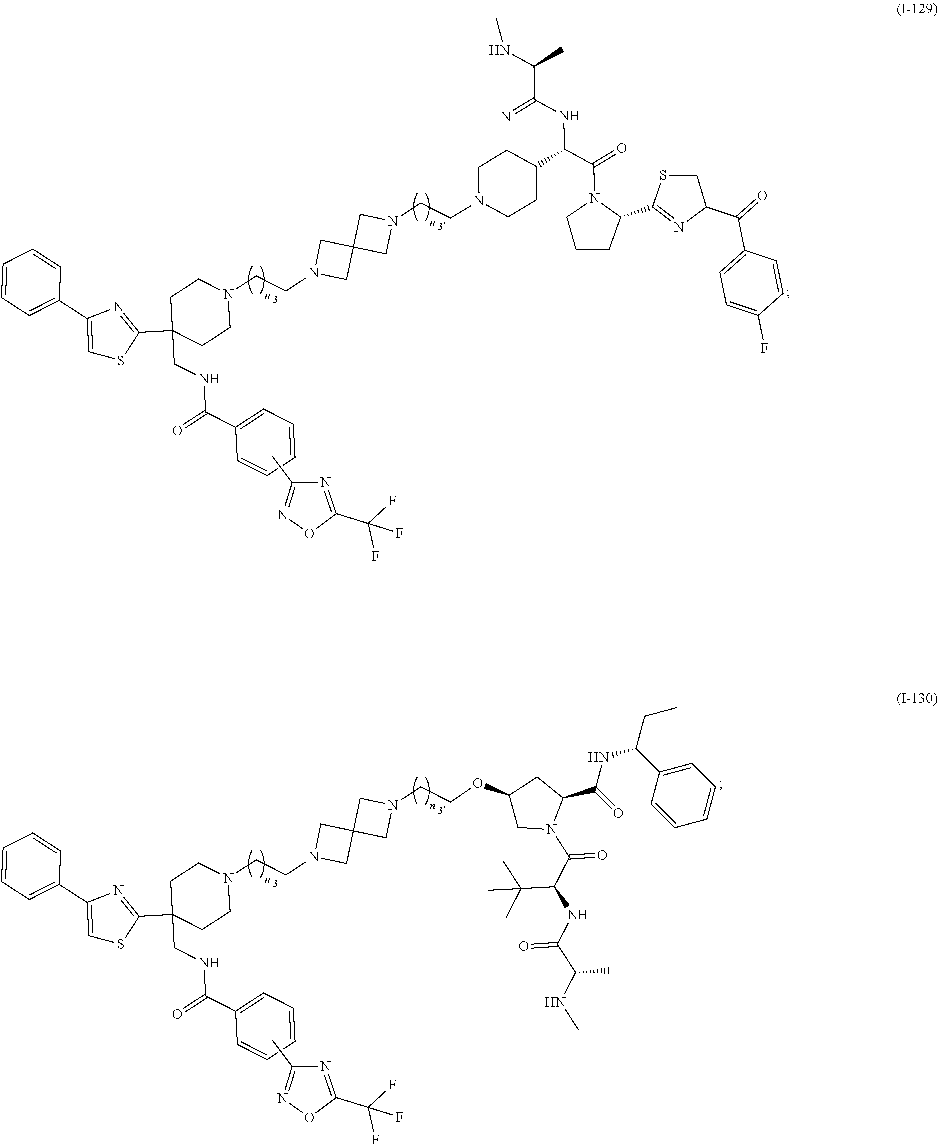 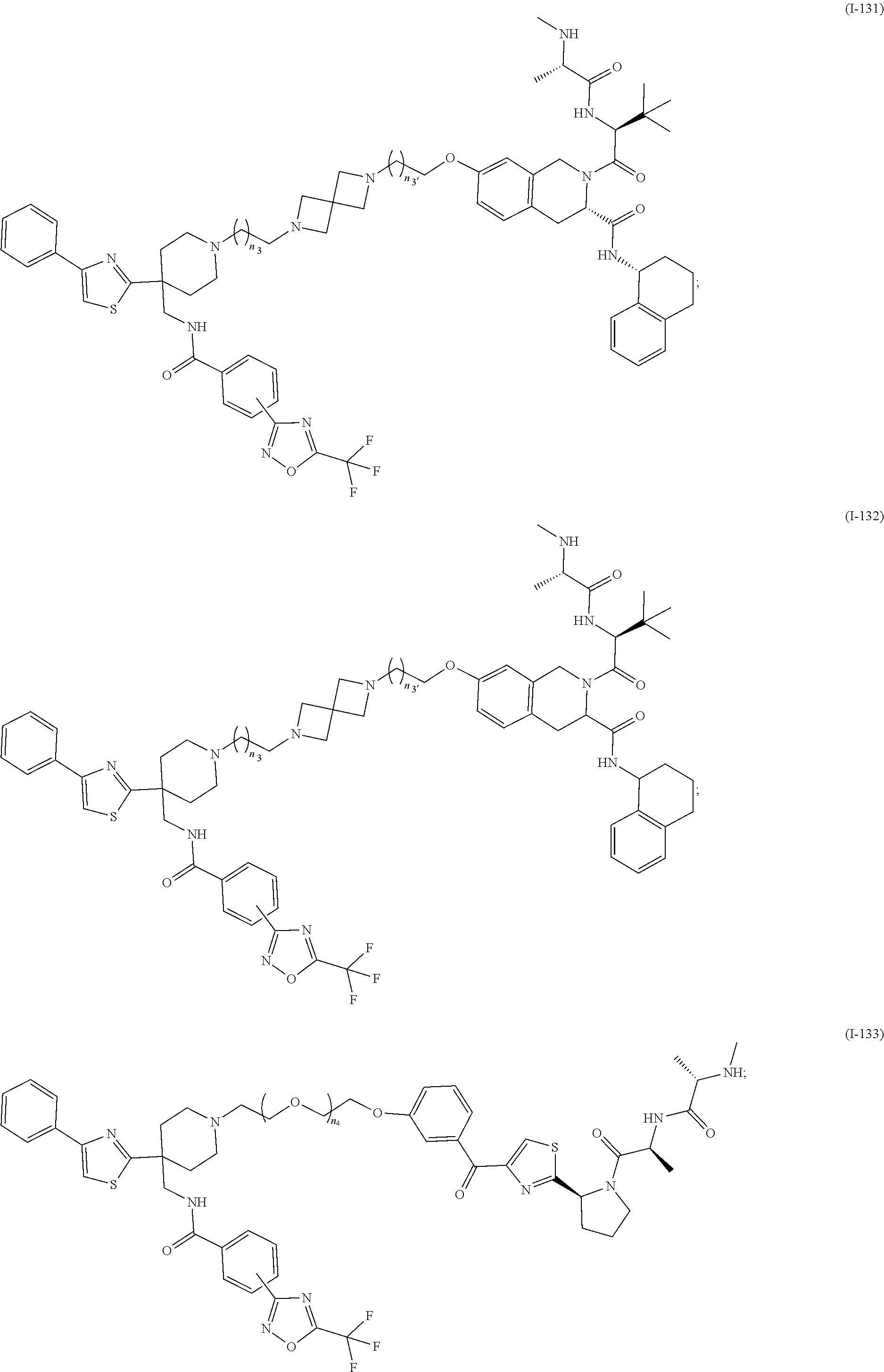 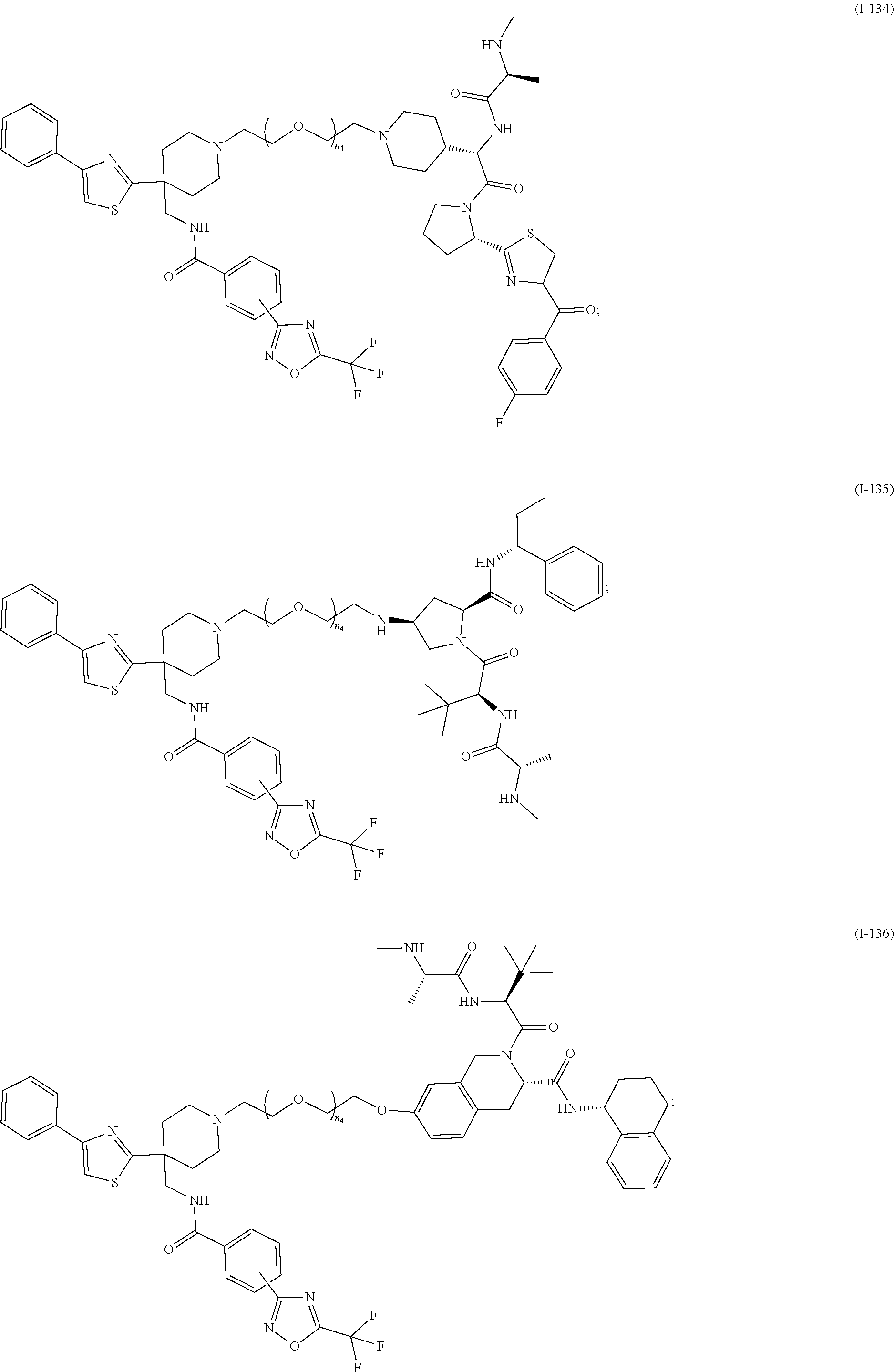 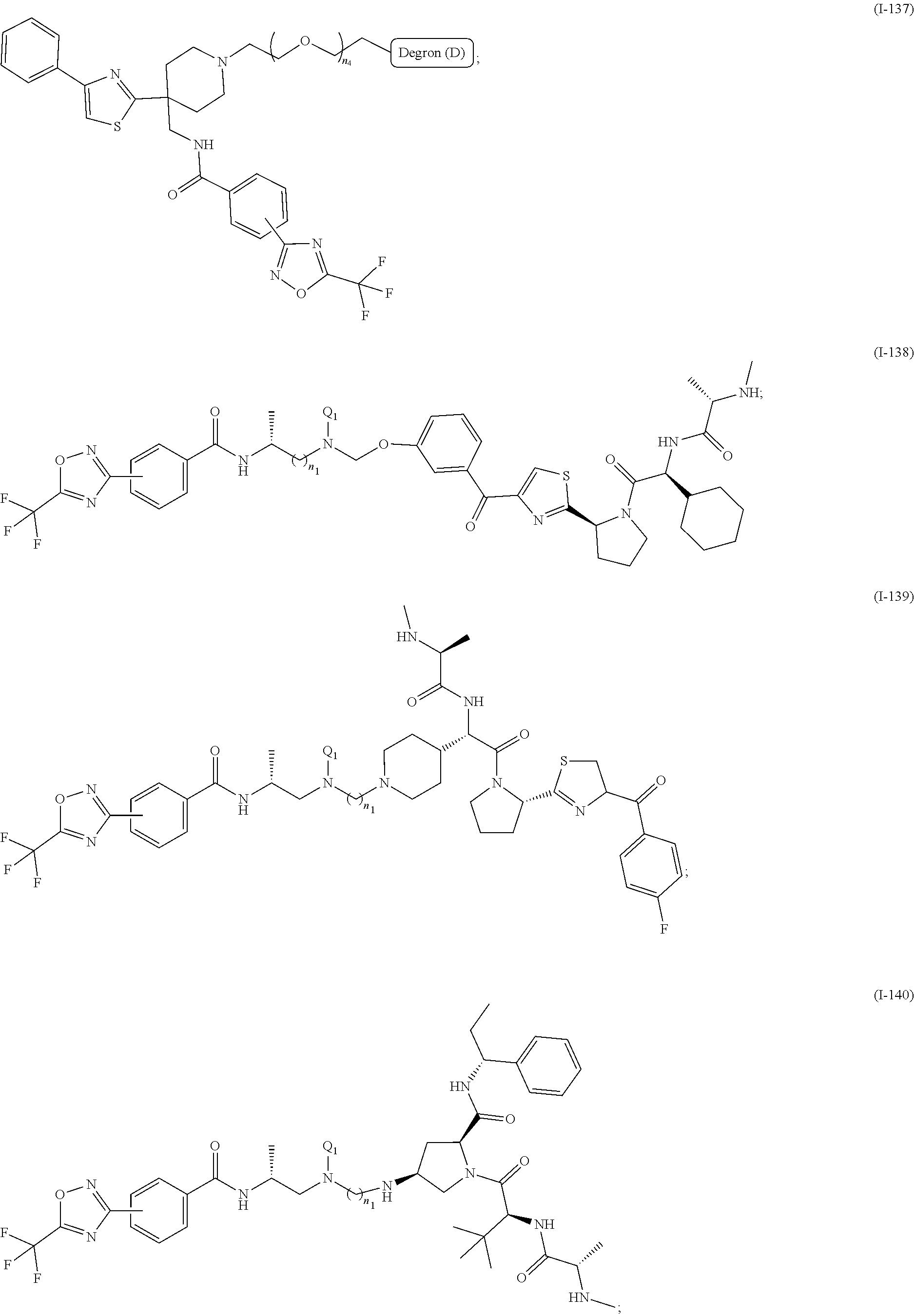 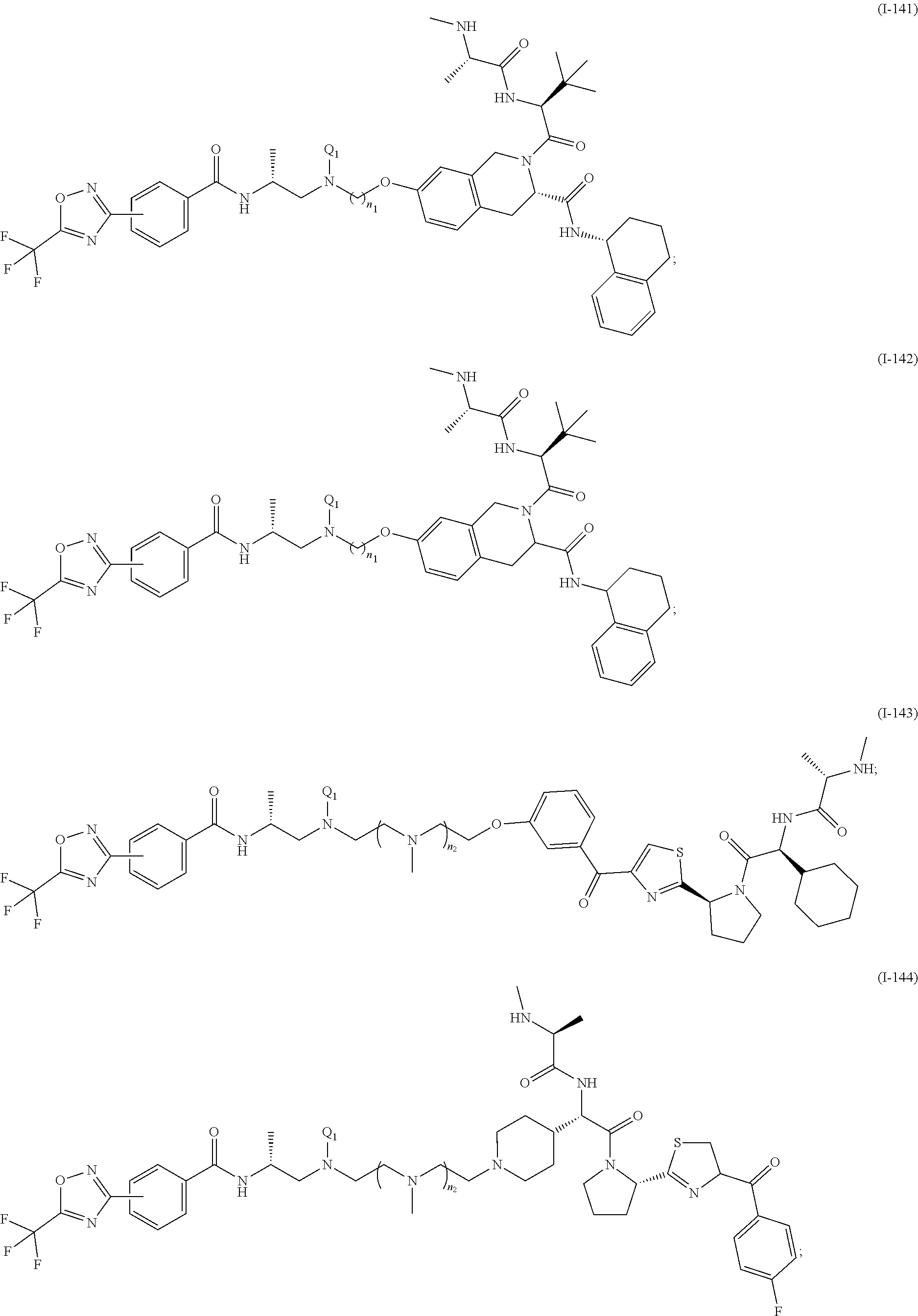 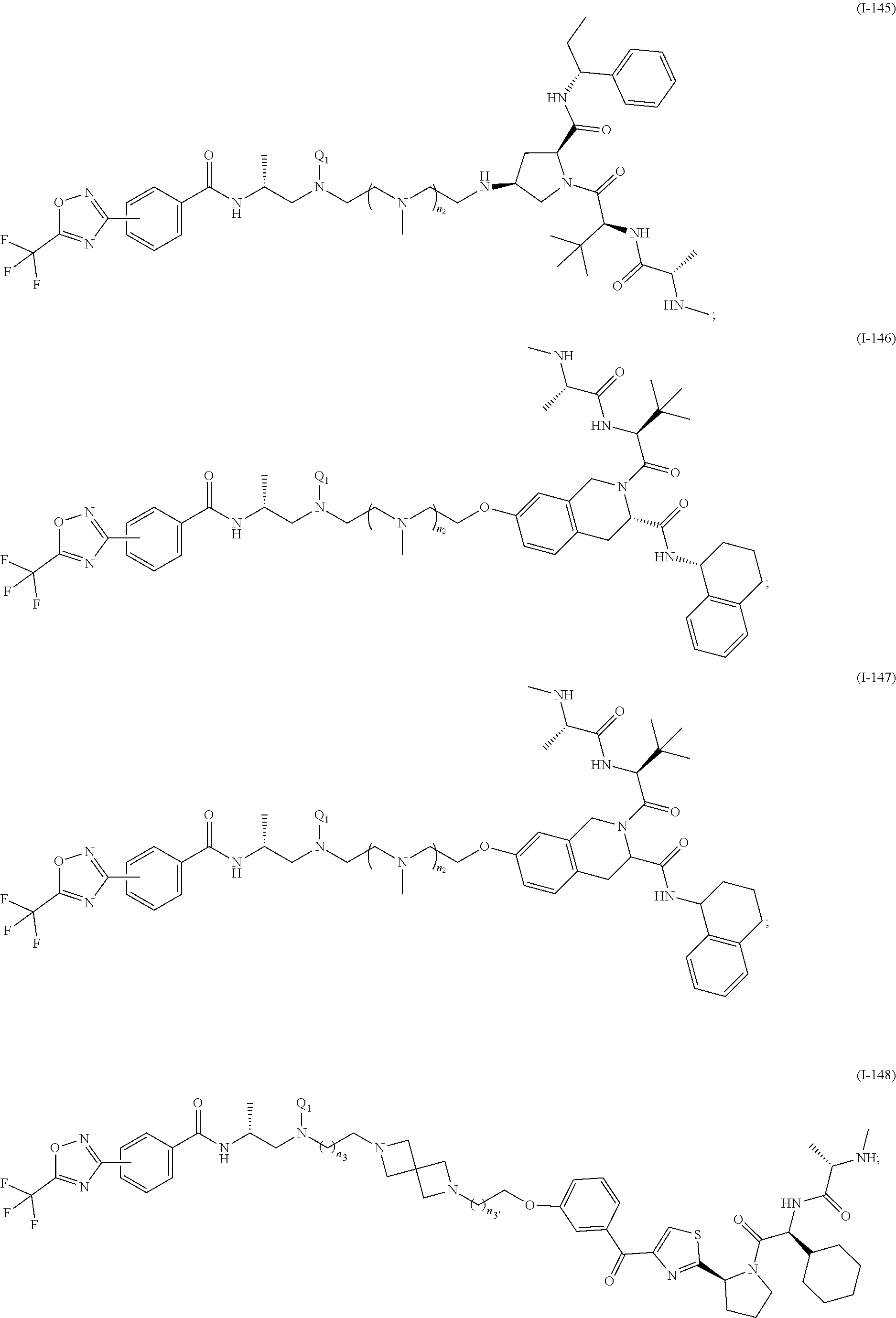 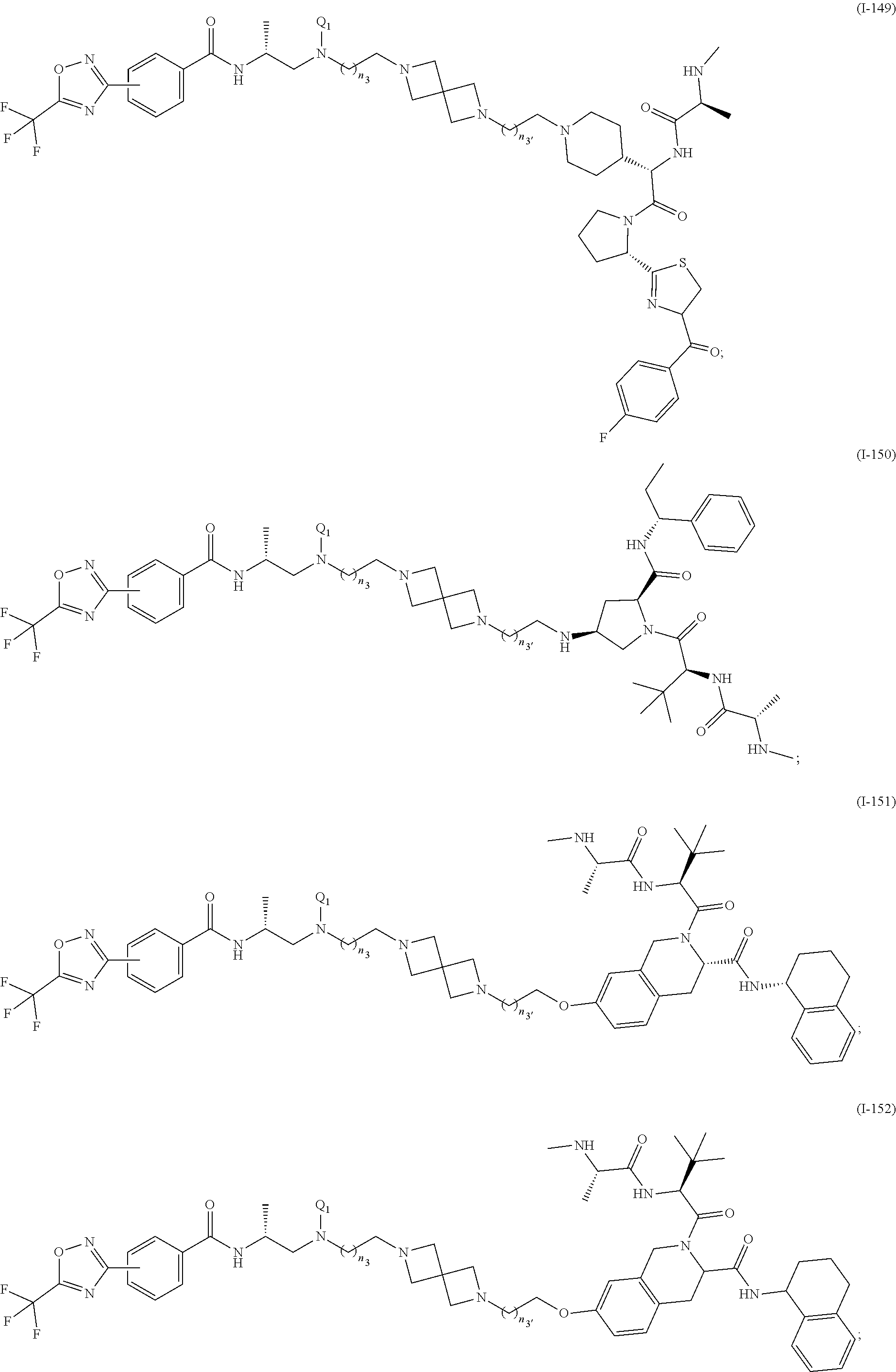 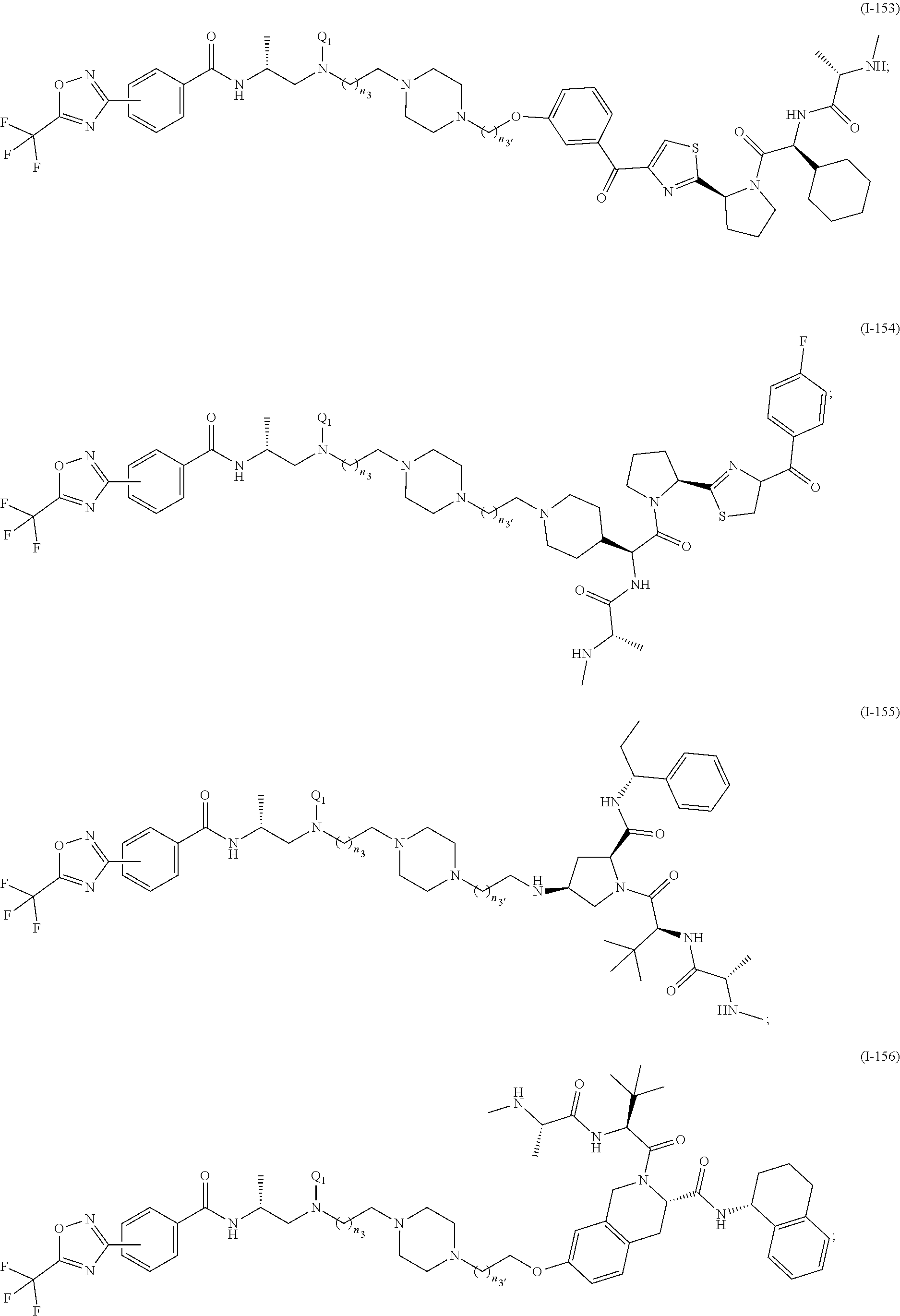 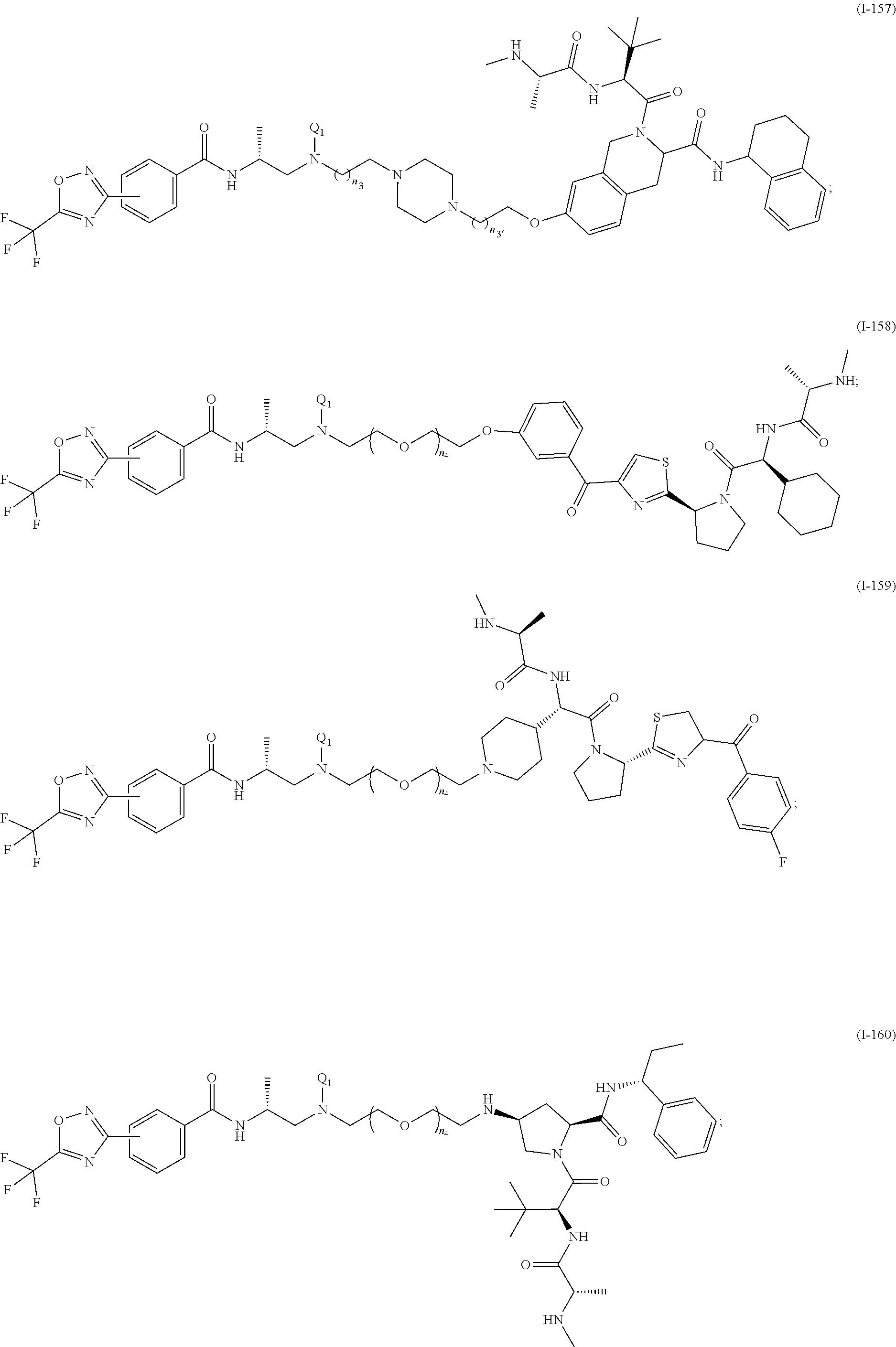 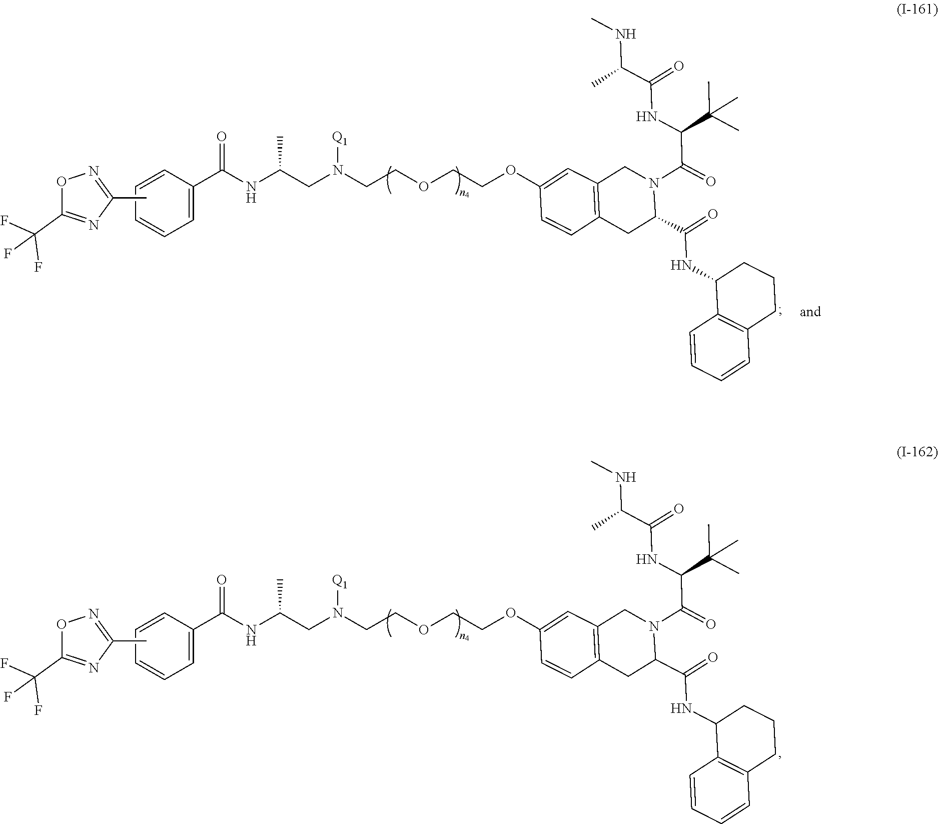
or a pharmaceutically acceptable salt or stereoisomer thereof.

23. The compound of claim 1, which is any one of structures (1) to (26):F 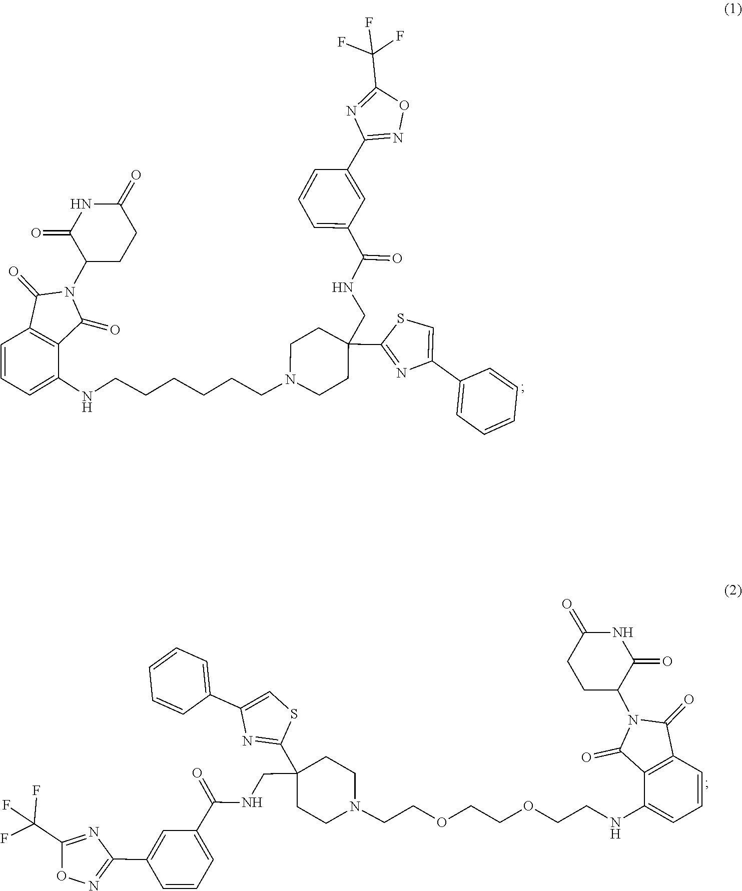 [SMILES: O=C1CCC(N2C(=O)c3cccc(NCCCCCCN4CCC(CNC(=O)c5cccc(-c6noc(C(F)(F)F)n6)c5)(c5nc(-c6ccccc6)cs5)CC4)c3C2=O)C(=O)N1 ; O=C1CCC(N2C(=O)c3cccc(NCCOCCOCCN4CCC(CNC(=O)c5cccc(-c6noc(C(F)(F)F)n6)c5)(c5nc(-c6ccccc6)cs5)CC4)c3C2=O)C(=O)N1] 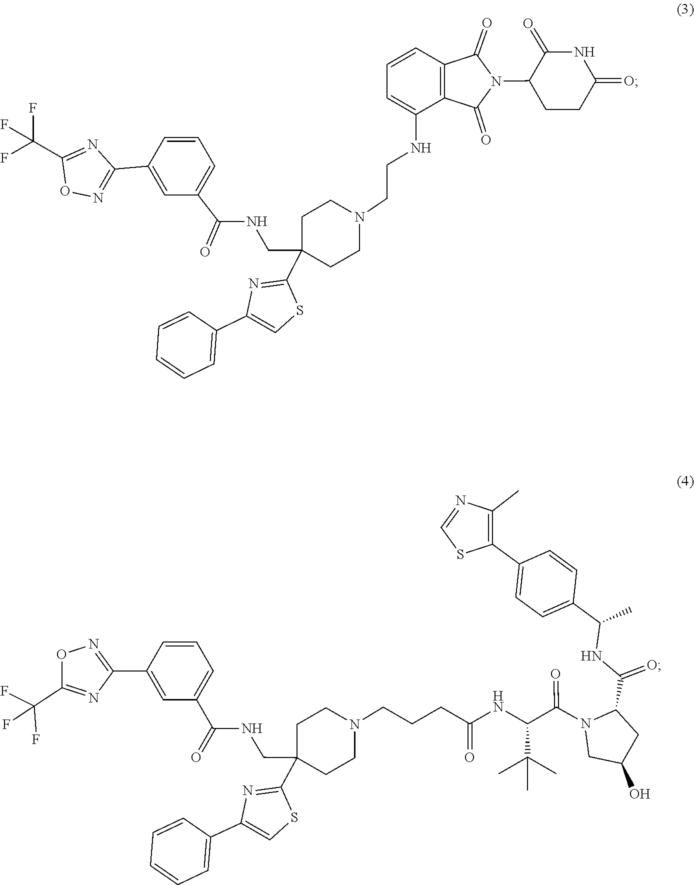 [SMILES: O=C1CCC(N2C(=O)c3cccc(NCCN4CCC(CNC(=O)c5cccc(-c6noc(C(F)(F)F)n6)c5)(c5nc(-c6ccccc6)cs5)CC4)c3C2=O)C(=O)N1 ; Cc1ncsc1-c1ccc([C@H](C)NC(=O)[C@@H]2C[C@@H](O)CN2C(=O)[C@@H](NC(=O)CCCN2CCC(CNC(=O)c3cccc(-c4noc(C(F)(F)F)n4)c3)(c3nc(-c4ccccc4)cs3)CC2)C(C)(C)C)cc1] 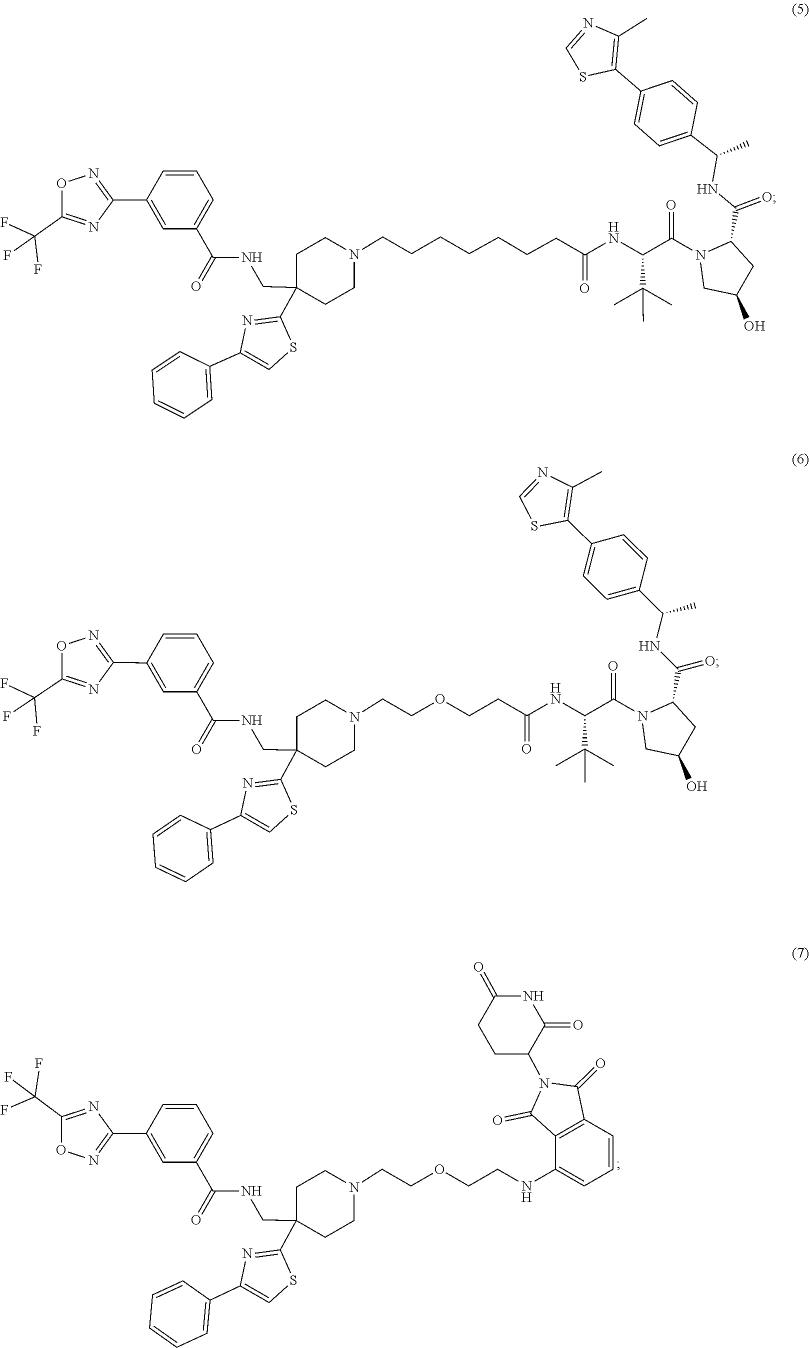 [SMILES: Cc1ncsc1-c1ccc([C@H](C)NC(=O)[C@@H]2C[C@@H](O)CN2C(=O)[C@@H](NC(=O)CCCCCCCN2CCC(CNC(=O)c3cccc(-c4noc(C(F)(F)F)n4)c3)(c3nc(-c4ccccc4)cs3)CC2)C(C)(C)C)cc1 ; Cc1ncsc1-c1ccc([C@H](C)NC(=O)[C@@H]2C[C@@H](O)CN2C(=O)[C@@H](NC(=O)CCOCCN2CCC(CNC(=O)c3cccc(-c4noc(C(F)(F)F)n4)c3)(c3nc(-c4ccccc4)cs3)CC2)C(C)(C)C)cc1 ; O=C1CCC(N2C(=O)c3cccc(NCCOCCN4CCC(CNC(=O)c5cccc(-c6noc(C(F)(F)F)n6)c5)(c5nc(-c6ccccc6)cs5)CC4)c3C2=O)C(=O)N1] 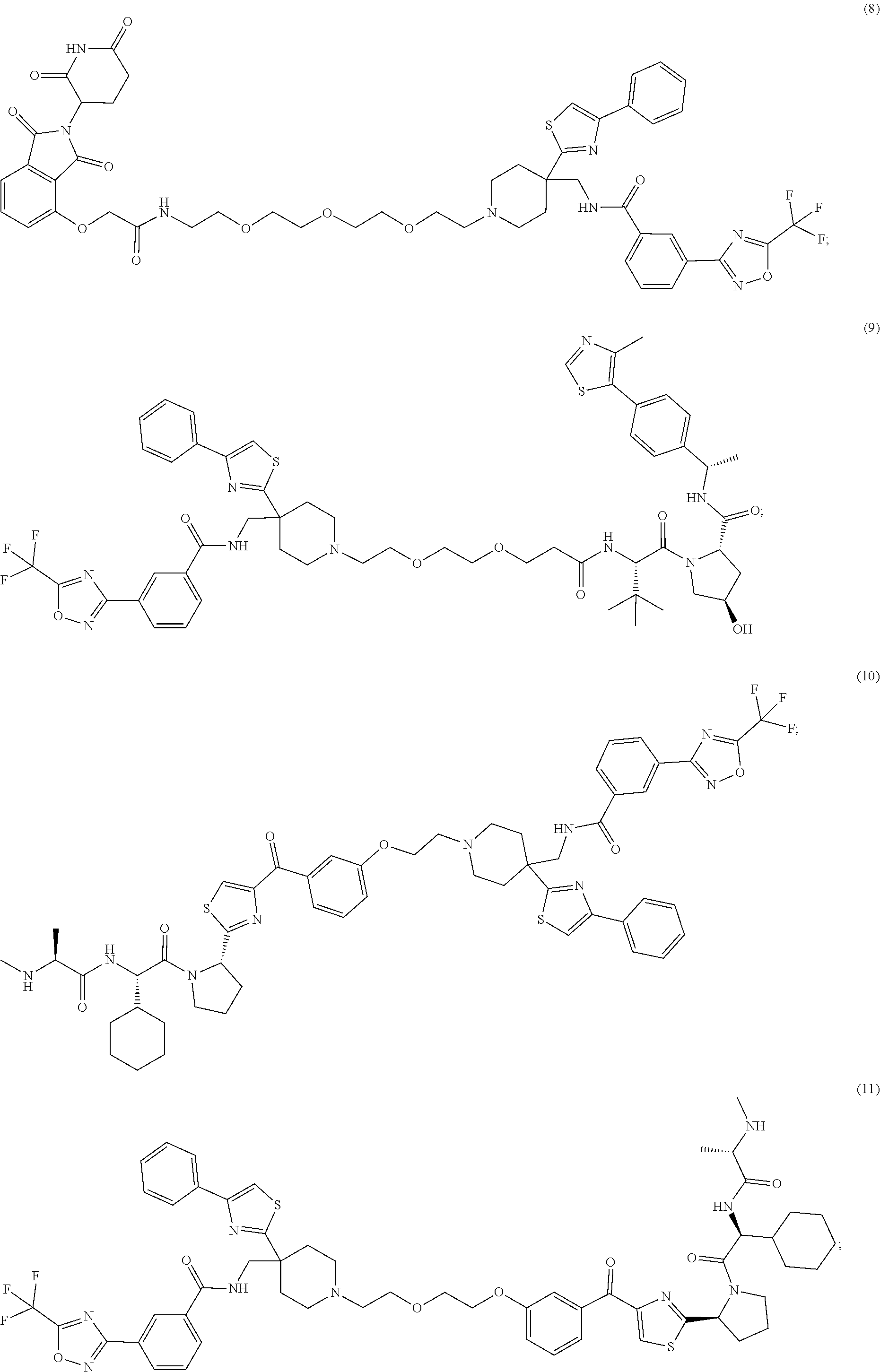 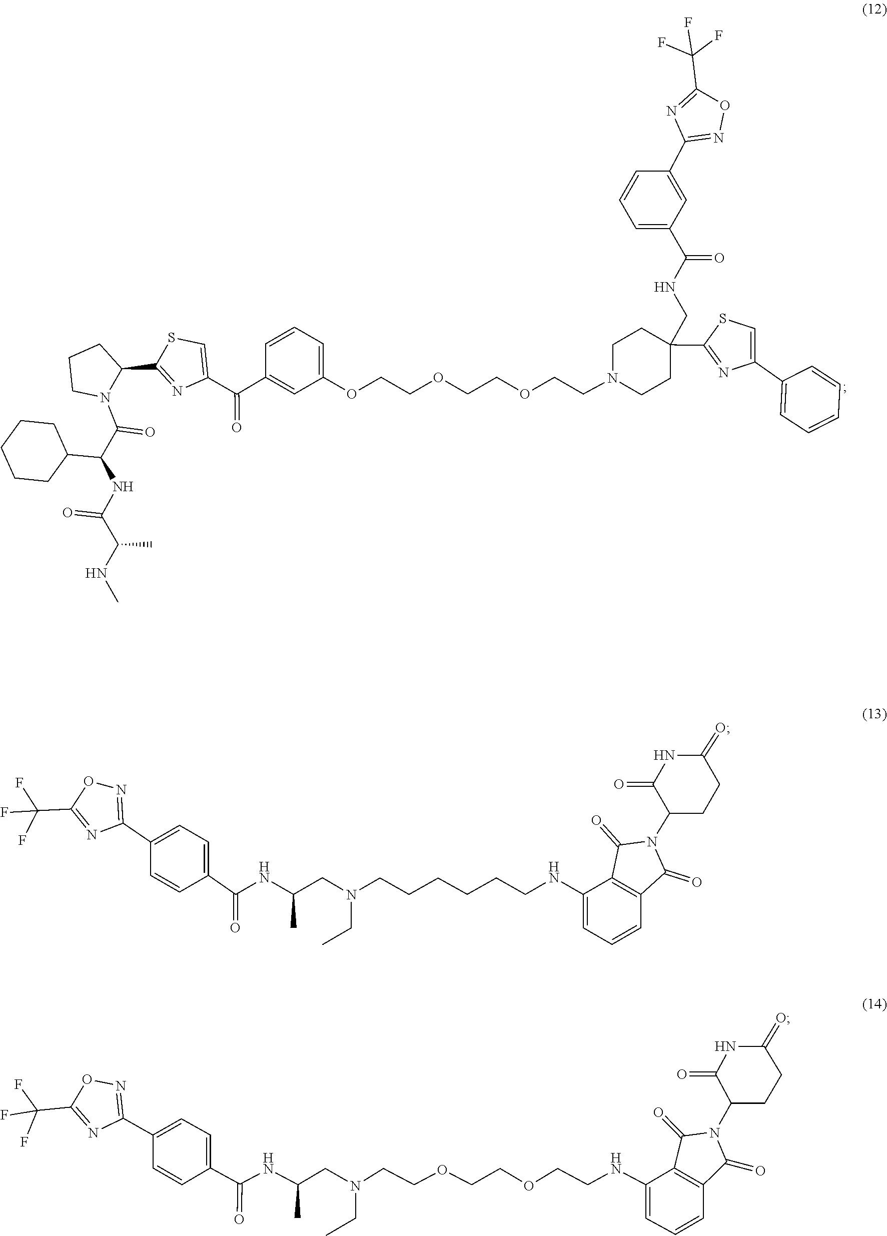 [SMILES: CN[C@@H](C)C(=O)N[C@H](C(=O)N1CCC[C@H]1c1nc(C(=O)c2cccc(OCCOCCOCCN3CCC(CNC(=O)c4cccc(-c5noc(C(F)(F)F)n5)c4)(c4nc(-c5ccccc5)cs4)CC3)c2)cs1)C1CCCCC1 ; CCN(CCCCCCNc1cccc2c1C(=O)N(C1CCC(=O)NC1=O)C2=O)C[C@@H](C)NC(=O)c1ccc(-c2noc(C(F)(F)F)n2)cc1 ; CCN(CCOCCOCCNc1cccc2c1C(=O)N(C1CCC(=O)NC1=O)C2=O)C[C@@H](C)NC(=O)c1ccc(-c2noc(C(F)(F)F)n2)cc1] 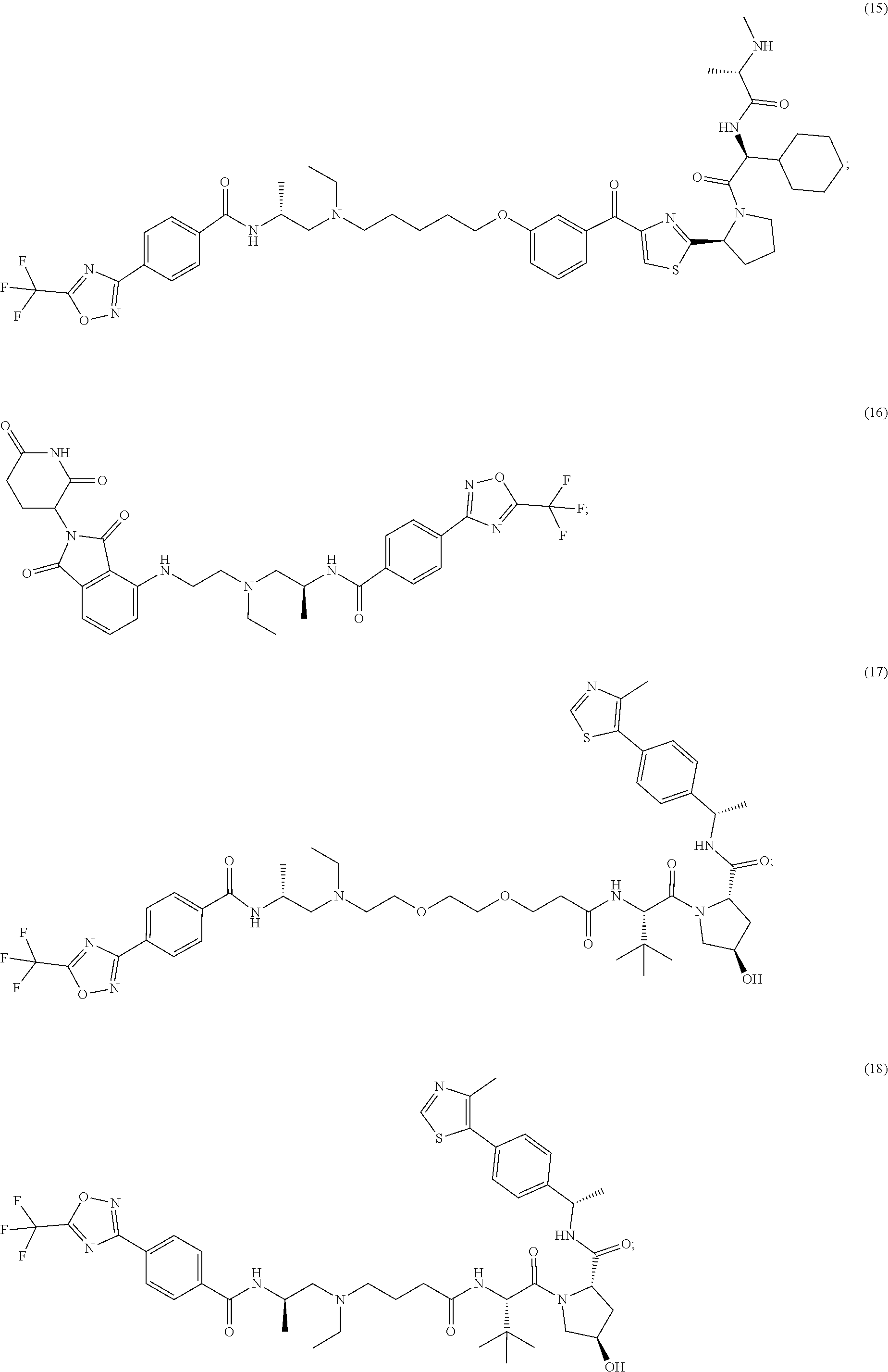 [SMILES: CCN(CCCCCOc1cccc(C(=O)c2csc([C@@H]3CCCN3C(=O)[C@@H](NC(=O)[C@H](C)NC)C3CCCCC3)n2)c1)C[C@@H](C)NC(=O)c1ccc(-c2noc(C(F)(F)F)n2)cc1 ; CCN(CCNc1cccc2c1C(=O)N(C1CCC(=O)NC1=O)C2=O)C[C@H](C)NC(=O)c1ccc(-c2noc(C(F)(F)F)n2)cc1 ; CCN(CCOCCOCCC(=O)N[C@H](C(=O)N1C[C@H](O)C[C@H]1C(=O)N[C@@H](C)c1ccc(-c2scnc2C)cc1)C(C)(C)C)C[C@@H](C)NC(=O)c1ccc(-c2noc(C(F)(F)F)n2)cc1 ; CCN(CCCC(=O)N[C@H](C(=O)N1C[C@H](O)C[C@H]1C(=O)N[C@@H](C)c1ccc(-c2scnc2C)cc1)C(C)(C)C)C[C@@H](C)NC(=O)c1ccc(-c2noc(C(F)(F)F)n2)cc1] 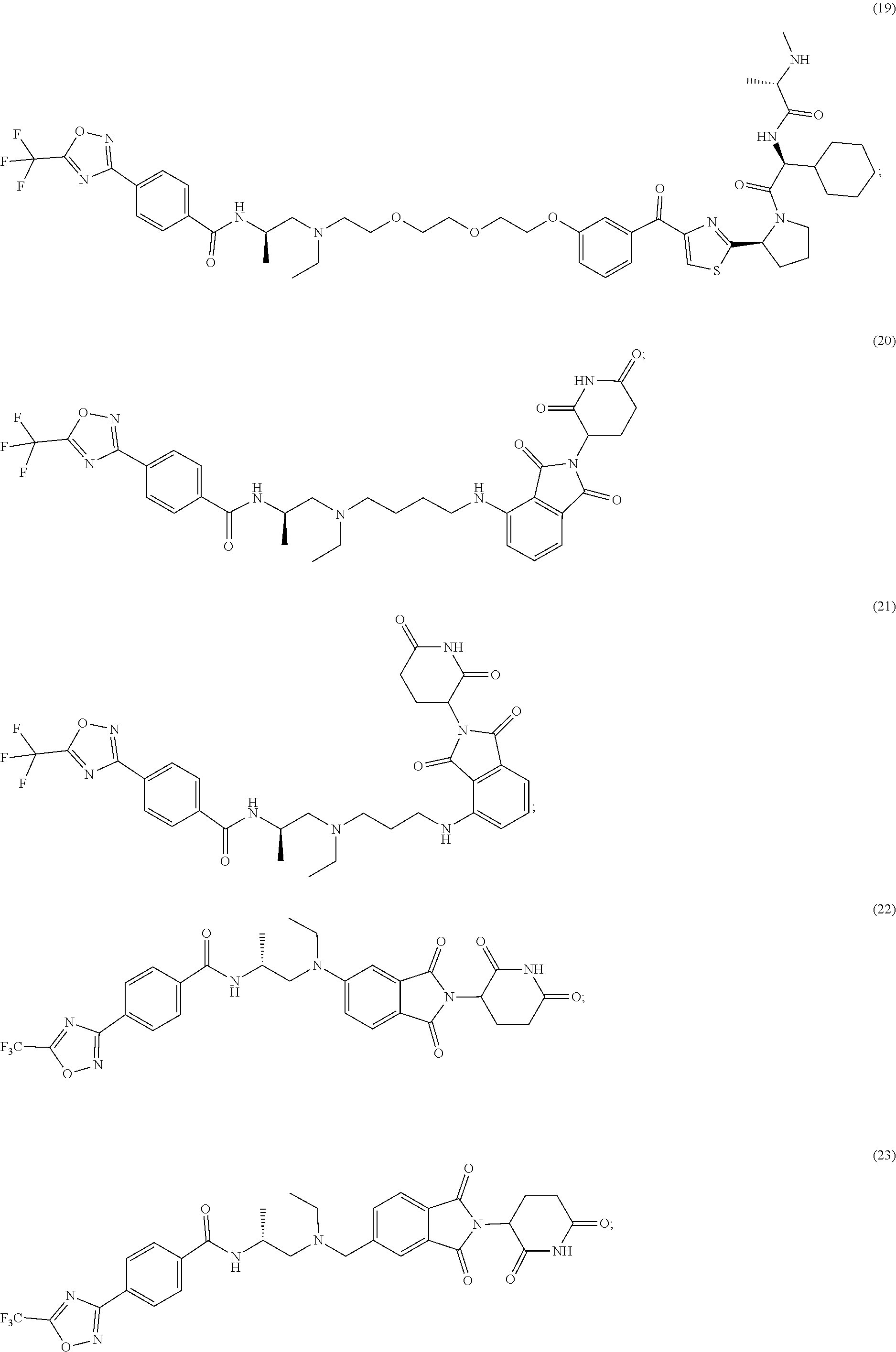 [SMILES: CCN(CCOCCOCCOc1cccc(C(=O)c2csc([C@@H]3CCCN3C(=O)[C@@H](NC(=O)[C@H](C)NC)C3CCCCC3)n2)c1)C[C@@H](C)NC(=O)c1ccc(-c2noc(C(F)(F)F)n2)cc1 ; CCN(CCCCNc1cccc2c1C(=O)N(C1CCC(=O)NC1=O)C2=O)C[C@@H](C)NC(=O)c1ccc(-c2noc(C(F)(F)F)n2)cc1 ; CCN(CCCNc1cccc2c1C(=O)N(C1CCC(=O)NC1=O)C2=O)C[C@@H](C)NC(=O)c1ccc(-c2noc(C(F)(F)F)n2)cc1 ; CCN(C[C@@H](C)NC(=O)c1ccc(-c2noc(C(F)(F)F)n2)cc1)c1ccc2c(c1)C(=O)N(C1CCC(=O)NC1=O)C2=O ; CCN(Cc1ccc2c(c1)C(=O)N(C1CCC(=O)NC1=O)C2=O)C[C@@H](C)NC(=O)c1ccc(-c2noc(C(F)(F)F)n2)cc1] 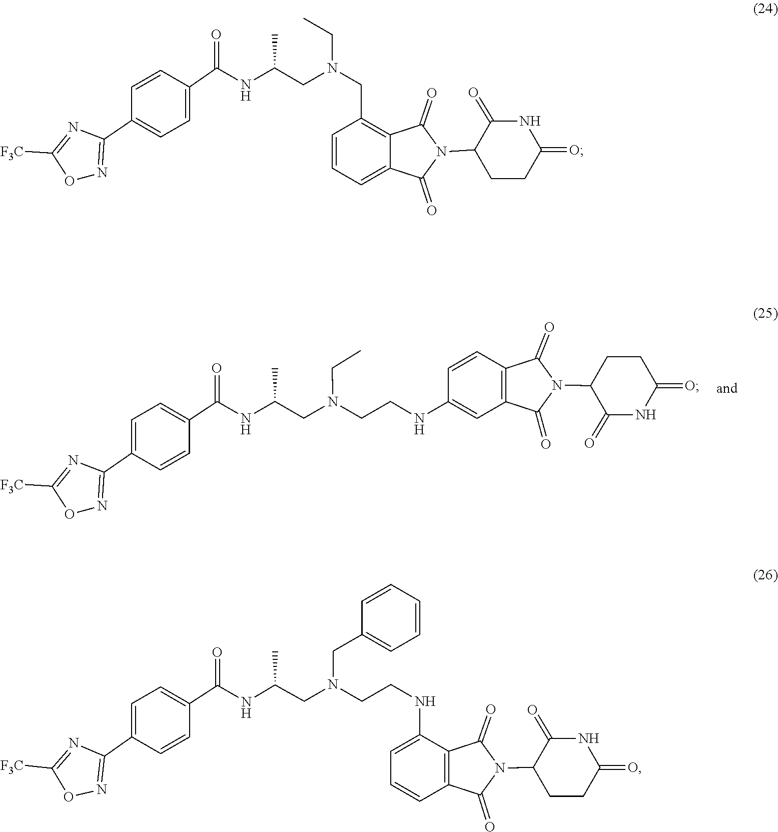 [SMILES: CCN(Cc1cccc2c1C(=O)N(C1CCC(=O)NC1=O)C2=O)C[C@@H](C)NC(=O)c1ccc(-c2noc(C(F)(F)F)n2)cc1 ; CCN(CCNc1ccc2c(c1)C(=O)N(C1CCC(=O)NC1=O)C2=O)C[C@@H](C)NC(=O)c1ccc(-c2noc(C(F)(F)F)n2)cc1 ; C[C@H](CN(CCNc1cccc2c1C(=O)N(C1CCC(=O)NC1=O)C2=O)Cc1ccccc1)NC(=O)c1ccc(-c2noc(C(F)(F)F)n2)cc1]
or a pharmaceutically acceptable salt or stereoisomer thereof.

24. A pharmaceutical composition, comprising a therapeutically effective amount of the compound or pharmaceutically acceptable salt or stereoisomer thereof of claim 1, and a pharmaceutically acceptable carrier.

25. A method of treating a disease or disorder that is characterized or mediated by aberrant activity of at least one class IIa HDAC, comprising administering to a subject in need thereof a therapeutically effective amount of the compound or pharmaceutically acceptable salt or stereoisomer thereof of claim 1.

26. The method of claim 25, wherein the disease or disorder is a neurodegenerative disease, alopecia, glucose homeostasis, muscular dystrophy, autoimmunity, or ischemic stroke.

27. The method of claim 26, wherein the neurodegenerative disease is Parkinson's disease, Alzheimer's disease, or Huntington's disease.

28. (canceled)
